(19) United States
(12) Patent Application Publication
(10) Pub. No.: US 2008/0280973 A1
(43) Pub. Date: Nov. 13, 2008

(54) Laulimalide Analogues as Therapeutic Agents

(76) Inventor: Paul A. Wender, Menlo Park, CA (US)

Correspondence Address: Robert E. Richards;King & Spalding, 1180 Peachtree Street, 34th Floor, Atlanta, GA, 30309, US

(21) Appl. No.: 11/630,858

(22) PCT Filed: Jun. 28, 2005
(86) PCT No.: PCT/US05/22965

§ 371 Date: Mar. 4, 2008

Related U.S. Application Data
(60) Provisional application No. 60/583,915, filed on Jun. 28, 2004.
(60) Provisional application No. 60/614,588, filed on Sep. 30, 2004.

(51) Int. Cl. A61K 31/395; C07D 407/06; C07D 413/06; C07D 323/00

(52) U.S. Cl. 514/450; 549/267; 540/454

(57) ABSTRACT

Laulimalide analogues useful as microtubule stabilizing agents, and in the treatment of abnormal cell proliferation, are disclosed. Methods of making the compounds, as well as methods of using such compounds in treating abnormal cell proliferation diseases are also described.

### CROSS REFERENCE TO RELATED APPLICATIONS

This application claims priority to U.S. Provisional application Nos. 60/583,915, filed Jun. 28, 2004 and 60/614,588, filed Sep. 30, 2004.

### FIELD OF THE INVENTION

This invention provides compounds particularly laulimalide analogues useful as microtubule stabilizing agents for use in the treatment of abnormal cell proliferation, compositions containing the compounds, and methods of making the compounds.

### DESCRIPTION OF RELATED ART

Important new targets for chemotherapeutic intervention against cancer and tumors are microtubules and tubulin, the basic subunit that makes up the microtubules. Microtubules are dynamic, polymeric structures which play an integral role in all eukaryotic cells (see, Microtubules, Hyams, J. S., Lloyd, C. W., eds, Wiley-Liss, New York, 1994, pp. 59-84 and 287-302). They are important in the development and maintenance of cell shape, in cell reproduction and division, in cell signaling, and in cellular movement (Lodish, H., et al., in Molecular Cell Biology, W.H. Freeman, New York: 1999). They also play a crucial role in mitosis. During mitosis, the dynamics of microtubule polymerization and depolymerization are finely controlled, and any variation in the rate of polymerization can affect cellular replication, and cause cells to enter into apoptosis. By affecting the rate of polymerization/depolymerization during this critical junction in the cell cycle, a new class of chemotherapeutic agents have emerged (Jordan, M. A., Curr. Med. Chem.—Anti-Cancer Agents, 2, pp. 1-17 (2002)).

There are two classes of chemotherapeutic agents which induce mitotic arrest by interfering with the microtubule dynamics; those that depolymerize tubulin, and those that stabilize tubulin polymers. Depolymerization agents, such as colchicine (Wilson, L., et al., Biochemistry, 6, pp. 3126-3135 (1967)), combretastatin A-4 (Pettit, G. R., et al., Anticancer Drug Design, 13, pp. 183-191 (1998); West, C. M., et al., Anticancer Drugs, 15, pp. 179-187 (2004)), vinblastine (Gupta, S., et al., Mol. Cell. Biochem., 253, pp. 41-47 (2003)) and vincristine (Sackett, D. L., Pharmacol. Ther., 59, pp. 163-170 (1993)) operate by inhibiting the formation of microtubule spindles or depolymerizing existing ones. The second class of chemotherapeutic agents operate by initiating tubulin polymerization as well as hyper-stabilizing existing microtubules (Schiff, P. B., et al., Proc. Natl. Acad. Sci. (USA), 77, pp. 1561-1565 (1980)). Such drugs increase the microtubular polymer mass in cells and inducing microtubule “bundling” (Rowinsky, E. K., et al., Cancer Res., 49, pp. 4093-4100 (1988)). The most well known of this class of tubulin stabilizing agents is Taxol® (paclitaxel). Taxol® (structure 1) was approved by the FDA in 1992 for the treatment of advanced ovarian cancer, and it is now indicated for breast cancer. In addition to enhancing tubulin polymerization and forming microtubule polymers, recent evidence suggests that Taxol® may bind to Bcl-2 in a second pathway which leads to programmed cell death (Chun, E., et al., Biochem. Biophys. Res. Commun., 315, pp. 771-779 (2004)). Both Bcl-2 and Bcl-x(L) may play an important role in mediating resistance to paclitaxel.

Although both Taxol® and its analog Taxotere® (structure 2) (docetaxel) are approved for the treatment of breast, ovarian, and lung carcinomas, they also exhibit several unfavorable properties. In addition to debilitating side effects, poor aqueous solubility which have made formulations difficult, and ineffectiveness against colon cancer and numerous other carcinomas, they are a target for P-glycoprotein (Pgp), an energy dependent drug efflux pump, which can induce multiple-drug-resistance (MDR) as well as drug-induced resistance-conferring tubulin mutations.

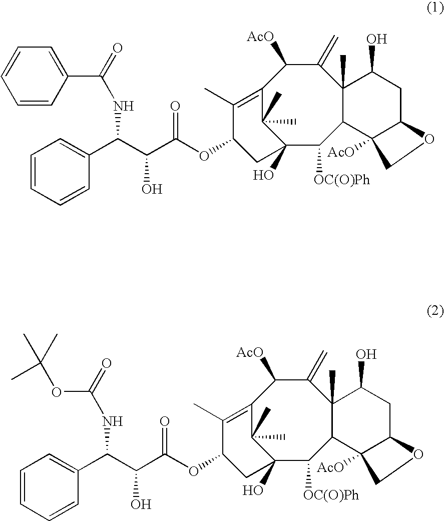

The clinical and commercial success of Taxol® and Taxotere® has sparked interest in finding other natural product antimitotic agents that exhibit a “Taxol-like” mechanism of action and that overcome the disadvantages of Taxol®. A number of novel natural products have been reported to exhibit Taxol-like properties, some of which are structurally less complex than Taxol. The extended family of microtubule stabilizing agents now includes the epothilones A (structure 3) and B (structure 4) and their analogues, eleutherobin, sarcodictyin, discodermolide, and WS9885B.

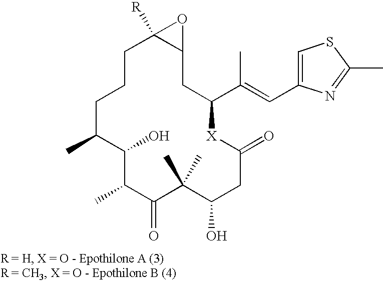

Another recently identified natural product which demonstrates potent microtubule-stabilizing properties is laulimalide. Laulimalide (structure 5), also known as figianolide B, is an 18-membered macrolide isolated from the marine chocolate sponge Cacospongia mycofjiensis (Quinoa, E., et al., J. Org. Chem., 53, pp. 3642-3644 (1988)), as well as from the Indonesian sponge Hyattella sp. (Corley, D. G., et al., J. Org. Chem., 53, pp. 3644-3646 (1988)). Later, this cytotoxic macrolide was found and isolated along with the compound neolaulimalide in the Okinawan sponge Fasciospongia rimosa (Jefford, C. W., et al., Tetrahedron Lett., 37, pp. 159-162 (1996); PCT publication No. WO 97/10242), and from a sponge in the genus Dactylospongia collected off the coast of the Vanuatu islands (Cutignano, A., et al., Eur. J. Org. Chem., 4, pp. 775-778 (2001)). This unique compound was shown to possess significant anti-tumor properties against a variety of cell lines (incl. KB, P388, A549, HT29 and MEL28) in the nanomolar range, and maintains a high level of potency against the multi-drug resistant cell line SKVLB-1 (IC₅₀=1.2 μM). Due to its notable antitumor properties, laulimalide has garnered significant attention in recent years.

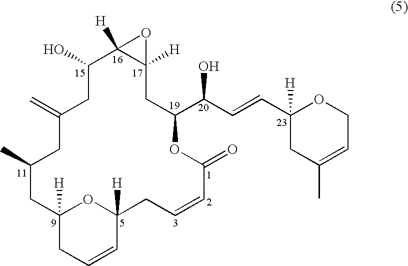

Synthetic work toward laulimalide began in 1996, after the absolute configuration of this macrolide was determined. Early efforts resulted in reports on fragment syntheses by the groups of Ghosh (Ghosh, A. K., et al., Tetrahedron Lett., 38, pp. 2427-2430 (1997)) and Nishiyama (Shimizu, A., et al., Tetrahedron Lett., 38, pp. 6011-6014 (1997); Shimizu, A., et al., Synlett, 11, pp. 1209-1210 (1998)). Following the identification of laulimalide as a member of the MSAA (microtubule-stabilizing antitumor agents) family in 1999 (Mooberry, S. L., et al., Cancer Res., 59, pp. 653-660 (1999)), synthetic interest grew. Since then, a number of total syntheses of laulimalide have been reported. See, Wender, P. A., et al., J. Am. Chem. Soc., 124, pp. 4956-4957 (2002); Ghosh, A. K., et al., J. Org. Chem., 66, pp. 8973-8982 (2001); Paterson, I., et al., Org. Letters, pp. 3149-3152 (2001); Enev, V. S., et al., J. Am. Chem. Soc., 123, pp. 10764-10765 (2001); and the review by Mulzer and Öhler, Chem. Rev., 103, pp. 3753-3786 (2003).

Mooberry and Davidson, in U.S. Pat. No. 6,414,015, describe and claim methods of inhibiting the proliferation of a hyperproliferative mammalian cell having a multiple drug resistant phenotype by contacting the cell with a laulimalide analog with a variation at the C₁₆-C₁₇ epoxide or at the C₂₀ position “so as to disrupt the dynamic state of microtubule polymerization”. Related patent applications WO 01/54689 and U.S. Patent Application No. 2002/0198256 A1 describe compositions comprising at least one synthetic laulimalide variant, wherein the C₂₀ position is OH, OCH₃, OC(O)CH₃, and OSi(i-Pr)₃, the compositions further including an anti-neoplastic agent.

Both U.S. Pat. No. 6,670,389 and U.S. Publication No. 2003/0195181, to Ashley, et al., describe a series of laulimalide analogues of the general formula shown below, synthetic intermediates to these analogues, and methods for their preparation. Also described are methods for the use of these compounds in the treatment of diseases characterized by cellular hyperproliferation, such as cancers, tumors, and inflammatory disorders. The analogues are primarily modified at the C₁₅-position, the C₂₃-position, and at the C₁₆-C₁₇ bond area.

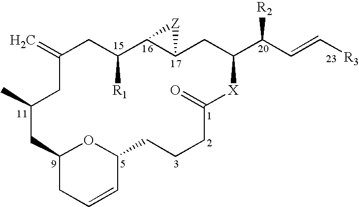

International Publication No. WO 03/024975, as well as European Patent Publication No. EP 1295886, to Schering Aktiengesellschaft describe a series of laulimalide derivatives as shown below, with modifications at the C₂-C₃, the hydropyran oxygen, C₁₁, C₁₅, C₁₆₋₁₇, C₂₀ and C₂₁-C₂₃ positions, and their use as a medicament for the treatment of cancer, autoimmune diseases, infectious diseases, and chronic neurodegenerative diseases, among other. Also described are processes for the production of such derivatives, as well as laulimalide itself, and the intermediates used for this production.

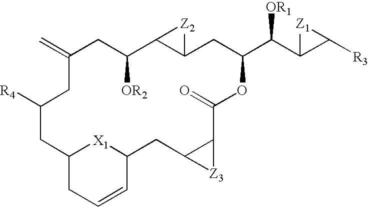

U.S. Publication No. 2003/0203929 A1 to Ghosh and corresponding international Publication No. WO 03/076445 to the University of Illinois describe both laulimalide and epothilone derivatives, such as those of structure below, for use as microtubule stabilizing agents and in the treatment of cancers. Also described are methods of making such compounds and using the compounds as therapeutic agents in the treatment of a variety of cancers. Derivatives are modified at the C₂-C₃ position, the tetrahydropyranyl ring (with five and six membered rings bearing sulfur, nitrogen or methylene in place of the ring oxygen), the C₁₁-position, the C₁₉-heteroatom position, the C₂₀-position, and at the C₂₃-position.

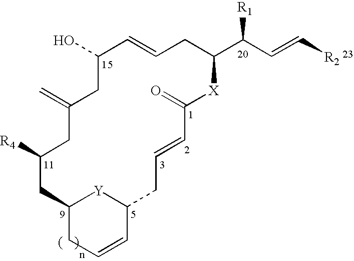

In addition, a limited number of analogues, directed to modifications of the C₁₅- and C₂₀-hydroxyls, the (Z)-enoate at C₂-C₃, or in removal of the epoxide at the C₁₆-C₁₇ position, have been reported to date in the literature. Cell screening of (−)-laulimalide and isolaulimalide, as well as trans-desoxylaulimalide was described in efforts to define structure-activity relationships of laulimalide (Pryor, D. E., et al., Biochemistry, 41, pp. 9109-9115 (2002)). This report suggested that, while laulimalide promotes abnormal tubulin polymerization and apoptosis in vitro similar to Taxol®, laulimalide binds to tubulin at a different site than Taxol®, resulting in both its unique biological profile and lack of inducement of MDR.

The Wender group at Stanford University described the synthesis of five laulimalide analogues (Wender, P. A., et al., Organic Letters, 5, pp. 3507-3509 (2003)), incorporating modifications at the C₁₆-C₁₇ epoxide, the C₂₀-alcohol, and at the C₁-C₃-enoate positions. The resultant laulimalide-based analogues exhibited a range of resistance values against HeLa cells, NCI/ADR cells, and the drug-sensitive MDA-MB-435 cell line, indicating that all of the analogues are poor substrates for Pgp and have potential to treat Taxol®-resistant tumor cells. Generally, it was reported that the des-epoxy analogue maintained strong biological activity (approximately 19-fold less potent than laulimalide), while the C₂₀-methoxy analogue exhibited a significant decrease in potency. It was also stated that modifications at the C₂₀-hydroxyl position are moderately tolerated when the C₁₆-C₁₇-epoxide is retained.

A total synthesis and biological evaluation of (−) laulimalide, as well as a series of analogues, was described by Gallagher, et al. (Bioorg. Med. Chem. Lett., 14, pp. 575-579 (2004)) offer a series of analogues based upon the synthetic route described for laulimalide itself. Specifically, the synthesis and biological evaluation of several des-epoxy, C₂₀, and C₁₅ analogues were described. Replacement of the C₁₆-C₁₇ epoxide with an alkene resulted in a loss of potency of two orders of magnitude, suggesting that that this functionality is either mechanistically or conformationally important for activity. Modification of the C₂₀ alcohol with various groups suggested the importance of the C₂₀ alcohol to participate in H-bonding interactions and/or that steric bulk at the C₂₀ position cannot be tolerated. Inversion of stereochemistry and/or modification of the hydroxyl at the C₁₅ position suggested both that the absolute stereochemistry at the C₁₅ position may be of minor importance, but the alcohol at this position appears to contribute to the overall biological potency of the molecule. Finally, inversion of geometry from (Z) to (E), converting the alkene to an alkyne, or modifying the degree of saturation at the C₂-C₃ enoate position reportedly plays a role in the potency of the molecule, although it is hypothesized by the authors that this portion of the molecule is necessary only for macrocycle conformation.

A series of known anticancer drugs, including laulimalide, were classified according to their structural features and a series of structure-activity relationships proposed and analyzed (Hayakawa, Y., Jpn. J. Cancer Chemother. (Gan To Kagaku Ryoho), 31, pp. 526-528 (2004)). According to the article, laulimalide displayed a high correlation to known tubulin binders, while other similar compounds exhibited an unexpected, unpredictable poor correlation to tubulin binders.

The synthesis of two 11-desmethyl analogues of laulimalide were described by Paterson, et al. (Organic Letters, 6, pp. 1293-1295 (2004)). These compounds were synthesized using the Nozaki-Kishi reaction, in order to obtain structurally simplified analogues. No biological evaluation of the compounds produced was reported.

In a report to the U.S. Army Medical Research and Material Command, B. S. Davidson (“Development of Laulimalide-Based Microtubule-Stabilizing Agents: New Chemistry for the Treatment of Breast Cancer”, Storming Media, July 2002; http://www.stormingmedia.com/) described research in adapting laulimalide onto solid phase supports. According to the report, the attachment of fragments of laulimalide onto a resin have been achieved, but the total solid-phase synthesis of laulimalide had not.

Five analogues of laulimalide, designed to exhibit enhanced chemical stability while retaining the biological activity of the parent compound, were described and evaluated by S. L. Mooberry, et al. (Proc. Natl. Acad. Sci. (USA), 101, pp. 8803-8808 (2004)) in an effort to advance the structural understanding of laulimalide's mode of action. While all of the synthetic analogues retained activity against drug-resistant cells, the effect of the various modifications provided important information on the structural basis for laulimalide's mode of action—namely, that the biological efficacy of laulimalide can be achieved with synthetic analogues that lack the chemically sensitive C₁₆-C₁₇ epoxide or contain a modified nucleophilic C₂₀-hydroxyl. The two most potent analogues, C₁₆-C₁₇-des-epoxy laulimalide and C₂₀-methoxy laulimalide, appeared to have a mechanism of action identical to laulimalide. The C₁₆-C₁₇-des-epoxy, C₂₀-methoxy laulimalide derivative, which incorporated both chemical changes of the most potent analogues, was significantly less potent and initiated the formation of unique interphase microtubules unlike that seen in the parent compound or other analogues. Two C₂-C₃-alkynoate derivatives had lower potency and initiated abnormal microtubule structures, but did not cause micronucleation or extensive G₂/M accumulation.

It is an object of the present invention to provide new compounds, methods, compositions, and strategies for use in treating abnormal cell proliferation, including tumors, cancer and angiogenesis-related disorders.

It is another object of the invention to provide new compounds, methods, compositions and strategies that exhibit strong activity against abnormally proliferating cells and exhibit a minimal effect on normal cells.

It is yet a further object of the present invention to provide new compounds, methods, compositions and strategies for the treatment of a host having a disorder caused by abnormal cell proliferation which are sufficiently stable to be stored until use in an appropriate compositions and administered by any desired mode.

### SUMMARY OF THE INVENTION

New compounds, methods, compositions, and strategies for use in treating abnormal cell proliferation, including tumors, cancer and angiogenesis-related disorders are provided. The compounds described herein, including in formulas (I)-(XI) bear unique modifications in the C₁-C₁₀ region of the molecule. This region has previously been considered the “scaffold” region and, as such, has not been a primary focus of research interest.

In one embodiment, the analogs do not have a ring at the C₅-C₉ position, are structurally more simple and may exhibit greater long term stability than laulimalide. In other embodiments, other heterocyclic and aromatic rings are substituted for the hydropyran ring.

In brief, the invention includes the following features:
(a) Laulimalide analogues of formulas (I)-(XI), described herein, and pharmaceutically acceptable salts, solvates, esters and prodrugs thereof as described further below, which can be in substantially pure form;
(b) Methods for the treatment or prophylaxis of a host (typically a mammal, and more typically a human) suffering from a disorder of abnormal cellular proliferation that includes administering an effective amount of one or more of the laulimalide analogues described herein;
(c) Pharmaceutical formulations comprising the laulimalide analogues and pharmaceutically acceptable salts, solvates, esters and prodrugs thereof with a pharmaceutically acceptable carrier or diluent, alone or in combination with other pharmaceutically active agents.

Illustrative disorders of abnormal cell proliferation that can be treated according to the present invention include tumors and cancers; unwanted angiogenesis, psoriasis, chronic eczema, atopic dermatitis, lichen planus, warts, pemphigus vulgaris, actinic keratosis, basal cell carcinoma and squamous cell carcinoma, blood vessel proliferation disorders, fibrotic disorders, autoimmune disorders, graft-versus-host rejection, disorders brought about by abnormal proliferation of mesangial cells (including human renal diseases, such as glomerulonephritis, diabetic nephropathy, malignant nephrosclerosis, thrombotic micro-angiopathy syndromes, transplant rejection, and glomerulopathies), rheumatoid arthritis, Behcet's syndrome, acute respiratory distress syndrome (ARDS), ischemic heart disease, post-dialysis syndrome, leukemia, acquired immune deficiency syndrome, vasculitis, lipid histiocytosis, septic shock, inflammation, Kaposi's sarcoma, haemangioma, acute and chronic nephropathies, atheroma, arterial restenosis, autoimmune diseases, endometriosis, dysfunctional uterine bleeding and ocular diseases with retinal vessel proliferation.

### DESCRIPTION OF THE FIGURES

FIG. 1 shows the preparation of allyl silane precursor 28.

FIG. 2 shows the preparation of a bis-TBS-protected C₁₅-C₂₇ fragment of the laulimalide analogues.

FIG. 3 shows the preparation of the C₂₁-C₂₂ olefin fragment of the laulimalide analogues.

FIG. 4 shows the preparation of des-epoxy C₅-amide analogs.

FIG. 5 shows the preparation of des-epoxy C₅-ester analogs.

### DETAILED DESCRIPTION OF THE INVENTION

Compounds, pharmaceutical compositions, methods and uses are provided for the treatment of a disorder of abnormal cellular proliferation in a host is provided, comprising at least one compound of principal embodiments (I)-(XI) below, or a pharmaceutically acceptable salt, solvate, ester or prodrug thereof, optionally with a pharmaceutically acceptable carrier; and optionally with one or more therapeutic agents.

### Compounds

In a first principle embodiment, a compound of Formula I, or a pharmaceutically acceptable salt, solvate, ester, or prodrug thereof, is provided,

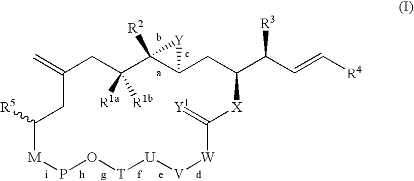

wherein:
R^{1a}, R^{1b}, and R⁵ are each independently H, C₁-C₁₀ alkyl, C₂-C₁₀ alkenyl, C₁-C₁₀ alkoxy, C₂-C₁₀ alkenoxy, C₂-C₁₀ alkynyl, C₂-C₁₀ alkynoxy, aryl, substituted aryl, heteroaryl, substituted heteroaryl, COR⁸, nitro, cyano, OH, CF₃, OCF₃, or halogen;
R² is absent (when “a” is a triple bond or when “a” is a single bond and “b” is a double bond) or is selected from the group consisting of H, C₁-C₁₀ alkyl, C₂-C₁₀ alkenyl, C₁-C₁₀ alkoxy, C₂-C₁₀ alkenoxy, C₂-C₁₀ alkynyl, C₂-C₁₀ alkynoxy, aryl, nitro, cyano, halogen, acyl, alkacyl, CHO, CO₂H, CO₂—C₁₋₁₀ alkyl, CF₃, OH, OR^{8′}, OCF₃, SH, SR^{8′}, NH₂, NHR^{8′}, NHR^{8′}R^{8′}, CON(R^{8′})₂, and CONHR^{8′};
“a” can be a single or double bond of either (E)- or (Z)-orientation, or “a” can be a triple bond when R², Y, “b” and “c” are absent;
“b” can be absent or a single bond (when R² is absent);
“c” can be absent, a single, or double bond of either (E)- or (Z)-orientation, such that only one of “a”, “b”, and “c” can be a double bond, when “b” and “c” are absent, then Y is absent; and
   when “a” is a single or double bond, one of “b” and “c” is a single bond and one is absent, then Y is H, a straight or branched substituted or unsubstituted alkyl, alkenyl, or alkynyl, CH₃, CH₂R⁸, CHR⁸R⁸, CR⁸R⁸R⁸, CH₂F, CH₂Cl, CH₂Br, CHF₂, CHCl₂, CHBr₂, CF₃, CCl₃, CBr₃, OH, OR^{8′}, SH, SR^{8′}, NH₂, NHR^{8′}, or NR^{8′}R^{8′};
   when “a”, “b”, and “c” are single bonds or when “a” is a single bond, one of “b” and “c” is a double bond and one is absent, then Y is CH₂, CHR⁸, CR⁸R⁸, CHF, CHCl, CHBr, CF₂, CCl₂, CBr₂, O, S, NH, or NR^{8′};
R³ is independently selected from H, C₁-C₁₀ alkyl, C₂-C₁₀ alkenyl, C₁-C₁₀ alkoxy, C₂-C₁₀ alkenoxy, C₂-C₁₀ alkynyl, C₂-C₁₀ alkynoxy, aryl, substituted aryl, heteroaryl, substituted heteroaryl, nitro, cyano, CF₃, OH, O-alkyl, hydroxylalkyl, O-acyl, OCF₃, SH, S-alkyl, thioalkyl, S-acyl, amine, alkylamine, NH₂, NHR⁸, NR⁸R⁸, and halogen;
R⁴ is selected from the group consisting of C₃-C₁₀ cycloalkyl, C₃-C₁₀ cycloalkenyl, heteroaryl, substituted heteroaryl, aryl, substituted aryl, C₃-C₁₀ heterocycloalkyl, adamantly, and C₃-C₁₀ heterocycloalkenyl;
X is CH₂, CHR⁸, CR⁸R⁸, N, NR^{8′}, O, or S; and
when “d” is a single bond, V is independently selected from the group consisting of CH₂, CHR⁸, CR⁸R⁸, NH, NR^{8′}, O, S, C═O, or C═Y², and W is independently selected from the group consisting of CH₂, CHR⁸, CR⁸R⁸, NH, NR^{8′}, O, or S;
   such that V and W are not both NH, NR^{8′}, O, S, C═O, or C═Y²; W is not NH, NR^{8′}, O, or S, when X is N, NR^{8′}, O, or S; and V is not C═O or C═Y², when W is N, NR⁸, O, or S;
when “d” is a double bond of either (E)- or (Z)-orientation, V and W are independently selected from the group consisting of CH, CR⁸, or N such that V and W are not both N, and X and W are not both N; or
when “d” is a triple bond, V and W are both carbon; or alternatively, V and W are taken together to form an optionally substituted or unsubstituted carbocyclic ring, such as a 3-6 membered cycloalkyl ring, or an optionally substituted or unsubstituted heterocyclic ring, such as a 3-6 membered heterocyclic ring,
   such that only 2 adjacent ring members joined via a single or double bond (i.e., “d” is a single bond or a double bond) are part of the macrocyclic ring system; and the ring member directly adjacent to the —(C═Y¹)— moiety of the macrocycle is not a heteroatom when X is N, NR⁵, O, or S;
when “e”, “f”, “g”, “h”, or “i” is a single bond (i.e., the bond between M and P, P and Q, T and U, or U and V, is a single bond), then the respective M, P, T, U, or Q is independently CH₂, CHR⁸, CR⁸R⁸, NH, NR^{8′}, O, S, C═O, or C═Y²;
   such that if one of M, P, T, U, V, or W is NH, NR^{8′}, O, or S, then its directly adjacent moieties cannot be NH, NR^{8′}, O, or S; and if one of M, P, T, U, V, or W is NH, NR^{8′}, O, or S, then its directly adjacent moieties both cannot be C═O or C═Y²; and, if one of M, P, T, U, or V is C═O or C═Y², then its directly adjacent moieties cannot be C═O or C═Y²; and if one of M, P, T, U, or V is C═O or C═Y², then its directly adjacent moieties both cannot be NH, NR^{8′}, O, or S; or alternatively,
when “e”, “f”, “g”, “h”, or “i” is a double bond of either (E)- or (Z)-orientation, then the respective M, P, T, U, or Q is independently CH, CR⁸, or N, such that, if one of M, P, T, U, V, or W is N, then its directly adjacent moieties cannot be N, NH, NR^{8′}, O, or S; and
when “e”, “f”, “g”, “h”, or “i” is a triple bond, then the respective M, P, T, U, or Q is a carbon; wherein
   when “h” and “i” are single bonds, P is CHR*, CR⁸R*, or NR*; when one of “h” and “i” is a double bond”, P is CR*; and when “g” and “f” are single bonds, T is CHR*′, CR⁸R*′, or NR*′; when one of “g” and “f” is a double bond”, T is CR*′; wherein R* and R*′ are taken together with Q to form an optionally substituted or unsubstituted carbocyclic ring, such as a 3-6 membered cycloalkyl ring, or an optionally substituted or unsubstituted heterocyclic ring, such as a 3-6 membered heterocyclic ring,
   such that the ring member directly adjacent to M is not a heteroatom when M is N, NR⁵, O, or S;
   with the proviso that when —V—W— is —CH═CH— or —C≡C—, then —P-Q-T- is not

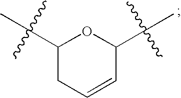

each Y¹ and Y² is independently O, S, NH, or NR^{8′};
each R⁸ is independently —H; an optionally substituted or unsubstituted straight or branched alkyl, such as a —C₁₋₈ straight or branched chain alkyl; an optionally substituted or unsubstituted straight or branched —C₂₋₈ alkenyl; an optionally substituted or unsubstituted straight or branched —C₂₋₈ alkynyl; —C₃₋₆ cycloalkyl; 3-7 membered heterocycle; -aryl; -aralkyl; -heteroaryl, -heteroarylalkyl, -halo (F, Cl, Br, I); -haloalkyl; —CF₃; —CN; —NO₂; -acyl (including but not limited to aldehydes, ketones, esters, carboxylic acids, amides, imides, thioesters), —(C═Y¹)-alkyl, —O(C═Y¹)-alkyl, —(C═Y¹)—OH, —(C═Y¹)—O-alkyl, —S—(C═Y¹)-alkyl, —(C═Y¹)—SH, —(C═Y¹)—S-alkyl, —NH(C═Y¹)-alkyl, —NR^{8′}(C═Y¹)-alkyl, —(C═Y¹)—NH₂, —(C═Y¹)—NH(alkyl), —(C═Y¹)—N(alkyl)₂, —COOH; —COOC₁₋₈ alkyl; —CONH₂; —CONH—C₁₋₅ alkyl; —CON(C₁₋₈ alkyl)₂; alkacyl, -alkyl-(C═Y¹)-alkyl, -alkyl-O(C═Y¹)-alkyl, -alkyl-(C═Y¹)—OH, -alkyl-(C═Y¹)—O-alkyl, -alkyl-S—(C═Y¹)-alkyl, -alkyl-(C═Y¹)—SH, -alkyl-(C═Y¹)—S-alkyl, -alkyl-NH(C═Y¹)-alkyl, -alkyl-NR^{8′}(C═Y¹)-alkyl, -alkyl-(C═Y¹)—NH₂, -alkyl-(C═Y¹)—NH(alkyl), -alkyl-(C═Y¹)—N(alkyl)₂, -alkyl-COOH; -alkyl-COOC₁₋₈ alkyl; -alkyl-CONH₂; -alkyl-CONH—C₁₋₈ alkyl; -alkyl-CON(C₁₋₈ alkyl)₂; amino, —NH₂; —NH—C₁₋₈ alkyl; —N(C₁₋₈ alkyl)₂; —NHC(O)—C₁₋₈ alkyl; alkylamino; hydroxyl, alkylhydroxyl, alkoxy, thio; alkylthio; thioalkyl;
each R^{8′} is independently an optionally substituted or unsubstituted straight or branched alkyl, such as a —C₁₋₈ straight or branched chain alkyl; an optionally substituted or unsubstituted straight or branched alkenyl, such as a —C₂₋₈ alkenyl; an optionally substituted or unsubstituted straight or branched alkynyl, such as a —C₂₋₈ alkynyl; a saturated or unsaturated carbocycle, such as a saturated or unsaturated —C₃₋₆ cycloalkyl; a heterocycle, such as a 3-7 membered heterocycle; aryl; or heteroaryl;
such that there is not a double or triple bond directly adjacent to a double or triple bond.

In a subembodiment, the compound of Formula I, or a pharmaceutically acceptable salt, solvate, ester, or prodrug thereof, is provided, wherein R^{1a}, R^{1b}, and R⁵ are either hydrogen, CH₃, or C₁-C₅ alkyl.

In subembodiment, the compound of Formula I, or a pharmaceutically acceptable salt, solvate, ester, or prodrug thereof, is provided, wherein “-M-P-Q-T-U—” is further selected from the group consisting of —(C═O)-Z-CH₂—CH₂—CH₂—, —(C═Y²)-Z-CH₂—CH₂—CH₂—, —(C═Y²)-Z-CHR⁸—CHR⁸—CHR⁸—, —CH₂—(C═O)-Z-CH₂—CH₂—, —CH₂—(C═Y²)-Z-CH₂—CH₂—, —CHR⁸—(C═Y²)-Z-CHR⁸—CHR⁸—, —CH₂—CH₂—(C═O)-Z-CH₂—, —CH₂—CH₂—(C═Y²)-Z-CH₂—, —CHR⁸—CHR⁸—(C═Y²)-Z-CHR⁸—, -Z-(C═O)—CH₂—CH₂—CH₂—, -Z-(C═Y²)—CH₂—CH₂—CH₂—, -Z-(C═Y²)—CHR⁸—CHR⁸—CHR⁸—, —CH₂-Z-(C═O)—CH₂—CH₂—, —CH₂-Z-(C═Y²)—CH₂—CH₂—, —CHR⁸-Z-(C═Y²)—CHR⁸—CHR⁸—, —CH₂—CH₂-Z-(C═O)—CH₂—, —CH₂—CH₂-Z-(C═Y²)—CH₂—, —CHR⁸—CHR⁸-Z-(C—Y²)—CHR⁸—, —(C═O)-Z-CH═CH—CH₂—, —(C═Y²)-Z-CH═CH—CH₂—, —(C═Y²)-Z-CR⁸═CR⁸—CHR⁸—, —(C═O)-Z-CH₂—CH═CH—, —(C═Y²)-Z-CH₂—CH═CH—, —(C═Y²)-Z-CHR⁸—CR⁸═CR⁸—, —CH═CH—(C═O)-Z-CH₂—, —CH═CH—(C═Y²)-Z-CH₂—, —CR⁸═CR⁸—(C═Y²)-Z-CHR⁸—, -Z-(C═O)—CH═CH—CH₂—, -Z-(C═Y²)—CH═CH—CH₂—, -Z-(C═Y²)—CR⁸═CR⁸—CHR⁸—, -Z-(C═O)—CH₂—CH═CH—, -Z-(C═Y²)—CH₂—CH═CH—, -Z-(C═Y²)—CHR⁸—CR⁸═CR⁸—, —CH═CH-Z-(C═O)—CH₂—, —CH═CH-Z-(C═Y²)—CH₂—, —CR⁸═CR⁸-Z-(C═Y²)—CHR⁸—, —(C═O)-Z-C≡C—CH₂—, —(C═Y²)-Z-C≡C—CH₂—, —(C═Y²)-Z-C≡C≡CHR⁸—, —(C═O)-Z-CH₂—C≡C—, —(C═Y²)-Z-CH₂—C≡C—, —(C—Y²)-Z-CHR⁸—C≡C—, —C≡C—(C═O)-Z-CH₂—, —C≡C—(C═Y²)-Z-CH₂—, —C≡C—(C═Y²)-Z-CHR⁸—, -Z-(C═O)—C≡C—CH₂—, -Z—(C═Y²)—C≡C—CH₂—, -Z-(C═Y²)—C≡C—C—CHR⁸—, -Z-(C═O)—CH₂—C≡C—, -Z-(C═Y²)—CH₂—C≡C—, -Z-(C═Y²)—CHR⁸—C≡C—, —C≡C-Z-(C═O)—C₂—, —C≡C-Z-(C═Y²)—C₂—, and —C≡C-Z-(C═Y²)—CHR⁸—, or
at least one of “-M-P—”, “—P-Q-”, “-Q-T-” or “-T-U—” is further selected from the group consisting of -Z-CHR^{8″}—, —CHR^{8″}-Z-, -Z′=CR^{8″}—, and —CR⁸″=Z′-, or
at least one of “-M-P-Q-”, “—P-Q-T-”, or “-Q-T-U—” is further selected from the group consisting of —CHR^{8″}-Z-CHR^{8″}, —CR^{8″}=Z′CHR^{8″}—, or —CHR^{8″}-Z′=CR^{8″},
Z is CH₂, CHR⁸, CR⁸R⁸, O, S, NH, or NR^{8′}; and
Z′ is CH, CR⁸, or N,
   provided that no heteroatom is directly adjacent to another heteroatom.

In yet another subembodiment, the compound of Formula I, or a pharmaceutically acceptable salt, solvate, ester, or prodrug thereof, is provided, wherein:

### “—V—W—” is —CH═CH—, —CR⁸═CR⁸, —C≡C—,

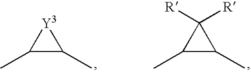

wherein Y³ is O, S, NH, or NR^{8′}, and each R′ is hydrogen, CH₃, CF₃, or halogen (F, Cl, Br, or I).

In a second principle embodiment, a compound of Formula II, or a pharmaceutically acceptable salt, solvate, ester, or prodrug thereof, is provided,

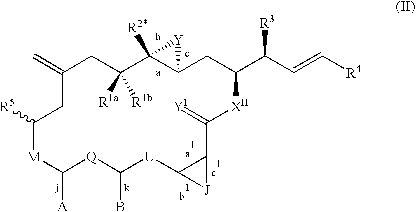

wherein:
R^{1a}, R^{1b}, R³, R⁴, R⁵, R⁸, R^{8′}, “a”, “b”, “c”, Y¹ and Y² are as defined above;
R²* is a radical selected from the group consisting of H, C₁-C₁₀ alkyl, C₂-C₁₀ alkenyl, C₁-C₁₀ alkoxy, C₂-C₁₀ alkenoxy, C₂-C₁₀ alkynyl, C₂-C₁₀ alkynoxy, aryl, nitro, cyano, halogen, CHO, CO₂H, CO₂—C₁₋₁₀ alkyl, CF₃, OCF₃, CON(R^{8′})₂, or CONHR^{8′};
X^{II} is CH₂, N, NR^{8′}, O, or S;
“a¹” can be a single bond, or double bond of either (E)- or (Z)-orientation, or a triple bond when J, “b¹” and “c¹” are absent;
“b¹” can be absent, a single bond, or double bond of either (E)- or (Z)-orientation;
“c¹” can be absent, a single, or double bond of either (E)- or (Z)-orientation; such that only one of “a¹”, “b¹”, and “c¹” can be a double bond;
   when “b¹” and “c¹” are absent, then J is absent; and
   when “a¹” is a single or double bond, one of “b¹” and “c¹” is a single bond and one is absent, then J is H, a straight or branched substituted or unsubstituted alkyl, alkenyl, or alkynyl, CH₃, CH₂R⁸, CHR⁸R⁸, CR⁸R⁸R⁸, CH₂F, CH₂Cl, CH₂Br, CHF₂, CHCl₂, CHBr₂, CF₃, CCl₃, CBr₃, OH, OR^{8′}, SH, SR^{8′}, NH₂, NHR^{8′}, or NR^{8′}R^{8′};
   when “a¹”, “b¹”, and “c¹” are single bonds or when “a¹” is a single bond, one of “b¹” and “c¹” is a double bond and one is absent, then J is CH₂, CHR⁸, CR⁸R⁸, CHF, CHCl, CHBr, CF₂, CCl₂, CBr₂, O, S, NH, or NR^{8′};
M and U are independently CH₂ or CHR⁸;
Q is CH₂, CHR⁸, NR^{8′}, O or S;
“j” can be a single, or double bond of either (E)- or (Z)-orientation; such that
   when “j” is a single bond, then A is H, a straight or branched substituted or unsubstituted alkyl, alkenyl, or alkynyl, CH₃, CH₂R⁸, CHR⁸R⁸, CR⁸R⁸R⁸, CH₂F, CH₂Cl, CH₂Br, CHF₂, CHCl₂, CHBr₂, CF₃, CCl₃, CBr₃, OH, OR^{8′}, SH, SR^{8′}, NH₂, NHR^{8′}, or NR^{8′}R^{8′};
   when “j” is a double bond, then A is CH₂, CHR⁸, CR⁸R⁸, CHF, CHCl, CHBr, CF₂, CCl₂, CBr₂, O, S, NH, or NR^{8′};
“k” can be a single, or double bond of either (E)- or (Z)-orientation; such that
   when “k” is a single bond, then B is H, a straight or branched substituted or unsubstituted alkyl, alkenyl, or alkynyl, CH₃, CH₂R⁸, CHR⁸R⁸, CR⁸R⁸R⁸, CH₂F, CH₂Cl, CH₂Br, CHF₂, CHCl₂, CHBr₂, CF₃, CCl₃, CBr₃, OH, OR^{8′}, SH, SR^{8′}, NH₂, NHR^{8′}, or NR^{8′}R^{8′};
   when “k” is a double bond, then B is CH₂, CHR⁸, CR⁸R⁸, CHF, CHCl, CHBr, CF₂, CCl₂, CBr₂, O, S, NH, or NR^{8′};
alternatively, A and B can join together with -(D-R₇)ₙ— to be -A-(D-R₇)ₙ—B—, to form a ring structure of the formula:

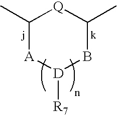

n=0 or 1;
D is CH, CR⁸, or N, or, when R₇ is absent, D is O or S; and
R₇ is H, C₁-C₁₀ alkyl, C₂-C₁₀ alkenyl, C₁-C₁₀ alkoxy, C₂-C₁₀ alkenoxy, C₂-C₁₀ alkynyl, C₂-C₁₀ alkynoxy, carbocyclic, heterocyclic, aryl, substituted aryl, heteroaryl, substituted heteroaryl, nitro, cyano, CF₃, OH, OCF₃, OR⁸′, SH, SR⁸′, NH₂, NHR⁸′, NR⁸′R⁸′, or halogen;
such that no heteroatom is directly adjacent to another heteroatom.

In a subembodiment, the compound of Formula II, or a pharmaceutically acceptable salt, solvate, ester, or prodrug thereof is provided, wherein R^{1a}, R^{1b}, and R⁵ are either hydrogen, CH₃, or C₁-C₅ alkyl.

In another subembodiment, the compound of Formula II, or a pharmaceutically acceptable salt, solvate, ester, or prodrug thereof, is provided, wherein “a¹”, “b¹”, and “c¹” are all single bonds and J is O, S, NH, NR^{8′}, CH₂, CHR′, or CR′R′, wherein each R′ is hydrogen, CH₃, CF₃, or halogen (F, Cl, Br, or I).

In another particular subembodiment, the compound of Formula II, or a pharmaceutically acceptable salt, solvate, ester, or prodrug thereof, is provided, wherein “a¹” is a double bond of either (E)- or (Z)-orientation, and one of “b¹” or “c¹” is a single bond and the other is absent.

In another subembodiment, the compound of Formula II, or a pharmaceutically acceptable salt, solvate, ester, or prodrug thereof, is provided, wherein “a” is a triple bond and both “b¹” or “c¹” are absent.

In a further subembodiment, the compound of Formula II, or a pharmaceutically acceptable salt, solvate, ester, or prodrug thereof, is provided, wherein “a”, “b”, and “c” are all single bonds and Y is O, S, NH, NR^{8′}, CH₂, CHR′, or CR′R′, wherein each R′ is hydrogen, CH₃, CF₃, or halogen (F, Cl, Br, or I).

In yet another subembodiment, the compound of Formula II, or a pharmaceutically acceptable salt, solvate, ester, or prodrug thereof, is provided, wherein “a” is a double bond of either (E)- or (Z)-orientation, and one of “b” or “c” is a single bond and the other is absent.

In a further subembodiment, the compound of Formula II, or a pharmaceutically acceptable salt, solvate, ester, or prodrug thereof, is provided, wherein one of “j” and “k” is a double bond of either (E)- or (Z)-orientation.

In another particular subembodiment, the compound of Formula II, or a pharmaceutically acceptable salt, solvate, ester, or prodrug thereof, is provided, wherein one, and only one, of “j” and “k” is a double bond of either (E)- or (Z)-orientation.

In another particular subembodiment, the compound of Formula II, or a pharmaceutically acceptable salt, solvate, ester, or prodrug thereof, is provided, wherein: one of “j” and “k” is a double bond of either (E)- or (Z)-orientation; and
if “j” is the double bond; then A is CH₂, CHR⁸, CR⁸R⁸, O, S, NH or NR⁸; or
if “k” is the double bond; then B is CH₂, CHR⁸, CR⁸R⁸, O, S, NH or NR^{8′}.

In another subembodiment, the compound of Formula II, or a pharmaceutically acceptable salt, solvate, ester, or prodrug thereof, is provided, wherein: one, and only one, of “j” and “k” is a double bond of either (E)- or (Z)-orientation;
if “j” is the double bond; then A is O, S, NH or NR^{8′}; or
if “k” is the double bond; then B is O, S, NH or NR^{8′}.

In another subembodiment, the compound of Formula II, or a pharmaceutically acceptable salt, solvate, ester, or prodrug thereof, is provided, wherein: one, and only one, of “j” and “k” is a double bond of either (E)- or (Z)-orientation;
if “j” is the double bond; then A is O; or
if “k” is the double bond; then B is O.

In a further subembodiment, the compound of Formula II, or a pharmaceutically acceptable salt, solvate, ester, or prodrug thereof, is provided, wherein both of “j” and “k” are single bonds; and at least one of A and B is a straight or branched substituted or unsubstituted alkenyl or alkynyl.

In another subembodiment, the compound of Formula II, or a pharmaceutically acceptable salt, solvate, ester, or prodrug thereof, is provided, wherein both of “j” and “k” are single bonds; and at least one of A and B is a C₂ to C₄ alk-1-ene, alk-2-ene, alk-1-yne, or alk-2-yne.

In a third principle embodiment, a compound of Formula III, or a pharmaceutically acceptable salt, solvate, ester, or prodrug thereof, is provided,

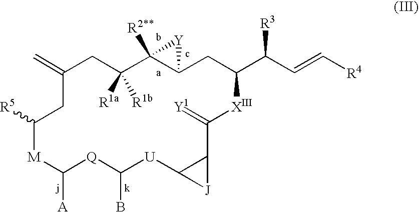

wherein:
R^{1a}, R^{1b}, R³, R⁴, R⁵, R⁸, R^{8′}, X, Y, Y¹, Y², “a”, “b”, and “c” are as defined previously;
R²** is a radical selected from the group consisting of H, C₁-C₁₀ alkyl, C₂-C₁₀ alkenyl, C₁-C₁₀ alkoxy, C₂-C₁₀ alkenoxy, C₂-C₁₀ alkynyl, C₂-C₁₀ alkynoxy, aryl, nitro, cyano, halogen, CHO, CO₂H, CO₂—C₁₋₁₀ alkyl, CF₃, OCF₃, CON(R⁶)₂, or CONHR⁶;
X^{III} is CH₂, N, NR⁵, O, or S;
each Y¹ and Y² is independently O, S, NH, or NR^{8′};
J is CH₂, CHR⁸, CR⁸R⁸, CHF, CHCl, CHBr, CF₂, CCl₂, CBr₂, O, S, NH, or NR^{8′};
M and U are independently selected from the group consisting of CH₂ or CHR⁸;
Q is CH₂, CHR⁸, NR^{8′}, O or S;
“j” can be a single, or double bond of either (E)- or (Z)-orientation; such that
   when “j” is a single bond, then A is H; a straight or branched substituted or unsubstituted alkyl, alkenyl, or alkynyl; CH₃, CH₂R⁸, CHR⁸R⁸, CR⁸R⁸R⁸, CH₂F, CH₂Cl, CH₂Br, CHF₂, CHCl₂, CHBr₂, CF₃, CCl₃, CBr₃, OH, OR^{8′}, SH, SR^{8′}, NH₂, NHR^{8′}, or NR^{8′}R^{8′};
   when “j” is a double bond, then A is CH₂, CHR⁸, CR⁸R⁸, CHF, CHCl, CHBr, CF₂, CCl₂, CBr₂, O, S, NH, or NR^{8′};
“k” can be absent, a single, or double bond of either (E)- or (Z)-orientation; such that
   when “k” is absent, then B is absent;
   when “k” is a single bond, then B is H; a straight or branched substituted or unsubstituted alkyl, alkenyl, or alkynyl; CH₃, CH₂R⁸, CHR⁸R⁸, CR⁸R⁸R⁸, CH₂F, CH₂Cl, CH₂Br, CHF₂, CHCl₂, CHBr₂, CF₃, CCl₃, CBr₃, OH, OR^{8′}, SH, SR^{8′}, NH₂, NHR^{8′}, or NR^{8′}R^{8′};
   when “k” is a double bond, then B is CH₂, CHR⁸, CR⁸R⁸, CHF, CHCl, CHBr, CF₂, CCl₂, CBr₂, O, S, NH, or NR^{8′}.

In a particular subembodiment, the compound of Formula III, or a pharmaceutically acceptable salt, solvate, ester, or prodrug thereof, is provided, wherein R^{1a}, R^{1b}, and R⁵ are either hydrogen, CH₃, or C₁-C₅ alkyl.

In another subembodiment, the compound of Formula III, or a pharmaceutically acceptable salt, solvate, ester, or prodrug thereof, is provided, wherein “a”, “b”, and “c” are all single bonds and Y is O, S, NH, NR^{8′}, CH₂, CHR′, or CR′R′, wherein each R′ is hydrogen, CH₃, CF₃, or halogen (F, Cl, Br, or I).

In another particular subembodiment, the compound of Formula III, or a pharmaceutically acceptable salt, solvate, ester, or prodrug thereof, is provided, wherein “a” is a double bond of either (E)- or (Z)-orientation, and one of “b” or “c” is a single bond and the other is absent.

In another particular subembodiment, the compound of Formula III, or a pharmaceutically acceptable salt, solvate, ester, or prodrug thereof, is provided, wherein one of “j” and “k” is a double bond of either (E)- or (Z)-orientation.

In another subembodiment, the compound of Formula III, or a pharmaceutically acceptable salt, solvate, ester, or prodrug thereof, is provided, wherein one, and only one, of “j” and “k” is a double bond of either (E)- or (Z)-orientation.

In another particular subembodiment, the compound of Formula III, or a pharmaceutically acceptable salt, solvate, ester, or prodrug thereof, is provided, wherein one of “j” and “k” is a double bond of either (E)- or (Z)-orientation; and
if “j” is the double bond; then A is CH₂, CHR⁸, CR⁸R⁸, O, S, NH or N′; or
if “k” is the double bond; then B is CH₂, CHR⁸, CR⁸R⁸, O, S, NH or NR^{8′}.

In another particular subembodiment, the compound of Formula III, or a pharmaceutically acceptable salt, solvate, ester, or prodrug thereof, is provided, wherein: one, and only one, of “j” and “k” is a double bond of either (E)- or (Z)-orientation;
if “j” is the double bond; then A is O, S, NH or NR^{8′}; or
if “k” is the double bond; then B is O, S, NH or NR^{8′}.

In another particular subembodiment, the compound of Formula III, or a pharmaceutically acceptable salt, solvate, ester, or prodrug thereof, is provided, wherein: one, and only one, of “j” and “k” is a double bond of either (E)- or (Z)-orientation;
if “j” is the double bond; then A is O; or
if “k” is the double bond; then B is O.

In another particular subembodiment, the compound of Formula III, or a pharmaceutically acceptable salt, solvate, ester, or prodrug thereof, is provided, wherein both of “j” and “k” are single bonds; and at least one of A and B is a straight or branched substituted or unsubstituted alkenyl or alkynyl.

In another particular subembodiment, the compound of Formula III, or a pharmaceutically acceptable salt, solvate, ester, or prodrug thereof, is provided, wherein both of “j” and “k” are single bonds; and at least one of A and B is a C₂ to C₄ alk-1-ene, alk-2-ene, alk-1-yne, or alk-2-yne.

In a fourth principle embodiment, a compound of Formula IV, or a pharmaceutically acceptable salt, solvate, ester, or prodrug thereof, is provided,

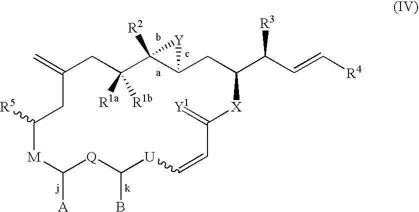

wherein:
R^{1a}, R^{1b}, R², R³, R⁴, R⁵, R⁸, R⁸′, “a”, “b”, “c”, M, Q, U, X, Y, Y and Y² are as defined previously;
“j” can be a single, or double bond of either (E)- or (Z)-orientation; such that
   when “j” is a single bond, then A is H; a straight or branched substituted or unsubstituted alkyl, alkenyl, or alkynyl; CH₃, CH₂R⁸, CHR⁸R⁸, CHR⁸R⁸R⁸, CH₂F, CH₂Cl, CH₂Br, CHF₂, CHCl₂, CHBr₂, CF₃, CCl₃, CBr₃, OH, OR^{8′}, SH, SR^{8′}, NH₂, NHR^{8′}, or NR^{8′}R^{8′};
   when “j” is a double bond, then A is CH₂, CHR⁸, CR⁸R⁸, CHF, CHCl, CHBr, CF₂, CCl₂, CBr₂, O, S, NH, or NR^{8′};
“k” can be absent, a single, or double bond of either (E)- or (Z)-orientation; such that
   when “k” is absent, then B is absent;
   when “k” is a single bond, then B is H; a straight or branched substituted or unsubstituted alkyl, alkenyl, or alkynyl; CH₃, CH₂R⁸, CHR⁸R⁸, CR⁸R⁸R⁸, CH₂F, CH₂Cl, CH₂Br, CHF₂, CHCl₂, CHBr₂, CF₃, CCl₃, CBr₃, OH, OR⁸, SH, SR^{8′}, NH₂, NHR^{8′}, or NR^{8′}R^{8′}; and
   when “k” is a double bond, then B is CH₂, CHR⁸, CR⁸R⁸, CHF, CHCl, CHBr, CF₂, CCl₂, CBr₂, O, S, NH, or NR^{8′}.

In a particular subembodiment, the compound of Formula IV, or a pharmaceutically acceptable salt, solvate, ester, or prodrug thereof, is provided, wherein R^{1a}, R^{1b}, and R⁵ are either hydrogen, CH₃, or C₁-C₅ alkyl.

In another particular subembodiment, the compound of Formula IV, or a pharmaceutically acceptable salt, solvate, ester, or prodrug thereof, is provided, wherein “a”, “b”, and “c” are all single bonds and Y is O, S, NH, NR⁸, CH₂, CHR′, or CR′R′, wherein each R′ is hydrogen, CH₃, CF₃, or halogen (F, Cl, Br, or I).

In a further subembodiment, the compound of Formula IV, or a pharmaceutically acceptable salt, solvate, ester, or prodrug thereof, is provided, wherein “a” is a double bond of either (E)- or (Z)-orientation, and one of “b” or “c” is a single bond and the other is absent.

In another particular subembodiment, the compound of Formula IV, or a pharmaceutically acceptable salt, solvate, ester, or prodrug thereof, is provided, wherein one of “j” and “k” is a double bond of either (E)- or (Z)-orientation.

In another particular subembodiment, the compound of Formula Iv, or a pharmaceutically acceptable salt, solvate, ester, or prodrug thereof, is provided, wherein one, and only one, of “j” and “k” is a double bond of either (E)- or (Z)-orientation.

In another particular subembodiment, the compound of Formula IV, or a pharmaceutically acceptable salt, solvate, ester, or prodrug thereof, is provided, wherein: one of “j” and “k” is a double bond of either (E)- or (Z)-orientation; and
if “j” is the double bond; then A is CH₂, CHR⁸, CR⁸R⁸, O, S, NH or NR⁸; or
if “k” is the double bond; then B is CH₂, CHR⁸, CR⁸R⁸, O, S, NH or NR^{8′}.

In another particular subembodiment, the compound of Formula IV, or a pharmaceutically acceptable salt, solvate, ester, or prodrug thereof, is provided, wherein: one, and only one, of “j” and “k” is a double bond of either (E)- or (Z)-orientation;
if “j” is the double bond; then A is O, S, NH or NR^{8′}; or
if “k” is the double bond; then B is O, S, NH or NR^{8′}.

In another particular subembodiment, the compound of Formula IV, or a pharmaceutically acceptable salt, solvate, ester, or prodrug thereof, is provided, wherein: one, and only one, of “j” and “k” is a double bond of either (E)- or (Z)-orientation;
if “j” is the double bond; then A is O; or
if “k” is the double bond; then B is O.

In another particular subembodiment, the compound of Formula IV, or a pharmaceutically acceptable salt, solvate, ester, or prodrug thereof, is provided, wherein both of “j” and “k” are single bonds; and at least one of A and B is a straight or branched substituted or unsubstituted alkenyl or alkynyl.

In another particular subembodiment, the compound of Formula IV, or a pharmaceutically acceptable salt, solvate, ester, or prodrug thereof, is provided, wherein both of “j” and “k” are single bonds; and at least one of A and B is a C₂ to C₄ alk-1-ene, alk-2-ene, alk-1-yne, or alk-2-yne.

In a fifth principle embodiment, a compound of Formula V, or a pharmaceutically acceptable salt, solvate, ester, or prodrug thereof, is provided,

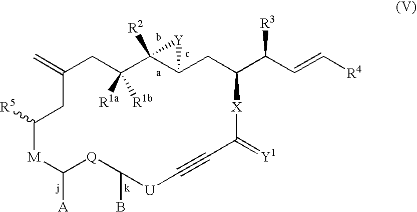

wherein:
R^{1a}, R^{1b}, R², R³, R⁴, R⁵, R⁸, R⁸′, “a”, “b”, “c”, “j”, “k”, M, Q, U, X, Y, Y¹ and Y² are as defined previously.

In a particular subembodiment, the compound of Formula V, or a pharmaceutically acceptable salt, solvate, ester, or prodrug thereof, is provided, wherein R^{1a}, R^{1b}, and R⁵ are either hydrogen, CH₃, or C₁-C₅ alkyl.

In another particular subembodiment, the compound of Formula V, or a pharmaceutically acceptable salt, solvate, ester, or prodrug thereof, is provided, wherein “a”, “b”, and “c” are all single bonds and Y is O, S, NH, NR^{8′}, CH₂, CHR′, or CR′R′, wherein each R′ is hydrogen, CH₃, CF₃, or halogen (F, Cl, Br, or I).

In yet another particular subembodiment, the compound of Formula V, or a pharmaceutically acceptable salt, solvate, ester, or prodrug thereof, is provided, wherein “a” is a double bond of either (E)- or (Z)-orientation, and one of “b” or “c” is a single bond and the other is absent.

In another particular subembodiment, the compound of Formula V, or a pharmaceutically acceptable salt, solvate, ester, or prodrug thereof, is provided, wherein one of “j” and “k” is a double bond of either (E)- or (Z)-orientation.

In another particular subembodiment, the compound of Formula V, or a pharmaceutically acceptable salt, solvate, ester, or prodrug thereof, is provided, wherein one, and only one, of “j” and “k” is a double bond of either (E)- or (Z)-orientation.

In another particular subembodiment, the compound of Formula V, or a pharmaceutically acceptable salt, solvate, ester, or prodrug thereof, is provided, wherein: one of “j” and “k” is a double bond of either (E)- or (Z)-orientation; and
if “j” is the double bond; then A is CH₂, CHR⁸, CR⁸R⁸, O, S, NH or NR^{8′}; or
if “k” is the double bond; then B is CH₂, CHR⁸, CR⁸R⁸, O, S, NH or NR^{8′}.

In another particular subembodiment, the compound of Formula V, or a pharmaceutically acceptable salt, solvate, ester, or prodrug thereof, is provided, wherein: one, and only one, of “j” and “k” is a double bond of either (E)- or (Z)-orientation;
if “j” is the double bond; then A is O, S, NH or NR^{8′}; or
if “k” is the double bond; then B is O, S, NH or NR^{8′}.

In another particular subembodiment, the compound of Formula V, or a pharmaceutically acceptable salt, solvate, ester, or prodrug thereof, is provided, wherein: one, and only one, of “j” and “k” is a double bond of either (E)- or (Z)-orientation;
if “j” is the double bond; then A is O; or
if “k” is the double bond; then B is O.

In another particular subembodiment, the compound of Formula V, or a pharmaceutically acceptable salt, solvate, ester, or prodrug thereof, is provided, wherein both of “j” and “k” are single bonds; and at least one of A and B is a straight or branched substituted or unsubstituted alkenyl or alkynyl.

In another particular subembodiment, the compound of Formula V, or a pharmaceutically acceptable salt, solvate, ester, or prodrug thereof, is provided, wherein both of “j” and “k” are single bonds; and at least one of A and B is a C₂ to C₄ alk-1-ene, alk-2-ene, alk-1-yne, or alk-2-yne.

In a sixth principle embodiment, a compound of Formula VI, or a pharmaceutically acceptable salt, solvate, ester, or prodrug thereof, is provided,

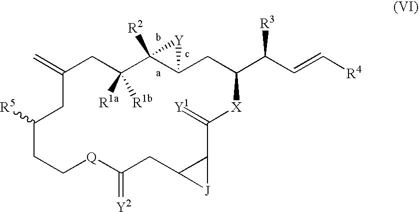

wherein:
“a”, “b”, “c”, R^{1a}, R^{1b}, R², R³, R⁴, R⁵, X, Y, Y¹, Y², J, Q, R⁸, and R^{8′} are as defined above.

In a particular subembodiment, the compound of Formula VI, or a pharmaceutically acceptable salt, solvate, ester, or prodrug thereof, is provided, wherein R^{1a}, R^{1b}, and R⁵ are either hydrogen, CH₃, or C₁-C₅ alkyl.

In another particular subembodiment, the compound of Formula VI, or a pharmaceutically acceptable salt, solvate, ester, or prodrug thereof, is provided, wherein “a”, “b”, and “c” are all single bonds and Y is O, S, NH, NR^{8′}, CH₂, CHR′, or CR′R′, wherein each R′ is hydrogen, CH₃, CF₃, or halogen (F, Cl, Br, or I).

In another particular subembodiment, the compound of Formula VI, or a pharmaceutically acceptable salt, solvate, ester, or prodrug thereof, is provided, wherein “a” is a double bond of either (E)- or (Z)-orientation, and one of “b” or “c” is a single bond and the other is absent.

In another particular subembodiment, the compound of Formula VI, or a pharmaceutically acceptable salt, solvate, ester, or prodrug thereof, is provided, wherein Q is O, S, NH, or NR⁸′.

In yet another particular subembodiment, the compound of Formula VI, or a pharmaceutically acceptable salt, solvate, ester, or prodrug thereof, is provided, wherein Q is O.

In another particular subembodiment, the compound of Formula VI, or a pharmaceutically acceptable salt, solvate, ester, or prodrug thereof, is provided, wherein Q is NH or NR^{8′}.

In a further subembodiment, the compound of Formula VI, or a pharmaceutically acceptable salt, solvate, ester, or prodrug thereof, is provided, wherein Q is NH.

In another particular subembodiment, the compound of Formula VI, or a pharmaceutically acceptable salt, solvate, ester, or prodrug thereof, is provided, wherein X is O.

In another particular subembodiment, the compound of Formula VI, or a pharmaceutically acceptable salt, solvate, ester, or prodrug thereof, is provided, wherein Y¹ is O.

In another particular subembodiment, the compound of Formula VI, or a pharmaceutically acceptable salt, solvate, ester, or prodrug thereof, is provided, wherein Y² is O.

In another particular subembodiment, the compound of Formula VI, or a pharmaceutically acceptable salt, solvate, ester, or prodrug thereof, is provided, wherein Y¹ and Y² are O.

In another particular subembodiment, the compound of Formula VI, or a pharmaceutically acceptable salt, solvate, ester, or prodrug thereof, is provided, wherein

### Q is O, S, NH, or NR^{8′};

### X is O; and

### Y¹ and Y² are O.

In another particular subembodiment, the compound of Formula VI, or a pharmaceutically acceptable salt, solvate, ester, or prodrug thereof, is provided, wherein:

### Q is O;

### X is O; and

### Y¹ and Y² are O.

In another particular subembodiment, the compound of Formula VI, or a pharmaceutically acceptable salt, solvate, ester, or prodrug thereof, is provided, wherein:

### Q is NH or NR^{8′};

### X is O; and

### Y¹ and Y² are O.

In another particular subembodiment, the compound of Formula VI, or a pharmaceutically acceptable salt, solvate, ester, or prodrug thereof, is provided, wherein:

### Q is NH;

### X is O; and

### Y¹ and Y² are O.

In a seventh principle embodiment, a compound of Formula VII, or a pharmaceutically acceptable salt, solvate, ester, or prodrug thereof, is provided,

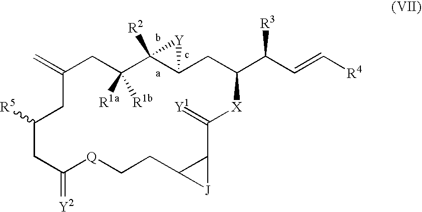

wherein:
“a”, “b”, “c”, R^{1a}, R^{1b}, R², R³, R⁴, R⁵, X, Y, Y¹, Y², J, Q, R⁸, and R^{8′} are as defined above.

In a particular subembodiment, the compound of Formula VII, or a pharmaceutically acceptable salt, solvate, ester, or prodrug thereof, is provided, wherein R^{1a}, R^{1b}, and R⁵ are either hydrogen, CH₃, or C₁-C₅ alkyl.

In another particular subembodiment, the compound of Formula VII, or a pharmaceutically acceptable salt, solvate, ester, or prodrug thereof, is provided, wherein “a”, “b”, and “c” are all single bonds and Y is O, S, NH, NR^{8′}, CH₂, CHR′, or CR′R′, wherein each R′ is hydrogen, CH₃, CF₃, or halogen (F, Cl, Br, or I).

In yet another particular subembodiment, the compound of Formula VII, or a pharmaceutically acceptable salt, solvate, ester, or prodrug thereof, is provided, wherein “a” is a double bond of either (E)- or (Z)-orientation, and one of “b” or “c” is a single bond and the other is absent.

In another particular subembodiment, the compound of Formula VII, or a pharmaceutically acceptable salt, solvate, ester, or prodrug thereof, is provided, wherein Q is O, S, NH, or NR^{8′}.

In another particular subembodiment, the compound of Formula VII, or a pharmaceutically acceptable salt, solvate, ester, or prodrug thereof, is provided, wherein Q is O.

In another particular subembodiment, the compound of Formula VII, or a pharmaceutically acceptable salt, solvate, ester, or prodrug thereof, is provided, wherein Q is NH or NR^{8′}.

In yet another particular subembodiment, the compound of Formula VII, or a pharmaceutically acceptable salt, solvate, ester, or prodrug thereof, is provided, wherein Q is NH.

In another particular subembodiment, the compound of Formula VII, or a pharmaceutically acceptable salt, solvate, ester, or prodrug thereof, is provided, wherein X is O.

In another particular subembodiment, the compound of Formula VII, or a pharmaceutically acceptable salt, solvate, ester, or prodrug thereof, is provided, wherein Y is O.

In another particular subembodiment, the compound of Formula VII, or a pharmaceutically acceptable salt, solvate, ester, or prodrug thereof, is provided, wherein Y² is O.

In another particular subembodiment, the compound of Formula VII, or a pharmaceutically acceptable salt, solvate, ester, or prodrug thereof, is provided, wherein Y¹ and Y² are O.

In another particular subembodiment, the compound of Formula VII, or a pharmaceutically acceptable salt, solvate, ester, or prodrug thereof, is provided, wherein

### Q is O, S, NH, or NR^{8′};

### X is O; and

### Y¹ and Y² are O.

In another particular subembodiment, the compound of Formula VII, or a pharmaceutically acceptable salt, solvate, ester, or prodrug thereof, is provided, wherein:

### Q is O;

### X is O; and

### Y¹ and Y² are O.

In another particular subembodiment, the compound of Formula VII, or a pharmaceutically acceptable salt, solvate, ester, or prodrug thereof, is provided, wherein:

### Q is NH or NR^{8′};

### X is O; and

### Y¹ and Y² are O.

In another particular subembodiment, the compound of Formula VII, or a pharmaceutically acceptable salt, solvate, ester, or prodrug thereof, is provided, wherein:

### Q is NH;

### X is O; and

### Y¹ and Y² are O.

In an eighth principle embodiment, a compound of Formula VIII, or a pharmaceutically acceptable salt, solvate, ester, or prodrug thereof, is provided,

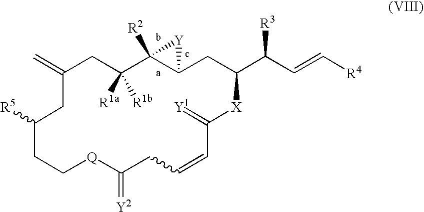

wherein:
“a”, “b”, “c” R^{1a}, R^{1b}, R², R³, R⁴, R⁵, X, Y, Y¹, Y², Q, R⁸, and R^{8′} are as defined above.

In a particular subembodiment, the compound of Formula VIII, or a pharmaceutically acceptable salt, solvate, ester, or prodrug thereof, is provided, wherein R^{1a}, R^{1b}, and R⁵ are either hydrogen, CH₃, or C₁-C₅ alkyl.

In another particular subembodiment, the compound of Formula VIII, or a pharmaceutically acceptable salt, solvate, ester, or prodrug thereof, is provided, wherein “a”, “b”, and “c” are all single bonds and Y is O, S, NH, NR^{8′}, CH₂, CHR′, or CR′R′, wherein each R′ is hydrogen, CH₃, CF₃, or halogen (F, Cl, Br, or I).

In another particular subembodiment, the compound of Formula VIII, or a pharmaceutically acceptable salt, solvate, ester, or prodrug thereof, is provided, wherein “a” is a double bond of either (E)- or (Z)-orientation, and one of “b” or “c” is a single bond and the other is absent.

In another particular subembodiment, the compound of Formula VIII, or a pharmaceutically acceptable salt, solvate, ester, or prodrug thereof, is provided, wherein: is O, S, NH, or NR^{8′}.

In another particular subembodiment, the compound of Formula VIII, or a pharmaceutically acceptable salt, solvate, ester, or prodrug thereof, is provided, wherein Q is O.

In another particular subembodiment, the compound of Formula VIII, or a pharmaceutically acceptable salt, solvate, ester, or prodrug thereof, is provided, wherein is NH or NR^{8′}.

In another particular subembodiment, the compound of Formula VIII, or a pharmaceutically acceptable salt, solvate, ester, or prodrug thereof, is provided, wherein Q is NH.

In another particular subembodiment, the compound of Formula VIII, or a pharmaceutically acceptable salt, solvate, ester, or prodrug thereof, is provided, wherein X is O.

In another particular subembodiment, the compound of Formula VIII, or a pharmaceutically acceptable salt, solvate, ester, or prodrug thereof, is provided, wherein Y¹ is O.

In another particular subembodiment, the compound of Formula VIII, or a pharmaceutically acceptable salt, solvate, ester, or prodrug thereof, is provided, wherein Y² is O.

In yet another particular subembodiment, the compound of Formula VIII, or a pharmaceutically acceptable salt, solvate, ester, or prodrug thereof, is provided, wherein Y¹ and Y² are O.

In another particular subembodiment, the compound of Formula VIII, or a pharmaceutically acceptable salt, solvate, ester, or prodrug thereof, is provided, wherein:

### Q is O, S, NH, or NR^{8′};

### X is O; and

### Y¹ and Y² are O.

In another particular subembodiment, the compound of Formula VIII, or a pharmaceutically acceptable salt, solvate, ester, or prodrug thereof, is provided, wherein:

### Q is O;

### X is O; and

### Y¹ and Y² are O.

In another particular subembodiment, the compound of Formula VIII, or a pharmaceutically acceptable salt, solvate, ester, or prodrug thereof, is provided, wherein:

### Q is NH or NR^{8′};

### X is O; and

### Y¹ and Y² are O.

In yet another particular subembodiment, the compound of Formula VIII, or a pharmaceutically acceptable salt, solvate, ester, or prodrug thereof, is provided, wherein:

### Q is NH;

### X is O; and

### Y¹ and Y² are O.

In a ninth principle embodiment, a compound of Formula IX, or a pharmaceutically acceptable salt, solvate, ester, or prodrug thereof, is provided,

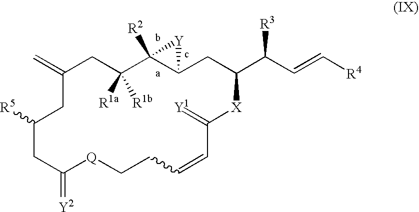

wherein:
“a”, “b”, “c”, R^{1a}, R^{1b}, R², R³, R⁴, R⁵, X, Y, Y¹, Y², Q, R¹, and R^{8′} are as defined above.

In a particular subembodiment, the compound of Formula IX, or a pharmaceutically acceptable salt, solvate, ester, or prodrug thereof, is provided, wherein R^{1a}, R^{1b}, and R⁵ are either hydrogen, CH₃, or C₁-C₅ alkyl.

In another particular subembodiment, the compound of Formula IX, or a pharmaceutically acceptable salt, solvate, ester, or prodrug thereof, is provided, wherein “a”, “b”, and “c” are all single bonds and Y is O, S, NH, NR^{8′}, CH₂, CHR′, or CR′R′, wherein each R′ is hydrogen, CH₃, CF₃, or halogen (F, Cl, Br, or I).

In yet another particular subembodiment, the compound of Formula IX, or a pharmaceutically acceptable salt, solvate, ester, or prodrug thereof, is provided, wherein “a” is a double bond of either (E)- or (Z)-orientation, and one of “b” or “c” is a single bond and the other is absent.

In another particular subembodiment, the compound of Formula IX, or a pharmaceutically acceptable salt, solvate, ester, or prodrug thereof, is provided, wherein Q is O, S, NH, or NR^{8′}.

In another particular subembodiment, the compound of Formula IX, or a pharmaceutically acceptable salt, solvate, ester, or prodrug thereof, is provided, wherein Q is O.

In another particular subembodiment, the compound of Formula IX, or a pharmaceutically acceptable salt, solvate, ester, or prodrug thereof, is provided, wherein Q is NH or NR⁸′.

In another particular subembodiment, the compound of Formula IX, or a pharmaceutically acceptable salt, solvate, ester, or prodrug thereof, is provided, wherein Q is NH.

In another particular subembodiment, the compound of Formula IX, or a pharmaceutically acceptable salt, solvate, ester, or prodrug thereof, is provided, wherein X is O.

In another particular subembodiment, the compound of Formula IX, or a pharmaceutically acceptable salt, solvate, ester, or prodrug thereof, is provided, wherein Y¹ is O.

In yet another subembodiment, the compound of Formula IX, or a pharmaceutically acceptable salt, solvate, ester, or prodrug thereof, is provided, wherein Y² is O.

In another particular subembodiment, the compound of Formula IX, or a pharmaceutically acceptable salt, solvate, ester, or prodrug thereof, is provided, wherein Y¹ and Y² are O.

In another particular subembodiment, the compound of Formula IX, or a pharmaceutically acceptable salt, solvate, ester, or prodrug thereof, is provided, wherein:

### Q is O, S, NH, or NR^{8′};

### X is O; and

### Y¹ and Y² are O.

In another particular subembodiment, the compound of Formula IX, or a pharmaceutically acceptable salt, solvate, ester, or prodrug thereof, is provided, wherein:

### Q is O;

### X is O; and

### Y¹ and Y² are O.

In yet another particular subembodiment, the compound of Formula IX, or a pharmaceutically acceptable salt, solvate, ester, or prodrug thereof, is provided, wherein:

### Q is NH or NR^{8′};

### X is O; and

### Y¹ and Y² are O.

In another particular subembodiment, the compound of Formula IX, or a pharmaceutically acceptable salt, solvate, ester, or prodrug thereof, is provided, wherein:

### Q is NH;

### X is O; and

### Y¹ and Y² are O.

In a tenth principle embodiment, a compound of Formula X, or a pharmaceutically acceptable salt, solvate, ester, or prodrug thereof, is provided,

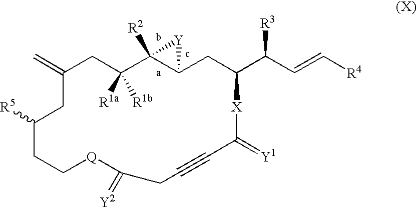

wherein:
“a”, “b”, “c”, R^{1a}, R^{1b}, R², R³, R⁴, R⁵, X, Y, Y¹, Y², Q, R⁸, and R^{8′} are as defined above.

In a particular subembodiment, the compound of Formula X, or a pharmaceutically acceptable salt, solvate, ester, or prodrug thereof, is provided, wherein R^{1a}, R^{1b}, and R⁵ are either hydrogen, CH₃, or C₁-C₅ alkyl.

In another particular subembodiment, the compound of Formula X, or a pharmaceutically acceptable salt, solvate, ester, or prodrug thereof, is provided, wherein “a”, “b”, and “c” are all single bonds and Y is O, S, NH, NR^{8′}, CH₂, CHR′, or CR′R′, wherein each R′ is hydrogen, CH₃, CF₃, or halogen (F, Cl, Br, or I).

In another particular subembodiment, the compound of Formula X, or a pharmaceutically acceptable salt, solvate, ester, or prodrug thereof, is provided, wherein “a” is a double bond of either (E)- or (Z)-orientation, and one of “b” or “c” is a single bond and the other is absent.

In yet another particular subembodiment, the compound of Formula X, or a pharmaceutically acceptable salt, solvate, ester, or prodrug thereof, is provided, wherein Q is O, S, NH, or NR^{8′}.

In another particular subembodiment, the compound of Formula X, or a pharmaceutically acceptable salt, solvate, ester, or prodrug thereof, is provided, wherein Q is O.

In another particular subembodiment, the compound of Formula X, or a pharmaceutically acceptable salt, solvate, ester, or prodrug thereof, is provided, wherein Q is NH or NR^{8′}.

In yet another particular subembodiment, the compound of Formula X, or a pharmaceutically acceptable salt, solvate, ester, or prodrug thereof, is provided, wherein Q is NH.

In another particular subembodiment, the compound of Formula X, or a pharmaceutically acceptable salt, solvate, ester, or prodrug thereof, is provided, wherein X is O.

In another particular subembodiment, the compound of Formula X, or a pharmaceutically acceptable salt, solvate, ester, or prodrug thereof, is provided, wherein Y¹ is O.

In another particular subembodiment, the compound of Formula X, or a pharmaceutically acceptable salt, solvate, ester, or prodrug thereof, is provided, wherein Y² is O.

In yet another particular subembodiment, the compound of Formula X, or a pharmaceutically acceptable salt, solvate, ester, or prodrug thereof, is provided, wherein Y¹ and Y² are O.

In another particular subembodiment, the compound of Formula X, or a pharmaceutically acceptable salt, solvate, ester, or prodrug thereof, is provided, wherein:

### Q is O, S, NH, or NR^{8′};

### X is O; and

### Y¹ and Y² are O.

In another particular subembodiment, the compound of Formula X, or a pharmaceutically acceptable salt, solvate, ester, or prodrug thereof, is provided, wherein:

### Q is O;

### X is O; and

### Y¹ and Y² are O.

In yet another particular subembodiment, the compound of Formula X, or a pharmaceutically acceptable salt, solvate, ester, or prodrug thereof, is provided, wherein:

### Q is NH or NR⁸′;

### X is O; and

### Y¹ and Y² are O.

In another particular subembodiment, the compound of Formula X, or a pharmaceutically acceptable salt, solvate, ester, or prodrug thereof, is provided, wherein:

### Q is NH;

### X is O; and

### Y¹ and Y² are O.

In an eleventh principle embodiment, a compound of Formula XI, or a pharmaceutically acceptable salt, solvate, ester, or prodrug thereof, is provided,

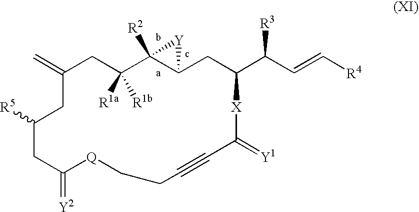

wherein:
“a”, “b”, “c”, R^{1a}, R^{1b}, R², R³, R⁴, R⁵, X, Y, Y¹, Y², Q, R⁸, and R^{8′} are as defined above.

In a particular subembodiment, the compound of Formula XI, or a pharmaceutically acceptable salt, solvate, ester, or prodrug thereof, is provided, wherein R^{1a}, R^{1b}, and R⁵ are either hydrogen, CH₃, or C₁-C₅ alkyl.

In another particular subembodiment, the compound of Formula XI, or a pharmaceutically acceptable salt, solvate, ester, or prodrug thereof, is provided, wherein “a”, “b”, and “c” are all single bonds and Y is O, S, NH, NR^{8′}, CH₂, CHR′, or CR′R′, wherein each R′ is hydrogen, CH₃, CF₃, or halogen (F, Cl, Br, or I).

In a further subembodiment, the compound of Formula XI, or a pharmaceutically acceptable salt, solvate, ester, or prodrug thereof, is provided, wherein “a” is a double bond of either (E)- or (Z)-orientation, and one of “b” or “c” is a single bond and the other is absent.

In another particular subembodiment, the compound of Formula XI, or a pharmaceutically acceptable salt, solvate, ester, or prodrug thereof, is provided, wherein Q is O, S, NH, or NR^{8′}.

In another particular subembodiment, the compound of Formula XI, or a pharmaceutically acceptable salt, solvate, ester, or prodrug thereof, is provided, wherein Q is O.

In another particular subembodiment, the compound of Formula XI, or a pharmaceutically acceptable salt, solvate, ester, or prodrug thereof, is provided, wherein Q is NH or NR^{8′}.

In another particular subembodiment, the compound of Formula XI, or a pharmaceutically acceptable salt, solvate, ester, or prodrug thereof, is provided, wherein Q is NH.

In another particular subembodiment, the compound of Formula XI, or a pharmaceutically acceptable salt, solvate, ester, or prodrug thereof, is provided, wherein X is O.

In yet another particular subembodiment, the compound of Formula XI, or a pharmaceutically acceptable salt, solvate, ester, or prodrug thereof, is provided, wherein Y¹ is O.

In another particular subembodiment, the compound of Formula XI, or a pharmaceutically acceptable salt, solvate, ester, or prodrug thereof, is provided, wherein Y² is O.

In another particular subembodiment, the compound of Formula XI, or a pharmaceutically acceptable salt, solvate, ester, or prodrug thereof, is provided, wherein Y¹ and Y² are O.

In another particular subembodiment, the compound of Formula XI, or a pharmaceutically acceptable salt, solvate, ester, or prodrug thereof, is provided, wherein: Q is O, S, NH, or NR^{8′};

### X is O; and

### Y¹ and Y² are O.

In another particular subembodiment, the compound of Formula XI, or a pharmaceutically acceptable salt, solvate, ester, or prodrug thereof, is provided, wherein:

### Q is O;

### X is O; and

### Y¹ and Y² are O.

In another particular subembodiment, the compound of Formula XI, or a pharmaceutically acceptable salt, solvate, ester, or prodrug thereof, is provided, wherein:

### Q is NH or NR^{8′};

### X is O; and

### Y¹ and Y² are O.

In another particular subembodiment, the compound of Formula XI, or a pharmaceutically acceptable salt, solvate, ester, or prodrug thereof, is provided, wherein:

### Q is NH;

### X is O; and

### Y¹ and Y² are O.

### DEFINITIONS

The terms “C₁-C₁₀ alkyl”, “C₂-C₁₀ alkenyl”, C₁-C₁₀ alkoxy, C₂-C₁₀ alkenoxy, C₂-C₁₀ alkynyl, and C₂-C₁₀ alkynoxy are considered to include, independently, each member of the group, such that, for example, C₁-C₁₀ alkyl includes straight, branched and where appropriate cyclic C₁, C₂, C₃, C₄, C₅, C₆, C₇, C₈, C₉ and C₁₀ alkyl functionalities; C₂-C₁₀ alkenyl includes straight, branched, and where appropriate cyclic C₂, C₃, C₄, C₅, C₆, C₇, C₈, C₉ and C₁₀ alkenyl functionalities; C₁-C₁₀ alkoxy includes straight, branched, and where appropriate cyclic C₁, C₂, C₃, C₄, C₅, C₆, C₇, C₈, C₉ and C₁₀ alkoxy functionalities; C₂-C₁₀ alkenoxy includes straight, branched, and where appropriate cyclic C₂, C₃, C₄, C₅, C₆, C₇, C₈, C₉ and C₁₀ alkenoxy functionalities; C₂-C₁₀ alkynyl includes straight, branched and where appropriate cyclic C₁, C₂, C₃, C₄, C₅, C₆, C₇, C₈, C₉ and C₁₀ alkynyl functionalities; and C₂-C₁₀ alkynoxy includes straight, branched, and where appropriate cyclic C₂, C₃, C₄, C₅, C₆, C₇, C₈, C₉ and C₁₀ alkynoxy functionalities.

Throughout this disclosure, when a range is specified (i.e. 1-10), then each individual element of that range is separately and independently included. For example, the term “C₁₋₁₀ alkyl” separately and independently includes C₁-alkyl, C₂-alkyl, C₃-alkyl, C₄-alkyl, C₅-alkyl, C₆-alkyl, C₇-alkyl, C₈-alkyl, C₉-alkyl and C₁₀-alkyl.

The term “alkyl”, alone or in combination, means an acyclic, saturated straight, branched, or cyclic, primary, secondary, or tertiary hydrocarbon, including those containing from 1 to 10 carbon atoms or from 1 to 6 carbon atoms. Said alkyl radicals may be optionally substituted with groups as defined below. The term alkyl specifically includes but is not limited to methyl, ethyl, propyl, isopropyl, cyclopropyl, butyl, isobutyl, t-butyl, sec-butyl, pentyl, cyclopentyl, isopentyl, neopentyl, hexyl, isohexyl, cyclohexyl, cyclohexylmethyl, 3-methylpentyl, 2,2-dimethylbutyl, and 2,3-dimethylbutyl, heptyl, octyl; nonyl, decyl, trifluoromethyl and difluoromethyl. The term includes both substituted and unsubstituted alkyl groups. Moieties with which the alkyl group can be substituted are, for example, alkyl, hydroxyl, halo, nitro, cyano, alkenyl, alkynyl, heteroaryl, heterocyclic, carbocycle, alkoxy, oxo, aryloxy, arylalkoxy, cycloalkyl, tetrazolyl, heteroaryloxy; heteroarylalkoxy, carbohydrate, amino acid, amino acid esters, amino acid amides, alditol, haloalkylthi, haloalkoxy, haloalkyl, hydroxyl, carboxyl, acyl, acyloxy, amino, aminoalkyl, aminoacyl, amido, alkylamino, dialkylamino, arylamino, nitro, cyano, thiol, imide, sulfonic acid, sulfate, sulfonate, sulfonyl, alkylsulfonyl, aminosulfonyl, alkylsulfonylamino, haloalkylsulfonyl, sulfanyl, sulfinyl, sulfamoyl, carboxylic ester, carboxylic acid, amide, phosphonyl, phosphinyl, phosphoryl, thioester, thioether, oxime, hydrazine, carbamate, phosphonic acid, phosphate, phosphonate, phosphinate, sulfonamido, carboxamido, hydroxamic acid, sulfonylimide or any other desired functional group that does not inhibit the pharmacological activity of this compound, either unprotected, or protected as necessary, as known to those skilled in the art, for example, as taught in Greene, et al., Protective Groups in Organic Synthesis, John Wiley and Sons, Third Edition, 1999, hereby incorporated by reference.

The term “alkenyl”, alone or in combination, means an acyclic, straight, branched, or cyclic, primary, secondary, or tertiary hydrocarbon, including those containing from 2 to 10 carbon atoms or from 2 to 6 carbon atoms, wherein the substituent contains at least one carbon-carbon double bond. Said alkenyl radicals may be optionally substituted. Examples of such radicals include but are not limited to are ethylene, methylethylene, and isopropylidene.

The term “alkynyl” refers to an unsaturated, acyclic hydrocarbon radical, linear or branched, in so much as it contains one or more triple bonds, including such radicals containing about 2 to 10 carbon atoms or having from 2 to 6 carbon atoms. The alkynyl radicals may be optionally substituted with groups as defined herein. Examples of suitable alkynyl radicals include ethynyl, propynyl, hydroxypropynyl, butyn-1-yl, butyn-2-yl, pentyn-1-yl, pentyn-2-yl, 4-methoxypentyn-2-yl, 3-methylbutyn-1-yl, hexyn-1-yl, hexyn-2-yl, hexyn-3-yl, 3,3-dimethylbutyn-1-yl radicals and the like.

The term “acyl”, alone or in combination, means a carbonyl or thionocarbonyl group bonded to a radical selected from, for example, hydrido, alkyl, alkenyl, alkynyl, haloalkyl, alkoxy, alkoxyalkyl, haloalkoxy, aryl, heterocyclyl, heteroaryl, alkylsulfinylalkyl, alkylsulfonylalkyl, aralkyl, cycloalkyl, cycloalkylalkyl, cycloalkenyl, alkylthio, arylthio, amino, alkylamino, dialkylamino, aralkoxy, arylthio, and alkylthioalkyl. Examples of “acyl” are formyl, acetyl, benzoyl, trifluoroacetyl, phthaloyl, malonyl, nicotinyl, and the like.

The terms “alkoxy” and “alkoxyalkyl” embrace linear or branched oxy-containing radicals each having alkyl portions of one to about ten carbon atoms, such as methoxy radical. The term “alkoxyalkyl” also embraces alkyl radicals having one or more alkoxy radicals attached to the alkyl radical, that is, to form monoalkoxyalkyl and dialkoxyalkyl radicals. Other alkoxy radicals are “lower alkoxy” radicals having one to six carbon atoms. Examples of such radicals include methoxy, ethoxy, propoxy, butoxy and tert-butoxy alkyls. The “alkoxy” radicals may be further substituted with one or more halo atoms, such as fluoro, chloro or bromo, to provide “haloalkoxy” radicals. Examples of such radicals include fluoromethoxy, chloromethoxy, trifluoromethoxy, difluoromethoxy, trifluoroethoxy, fluoroethoxy, tetrafluoroethoxy, pentafluoroethoxy, and fluoropropoxy.

The term “alkylamino” denotes “monoalkylamino” and “dialkylamino” containing one or two alkyl radicals, respectively, attached to an amino radical. The terms arylamino denotes “monoarylamino” and “diarylamino” containing one or two aryl radicals, respectively, attached to an amino radical. The term “aralkylamino”, embraces aralkyl radicals attached to an amino radical. The term aralkylamino denotes “monoaralkylamino” and “diaralkylamino” containing one or two aralkyl radicals, respectively, attached to an amino radical. The term aralkylamino further denotes “monoaralkyl monoalkylamino” containing one aralkyl radical and one alkyl radical attached to an amino radical.

The term “alkoxy” is defined as —OR, wherein R is alkyl, including cycloalkyl.

The term “alkoxyalkyl” is defined as an alkyl group wherein a hydrogen has been replaced by an alkoxy group. The term “(alkylthio)alkyl” is defined similarly as alkoxyalkyl, except a sulfur atom, rather than an oxygen atom, is present.

The term “alkylthio” and “arylthio” are defined as —SR, wherein R is alkyl or aryl, respectively.

The term “alkylsulfinyl” is defined as R—SO₂, wherein R is alkyl.

The term “alkylsulfonyl” is defined as R—SO₃, wherein R is alkyl.

The term “aryl”, alone or in combination, means a carbocyclic aromatic system containing one, two or three rings wherein such rings may be attached together in a pendent manner or may be fused. Examples of aryl groups include phenyl, benzyl and biphenyl. The “aryl” group can be optionally substituted where possible with one or more of the moieties selected from the group consisting of alkyl, hydroxyl, halo, nitro, cyano, alkenyl, alkynyl, heteroaryl, heterocyclic, carbocycle, alkoxy, oxo, aryloxy, arylalkoxy, cycloalkyl, tetrazolyl, heteroaryloxy; heteroarylalkoxy, carbohydrate, amino acid, amino acid esters, amino acid amides, alditol, haloalkylthio, haloalkoxy, haloalkyl, hydroxyl, carboxyl, acyl, acyloxy, amino, aminoalkyl, aminoacyl, amido, alkylamino, dialkylamino, arylamino, nitro, cyano, thiol, imide, sulfonic acid, sulfate, sulfonate, sulfonyl, alkylsulfonyl, aminosulfonyl, alkylsulfonylamino, haloalkylsulfonyl, sulfanyl, sulfinyl, sulfamoyl, carboxylic ester, carboxylic acid, amide, phosphonyl, phosphinyl, phosphoryl, thioester, thioether, oxime, hydrazine, carbamate, phosphonic acid, phosphate, phosphonate, phosphinate, sulfonamido, carboxamido, hydroxamic acid, sulfonylimide or any other desired functional group that does not inhibit the pharmacological activity of this compound, either unprotected, or protected as necessary, as known to those skilled in the art. In addition, adjacent groups on an “aryl” ring may combine to form a 5- to 7-membered saturated or partially unsaturated carbocyclic, aryl, heteroaryl or heterocyclic ring, which in turn may be substituted as above.

The term “halo” is defined herein to include fluoro, bromo, chloro, and iodo.

The term “heterocyclic” refers to a nonaromatic cyclic group that may be partially (contains at least one double bond) or fully saturated and wherein there is at least one heteroatom, such as oxygen, sulfur, nitrogen, or phosphorus in the ring. The term heteroaryl or heteroaromatic, as used herein, refers to an aromatic that includes at least one sulfur, oxygen, nitrogen or phosphorus in the aromatic ring. Nonlimiting examples of heterocylics and heteroaromatics are pyrrolidinyl, tetrahydrofuryl, piperazinyl, piperidinyl, morpholino, thiomorpholino, tetrahydropyranyl, imidazolyl, pyrrolinyl, pyrazolinyl, indolinyl, dioxolanyl, or 1,4-dioxanyl, aziridinyl, furyl, furanyl, pyridyl, pyrimidinyl, benzoxazolyl, 1,2,4-oxadiazolyl, 1,3,4-oxadiazolyl, 1,3,4-thiadiazole, indazolyl, 1,3,5-triazinyl, thienyl, isothiazolyl, imidazolyl, tetrazolyl, pyrazinyl, benzofuranyl, quinolyl, isoquinolyl, benzothienyl, isobenzofuryl, pyrazolyl, indolyl, isoindolyl, benzimidazolyl, purinyl, carbazolyl, oxazolyl, thiazolyl, benzothiazolyl, isothiazolyl, 1,2,4-thiadiazolyl, isooxazolyl, pyrrolyl, quinazolinyl, cinnolinyl, phthalazinyl, xanthinyl, hypoxanthinyl, pyrazole, imidazole, 1,2,3-triazole, 1,2,4-triazole, 1,2,3-oxadiazole, thiazine, pyridazine, or pteridinyl wherein said heteroaryl or heterocyclic group can be optionally substituted with one or more substituent selected from the same substituents as set out above for aryl groups. Functional oxygen and nitrogen groups on the heteroaryl group can be protected as necessary or desired. Suitable protecting groups can include trimethylsilyl, dimethylhexylsilyl, t-butyldimethylsilyl, and t-butyldiphenylsilyl, trityl or substituted trityl, alkyl groups, acyl groups such as acetyl and propionyl, methanesulfonyl, and p-toluenelsulfonyl.

In the structures herein, for a bond lacking a substituent, the substituent is methyl or methylene, for example,

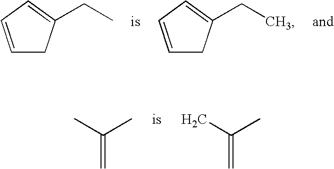

When no substituent is indicated as attached to a carbon atom on a ring, it is understood that the carbon atom contains the appropriate number of hydrogen atoms. In addition, when no substituent is indicated as attached to a carbonyl group or a nitrogen atom, for example, the substituent is understood to be hydrogen, e.g.,

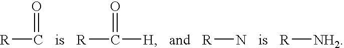

The notation N(Rb)₂ is used to denote two Rb groups attached to a common nitrogen atom. When used in such notation, the Rb group can be the same or different, and is selected from the group as defined by the Rb group.

Nonlimiting examples of cycloalkyl, cycloalkenyl, heterocycloalkyl, and heterocycloalkenyl ring systems useful in compounds of the present invention include, but are not limited to,

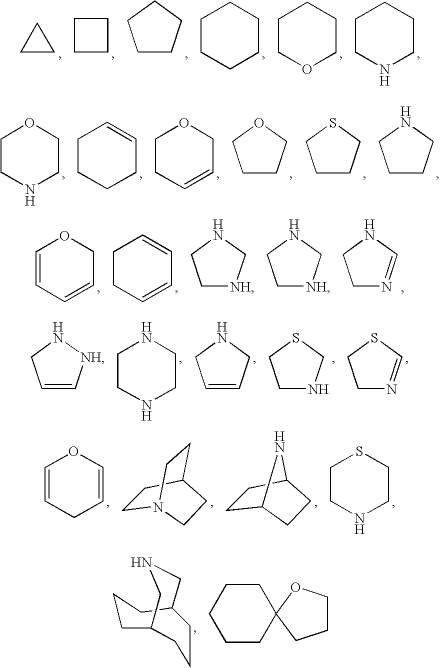

The terms “protecting group” or “protected” refers to a substituent that protects various sensitive or reactive groups present, so as to prevent said groups from interfering with a reaction. Such protection may be carried out in a well-known manner as taught by Greene, et al., Protective Groups in Organic Synthesis, John Wiley and Sons, Third Edition, 1999 or the like. The protecting group may be removed after the reaction in any manner known by those skilled in the art. Non-limiting examples of protecting groups suitable for use within the present invention include but are not limited to allyl, benzyl (Bn), tertiary-butyl (t-Bu), methoxymethyl (MOM), p-methoxybenzyl (PMB), trimethylsilyl (TMS), dimethylhexylsily (TDS)l, t-butyldimethylsilyl (TBS or TBDMS), and t-butyldiphenylsilyl (TBDPS), tetrahydropyranyl (THP), trityl (Trt) or substituted trityl, alkyl groups, acyl groups such as acetyl (Ac) and propionyl, methanesulfonyl (Ms), and p-toluenesulfonyl (Ts). Such protecting groups can form, for example in the instances of protecting hydroxyl groups on a molecule: ethers such as methyl ethers, substituted methyl ethers, substituted alkyl ethers, benzyl and substituted benzyl ethers, and silyl ethers; and esters such as formate esters, acetate esters, benzoate esters, silyl esters and carbonate esters, as well as sulfonates, and borates.

The term “prodrug” as used herein refers to compounds that are transformed in vivo to a compound of the present invention, for example, by hydrolysis. Prodrug design is discussed generally in Hardma et al. (Eds.), Goodman and Gilman's The Pharmacological Basis of Therapeutics, 9th ed., pp. 11-16 (1996). A thorough discussion is also provided by Higuchi, et al., in Prodrugs as Novel Delivery Systems, Vol. 14, ASCD Symposium Series, and in Roche (ed.), Bioreversible Carriers in Drug Design, American Pharmaceutical Association and Pergamon Press (1987). Typically, administration of a drug is followed by elimination from the body or some biotransformation whereby the biological activity of the drug is reduced or eliminated. Alternatively, a biotransformation process can lead to a metabolic by-product that is more or equally active compared to the drug initially administered. Increased understanding of these biotransformation processes permits the design of so-called “prodrugs,” which, following a biotransformation, become more physiologically active in their altered state. Prodrugs, therefore, as used within the scope of the present disclosure, encompass compounds that are converted by some means to pharmacologically active metabolites. To illustrate, prodrugs can be converted into a pharmacologically active form through hydrolysis of, for example, an ester or amide linkages thereby introducing or exposing a functional group on the resultant product. The prodrugs can be designed to react with an endogenous compound to form a water-soluble conjugate that further enhances the pharmacological properties of the compound, for example, increased circulatory half-life. Alternatively, prodrugs can be designed to undergo covalent modification on a functional group with, for example, glucuronic acid, sulfate, glutathione, an amino acid, or acetate. The resulting conjugate can be inactivated and excreted in the urine, or rendered more potent than the parent compound. High molecular weight conjugates also can be excreted into the bile, subjected to enzymatic cleavage, and released back into the circulation, thereby effectively increasing the biological half-life of the originally administered compound.

A “therapeutically effective dose” refers to that amount of the compound that results in achieving the desired effect. Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, which is expressed as the ratio of LD₅₀ to ED₅₀. Compounds that exhibit high therapeutic indices (i.e., a toxic dose that is substantially higher than the effective dose) are preferred. The data obtained can be used in formulating a dosage range for use in humans. The dosage of such compounds preferably lies within a range of circulating concentrations that include the ED₅₀ with little or no toxicity. The dosage can vary within this range depending upon the dosage form employed, and the route of administration utilized.

The term “host”, as used herein, refers to a cell or organism that exhibits the properties associated with abnormal cell proliferation. The hosts are typically vertebrates, including both birds and mammals. It is preferred that the mammal, as a host or patient in the present disclosure, is from the family of Primates, Carnivora, Proboscidea, Perissodactyla, Artiodactyla, Rodentia, and Lagomorpha. It is even more preferable that the mammal vertebrate of the present invention be Canis familiaris (dog), Felis catus (cat), Elephas maximus (elephant), Equus caballus (horse), Sus domesticus (pig), Camelus dromedarious (camel), Cervus axis (deer), Giraffa camelopardalis (giraffe), Bos taurus (cattle/cows), Capra hircus (goat), Ovis aries (sheep), Mus musculus (mouse), Lepus brachyurus (rabbit), Mesocricetus auratus (hamster), Cavia porcellus (guinea pig), Meriones unguiculatus (gerbil), and Homo sapiens (human). Most preferably, the host or patient as used within the present invention is Homo sapiens (human). Birds suitable as hosts within the confines of the present invention include Gallus domesticus (chicken) and Meleagris gallopavo (turkey).

### Pharmaceutical Compositions

Hosts, including mammals and particularly humans, suffering from any of the disorders described herein, including abnormal cell proliferation, can be treated by administering to the host an effective amount of a laulimalide analogue as described herein, or a pharmaceutically acceptable prodrug, solvate, ester, and/or salt thereof, optionally in the presence of a pharmaceutically acceptable carrier or diluent. The active materials can be administered by any appropriate route, for example, orally, parenterally, intravenously, intradermally, subcutaneously, transdermally, bronchially, pharyngolaryngeal, intranasally, topically, rectally, intracistemally, intravaginally, intraperitoneally, bucally or as an oral or nasal spray.

The active compound is included in the pharmaceutically acceptable carrier or diluent in an amount sufficient to deliver to the host a therapeutically effective amount of compound to treat abnormal cell proliferation in vivo, without causing serious toxic effects in the host treated. It is to be understood that for any particular subject, specific is dosage regimens can be adjusted over time according to the individual need and the professional judgment of the person administering or supervising the administration of the compositions. The active ingredient may be administered at once, or may be divided into a number of smaller doses to be administered at varying intervals of time.

The term “pharmaceutically acceptable prodrug” or “prodrug,” as used herein, represents those prodrugs of the compounds of the present invention which are, within the scope of sound medical judgment, suitable for use in contact with the tissues of hosts, such as humans and mammals without undue toxicity, irritation, allergic response, and the like, commensurate with a reasonable benefit/risk ratio, and effective for their intended use. Prodrugs of the present invention may be rapidly transformed in vivo to a parent compound of formula (I), for example, by hydrolysis in blood. A thorough discussion is provided in T. Higuchi and V. Stella, Pro-drugs as Novel Delivery Systems, V. 14 of the A.C.S. Symposium Series, and in Edward B. Roche, ed., Bioreversible Carriers in Drug Design, American Pharmaceutical Association and Pergamon Press (1987).

Dosage forms for topical administration of a compound of this invention include powders, sprays, ointments and inhalants. The active compound is mixed under sterile conditions with a pharmaceutically acceptable carrier and any needed preservatives, buffers or propellants which can be required. Opthalmic formulations, eye ointments, powders and solutions are also contemplated as being within the scope of this invention.

Actual dosage levels of active ingredients in the pharmaceutical compositions of this invention can be varied so as to obtain an amount of the active compound(s) which is effective to achieve the desired therapeutic response for a particular host, compositions and mode of administration. The selected dosage level will depend upon the activity of the particular compound, the route of administration, the severity of the condition being treated and the condition and prior medical history of the host being treated. However, it is within the skill of the art to start doses of the compound at levels lower than required to achieve the desired therapeutic effect and to gradually increase the dosage until the desired effect is achieved.

In the treatment or prevention of conditions which require abnormal cellular proliferation inhibition, an appropriate dosage level will generally be about 0.01 to 500 mg per kg host body weight per day which can be administered in single or multiple doses. Preferably, the dosage level will be about 0.1 to about 250 mg/kg per day; more preferably about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions are preferably provided in the form of tablets containing 1.0 to 1000 milligrams of the active ingredient, particularly 1.0, 5.0, 10.0, 15.0. 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient for the symptomatic adjustment of the dosage to the host to be treated. The compounds may be administered on a regimen of 1 to 4 times per day, preferably once or twice per day.

It will be understood, however, that the specific dose level and frequency of dosage for any particular host may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

The compositions of the present invention can also be used as coatings on stents, including intraluminal stents, such as described in, for example, U.S. Pat. Nos. 6,544,544; 6,403,635; 6,273,913; 6,171,609; and 5,716,981.

The compound or a pharmaceutically acceptable ester, salt, solvate or prodrug can be mixed with other active materials that do not impair the desired action, or with materials that supplement the desired action, including other drugs against abnormal cell proliferation. Solutions or suspensions used for parenteral, intradermal, subcutaneous, or topical application can include for example the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating is agents such as ethylenediaminetetraacetic acid; buffers such as acetates, citrates or phosphates and agents for the adjustment of tonicity such as sodium chloride or dextrose. The parental preparation can be enclosed in ampoules, disposable syringes or multiple dose vials made of glass or plastic.

If administered intravenously, preferred carriers are physiological saline or phosphate buffered saline (PBS).

Pharmaceutical compositions of this invention for parenteral injection comprise pharmaceutically acceptable sterile aqueous or nonaqueous solutions, dispersions, suspensions or emulsions and sterile powders for reconstitution into sterile injectable solutions or dispersions. Examples of suitable aqueous and nonaqueous carriers, diluents, solvents or vehicles include water, ethanol, polyols (propylene glycol, polyethylene glycol, glycerol, and the like), suitable mixtures thereof, vegetable oils (such as olive oil) and injectable organic esters such as ethyl oleate. Proper fluidity may be maintained, for example, by the use of a coating such as lecithin, by the maintenance of the required particle size in the case of dispersions, and by the use of surfactants.

These compositions may also contain adjuvants including immunostimulating factors (including immunostimulatory nucleic acid sequences, including those with CpG sequences), preservative agents, wetting agents, emulsifying agents, and dispersing agents. Prevention of the action of microorganisms may be ensured by various antibacterial and antifungal agents, for example, parabens, chlorobutanol, phenol, sorbic acid, and the like. It may also be desirable to include isotonic agents, for example, sugars, sodium chloride and the like. Prolonged absorption of the injectable pharmaceutical form may be brought about by the use of agents delaying absorption, for example, aluminum monostearate and gelatin.

In some cases, in order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Suspensions, in addition to the active compounds, may contain suspending agents, as, for example, ethoxylated isostearyl alcohols, polyoxyethylene sorbitol and sorbitan esters, microcrystalline cellulose, aluminum metahydroxide, bentonite, agar-agar, tragacanth, and mixtures thereof.

The active compounds can also be in micro- or nano-encapsulated form, if appropriate, with one or more excipients.

Injectable depot forms are made by forming microencapsulated matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

The injectable formulations can be sterilized, for example, by filtration through a bacterial-retaining filter or by incorporating sterilizing agents in the form of sterile solid compositions which can be dissolved or dispersed in sterile water or other sterile injectable medium just prior to use. Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic, parenterally acceptable diluent or solvent such as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and salicylic acid; b) binders such as carboxymethylcellulose, alginates, gelatin, polyvinylpyrrolidinone, sucrose, and acacia; c) humectants such as glycerol; d) disintegrating agents such as agar-agar, calcium carbonate, potato or tapioca starch, alginic acid, certain silicates, and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay; and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract in a delayed manner. Examples of embedding compositions which can be used include polymeric substances and waxes.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, and perfuming agents.

Dosage forms for topical or transdermal administration of a compound of this invention include ointments, pastes, creams, lotions, gels, powders, solutions, sprays, inhalants or patches, optionally mixed with degradable or nondegradable polymers. The active component is admixed under sterile conditions with a pharmaceutically acceptable carrier and any needed preservatives or buffers as may be required. Ophthalmic formulation, ear drops, eye ointments, powders and solutions are also contemplated as being within the scope of this invention.

The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons.

Compounds of the present invention may be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes may be used. The present compositions in liposome form may contain, in addition to the compounds of the present invention, stabilizers, preservatives, excipients, and the like. The preferred lipids are the natural and synthetic phospholipids and phosphatidylcholines (lecithins) used separately or together. Methods to form liposomes are known in the art. See, for example, Prescott, Ed., Methods in Cell Biology, Volume XIV, Academic Press, New York, N.Y., (1976), p 33 et seq. and U.S. Pat. No. 4,522,811. For example, liposome formulations may be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of the active compound or its monophosphate, diphosphate, and/or triphosphate derivatives is then introduced into the container. The container is then swirled by hand to free lipid material from the sides of the container and to disperse lipid aggregates, thereby forming the liposomal suspension.

### Controlled Release Formulations

In one embodiment, the active compounds are prepared with carriers that will protect the compound against rapid elimination from the body or rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylacetic acid. Methods for preparation of such formulations will be apparent to those skilled in the art.

The field of biodegradable polymers has developed rapidly since the synthesis and biodegradability of polylactic acid was reported by Kulkarni et al. (“Polylactic acid for surgical implants,” Arch. Surg, 1966, 93, 839). Examples of other polymers which have been reported as useful as a matrix material for delivery devices include polyanhydrides, polyesters such as polyglycolides and polylactide-co-glycolides, polyamino acids such as polylysine, polymers and copolymers of polyethylene oxide, acrylic terminated polyethylene oxide, polyamides, polyurethanes, polyorthoesters, polyacrylonitriles, and polyphosphazenes. See, for example, U.S. Pat. Nos. 4,891,225 and 4,906,474 to Langer (polyanhydrides), 4,767,628 to Hutchinson (polylactide, polylactide-co-glycolide acid), and 4,530,840 to Tice, et al. (polylactide, polyglycolide, and copolymers). See also U.S. Pat. No. 5,626,863 to Hubbell, et al which describes photopolymerizable biodegradable hydrogels as tissue contacting materials and controlled release carriers (hydrogels of polymerized and crosslinked macromers comprising hydrophilic oligomers having biodegradable monomeric or oligomeric extensions, which are end capped monomers or oligomers capable of polymerization and crosslinking); and PCT WO 97/05185 filed by Focal, Inc. directed to multiblock biodegradable hydrogels for use as controlled release agents for drug delivery and tissue treatment agents.

Degradable materials of biological origin are well known, for example, crosslinked gelatin. Hyaluronic acid has been crosslinked and used as a degradable swelling polymer for biomedical applications (U.S. Pat. No. 4,957,744 to Della Valle et. al.; “Surface modification of polymeric biomaterials for reduced thrombogenicity,” Polym. Mater. Sci. Eng., 1991, 62, 731-735]).

Many dispersion systems are currently in use as, or being explored for use as, carriers of substances, particularly biologically active compounds. Dispersion systems used for pharmaceutical and cosmetic formulations can be categorized as either suspensions or emulsions. Suspensions are defined as solid particles ranging in size from a few manometers up to hundreds of microns, dispersed in a liquid medium using suspending agents. Solid particles include microspheres, microcapsules, and nanospheres. Emulsions are defined as dispersions of one liquid in another, stabilized by an interfacial film of emulsifiers such as surfactants and lipids. Emulsion formulations include water in oil and oil in water emulsions, multiple emulsions, microemulsions, microdroplets, and liposomes. Microdroplets are unilamellar phospholipid vesicles that consist of a spherical lipid layer with an oil phase inside, as defined in U.S. Pat. Nos. 4,622,219 and 4,725,442 issued to Haynes. Liposomes are phospholipid vesicles prepared by mixing water-insoluble polar lipids with an aqueous solution. The unfavorable entropy caused by mixing the insoluble lipid in the water produces a highly ordered assembly of concentric closed membranes of phospholipid with entrapped aqueous solution.

U.S. Pat. No. 4,938,763 to Dunn, et al., discloses a method for forming an implant in situ by dissolving a nonreactive, water insoluble thermoplastic polymer in a biocompatible, water soluble solvent to form a liquid, placing the liquid within the body, and allowing the solvent to dissipate to produce a solid implant. The polymer solution can be placed in the body via syringe. The implant can assume the shape of its surrounding cavity. In an alternative embodiment, the implant is formed from reactive, liquid oligomeric polymers which contain no solvent and which cure in place to form solids, usually with the addition of a curing catalyst.

U.S. Pat. No. 5,718,921 discloses microspheres comprising polymer and drug dispersed there within. U.S. Pat. No. 5,629,009 discloses a delivery system for the controlled release of bioactive factors. U.S. Pat. No. 5,578,325 discloses nanoparticles and microparticles of non-linear hydrophilic hydrophobic multiblock copolymers. U.S. Pat. No. 5,545,409 discloses a delivery system for the controlled release of bioactive factors. U.S. Pat. No. 5,494,682 discloses ionically cross-linked polymeric microcapsules.

U.S. Pat. No. 5,728,402 to Andrx Pharmaceuticals, Inc. describes a controlled release formulation that includes an internal phase which comprises the active drug, its salt, ester or prodrug, in admixture with a hydrogel forming agent, and an external phase which comprises a coating which resists dissolution in the stomach. U.S. Pat. Nos. 5,736,159 and 5,558,879 to Andrx Pharmaceuticals, Inc. discloses a controlled release formulation for drugs with little water solubility in which a passageway is formed in situ. U.S. Pat. No. 5,567,441 to Andrx Pharmaceuticals, Inc. discloses a once-a-day controlled release formulation. U.S. Pat. No. 5,508,040 discloses a multiparticulate pulsatile drug delivery system. U.S. Pat. No. 5,472,708 discloses a pulsatile particle based drug delivery system. U.S. Pat. No. 5,458,888 describes a controlled release tablet formulation which can be made using a blend having an internal drug containing phase and an external phase which comprises a polyethylene glycol polymer which has a weight average molecular weight of from 3,000 to 10,000. U.S. Pat. No. 5,419,917 discloses methods for the modification of the rate of release of a drug to form a hydrogel which is based on the use of an effective amount of a pharmaceutically acceptable ionizable compound that is capable of providing a substantially zero-order release rate of drug from the hydrogel. U.S. Pat. No. 5,458,888 discloses a controlled release tablet formulation.

U.S. Pat. No. 5,641,745 to Elan Corporation, plc discloses a controlled release pharmaceutical formulation which comprises the active drug in a biodegradable polymer to form microspheres or nanospheres. The biodegradable polymer is suitably poly-D,L-lactide or a blend of poly-D,L-lactide and poly-D,L-lactide-co-glycolide. U.S. Pat. No. 5,616,345 to Elan Corporation plc describes a controlled absorption formulation for once a day administration that includes the active compound in association with an organic acid, and a multi-layer membrane surrounding the core and containing a major proportion of a pharmaceutically acceptable film-forming, water insoluble synthetic polymer and a minor proportion of a pharmaceutically acceptable film-forming water soluble synthetic polymer. U.S. Pat. No. 5,641,515 discloses a controlled release formulation based on biodegradable nanoparticles. U.S. Pat. No. 5,637,320 discloses a controlled absorption formulation for once a day administration. U.S. Pat. Nos. 5,580,580 and 5,540,938 are directed to formulations and their use in the treatment of neurological diseases. U.S. Pat. No. 5,533,995 is directed to a passive transdermal device with controlled drug delivery. U.S. Pat. No. 5,505,962 describes a controlled release pharmaceutical formulation.

In one embodiment of the invention, stents are provided which comprise a generally tubular structure, which contains or is coated, filled or interspersed with compounds of the present invention, optionally with one or more other anti-angiogenic compounds and/or compositions. Methods are also provided for expanding the lumen of a body passageway, comprising inserting the stent into the passageway, such that the passageway is expanded.

The stents can be provided for eliminating biliary obstructions by inserting a biliary stent into a biliary passageway; for eliminating urethral obstructions by inserting a urethral stent into a urethra; for eliminating esophageal obstructions by inserting an esophageal stent into an esophagus; and for eliminating trachealibronchial obstructions by inserting a tracheal/bronchial stent into the trachea or bronchi.

In one embodiment of the present invention, the compound of the present invention is delivered to the site of arterial injury via a stent. In one approach, the therapeutic agent is incorporated into a polymer material which is then coated on or delivered onto or incorporated into at least a portion of the stent structure. To improve the clinical performance of stents, a therapeutic agent can be applied as a coating to the stent, attached to a covering or membrane, embedded on the surface material via ion bombardment or dripped onto the stent or to holes or reservoirs in a part of the stent that act as reservoirs. Therefore, in one embodiment of the present invention, the compounds are applied, attached, dripped and/or embedded to the stent by known methods.

The stents can be designed from a single piece of metal, such as from wire coil or thin walled metal cylinders, or from multiple pieces of metal. In a separate embodiment, the stents are designed from biodegradable materials such as polymers or organic fabrics. In one embodiment, the surface of the stent is solid. The stent is generally thin walled and can include a number of struts and optionally a number of hinges between the struts that are capable of focusing stresses.

In one embodiment, the stent structure includes a plurality of holes or, in a separate embodiment, a plurality of recesses which can act as reservoirs and may be loaded with the drug. The stent can be designed with particular sites that can incorporate the drug, or multiple drugs, optionally with a biodegradable or non-biodegradable matrix. The sites can be holes, such as laser drilled holes, or recesses in the stent structure that may be filled with the drug or may be partially filled with the drug. In one embodiment, a portion of the holes are filled with other therapeutic agents, or with materials that regulate the release of the drug or drugs. One advantage of this system is that the properties of the coating can be optimized for achieving superior biocompatibility and adhesion properties, without the addition requirement of being able to load and release the drug. The size, shape, position, and number of reservoirs can be used to control the amount of drug, and therefore the dose delivered.

In another embodiment, the surface of the stent can be coated with one or more compositions containing the compound of the invention. In one embodiment, a coating or membrane of biocompatible material could be applied over the reservoirs which would control the diffusion of the drug from the reservoirs to the artery wall. The coating may also be a sheath covering the surface of the stent. The coating may also be interspersed on the surface of the stent. Coatings or fillings are generally accomplished by dipping, spraying or printing the drug on or into the stent, for example through ink jet type techniques.

The compounds of the present invention are optionally applied in non-degradable microparticulates or nanoparticulates or biodegradable microparticulates or nanoparticulates. In one embodiment, the microparticles or nanoparticles are formed of a polymer containing matrix that biodegrades by random, nonenzymatic, hydrolytic scissioning, such as a structure formed from a mixture of thermoplastic polyesters (e.g., polylactide or polyglycolide) or a copolymer of lactide and glycolide components. The lactide/glycolide structure has the added advantage that biodegradation thereof forms lactic acid and glycolic acid, both normal metabolic products of mammals.

The present invention also provides therapeutic methods and therapeutic dosage forms involving administration of the compounds of the invention in combination with an inhibitor of vascular smooth muscle cell contraction to a vascular lumen, allowing the normal hydrostatic pressure to dilate the vascular lumen. Such contraction inhibition may be achieved by actin inhibition, which is preferably achievable and sustainable at a lower dose level than that necessary to inhibit protein synthesis. Consequently, the vascular smooth muscle cells synthesize protein required to repair minor cell trauma and secrete interstitial matrix, thereby facilitating the fixation of the vascular lumen in a dilated state near its maximal systolic diameter. This phenomenon constitutes a biological stenting effect that diminishes or prevents the undesirable recoil mechanism that occurs in up to 25% of the angioplasty procedures classified as successful based on an initial post-procedural angiogram. Cytochalasins (which inhibit the polymerization of G- to F-actin which, in turn, inhibits the migration and contraction of vascular smooth muscle cells) are the preferred therapeutic agents for use in this embodiment of the present invention. Free therapeutic agent protocols of this type effect a reduction, a delay, or an elimination of stenosis after angioplasty or other vascular surgical procedures. Preferably, free therapeutic agent is administered directly or substantially directly to vascular smooth muscle tissue. Such administration is preferably effected by an infusion catheter, to achieve a 10⁻³ M to 10⁻¹² M concentration of said therapeutic agent at the site of administration in a blood vessel.

The compounds of the present invention can be used in the form of pharmaceutically acceptable salts derived from inorganic or organic acids. By “pharmaceutically acceptable salt” is meant those salts which are, within the scope of sound medical judgement, suitable for use in contact with the tissues of humans and lower animals without undue toxicity, irritation, allergic response and the like and are commensurate with a reasonable benefit/risk ratio. Pharmaceutically acceptable salts are well-known in the art. For example, P. H. Stahl, et al. describe pharmaceutically acceptable salts in detail in “Handbook of Pharmaceutical Salts: Properties, Selection, and Use” (Wiley VCH, Zürich, Switzerland: 2002). The salts can be prepared in situ during the final isolation and purification of the compounds of the present invention or separately by reacting a free base function with a suitable organic acid. Representative acid addition salts include, but are not limited to acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, fumarate, hydrochloride, hydrobromide, hydroiodide, 2-hydroxyethansulfonate (isethionate), lactate, maleate, methanesulfonate, nicotinate, 2-naphthalenesulfonate, oxalate, pamoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups can be quaternized with such agents as lower alkyl halides such as methyl, ethyl, propyl, and butyl chlorides, bromides and iodides; dialkyl sulfates like dimethyl, diethyl, dibutyl and diamyl sulfates; long chain halides such as decyl, lauryl, myristyl and stearyl chlorides, bromides and iodides; arylalkyl halides like benzyl and phenethyl bromides and others. Water or oil-soluble or dispersible products are thereby obtained. Examples of acids which can be employed to form pharmaceutically acceptable acid addition salts include such inorganic acids as hydrochloric acid, hydrobromic acid, sulphuric acid and phosphoric acid and such organic acids as oxalic acid, maleic acid, succinic acid and citric acid.

Basic addition salts can be prepared in situ during the final isolation and purification of compounds of this invention by reacting a carboxylic acid-containing moiety with a suitable base such as the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as lithium, sodium, potassium, calcium, magnesium and aluminum salts and the like and nontoxic quaternary ammonia and amine cations including ammonium, tetramethylammonium, tetraethylammonium, methylamine, dimethylamine, trimethylamine, triethylamine, diethylamine, ethylamine and the like. Other representative organic amines useful for the formation of base addition salts include ethylenediamine, ethanolamine, diethanolamine, piperidine, piperazine and the like. Preferred salts of the compounds of the present invention include phosphate, tris and acetate.

### Combination Therapy

Compounds of the present invention can be used in combination or alternation with radiation and chemotherapy treatment, including induction chemotherapy, primary (neoadjuvant) chemotherapy, and both adjuvant radiation therapy and adjuvant chemotherapy. In addition, radiation and chemotherapy are frequently indicated as adjuvants to surgery in the treatment of cancer. The goal of radiation and chemotherapy in the adjuvant setting is to reduce the risk of recurrence and enhance disease-free survival when the primary tumor has been controlled. Chemotherapy is utilized as a treatment adjuvant for lung and breast cancer, frequently when the disease is metastatic. Adjuvant radiation therapy is indicated in several diseases including lung and breast cancers. Compounds of the present invention also are useful following surgery in the treatment of cancer in combination with radio- and/or chemotherapy.

Active agents that can be used in combination with a microtubule stabilizer of the present invention include, but are not limited to, alkylating agents, antimetabolites, hormones and antagonists, microtubule stabilizers, radioisotopes, antibodies, as well as natural products, and combinations thereof. For example, a compound of the present invention can be administered with antibiotics, such as doxorubicin and other anthracycline analogs, nitrogen mustards, such as cyclophosphamide, pyrimidine analogs such as 5-fluorouracil, cisplatin, hydroxyurea, and the like. As another example, in the case of mixed tumors, such as adenocarcinoma of the breast, where the tumors include gonadotropin-dependent and gonadotropin-independent cells, the compound can be administered in conjunction with leuprolide or goserelin (synthetic peptide analogs of LH-RH) Other antineoplastic protocols include the use of an inhibitor compound with another treatment modality, e.g., surgery or radiation, also referred to herein as “adjunct anti-neoplastic modalities.”

More specific examples of active agents useful for combination with compounds of the present invention, in both compositions and the methods of the present invention, include but are not limited to alkylating agents, such as nitrogen mustards (e.g., mechlorethamine, cyclophosphamide, ifosfamide, melphalan, and chlorambucil); nitrosureas, alkyl sulfonates, such as busulfan; triazines, such as dacarbazine (DTIC); antimetabolites; folic acid analogs, such as methotrexate and trimetrexate; pyrimidine analogs, such as 5-fluorouracil, fluorodeoxyuridine, gemcitabin, cytosine arabinoside (AraC, cytarabine), 5-azacytidine, and 2,2′-difluorodeoxycytidine; purine analogs, such as 6-mercaptopurine, 6-thioguanine, azathioprine, 2′-deoxycoformycin (pentostatin), erythrohydroxynonyladenine (EHNA), fludarabine phosphate, and 2 chlorodeoxyadenosine (cladribine, 2-CdA); natural products, including antimitotic drugs such as paclitaxel (Taxol®), vinca alkaloids (e.g., vinblastine (VLB), vincristine, and vinorelbine), Taxotere® (docetaxel), estramustine, estramustine phosphate, colchicine, bryostatin, combretastatin (e.g., combretastatin A-4 phosphate, combretastatin A-1 and combretastatin A-3, and their phosphates), dolastatins 10-15, podophyllotoxin, and epipodophylotoxins (e.g., etoposide and teniposide); antibiotics, such as actimomycin D, daunomycin (rubidomycin), doxorubicin (adriamycin), mitoxantrone, idarubicin, bleomycins, plicamycin (mithramycin), mitomycinC, dactinomycin, and tobramycin; enzymes, such as L-asparaginase; biological response modifiers, such as interferon-alpha, IL-2, G-CSF, and GM-CSF; differentiation agents; retinoic acid derivatives; radiosensitizers, such as metronidazole, misonidazole, desmethylmisonidazole, pimonidazole, etanidazole, nimorazole, RSU 1069, EO9, RB 6145, SR4233, nicotinamide, 5-bromodeozyuridine, 5-iododeoxyuridine, and bromodeoxycytidine; platinum coordination complexes such as cisplatin and carboplatin; anthracenedione; mitoxantrone; substituted ureas, such as hydroxyurea, methylhydrazine derivatives, such as N-methylhydrazine (MIH) and procarbazine; adrenalcortical suppressants, such as mitotane (o,p′-DDD), aminoglutethimide; cytokines, such as interferon alpha, beta, and gamma and Interleukin 2 (IL-2); hormones and hormone antagonists, including adrenocorticosteroids/antagonists such as prednisone and its equivalents, dexamethasone, and aminoglutethimide; progestins, such as hydroxyprogesterone, caproate, medroxyprogesterone acetate, and megesterol acetate; estrogens, such as diethylstilbestrol, ethynyl estradiol, and their equivalents; antiestrogens, such as tamoxifen; androgens, such as testosterone propionate and fluoxymesterone, as well as their equivalents; antiandrogens, such as flutamide; gonadotropin-releasing hormone analogs, such as leuprolide; nonsteroidal antiandrogens, such as flutamide, and photosensitizers, such as hematoporphyrin and its derivatives, Photofrin®, benzoporphyrin and its derivatives, Npe6, tin etioporphyrin (SnET2), pheoboride-α, bacteriochlorophyll-α, naphthalocyanines, phthalocyanines, and zinc phthalocyanines.

In one particular embodiment, the compounds of the invention are administered in combination or alternation with a second agent selected from paclitaxel and an estrogen. In one embodiment, the estrogen or its equivalent is an estrogen metabolite and in a subembodiment it is 2-methoxyestradiol. In a specific embodiment, the compound of the invention is administered in combination or alternation with paclitaxel. In another embodiment, the compound is administered in combination or alternation with 2-methoxyestradiol.

### Abnormal Cellular Proliferation

The compounds described herein are useful to treat or prevent abnormal cellular proliferation. Cellular differentiation, growth, function and death are regulated by a complex network of mechanisms at the molecular level in a multicellular organism. In the healthy animal or human, these mechanisms allow the cell to carry out its designed function and then die at a programmed rate. Abnormal cellular proliferation, notably hyperproliferation, can occur as a result of a wide variety of factors, including genetic mutation, infection, exposure to toxins, autoimmune disorders, and benign or malignant tumor induction.

There are a number of skin disorders associated with cellular hyperproliferation. Psoriasis, for example, is a benign disease of human skin generally characterized by plaques covered by thickened scales. The disease is caused by increased proliferation of epidermal cells of unknown cause. Chronic eczema is also associated with significant hyperproliferation of the epidermis. Other diseases caused by hyperproliferation of skin cells include atopic dermatitis, lichen planus, warts, pemphigus vulgaris, actinic keratosis, basal cell carcinoma and squamous cell carcinoma.

Other hyperproliferative cell disorders include blood vessel proliferation disorders, fibrotic disorders, autoimmune disorders, graft-versus-host rejection, tumors and cancers.

Blood vessel proliferative disorders include angiogenic and vasculogenic disorders. Proliferation of smooth muscle cells in the course of development of plaques in vascular tissue cause, for example, restenosis, retinopathies and atherosclerosis. The advanced lesions of atherosclerosis result from an excessive inflammatory-proliferative response to an insult to the endothelium and smooth muscle of the artery wall (Ross, R. Nature, 362:801-809 (1993)). Both cell migration and cell proliferation play a role in the formation of atherosclerotic lesions.

Fibrotic disorders are often due to the abnormal formation of an extracellular matrix. Examples of fibrotic disorders include hepatic cirrhosis and mesangial proliferative cell disorders. Hepatic cirrhosis is characterized by the increase in extracellular matrix constituents resulting in the formation of a hepatic scar. Hepatic cirrhosis can cause diseases such as cirrhosis of the liver. An increased extracellular matrix resulting in a hepatic scar can also be caused by viral infection such as hepatitis. Lipocytes appear to play a major role in hepatic cirrhosis.

Mesangial disorders are brought about by abnormal proliferation of mesangial cells. Mesangial hyperproliferative cell disorders include various human renal diseases, such as glomerulonephritis, diabetic nephropathy, malignant nephrosclerosis, thrombotic micro-angiopathy syndromes, transplant rejection, and glomerulopathies. Another disease with a proliferative component is rheumatoid arthritis. Rheumatoid arthritis is generally considered an autoimmune disease that is thought to be associated with activity of autoreactive T cells (See, e.g., Harris, E. D., Jr., The New England Journal of Medicine, 322: pp. 1277-1289 (1990)), and to be caused by autoantibodies produced against collagen and IgE.

Other disorders that can include an abnormal cellular proliferative component include Behcet's syndrome, acute respiratory distress syndrome (ARDS), ischemic heart disease, post-dialysis syndrome, leukemia, acquired immune deficiency syndrome, vasculitis, lipid histiocytosis, septic shock and inflammation in general.

A tumor, also called a neoplasm, is a new growth of tissue in which the multiplication of cells is uncontrolled and progressive. A benign tumor is one that lacks the properties of invasion and metastasis and is usually surrounded by a fibrous capsule. A malignant tumor (i.e., cancer) is one that is capable of both invasion and metastasis. Malignant tumors also show a greater degree of anaplasia (i.e., loss of differentiation of cells and of their orientation to one another and to their axial framework) than benign tumors.

Nonlimiting examples of neoplastic diseases or malignancies (e.g., tumors) treatable with the compounds of the present invention include those listed in Table 1.

TABLE 1

| Organ System | Malignancy/Cancer type |
|---|---|
| Skin | Basal cell carcinoma, melanoma, squamous cell carcinoma; cutaneous |
| | T cell lymphoma; Kaposi's sarcoma. |
| Hematological | Acute leukemia, chronic leukemia and myelodysplastic |
| | syndromes. |
| Urogenital | Prostatic, renal and bladder carcinomas, anogenital carcinomas including |
| | cervical, ovarian, uterine, vulvar, vaginal, and those |
| | associated with human papilloma virus infection. |
| Neurological | Gliomas including glioblastomas, astrocytoma, ependymoma, |
| | medulloblastoma, oligodendroma; meningioma, pituitary |
| | adenoma, neuroblastoma, craniopharyngioma. |
| Gastrointestinal | Colon, colorectal, gastric, esophageal, mucocutaneous |
| | carcinomas. |
| Breast | Breast cancer including estrogen receptor and progesterone |
| | Receptor positive or negative subtypes, soft tissue tumors. |
| Lung | small cell lung cancer, non-small cell lung cancer, mesothelioma |
| Metastasis | Metastases resulting from the neoplasms. |
| Skeletal | Osteoma; osteoblastoma; osteosarcoma; intermedullary |
| | osteosarcoma; osteochondroma, enchondroma; |
| | Enchondromatosis (Ollier's Disease); Mafucci Syndrome; |
| | malignant fibrous histeocytoma; chondrosarcoma; |
| | rhabdomyosarcoma; leiomyosarcoma, myeloma; fibrous |
| | dysplasia; desmoplastic fibroma; Extragnathic Fibromyxoma; |
| | Benign Fibrous Histiocytoma; solitary fibrous tumor |
| Diffuse Tumors | Lymphoma (non-Hodgkin's or Hodgkin's), sickle cell anemia. |
| Liver/Kidneys | Heptoma, cholangiocarcinoma; lymphedema; renal cell cancer; transitional |
| | cell cancer; Wilm's tumour |
| Other | Angiomata, angiogenesis associated with the neoplasms. |

### Antiangiogenesis

The compounds described herein are also useful as anti-angiogenesis agents. Normal angiogenesis plays an important role in a variety of processes including embryonic development, wound healing and several components of female reproductive function. Undesirable or pathological angiogenesis has been associated with disease states including diabetic retinopathy, psoriasis, cancer, rheumatoid arthritis, atheroma, Kaposi's sarcoma and haemangioma (Fan, et al, Trends Pharmacol. Sci. 16: pp. 57-66 (1995); Folkman, Nature Medicine 1: pp. 27-31 (1995)). Formation of new vasculature by angiogenesis is a key pathological feature of several diseases (J. Folkman, New England Journal of Medicine, 333, pp. 1757-1763 (1995)). For example, for a solid tumor to grow it must develop its own blood supply upon which it depends critically for the provision of oxygen and nutrients; if this blood supply is mechanically shut off the tumor undergoes necrotic death. Neovascularisation is also a clinical feature of skin lesions in psoriasis, of the invasive pannus in the joints of rheumatoid arthritis hosts and of atherosclerotic plaques. Retinal neovascularisation is pathological in macular degeneration and in diabetic retinopathy.

Reversal of neovascularisation by damaging the newly-formed vascular endothelium is expected to have a beneficial therapeutic effect. In one aspect, the present invention is based on the discovery that laulimalide is a potent antiangiogenic compound. Consequently, in one embodiment of the present disclosure, laulimalide analogs, such as the compounds of principal embodiments I-XI and formulas I-XXIII described herein, are expected to specifically damage or otherwise inhibit newly formed vasculature without affecting the normal, established vascular endothelium of the host species, a property of value in the treatment of disease states associated with angiogenesis.

In accordance with such antiangiogenic behavior, it is expected that compounds of the present invention can be used in the treatment of angiogenic-related diseases including but not limited to: diseases associated with M-protein; cancers and tumors, such as those described previously and listed in Table 1; liver diseases; von-Hippel-Lindau disease; VEGF-related diseases and disorders; and numerous vascular (blood-vessel) diseases, which include but are not limited to abetalipoproteinemia; aneurysms; angina (angina pectoris), antiphospholipid syndrome; aortic stenosis; aortitis; arrhythmias; atherosclerosis, arteriosclerosis; arteritis; Asymmetric Septal Hypertrophy (ASH); atherosclerosis; athletic heart syndrome; atrial fibrillation; bacterial endocarditis; Barlow's Syndrome (Mitral Valve Prolapse); bradycardia; Buerger's Disease (Thromboangitis Obliterans); cardiac arrest; cardiomegaly; cardiomyopathy; carditis; carotid artery disease; high blood cholesterol; coarctation of the aorta; congenital heart diseases (congenital heart defects); congestive heart failure; coronary artery disease; coronary heart disease; Eisenmenger's Syndrome; embolism; endocarditis; erythromelalgia; fibrillation; myocardial infarction; congential heart disease; heart murmurs; hemangiomas; hypercholesterolemia; hyperlipidemia; hyperipoproteinemia; hypertriglyceridemia; hypertension; hypercholesterolemia Familial; renovascular hypertension; steroid hypertension; hypobetalipoproteinema; hypolipoproteinemia; hypotension (low blood pressure); idiopathic infantile arterial calcification; Kawasaki Disease (Mucocutaneous Lymph Node Syndrome, Mucocutaneous Lymph Node Disease, Infantile Polyarteritis); lipid transport disorders; metabolic syndrome; microvascular angina; myocarditis; paroxysmal atrial tachycardia (PAT); periarteritis nodosa (Polyarteritis, Polyarteritis Nodosa); Pericardial Tamponade; pericarditis; peripheral vascular disease; pheochromocytoma; phlebitis; pulmonary valve stenosis; Raynaud's disease; renal artery stenosis; rheumatic heart disease; septal defects; silent ischemia; sudden cardiac death; syndrome X; tachycardia; Takayasu's arteritis; Tetralogy of Fallot; thromboembolism; thrombosis; transposition of the Great Vessels; tricuspid atresia; truncus arteriosus; varicose ulcers; varicose veins; vasculitis; ventricular septal defect; Wolff-Parkinson-White Syndrome; and Xanthomatosis (Familial hypercholesterolemia and Type II hyperlipoproteinemia; Hypercholesterolemic Xanthomatosis).

### Preparation of Compounds

### Stereoisomerism and Polymorphism

It is appreciated that compounds of the present invention have chiral centers and may exist in and be isolated in optically active and racemic forms. Some compounds may exhibit polymorphism. It is to be understood that the present invention encompasses any racemic, optically-active, diastereomeric, polymorphic, or stereoisomeric form, or mixtures thereof, of a compound of the invention, which possess the useful properties described herein. It is now well known in the art how to prepare optically active forms (for example, by resolution of the racemic form by recrystallization techniques, by synthesis from optically-active starting materials, by chiral synthesis, or by chromatographic separation using a chiral stationary phase).

Examples of methods to obtain optically active materials include at least the following.
i) physical separation of crystals—a technique whereby macroscopic crystals of the individual enantiomers are manually separated. This technique can be used if crystals of the separate enantiomers exist, i.e., the material is a conglomerate, and the crystals are visually distinct;
ii) simultaneous crystallization—a technique whereby the individual enantiomers are separately crystallized from a solution of the racemate, possible only if the latter is a conglomerate in the solid state;
iii) enzymatic resolutions—a technique whereby partial or complete separation of a racemate by virtue of differing rates of reaction for the enantiomers with an enzyme;
iv) enzymatic asymmetric synthesis—a synthetic technique whereby at least one step of the synthesis uses an enzymatic reaction to obtain an enantiomerically pure or enriched synthetic precursor of the desired enantiomer;
v) chemical asymmetric synthesis—a synthetic technique whereby the desired enantiomer is synthesized from an achiral precursor under conditions that produce asymmetry (i.e., chirality) in the product, which may be achieved using chiral catalysts or chiral auxiliaries;
vi) diastereomer separations—a technique whereby a racemic compound is reacted with an enantiomerically pure reagent (the chiral auxiliary) that converts the individual enantiomers to diastereomers. The resulting diastereomers are then separated by chromatography or crystallization by virtue of their now more distinct structural differences and the chiral auxiliary later removed to obtain the desired enantiomer;
vii) first- and second-order asymmetric transformations—a technique whereby diastereomers from the racemate equilibrate to yield a preponderance in solution of the diastereomer from the desired enantiomer or where preferential crystallization of the diastereomer from the desired enantiomer perturbs the equilibrium such that eventually in principle all the material is converted to the crystalline diastereomer from the desired enantiomer. The desired enantiomer is then released from the diastereomer;
viii) kinetic resolutions—this technique refers to the achievement of partial or complete resolution of a racemate (or of a further resolution of a partially resolved compound) by virtue of unequal reaction rates of the enantiomers with a chiral, non-racemic reagent or catalyst under kinetic conditions;
ix) enantiospecific synthesis from non-racemic precursors—a synthetic technique whereby the desired enantiomer is obtained from non-chiral starting materials and where the stereochemical integrity is not or is only minimally compromised over the course of the synthesis;
x) chiral liquid chromatogaphy—a technique whereby the enantiomers of a racemate are separated in a liquid mobile phase by virtue of their differing interactions with a stationary phase. The stationary phase can be made of chiral material or the mobile phase can contain an additional chiral material to provoke the differing interactions;
xi) chiral gas chromatogaphy—a technique whereby the racemate is volatilized and enantiomers are separated by virtue of their differing interactions in the gaseous mobile phase with a column containing a fixed non-racemic chiral adsorbent phase;
xii) extraction with chiral solvents—a technique whereby the enantiomers are separated by virtue of preferential dissolution of one enantiomer into a particular chiral solvent;
xiii) transport across chiral membranes—a technique whereby a racemate is placed in contact with a thin membrane barrier. The barrier typically separates two miscible fluids, one containing the racemate, and a driving force such as concentration or pressure differential causes preferential transport across the membrane barrier. Separation occurs as a result of the non-racemic chiral nature of the membrane which allows only one enantiomer of the racemate to pass through.

Generally, compounds of the present invention can be prepared according to the synthetic schemes set forth below and in the associated Figures. In the schemes described herein, it is understood in the art that protecting groups can be employed where necessary in accordance with general principles of synthetic chemistry. Such protecting groups are described, for example, in the text by T. W. Greene and P. M. G. Wuts (Protective Groups in Organic Synthesis, 3^{rd} Edition; Wiley Interscience, 1999). These protecting groups are removed in the final steps of the synthesis under, for example, basic, acidic, photolytic, or hydrogenolytic conditions which are readily apparent to those skilled in the art. By employing appropriate manipulation and protection of any chemical functionalities, synthesis of compounds of the present invention not specifically set forth herein can be accomplished by methods analogous to the schemes set forth below. That is, employing different appropriate protecting groups than those described herein would allow one of skill in the art to achieve the products described herein.

The terms “solvent”, “inert organic solvent” or “inert solvent” means a solvent that is inert under the conditions of the reaction being described [including, for example, benzene, toluene, acetonitrile, tetrahydrofuran (“THF”), dimethylformamide (“DMF”), chloroform, methylene chloride (or dichloromethane), diethyl ether, methanol, pyridine and the like]. Unless specified to the contrary, the solvents used in the reactions of the present invention are inert organic solvents.

The synthesis of several of the various compounds of the present invention are set forth below:

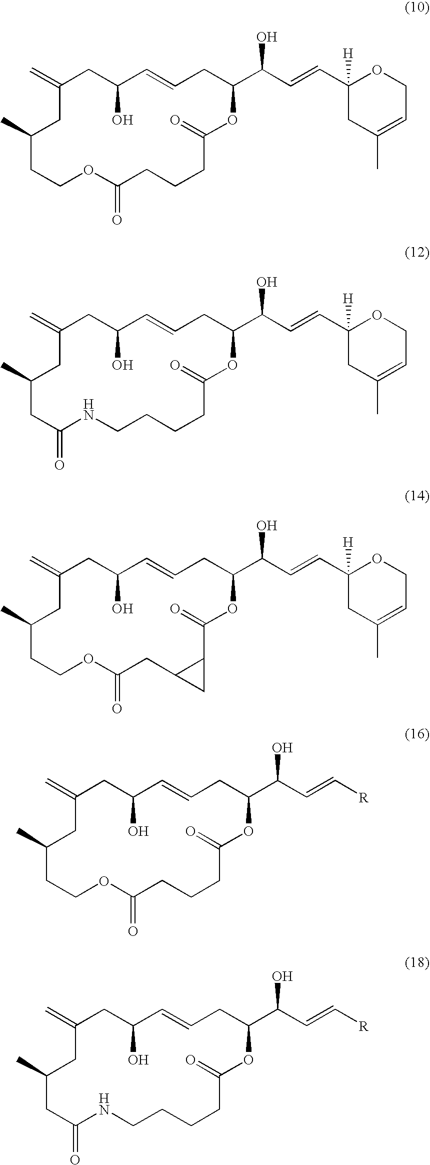

The detailed synthesis of various intermediates and precursors described herein can be found in Wender, P. A., et al., J. Am. Chem. Soc., et al., 124, pp. 4956-4957 (2002), and references cited therein, which is incorporated herein by reference.

Laulimalide analogs (10, 12, 14, 16 and 18) are prepared from the corresponding C₁₅-C₂₇ hydroxyl-protected fragment and the basic C₉-C₁₄ protected fragment, which are prepared via a Sakurai coupling of the alkene (22a or 22b) and the allyl silane (28), as shown in Scheme 1. This would allow for late-stage diversification from the carboxylic acid (30a,b), which is obtainable via intermediate (29a,b).

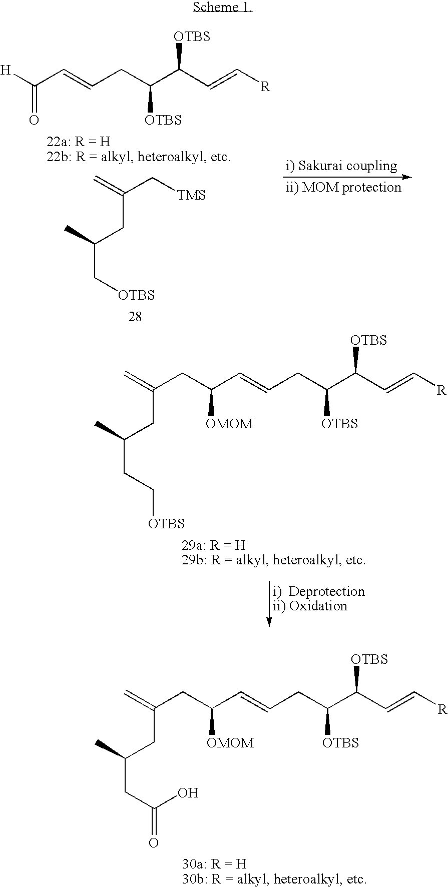

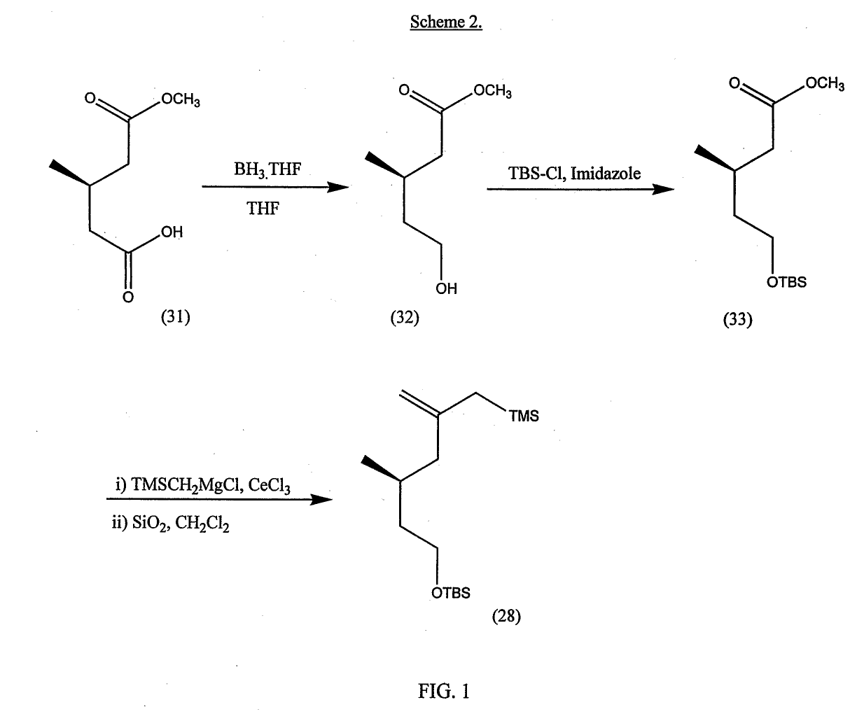

Allyl silane (28) can be prepared by the route shown in FIG. 1 (Scheme 2). Standard borane reduction of commercially available carboxylic acid (31) produces alcohol (32) in good yield. The primary alcohol functionality of alcohol (32) is protected as a tert-butyldimethylsilyl ether (TBS) using TBS-Cl and imidazole (Corey, E. J., et al., J. Am. Chem. Soc., 94, p. 6190 (1972)) to afford silyl ether (33). Elaboration of (33) to allyl silane (28) is facilitated using a cerium-mediated double addition of trimethylsilylmethyl magnesium chloride, followed by a silica-gel catalyzed Peterson olefination.

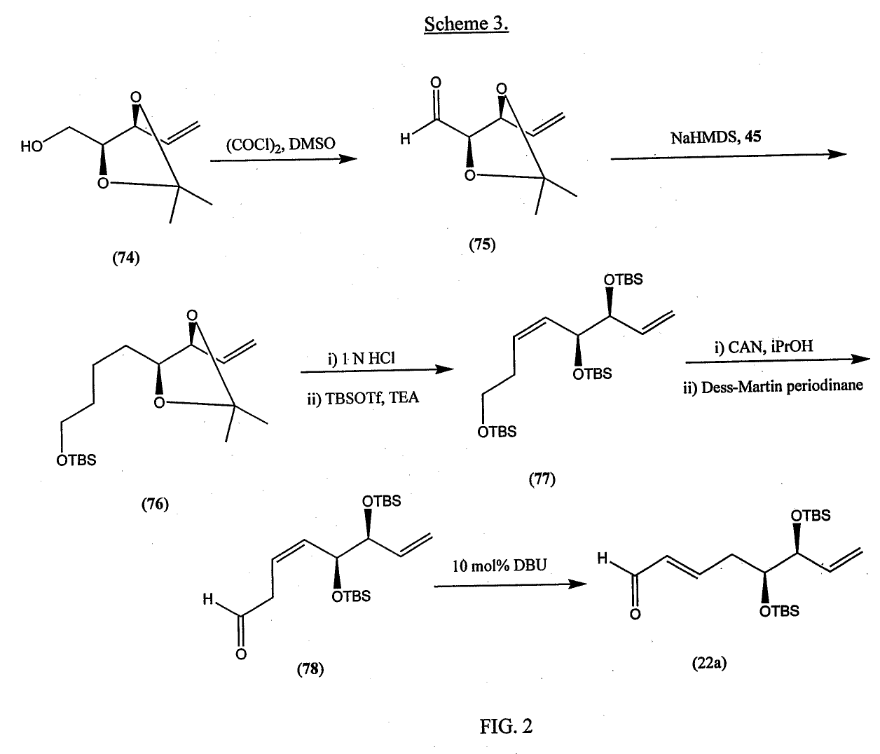

The C₁₅-C₂₃ “top piece” fragment can be prepared from known tartrate compound (74) as shown in Scheme 3 (FIG. 2), providing a facile route to alkene C₂₁-C₂₂ alkene analogues, as well as other diversity analogues via a metathesis reaction, which is described in more detail below. Swern oxidation of alcohol (74) with oxalyl chloride in DMSO provides aldehyde (75). Treatment of aldehyde (75) with phosphonium salt (45) (obtained in 3 steps from 1-chloropropanol) under Wittig conditions using sodium hexamethyldisilazane to produce olefin (76) in good yield. Global deprotection with 2N HCl, followed by subsequent silylation using TBSOTf generates the tris-silyl ether (77). Cerium ammonium nitrate (CAN) in 2-propanol selectively removes the primary silyl group to provide the homoallylic alcohol, which is subsequently oxidized under buffered Dess-Martin conditions to provide aldehyde (78). Aldehyde (78) then underwent base-induced isomerisation to afford the β,γ-unsaturated aldehyde (22a).

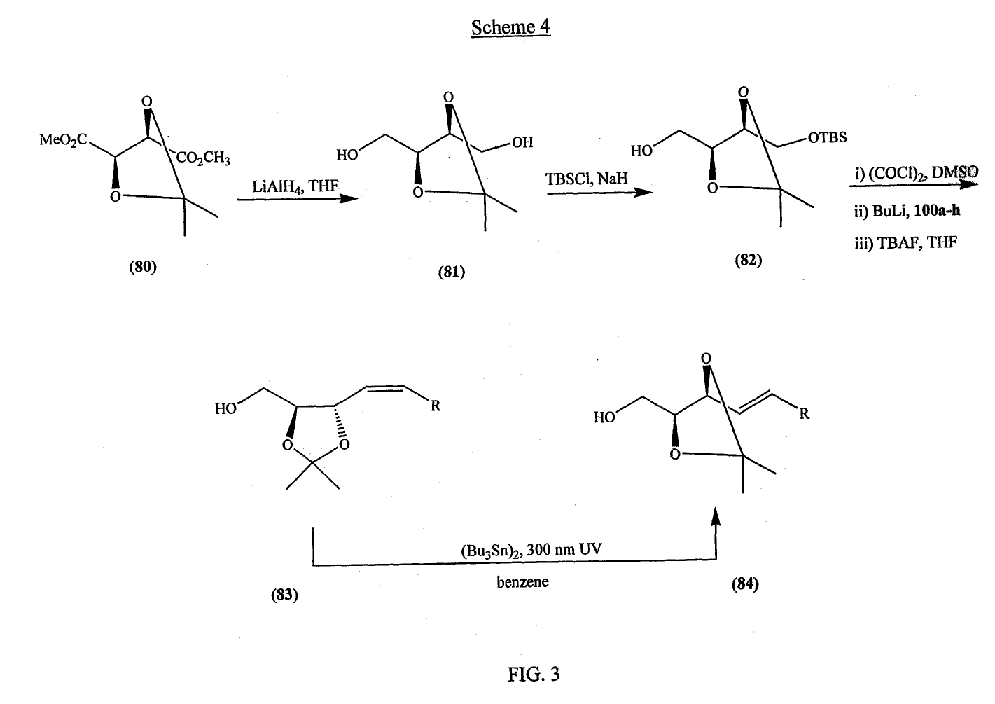

The C₁₅-C₂₇ “top piece” fragment is also prepared from commercially available dimethyl L-tartrate derivative (80) as shown in Scheme 4 (FIG. 3), so as to introduce diversity at the C₂₃-position. A standard lithium aluminum hydride (LiAlH₄) reduction of 2,3-o-isopropylidene-L-tartrate (Aldrich Chemical Co.) in THF, followed by silylation of the resultant diol (81) with t-butyldimethylsilyl chloride and sodium hydride produced mono-silyl ether (82) in high yield. Swern oxidation to the aldehyde, followed by a Wittig olefination with a phosphonium salt (e.g., 100a-h below, obtainable by known processes from the aldehydes, for example) and subsequent deprotection using n-tetrabutylammonium fluoride (TBAF) provides a 4.5:1 mixture of C₂₁-C₂₂ Z-, E-isomers (83) and (84). Irradiation of the Z-isomer under 300 nm UV light in the presence of 20 mol % hexabutyl distannane in benzene at room temperature generates the desired E-isomer (84). In a manner similar to that outlined above for the synthesis of compound (22a), alcohol (84) is then oxidized using Swern conditions to produce aldehyde intermediate (85), which is then treated with phosphonium salt 45 (generated in 3 steps from 1-chloropropanol (Molander, G. A., et al., J. Org. Chem., 61, pp. 5885-5894 (1996)) under Wittig olefination conditions to provide diene (86) in 84% yield over two steps. Global deprotection of (86) with 3N HCl and subsequent silylation using tert-butyldimethylsilyl triflate (TBSOTf) generated tris-silyl ether (41). Cerium ammonium nitrate (CAN) in 2-propanol selectively removed the primary silyl group, providing homoallylic alcohol (42) in satisfactory yields. Oxidation of alcohol (42) under buffered Dess-Martin conditions (Meyer, S. D., et al., J. Org. Chem., 59, pp. 7549-7552 (1994)) produces aldehyde (43) which undergoes base-induced isomerisation to afford β,γ-unsaturated aldehyde (22b).

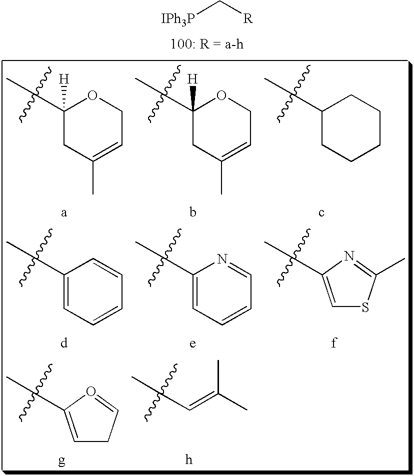

The asymmetric coupling of allyl silane 28 and aldehyde 22a or 22b is carried out using a modification of the asymmetric Sakurai reaction described in the Wender synthesis of laulimalide (Wender, P. A., et al., J. Am. Chem. Soc., 124, 4956-4957 (2002)). As shown in Scheme 5, below, aldehyde 22a or 22b is contacted with the active D-tartrate-derived “CAB” ligand complex (prepared according to Yamamoto, H., et. al., J. Am. Chem. Soc., 115, pp. 11490 (1993)) to afford the coupling product in excellent diastereoselective yield (>20:1). Protection of the C₁₅-hydroxyl as the methoxymethyl (MOM) ether is effected using MOMCl and diisopropylethylamine under standard conditions (Stork, G., et al., J. Am. Chem. Soc., 99, p. 1275 (1977)) to produce compounds 29a or 29b.

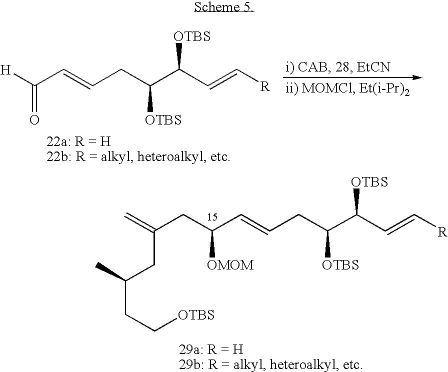

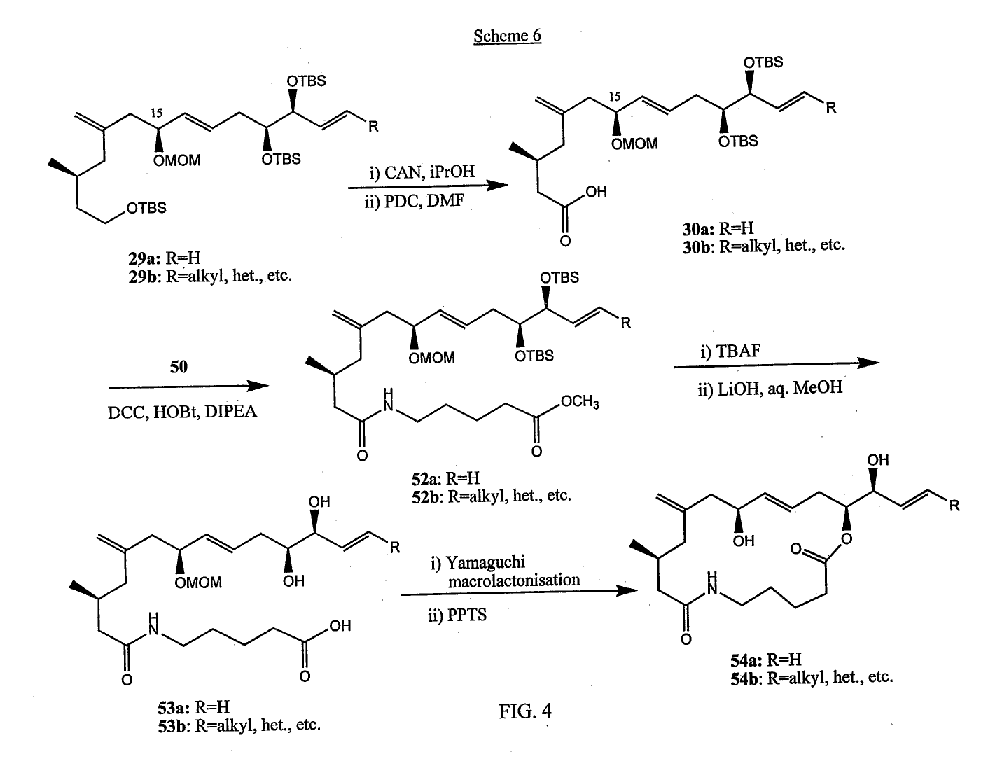

Following successful coupling of the two segments to form the “top piece”, the remaining synthesis of the analogues proceeds smoothly. As shown in Scheme 6 (FIG. 4), selective primary TBS ether deprotection of 29a or 29b affords the primary alcohol, which is oxidized using PDC in DMF to give carboxylic acid 30 or 30b, respectively. Coupling of 30a or 30b with amino ester hydrochloride 50 (available via methylation of 5-aminopentanoic acid (Sigma-Aldrich) using thionyl chloride and methanol, as shown below) using DCC-mediated conditions with the addition of HOBt,

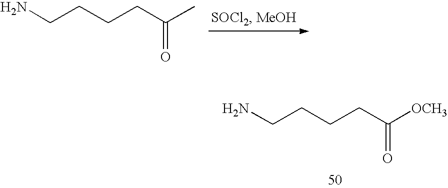

provides amide 52a or 52b in good yield, although any of the known amide-coupling protocols and reagents (see, for example, Han, S-Y., et al., Tetrahedron, 60, pp. 2447-2467 (2004)) are envisioned to be suitable for conducting this reaction. Removal of the secondary TBS ether functionalities from 52 is accomplished using TBAF (1.0 M in THF), followed by saponification using lithium hydroxide to afford diol 53a or 53b. Finally, macrolactonisation is accomplished using the Yamaguchi protocol (Inanaga, J., et al., Bull. Chem. Soc. Jpn., 53, p. 1989 (1979)) of 2,4,6-trichlorobenzoyl chloride (Yamaguchi reagent) with triethylamine and DMAP to give, after purification and acid-catalysed removal of the MOM protecting group (PPTS, tert-BuOH), the Cl₉ macrolides 54a or 54b. When R is a vinyldihydropyranone in compound 54b, then 54b is compound (12).

Compound 54a, wherein R is H, can be transformed into any number of desired C₂₃-analogues by way of a cross-metathesis reaction of the vinyl group, as shown in Scheme 7, below. Generally, compounds such as 54a are reacted with an excess of alkene, such as vinylcyclohexane, in the presence of Grubbs catalyst, second generation (2,1,3-(Bis(mesityl)-2-imidazolidinylidene)dichloro(phenylmethylene)-(tricyclohexylphosphine)ruthenium) in dichloromethane. Following workup, the target C₂₃-laulimalide analogue (55) is obtained in good yield.

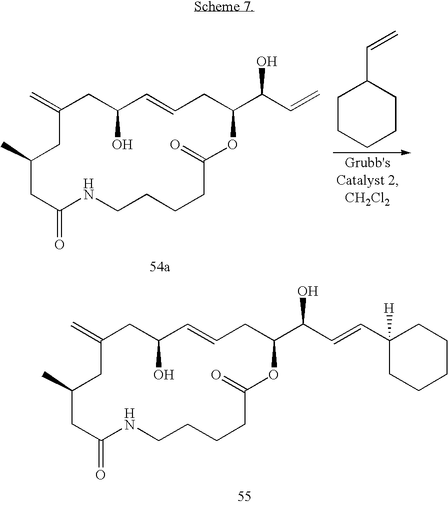

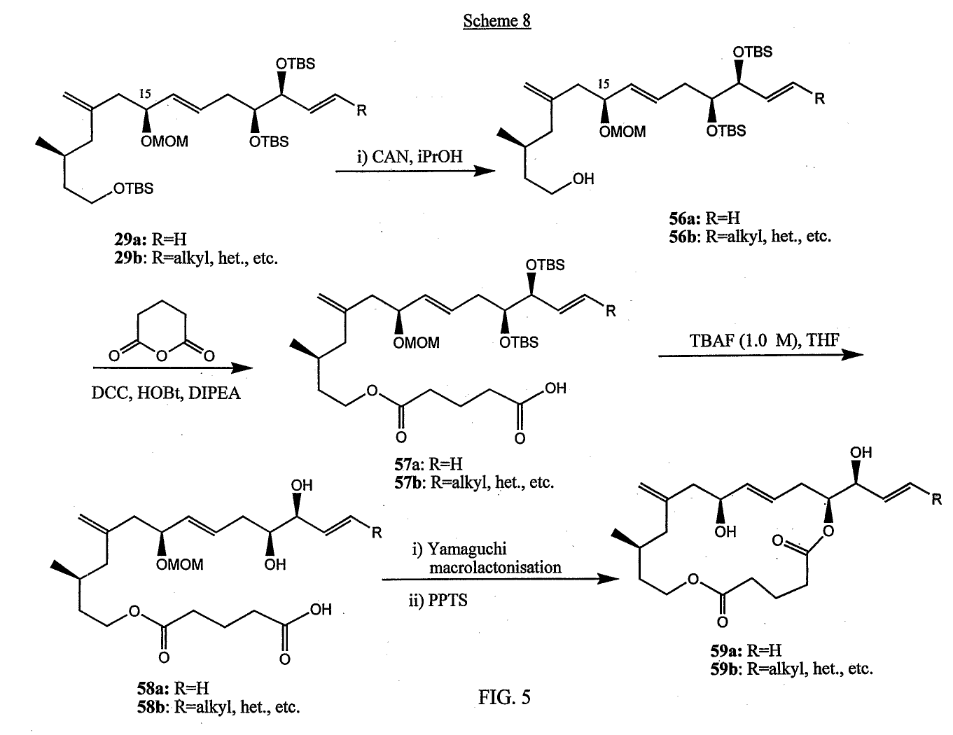

In a similar manner, compound (10) can be prepared, as shown in scheme 8 (FIG. 5). Compound (29a) or (29b) is reacted with CAN in isopropanol to generate alcohol (56). Reaction with glutaric anhydride and triethylamine with a catalytic amount of DMAP provides ester (57). Removal of the secondary TBS ether functionalities from (57) is accomplished using TBAF (1.0 M in THF) to afford diol 58a or 58b. Finally, macrolactonisation is accomplished using the Yamaguchi protocol as before to give, after purification and acid-catalysed removal of the MOM protecting group, the C₁₉ macrolides 59a or 59b. When R is a vinyldihydropyranone in compound 59b, then 59b is compound (10).

As above, compound 59a, wherein R is H, can be transformed into any number of desired C₂₃-analogues, such as compound (10), by way of a cross-metathesis reaction of the vinyl group, as shown in Scheme 7 above. Similarly, various compounds containing the C₁₆-C₁₇ cis-olefin geometry can be prepared from common “top pieces” (29a) and (29b). Laulimalide analogues having an epoxide or other, suitable functionality (such as a cyclopropane ring by way of a Simmons-Smith reaction), can be prepared as generally outlined in scheme 9, below. For example, a C₁₆-C₁₇ epoxide can be incorporated into the analogue (10) or (12) using Sharpless epoxidation conditions (Paterson, I., et al., Org. Lett., 3, pp. 3149-3152 (2002)) to generate the regio- and diastereoselective analogues (11) and (13), respectively.

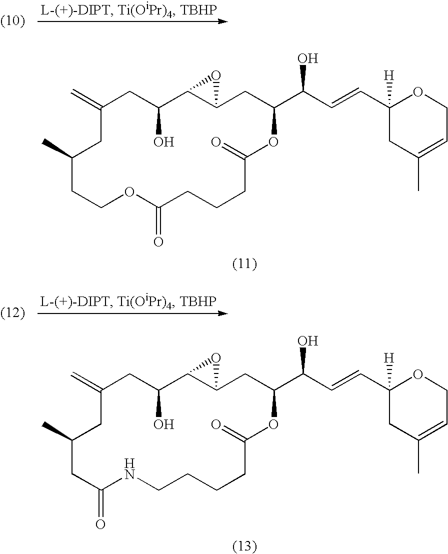

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the scope of the invention.

### EXAMPLE 1

The following macrocycles of Formula XII are prepared, using appropriate reagents and conditions as described herein.

| | |
|---|---|
| | (XII) |
| 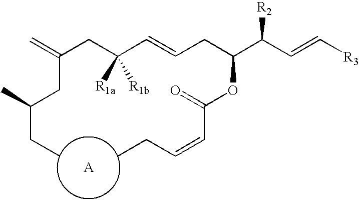 | |

| Compound | R1a/R1b | R2 | R3 | A |
|---|---|---|---|---|
| 201 | OH/H | OH | 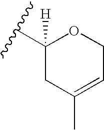 | 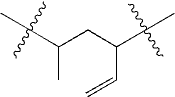 |
| 202 | OH/H | OH | 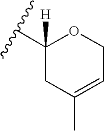 | 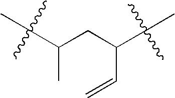 |
| 203 | OH/H | OH | 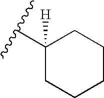 | 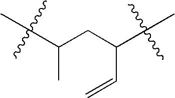 |
| 204 | OH/H | OH | 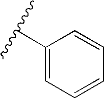 | 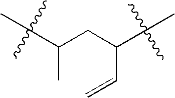 |
| 205 | OH/H | OH | 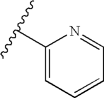 | 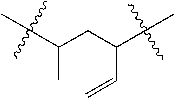 |
| 206 | OH/H | OH | 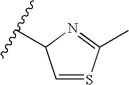 | 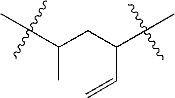 |
| 207 | OH/H | OH | 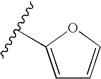 | 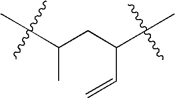 |
| 208 | OH/H | OH | 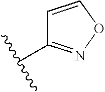 | 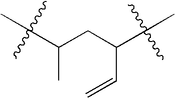 |
| 209 | OH/H | OH | 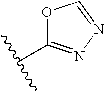 | 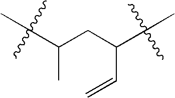 |
| 210 | OH/H | OH | 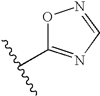 | 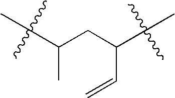 |
| 211 | OH/H | OH | 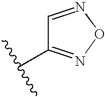 | 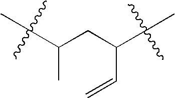 |
| 212 | OH/H | OH | 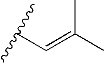 | 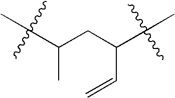 |
| 213 | OH/H | OH | 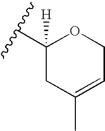 | 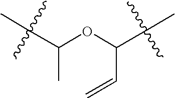 |
| 214 | OH/H | OH | 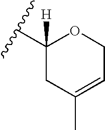 | 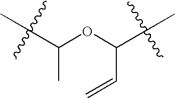 |
| 215 | OH/H | OH | 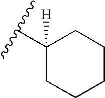 | 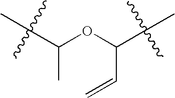 |
| 216 | OH/H | OH | 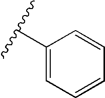 | 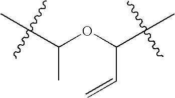 |
| 217 | OH/H | OH | 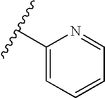 | 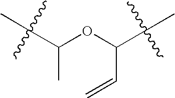 |
| 218 | OH/H | OH | 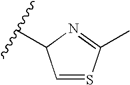 | 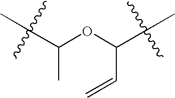 |
| 219 | OH/H | OH | 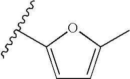 | 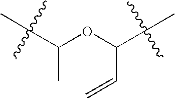 |
| 220 | OH/H | OH | 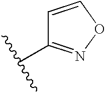 | 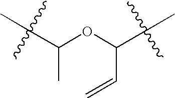 |
| 221 | OH/H | OH | 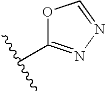 | 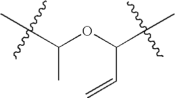 |
| 222 | OH/H | OH | 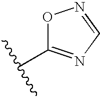 | 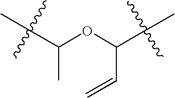 |
| 223 | OH/H | OH | 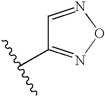 | 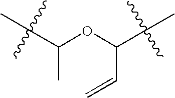 |
| 224 | OH/H | OH | 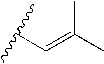 | 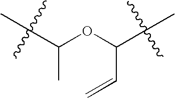 |
| 225 | OH/H | OH | 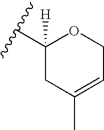 | 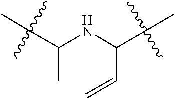 |
| 226 | OH/H | OH | 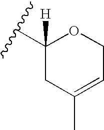 | 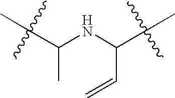 |
| 227 | OH/H | OH | 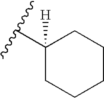 | 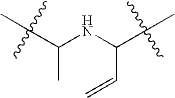 |
| 228 | OH/H | OH | 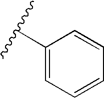 | 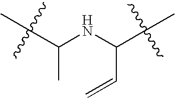 |
| 229 | OH/H | OH | 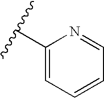 | 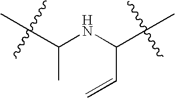 |
| 230 | OH/H | OH | 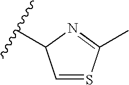 | 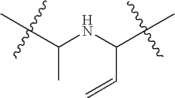 |
| 231 | OH/H | OH | 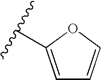 | 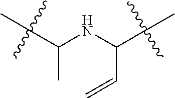 |
| 232 | OH/H | OH | 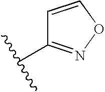 | 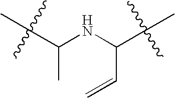 |
| 233 | OH/H | OH | 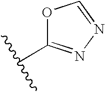 | 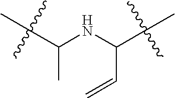 |
| 234 | OH/H | OH | 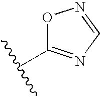 | 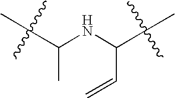 |
| 235 | OH/H | OH | 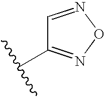 | 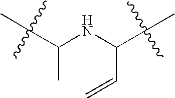 |
| 236 | OH/H | OH | 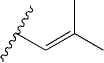 | 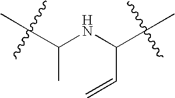 |
| 237 | OH/H | OH | 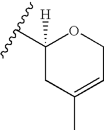 | 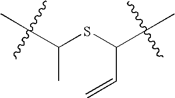 |
| 238 | OH/H | OH | 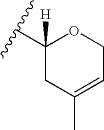 | 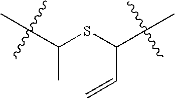 |
| 239 | OH/H | OH | 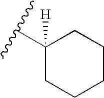 | 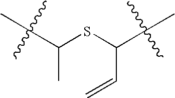 |
| 240 | OH/H | OH | 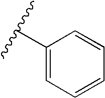 | 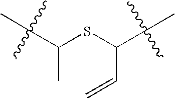 |
| 241 | OH/H | OH | 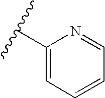 | 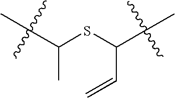 |
| 242 | OH/H | OH | 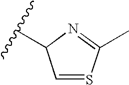 | 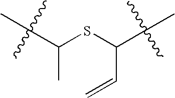 |
| 243 | OH/H | OH | 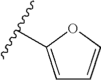 | 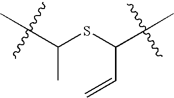 |
| 244 | OH/H | OH | 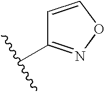 | 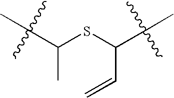 |
| 245 | OH/H | OH | 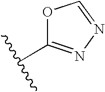 | 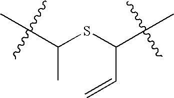 |
| 246 | OH/H | OH | 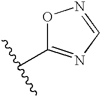 | 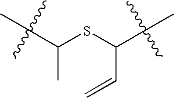 |
| 247 | OH/H | OH | 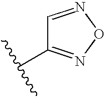 | 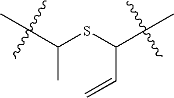 |
| 248 | OH/H | OH | 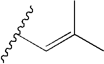 | 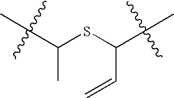 |
| 249 | OH/H | OH | 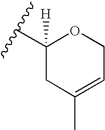 | 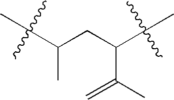 |
| 250 | OH/H | OH | 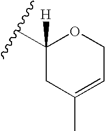 | 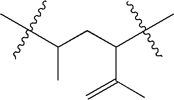 |
| 251 | OH/H | OH | 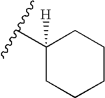 | 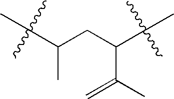 |
| 252 | OH/H | OH | 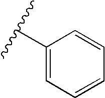 | 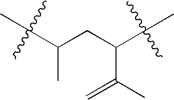 |
| 253 | OH/H | OH | 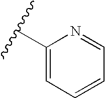 | 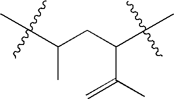 |
| 254 | OH/H | OH | 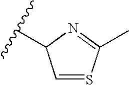 | 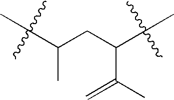 |
| 255 | OH/H | OH | 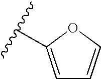 | 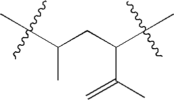 |
| 256 | OH/H | OH | 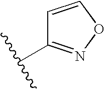 | 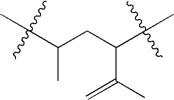 |
| 257 | OH/H | OH | 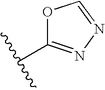 | 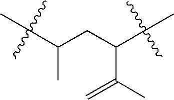 |
| 258 | OH/H | OH | 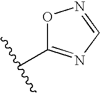 | 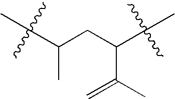 |
| 259 | OH/H | OH | 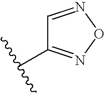 | 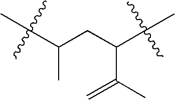 |
| 260 | OH/H | OH | 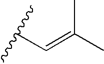 | 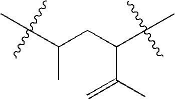 |
| 261 | OH/H | OH | 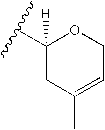 | 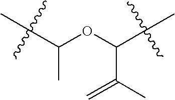 |
| 262 | OH/H | OH | 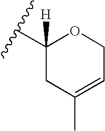 | 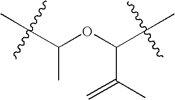 |
| 263 | OH/H | OH | 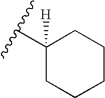 | 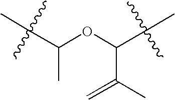 |
| 264 | OH/H | OH | 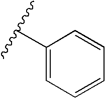 | 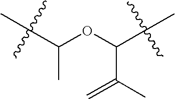 |
| 265 | OH/H | OH | 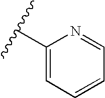 | 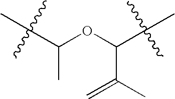 |
| 266 | OH/H | OH | 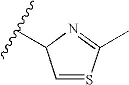 | 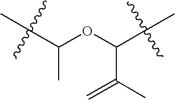 |
| 267 | OH/H | OH | 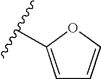 | 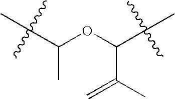 |
| 268 | OH/H | OH | 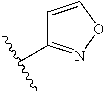 | 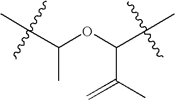 |
| 269 | OH/H | OH | 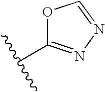 | 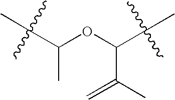 |
| 270 | OH/H | OH | 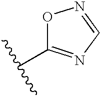 | 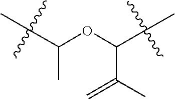 |
| 271 | OH/H | OH | 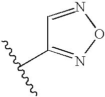 | 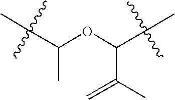 |
| 272 | OH/H | OH | 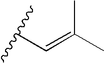 | 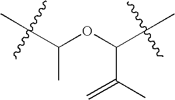 |
| 273 | OH/H | OH | 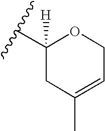 | 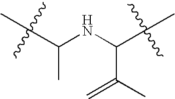 |
| 274 | OH/H | OH | 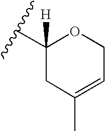 | 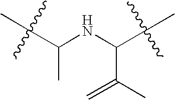 |
| 275 | OH/H | OH | 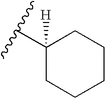 | 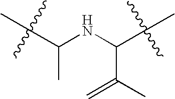 |
| 276 | OH/H | OH | 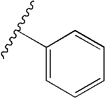 | 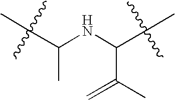 |
| 277 | OH/H | OH | 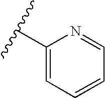 | 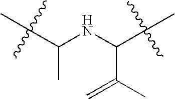 |
| 278 | OH/H | OH | 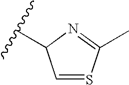 | 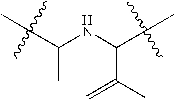 |
| 279 | OH/H | OH | 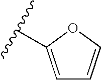 | 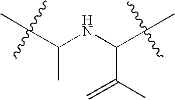 |
| 280 | OH/H | OH | 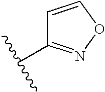 | 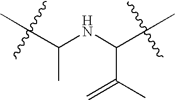 |
| 281 | OH/H | OH | 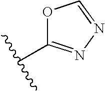 | 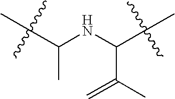 |
| 282 | OH/H | OH | 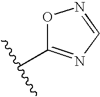 | 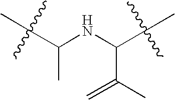 |
| 283 | OH/H | OH | 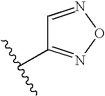 | 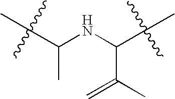 |
| 284 | OH/H | OH | 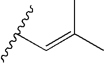 | 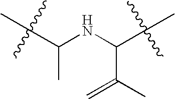 |
| 285 | OH/H | OH | 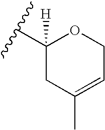 | 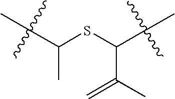 |
| 286 | OH/H | OH | 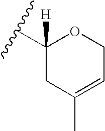 | 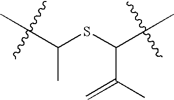 |
| 287 | OH/H | OH | 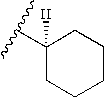 | 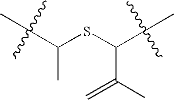 |
| 288 | OH/H | OH | 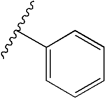 | 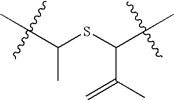 |
| 289 | OH/H | OH | 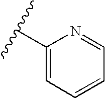 | 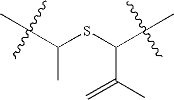 |
| 290 | OH/H | OH | 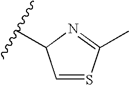 | 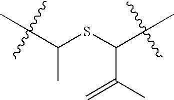 |
| 291 | OH/H | OH | 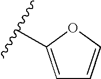 | 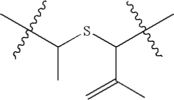 |
| 292 | OH/H | OH | 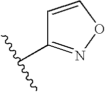 | 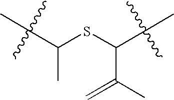 |
| 293 | OH/H | OH | 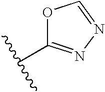 | 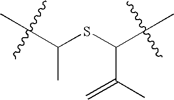 |
| 294 | OH/H | OH | 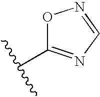 | 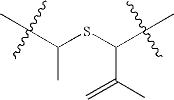 |
| 295 | OH/H | OH | 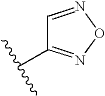 | 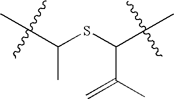 |
| 296 | OH/H | OH | 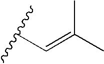 | 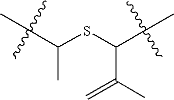 |
| 297 | OH/H | OH | 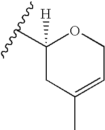 | 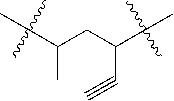 |
| 298 | OH/H | OH | 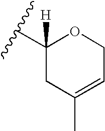 | 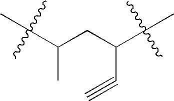 |
| 299 | OH/H | OH | 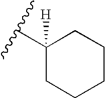 | 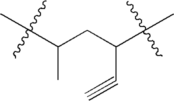 |
| 300 | OH/H | OH | 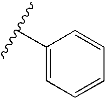 | 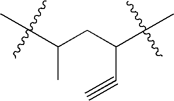 |
| 301 | OH/H | OH | 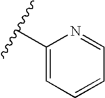 | 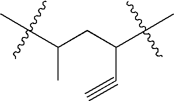 |
| 302 | OH/H | OH | 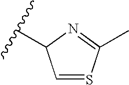 | 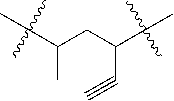 |
| 303 | OH/H | OH | 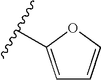 | 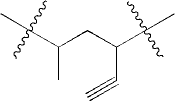 |
| 304 | OH/H | OH | 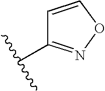 | 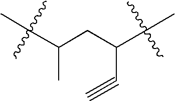 |
| 305 | OH/H | OH | 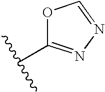 | 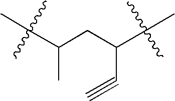 |
| 306 | OH/H | OH | 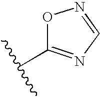 | 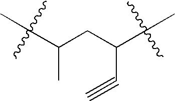 |
| 307 | OH/H | OH | 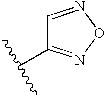 | 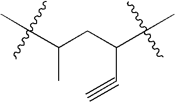 |
| 308 | OH/H | OH | 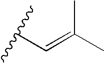 | 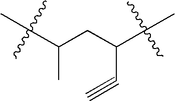 |
| 309 | OH/H | OH | 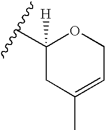 | 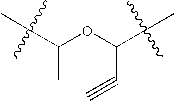 |
| 310 | | | 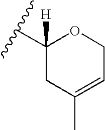 | 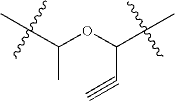 |
| 311 | | | 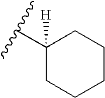 | 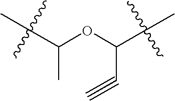 |
| 312 | | | 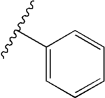 | 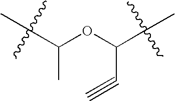 |
| 313 | | | 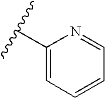 | 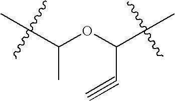 |
| 314 | OH/H | OH | 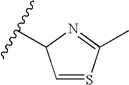 | 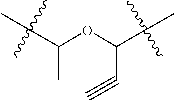 |
| 315 | OH/H | OH | 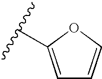 | 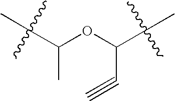 |
| 316 | OH/H | OH | 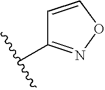 | 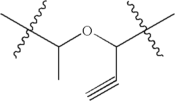 |
| 317 | OH/H | OH | 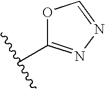 | 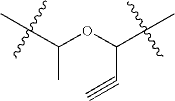 |
| 318 | OH/H | OH | 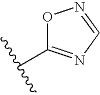 | 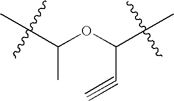 |
| 319 | OH/H | OH | 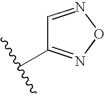 | 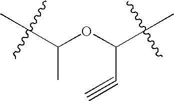 |
| 320 | OH/H | OH | 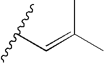 | 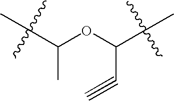 |
| 321 | OH/H | OH | 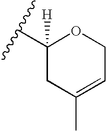 | 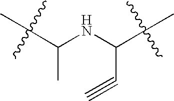 |
| 322 | OH/H | OH | 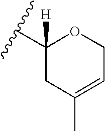 | 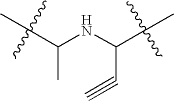 |
| 323 | OH/H | OH | 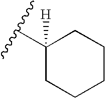 | 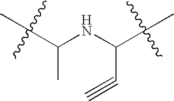 |
| 324 | OH/H | OH | 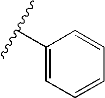 | 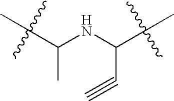 |
| 325 | OH/H | OH | 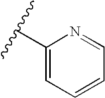 | 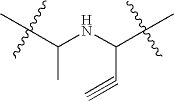 |
| 326 | OH/H | OH | 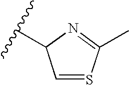 | 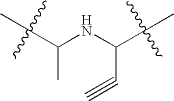 |
| 327 | OH/H | OH | 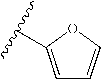 | 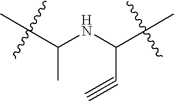 |
| 328 | OH/H | OH | 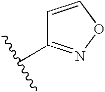 | 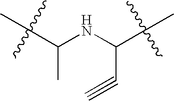 |
| 329 | OH/H | OH | 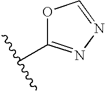 | 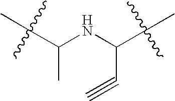 |
| 330 | OH/H | OH | 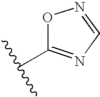 | 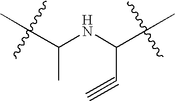 |
| 331 | OH/H | OH | 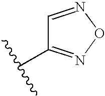 | 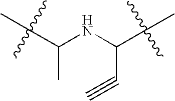 |
| 332 | OH/H | OH | 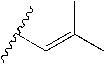 | 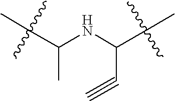 |
| 333 | OH/H | OH | 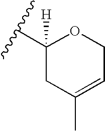 | 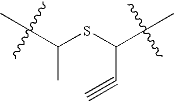 |
| 334 | OH/H | OH | 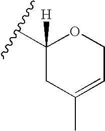 | 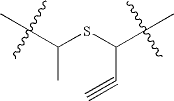 |
| 335 | OH/H | OH | 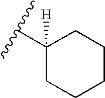 | 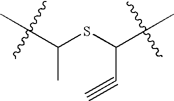 |
| 336 | OH/H | OH | 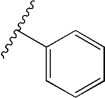 | 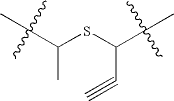 |
| 337 | OH/H | OH | 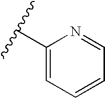 | 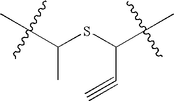 |
| 338 | OH/H | OH | 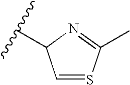 | 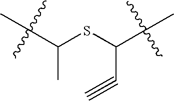 |
| 339 | OH/H | OH | 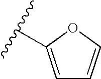 | 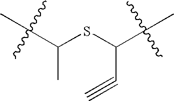 |
| 340 | OH/H | OH | 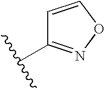 | 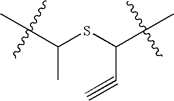 |
| 341 | OH/H | OH | 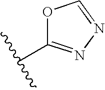 | 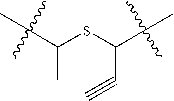 |
| 342 | OH/H | OH | 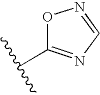 | 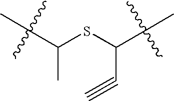 |
| 343 | OH/H | OH | 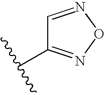 | 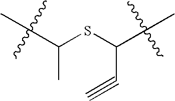 |
| 344 | OH/H | OH | 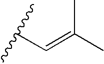 | 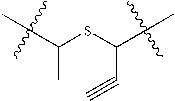 |
| 345 | OH/H | OH | 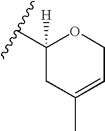 | 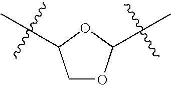 |
| 346 | OH/H | OH | 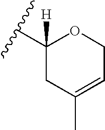 | 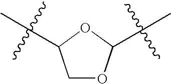 |
| 347 | OH/H | OH | 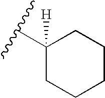 | 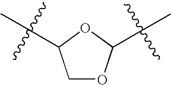 |
| 348 | OH/H | OH | 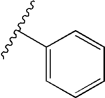 | 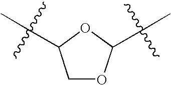 |
| 349 | OH/H | OH | 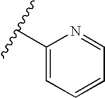 | 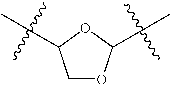 |
| 350 | OH/H | OH | 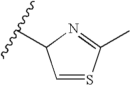 | 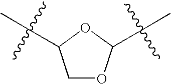 |
| 351 | OH/H | OH | 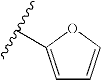 | 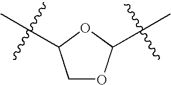 |
| 352 | OH/H | OH | 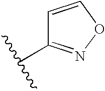 | 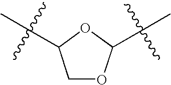 |
| 353 | OH/H | OH | 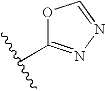 | 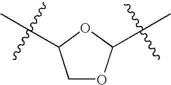 |
| 354 | OH/H | OH | 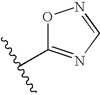 | 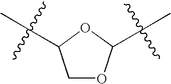 |
| 355 | OH/H | OH | 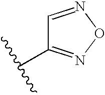 | 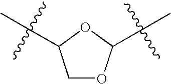 |
| 356 | OH/H | OH | 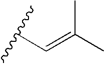 | 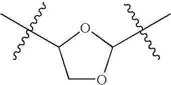 |
| 357 | OH/H | OH | 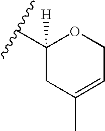 | 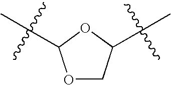 |
| 358 | OH/H | OH | 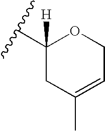 | 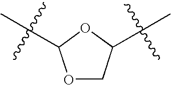 |
| 359 | OH/H | OH | 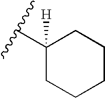 | 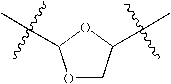 |
| 360 | OH/H | OH | 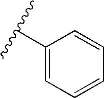 | 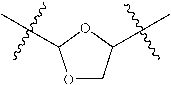 |
| 361 | OH/H | OH | 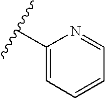 | 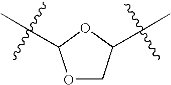 |
| 362 | OH/H | OH | 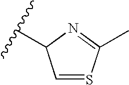 | 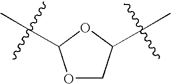 |
| 363 | OH/H | OH | 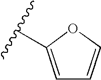 | 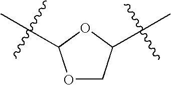 |
| 364 | OH/H | OH | 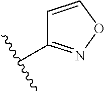 | 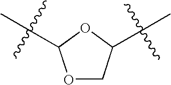 |
| 365 | OH/H | OH | 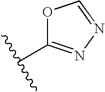 | 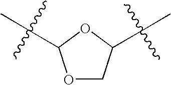 |
| 366 | OH/H | OH | 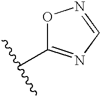 | 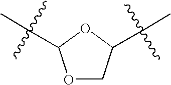 |
| 367 | OH/H | OH | 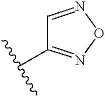 | 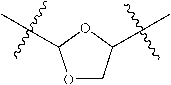 |
| 368 | OH/H | OH | 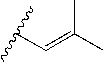 | 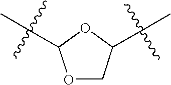 |
| 369 | OH/H | OH | 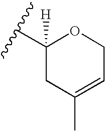 | 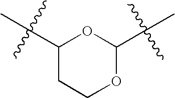 |
| 370 | OH/H | OH | 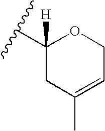 | 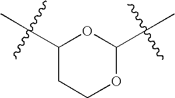 |
| 371 | OH/H | OH | 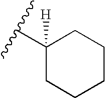 | 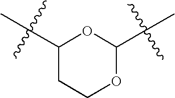 |
| 372 | OH/H | OH | 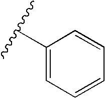 | 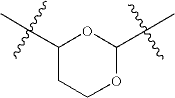 |
| 373 | OH/H | OH | 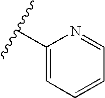 | 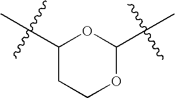 |
| 374 | OH/H | OH | 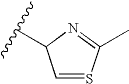 | 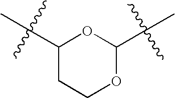 |
| 375 | OH/H | OH | 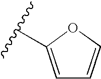 | 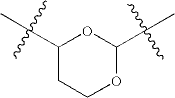 |
| 376 | OH/H | OH | 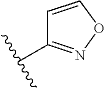 | 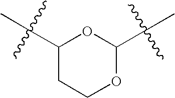 |
| 377 | OH/H | OH | 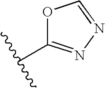 | 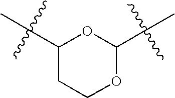 |
| 378 | OH/H | OH | 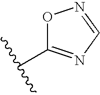 | 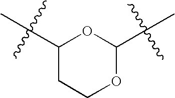 |
| 379 | OH/H | OH | 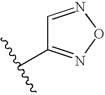 | 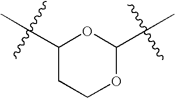 |
| 380 | OH/H | OH | 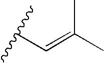 | 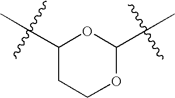 |
| 381 | OH/H | OH | 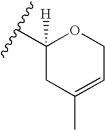 | 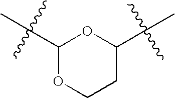 |
| 382 | OH/H | OH | 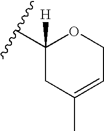 | 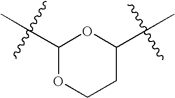 |
| 383 | OH/H | OH | 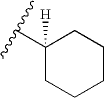 | 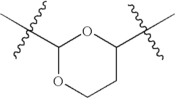 |
| 384 | OH/H | OH | 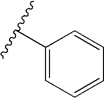 | 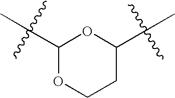 |
| 385 | OH/H | OH | 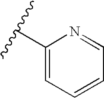 | 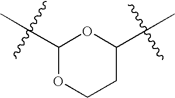 |
| 386 | OH/H | OH | 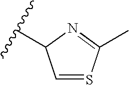 | 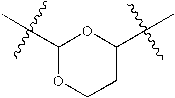 |
| 387 | OH/H | OH | 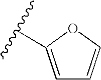 | 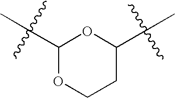 |
| 388 | OH/H | OH | 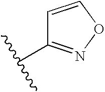 | 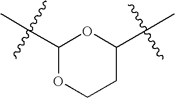 |
| 389 | OH/H | OH | 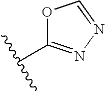 | 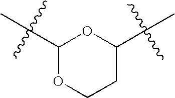 |
| 390 | OH/H | OH | 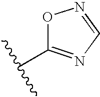 | 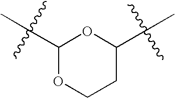 |
| 391 | OH/H | OH | 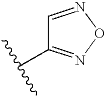 | 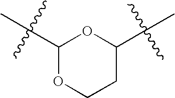 |
| 392 | OH/H | OH | 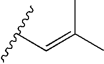 | 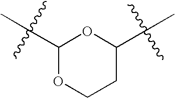 |
| 393 | OH/H | OH | 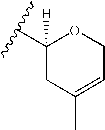 | 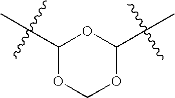 |
| 394 | OH/H | OH | 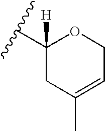 | 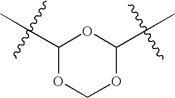 |
| 395 | OH/H | OH | 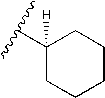 | 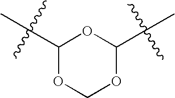 |
| 396 | OH/H | OH | 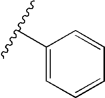 | 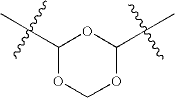 |
| 397 | OH/H | OH | 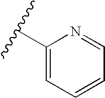 | 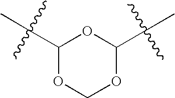 |
| 398 | OH/H | OH | 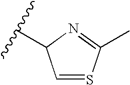 | 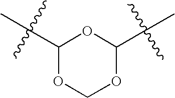 |
| 399 | OH/H | OH | 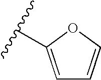 | 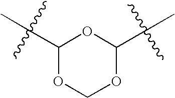 |
| 400 | OH/H | OH | 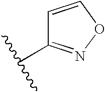 | 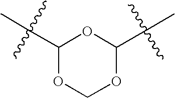 |
| 401 | OH/H | OH | 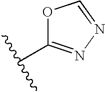 | 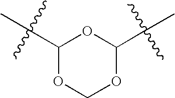 |
| 402 | OH/H | OH | 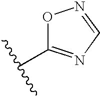 | 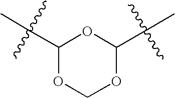 |
| 403 | OH/H | OH | 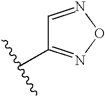 | 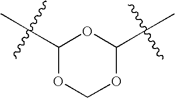 |
| 404 | OH/H | OH | 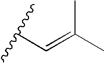 | 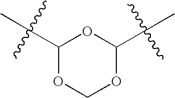 |
| 405 | OH/H | OH | 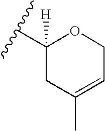 | 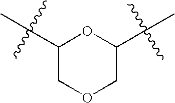 |
| 406 | OH/H | OH | 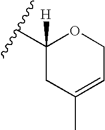 | 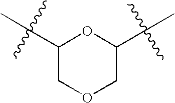 |
| 407 | OH/H | OH | 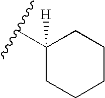 | 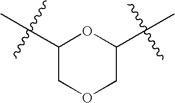 |
| 408 | OH/H | OH | 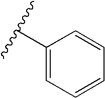 | 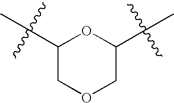 |
| 409 | OH/H | OH | 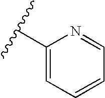 | 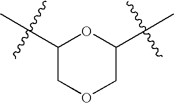 |
| 410 | OH/H | OH | 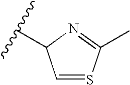 | 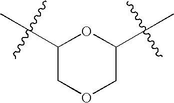 |
| 411 | OH/H | OH | 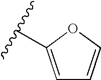 | 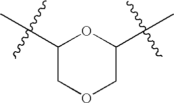 |
| 412 | OH/H | OH | 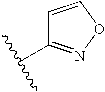 | 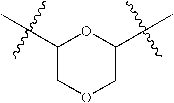 |
| 413 | OH/H | OH | 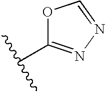 | 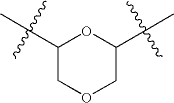 |
| 414 | OH/H | OH | 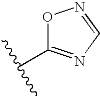 | 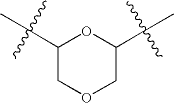 |
| 415 | OH/H | OH | 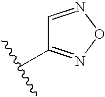 | 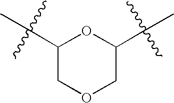 |
| 416 | OH/H | OH | 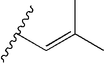 | 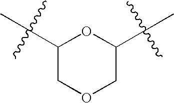 |
| 417 | OH/H | OH | 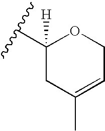 | 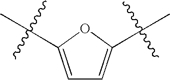 |
| 418 | OH/H | OH | 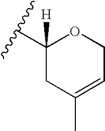 | 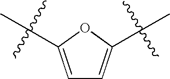 |
| 419 | OH/H | OH | 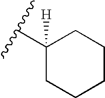 | 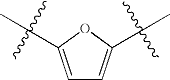 |
| 420 | OH/H | OH | 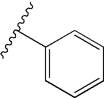 | 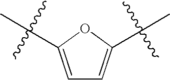 |
| 421 | OH/H | OH | 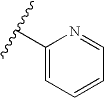 | 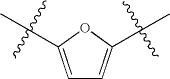 |
| 422 | OH/H | OH | 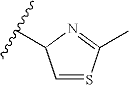 | 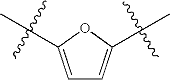 |
| 423 | OH/H | OH | 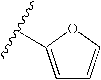 | 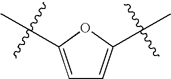 |
| 424 | OH/H | OH | 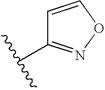 | 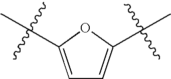 |
| 425 | OH/H | OH | 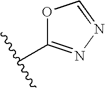 | 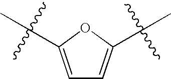 |
| 426 | OH/H | OH | 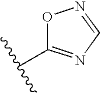 | 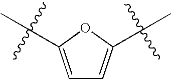 |
| 427 | OH/H | OH | 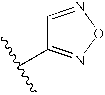 | 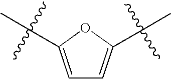 |
| 428 | OH/H | OH | 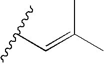 | 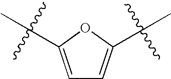 |
| 429 | OH/H | OH | 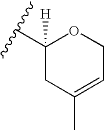 | 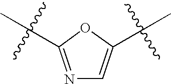 |
| 430 | OH/H | OH | 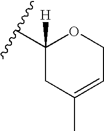 | 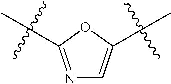 |
| 431 | OH/H | OH | 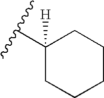 | 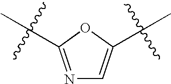 |
| 432 | OH/H | OH | 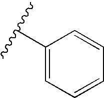 | 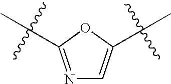 |
| 433 | OH/H | OH | 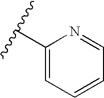 | 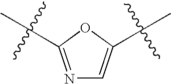 |
| 434 | OH/H | OH | 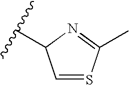 | 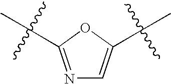 |
| 435 | OH/H | OH | 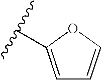 | 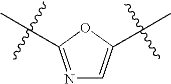 |
| 436 | OH/H | OH | 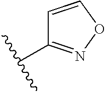 | 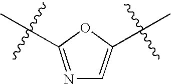 |
| 437 | OH/H | OH | 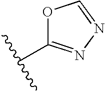 | 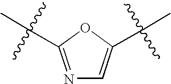 |
| 438 | OH/H | OH | 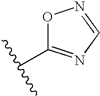 | 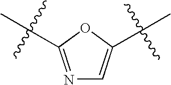 |
| 439 | OH/H | OH | 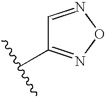 | 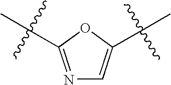 |
| 440 | OH/H | OH | 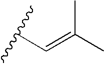 | 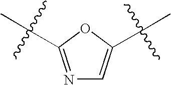 |
| 441 | OH/H | OH | 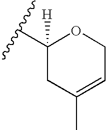 | 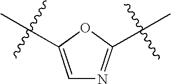 |
| 442 | OH/H | OH | 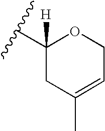 | 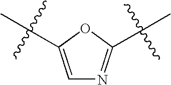 |
| 443 | OH/H | OH | 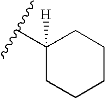 | 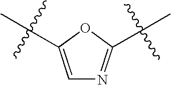 |
| 444 | OH/H | OH | 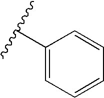 | 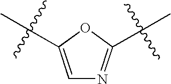 |
| 445 | OH/H | OH | 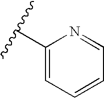 | 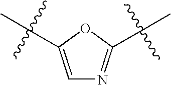 |
| 446 | OH/H | OH | 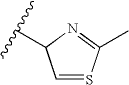 | 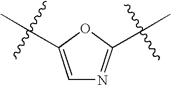 |
| 447 | OH/H | OH | 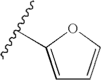 | 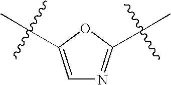 |
| 448 | OH/H | OH | 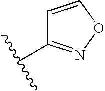 | 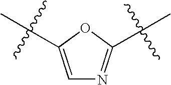 |
| 449 | OH/H | OH | 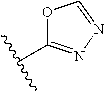 | 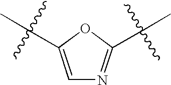 |
| 450 | OH/H | OH | 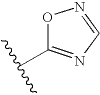 | 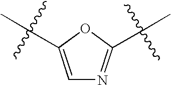 |
| 451 | OH/H | OH | 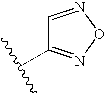 | 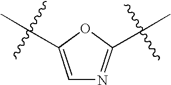 |
| 452 | OH/H | OH | 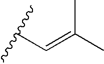 | 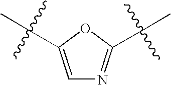 |
| 453 | OH/H | OH | 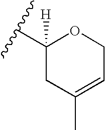 | 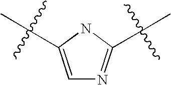 |
| 454 | OH/H | OH | 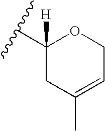 | 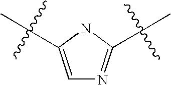 |
| 455 | OH/H | OH | 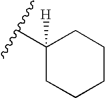 | 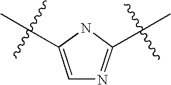 |
| 456 | OH/H | OH | 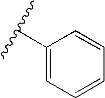 | 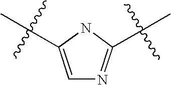 |
| 457 | OH/H | OH | 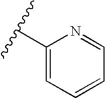 | 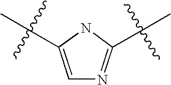 |
| 458 | OH/H | OH | 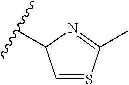 | 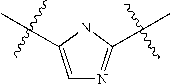 |
| 459 | OH/H | OH | 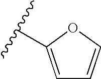 | 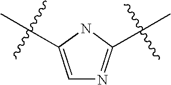 |
| 460 | OH/H | OH | 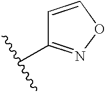 | 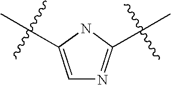 |
| 461 | OH/H | OH | 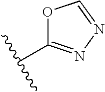 | 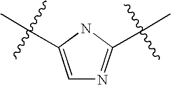 |
| 462 | OH/H | OH | 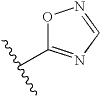 | 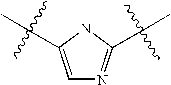 |
| 463 | OH/H | OH | 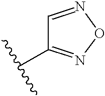 | 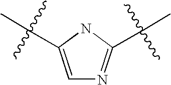 |
| 464 | OH/H | OH | 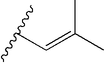 | 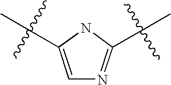 |
| 465 | OH/H | OH | 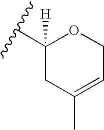 | 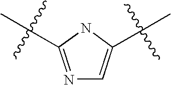 |
| 466 | OH/H | OH | 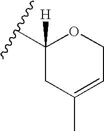 | 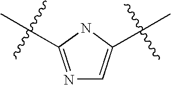 |
| 467 | OH/H | OH | 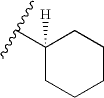 | 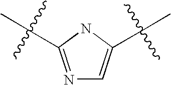 |
| 468 | OH/H | OH | 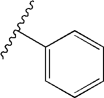 | 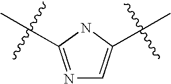 |
| 469 | OH/H | OH | 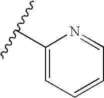 | 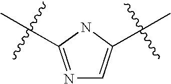 |
| 470 | OH/H | OH | 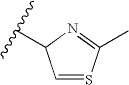 | 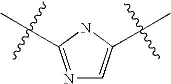 |
| 471 | OH/H | OH | 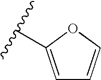 | 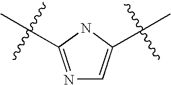 |
| 472 | OH/H | OH | 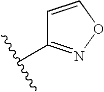 | 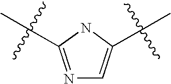 |
| 473 | OH/H | OH | 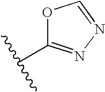 | 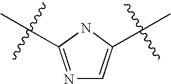 |
| 474 | OH/H | OH | 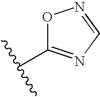 | 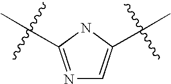 |
| 475 | OH/H | OH | 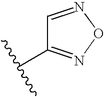 | 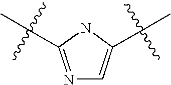 |
| 476 | OH/H | OH | 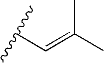 | 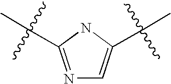 |
| 477 | OH/H | OH | 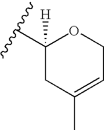 | 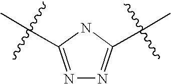 |
| 478 | OH/H | OH | 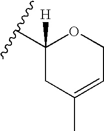 | 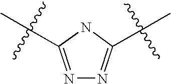 |
| 479 | OH/H | OH | 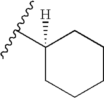 | 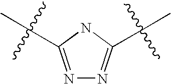 |
| 480 | OH/H | OH | 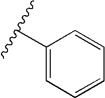 | 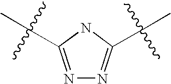 |
| 481 | OH/H | OH | 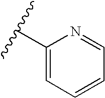 | 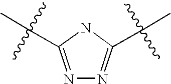 |
| 482 | OH/H | OH | 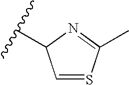 | 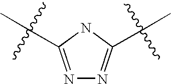 |
| 483 | OH/H | OH | 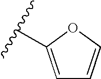 | 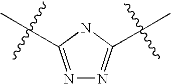 |
| 484 | OH/H | OH | 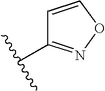 | 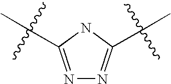 |
| 485 | OH/H | OH | 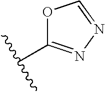 | 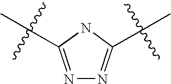 |
| 486 | OH/H | OH | 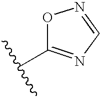 | 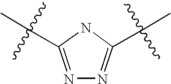 |
| 487 | OH/H | OH | 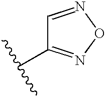 | 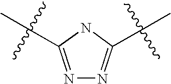 |
| 488 | OH/H | OH | 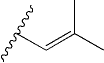 | 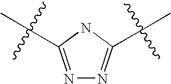 |
| 489 | OH/H | OH | 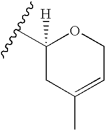 | 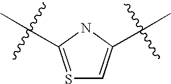 |
| 490 | OH/H | OH | 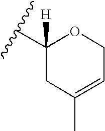 | 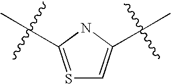 |
| 491 | OH/H | OH | 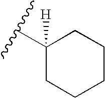 | 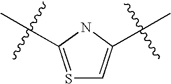 |
| 492 | OH/H | OH | 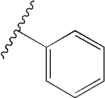 | 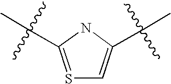 |
| 493 | OH/H | OH | 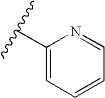 | 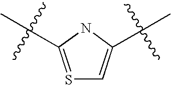 |
| 494 | OH/H | OH | 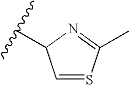 | 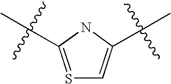 |
| 495 | OH/H | OH | 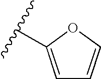 | 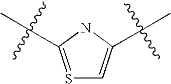 |
| 496 | OH/H | OH | 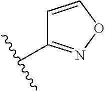 | 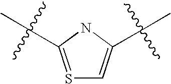 |
| 497 | OH/H | OH | 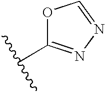 | 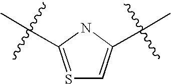 |
| 498 | OH/H | OH | 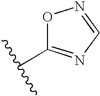 | 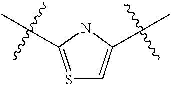 |
| 499 | OH/H | OH | 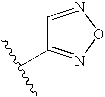 | 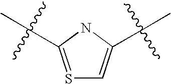 |
| 500 | OH/H | OH | 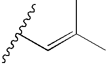 | 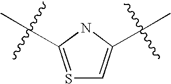 |
| 501 | OH/H | OH | 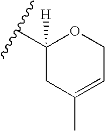 | 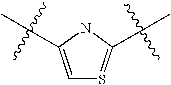 |
| 502 | OH/H | OH | 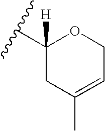 | 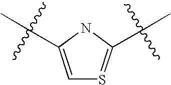 |
| 503 | OH/H | OH | 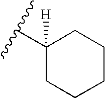 | 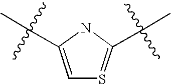 |
| 504 | OH/H | OH | 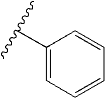 | 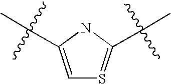 |
| 505 | OH/H | OH | 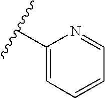 | 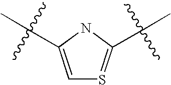 |
| 506 | OH/H | OH | 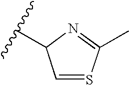 | 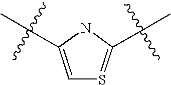 |
| 507 | OH/H | OH | 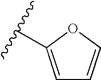 | 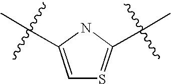 |
| 508 | OH/H | OH | 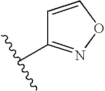 | 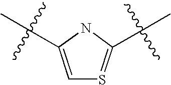 |
| 509 | OH/H | OH | 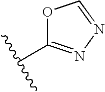 | 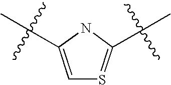 |
| 510 | OH/H | OH | 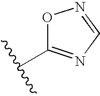 | 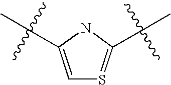 |
| 511 | OH/H | OH | 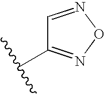 | 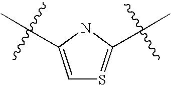 |
| 512 | OH/H | OH | 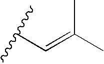 | 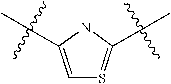 |
| 513 | OH/H | OH | 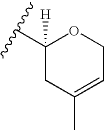 | 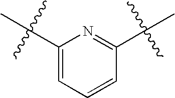 |
| 514 | OH/H | OH | 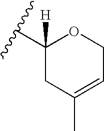 | 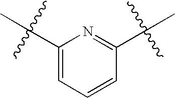 |
| 515 | OH/H | OH | 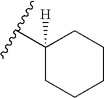 | 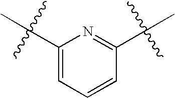 |
| 516 | OH/H | OH | 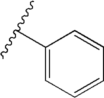 | 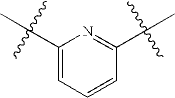 |
| 517 | OH/H | OH | 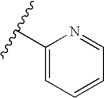 | 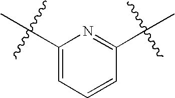 |
| 518 | OH/H | OH | 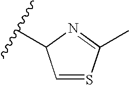 | 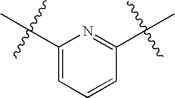 |
| 519 | OH/H | OH | 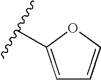 | 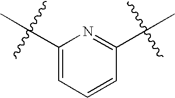 |
| 520 | OH/H | OH | 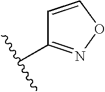 | 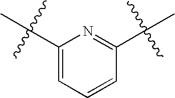 |
| 521 | OH/H | OH | 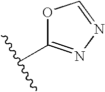 | 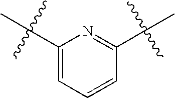 |
| 522 | OH/H | OH | 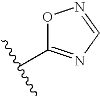 | 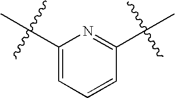 |
| 523 | OH/H | OH | 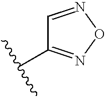 | 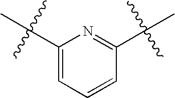 |
| 524 | OH/H | OH | 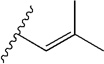 | 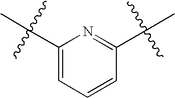 |
| 525 | OH/H | OH | 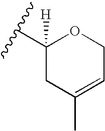 | 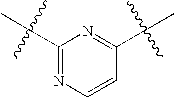 |
| 526 | OH/H | OH | 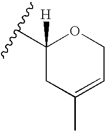 | 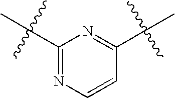 |
| 527 | OH/H | OH | 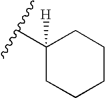 | 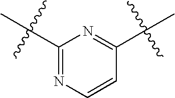 |
| 528 | OH/H | OH | 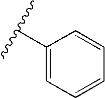 | 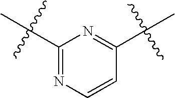 |
| 529 | OH/H | OH | 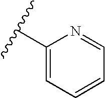 | 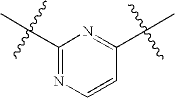 |
| 530 | OH/H | OH | 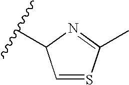 | 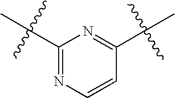 |
| 531 | OH/H | OH | 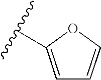 | 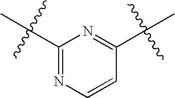 |
| 532 | OH/H | OH | 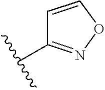 | 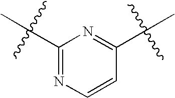 |
| 533 | OH/H | OH | 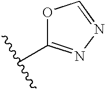 | 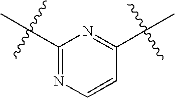 |
| 534 | OH/H | OH | 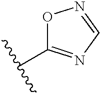 | 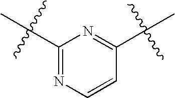 |
| 535 | OH/H | OH | 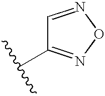 | 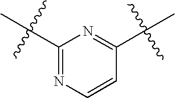 |
| 536 | OH/H | OH | 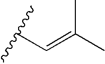 | 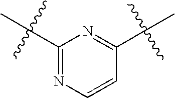 |
| 537 | OH/H | OH | 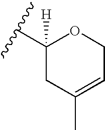 | 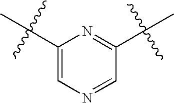 |
| 538 | OH/H | OH | 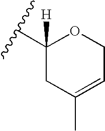 | 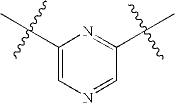 |
| 539 | OH/H | OH | 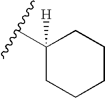 | 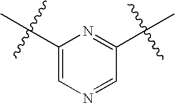 |
| 540 | OH/H | OH | 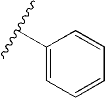 | 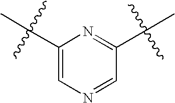 |
| 541 | OH/H | OH | 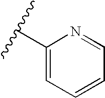 | 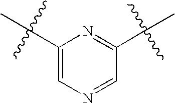 |
| 542 | OH/H | OH | 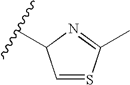 | 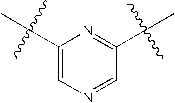 |
| 543 | OH/H | OH | 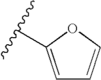 | 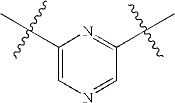 |
| 544 | OH/H | OH | 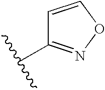 | 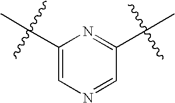 |
| 545 | OH/H | OH | 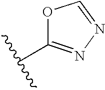 | 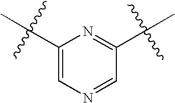 |
| 546 | OH/H | OH | 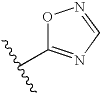 | 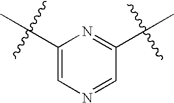 |
| 547 | OH/H | OH | 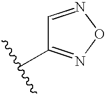 | 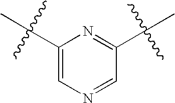 |
| 548 | OH/H | OH | 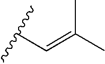 | 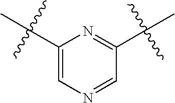 |
| 549 | OH/H | OH | 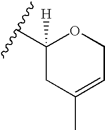 | 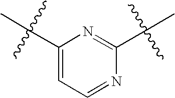 |
| 550 | OH/H | OH | 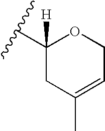 | 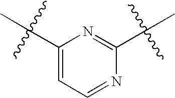 |
| 551 | OH/H | OH | 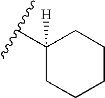 | 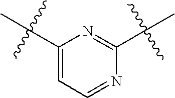 |
| 552 | OH/H | OH | 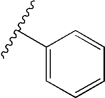 | 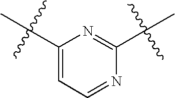 |
| 553 | OH/H | OH | 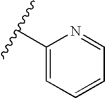 | 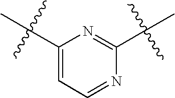 |
| 554 | OH/H | OH | 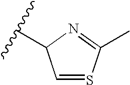 | 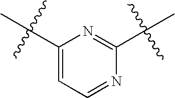 |
| 555 | OH/H | OH | 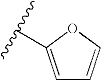 | 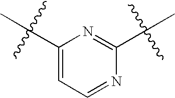 |
| 556 | OH/H | OH | 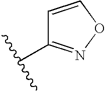 | 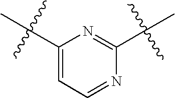 |
| 557 | OH/H | OH | 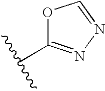 | 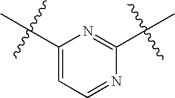 |
| 558 | OH/H | OH | 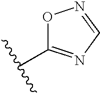 | 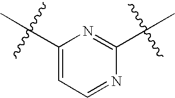 |
| 559 | OH/H | OH | 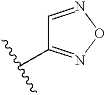 | 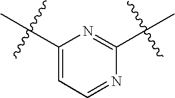 |
| 560 | OH/H | OH | 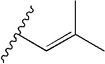 | 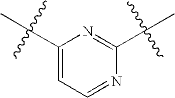 |
| 561 | OH/H | OH | 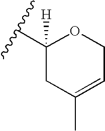 | 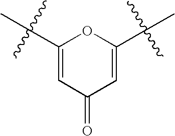 |
| 562 | OH/H | OH | 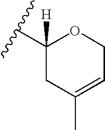 | 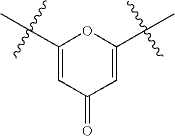 |
| 563 | OH/H | OH | 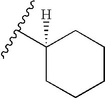 | 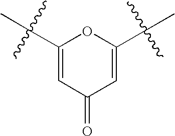 |
| 564 | OH/H | OH | 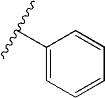 | 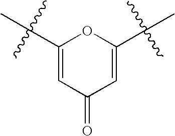 |
| 565 | OH/H | OH | 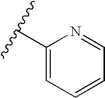 | 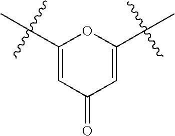 |
| 566 | OH/H | OH | 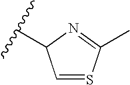 | 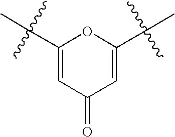 |
| 567 | OH/H | OH | 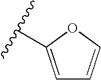 | 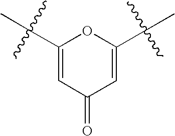 |
| 568 | OH/H | OH | 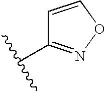 | 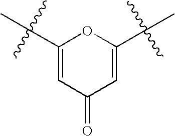 |
| 569 | OH/H | OH | 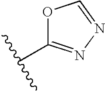 | 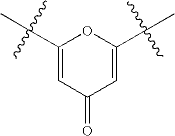 |
| 570 | OH/H | OH | 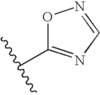 | 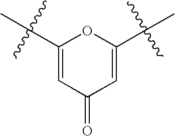 |
| 571 | OH/H | OH | 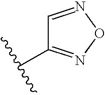 | 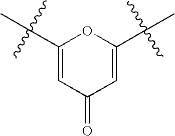 |
| 572 | OH/H | OH | 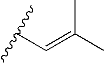 | 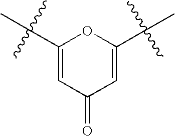 |

### EXAMPLE 2

The following macrocycles of Formula XIII are prepared, using appropriate reagents and according generally to the methods described herein.

| | |
|---|---|
| | (XIII) |
| 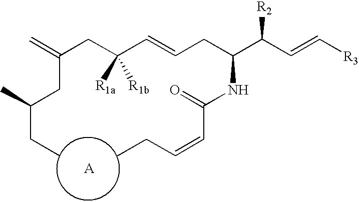 | |

| Compound | R1a/R1b | R2 | R3 | A |
|---|---|---|---|---|
| 573 | OH/H | OH | 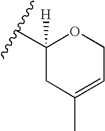 | 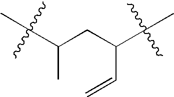 |
| 574 | OH/H | OH | 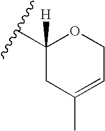 | 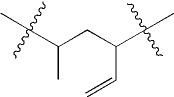 |
| 575 | OH/H | OH | 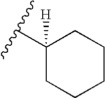 | 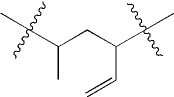 |
| 576 | OH/H | OH | 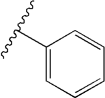 | 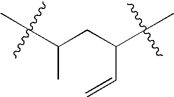 |
| 577 | OH/H | OH | 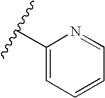 | 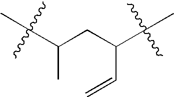 |
| 578 | OH/H | OH | 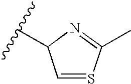 | 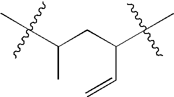 |
| 579 | OH/H | OH | 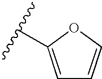 | 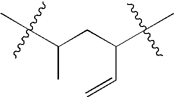 |
| 580 | OH/H | OH | 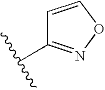 | 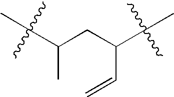 |
| 581 | OH/H | OH | 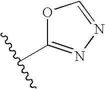 | 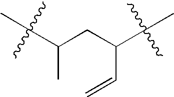 |
| 582 | OH/H | OH | 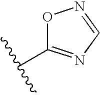 | 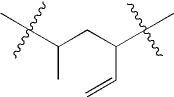 |
| 583 | OH/H | OH | 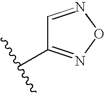 | 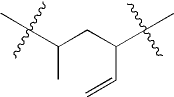 |
| 584 | OH/H | OH | 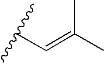 | 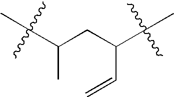 |
| 585 | OH/H | OH | 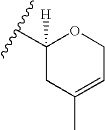 | 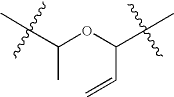 |
| 586 | OH/H | OH | 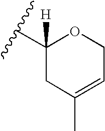 | 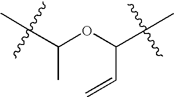 |
| 587 | OH/H | OH | 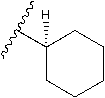 | 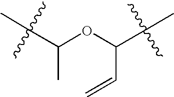 |
| 588 | OH/H | OH | 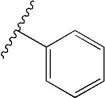 | 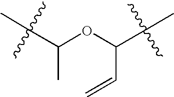 |
| 589 | OH/H | OH | 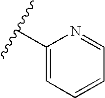 | 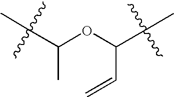 |
| 590 | OH/H | OH | 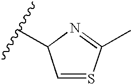 | 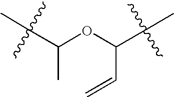 |
| 591 | OH/H | OH | 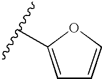 | 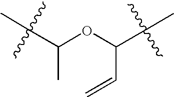 |
| 592 | OH/H | OH | 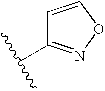 | 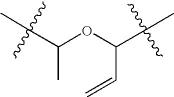 |
| 593 | OH/H | OH | 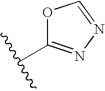 | 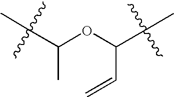 |
| 594 | OH/H | OH | 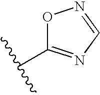 | 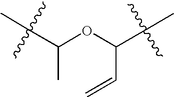 |
| 595 | OH/H | OH | 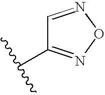 | 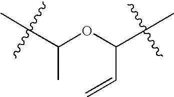 |
| 596 | OH/H | OH | 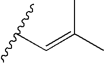 | 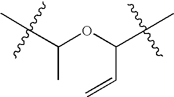 |
| 597 | OH/H | OH | 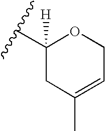 | 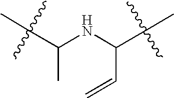 |
| 598 | OH/H | OH | 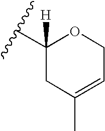 | 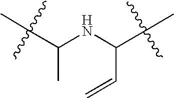 |
| 599 | OH/H | OH | 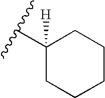 | 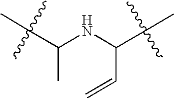 |
| 600 | OH/H | OH | 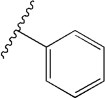 | 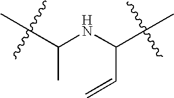 |
| 601 | OH/H | OH | 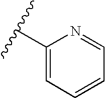 | 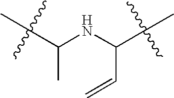 |
| 602 | OH/H | OH | 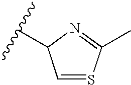 | 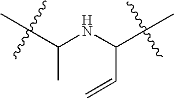 |
| 603 | OH/H | OH | 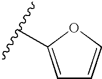 | 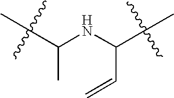 |
| 604 | OH/H | OH | 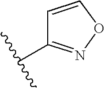 | 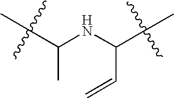 |
| 605 | OH/H | OH | 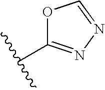 | 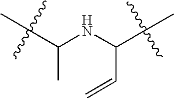 |
| 606 | OH/H | OH | 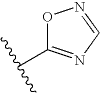 | 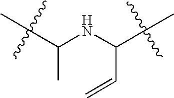 |
| 607 | OH/H | OH | 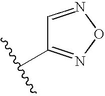 | 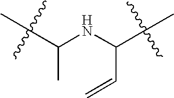 |
| 608 | OH/H | OH | 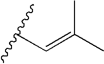 | 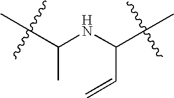 |
| 610 | OH/H | OH | 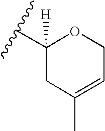 | 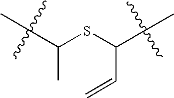 |
| 611 | OH/H | OH | 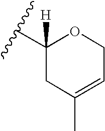 | 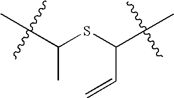 |
| 612 | OH/H | OH | 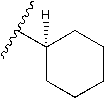 | 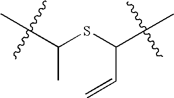 |
| 613 | OH/H | OH | 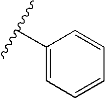 | 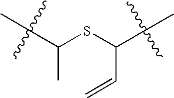 |
| 614 | OH/H | OH | 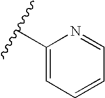 | 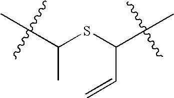 |
| 615 | OH/H | OH | 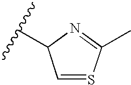 | 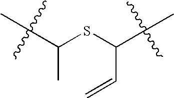 |
| 616 | OH/H | OH | 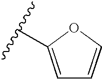 | 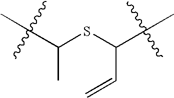 |
| 617 | OH/H | OH | 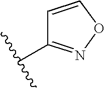 | 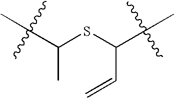 |
| 618 | OH/H | OH | 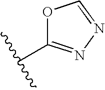 | 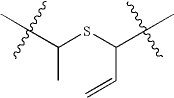 |
| 619 | OH/H | OH | 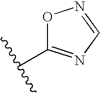 | 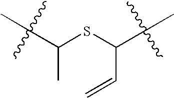 |
| 620 | OH/H | OH | 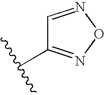 | 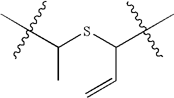 |
| 621 | OH/H | OH | 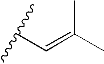 | 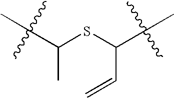 |
| 622 | OH/H | OH | 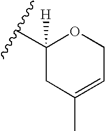 | 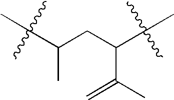 |
| 623 | OH/H | OH | 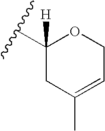 | 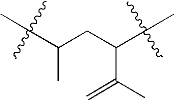 |
| 624 | OH/H | OH | 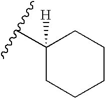 | 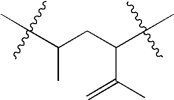 |
| 625 | OH/H | OH | 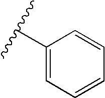 | 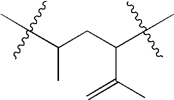 |
| 626 | OH/H | OH | 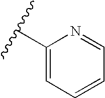 | 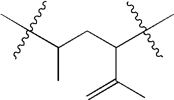 |
| 627 | OH/H | OH | 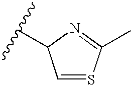 | 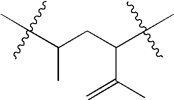 |
| 628 | OH/H | OH | 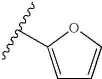 | 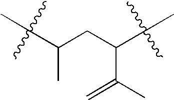 |
| 629 | OH/H | OH | 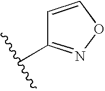 | 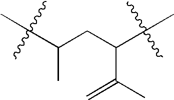 |
| 630 | OH/H | OH | 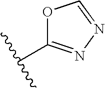 | 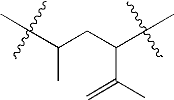 |
| 631 | OH/H | OH | 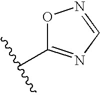 | 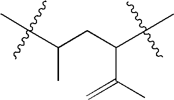 |
| 632 | OH/H | OH | 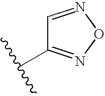 | 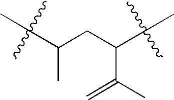 |
| 633 | OH/H | OH | 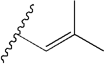 | 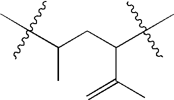 |
| 634 | OH/H | OH | 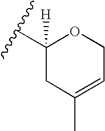 | 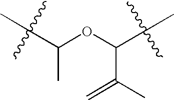 |
| 635 | OH/H | OH | 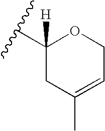 | 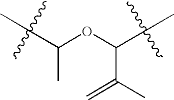 |
| 636 | OH/H | OH | 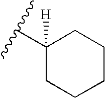 | 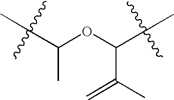 |
| 637 | OH/H | OH | 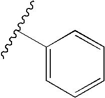 | 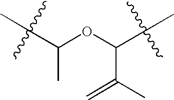 |
| 638 | OH/H | OH | 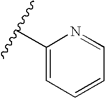 | 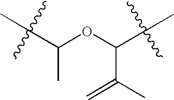 |
| 639 | OH/H | OH | 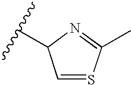 | 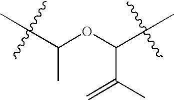 |
| 640 | OH/H | OH | 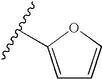 | 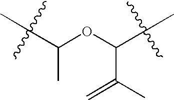 |
| 641 | OH/H | OH | 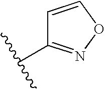 | 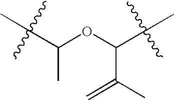 |
| 642 | OH/H | OH | 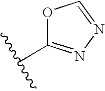 | 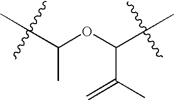 |
| 643 | OH/H | OH | 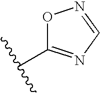 | 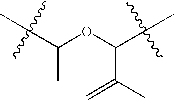 |
| 644 | OH/H | OH | 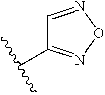 | 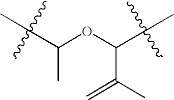 |
| 645 | OH/H | OH | 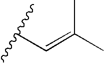 | 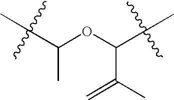 |
| 646 | OH/H | OH | 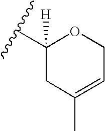 | 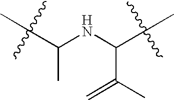 |
| 647 | OH/H | OH | 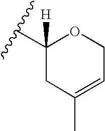 | 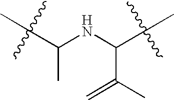 |
| 648 | OH/H | OH | 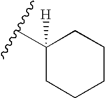 | 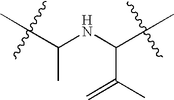 |
| 649 | OH/H | OH | 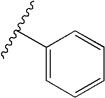 | 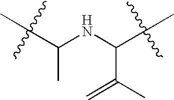 |
| 650 | OH/H | OH | 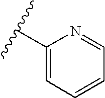 | 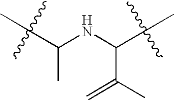 |
| 651 | OH/H | OH | 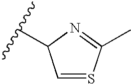 | 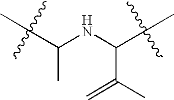 |
| 652 | OH/H | OH | 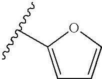 | 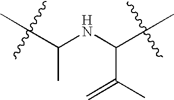 |
| 653 | OH/H | OH | 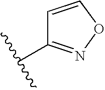 | 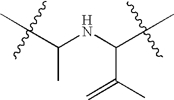 |
| 654 | OH/H | OH | 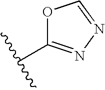 | 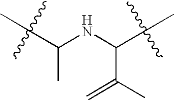 |
| 655 | OH/H | OH | 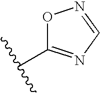 | 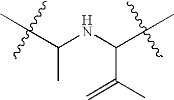 |
| 656 | OH/H | OH | 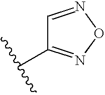 | 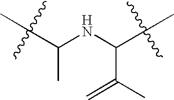 |
| 657 | OH/H | OH | 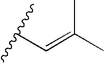 | 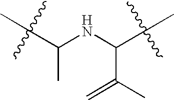 |
| 658 | OH/H | OH | 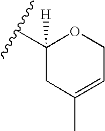 | 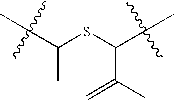 |
| 659 | OH/H | OH | 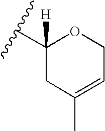 | 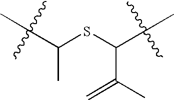 |
| 660 | OH/H | OH | 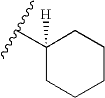 | 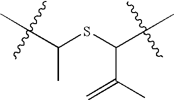 |
| 661 | OH/H | OH | 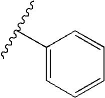 | 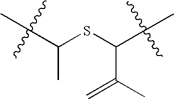 |
| 662 | OH/H | OH | 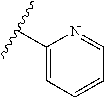 | 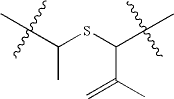 |
| 663 | OH/H | OH | 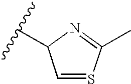 | 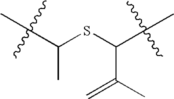 |
| 664 | OH/H | OH | 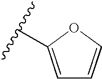 | 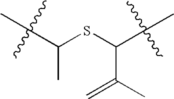 |
| 665 | OH/H | OH | 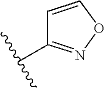 | 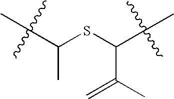 |
| 666 | OH/H | OH | 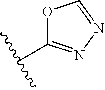 | 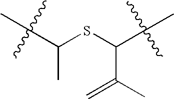 |
| 667 | OH/H | OH | 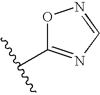 | 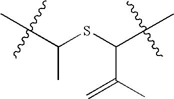 |
| 668 | OH/H | OH | 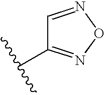 | 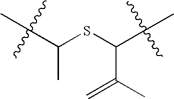 |
| 669 | OH/H | OH | 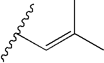 | 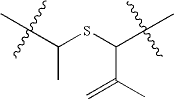 |
| 670 | OH/H | OH | 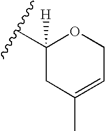 | 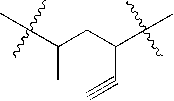 |
| 671 | OH/H | OH | 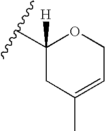 | 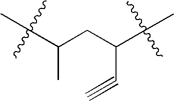 |
| 672 | OH/H | OH | 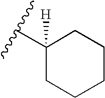 | 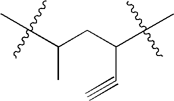 |
| 673 | OH/H | OH | 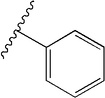 | 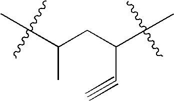 |
| 674 | OH/H | OH | 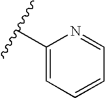 | 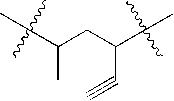 |
| 675 | OH/H | OH | 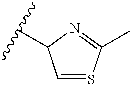 | 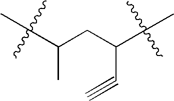 |
| 676 | OH/H | OH | 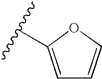 | 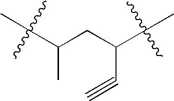 |
| 677 | OH/H | OH | 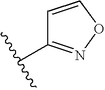 | 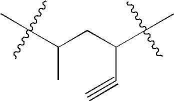 |
| 678 | OH/H | OH | 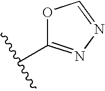 | 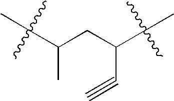 |
| 679 | OH/H | OH | 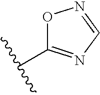 | 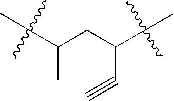 |
| 680 | OH/H | OH | 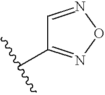 | 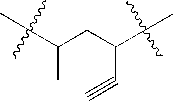 |
| 681 | OH/H | OH | 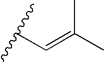 | 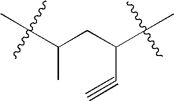 |
| 682 | OH/H | OH | 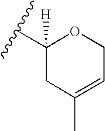 | 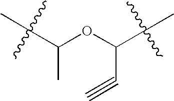 |
| 683 | | | 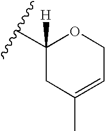 | 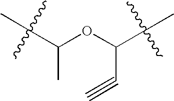 |
| 684 | | | 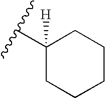 | 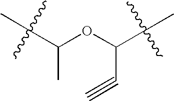 |
| 685 | | | 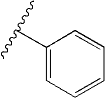 | 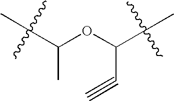 |
| 686 | | | 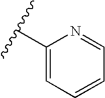 | 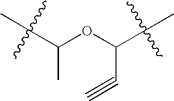 |
| 687 | OH/H | OH | 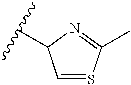 | 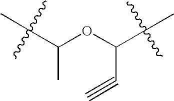 |
| 688 | OH/H | OH | 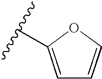 | 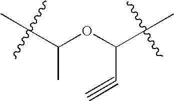 |
| 689 | OH/H | OH | 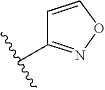 | 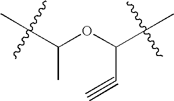 |
| 690 | OH/H | OH | 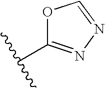 | 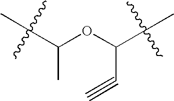 |
| 691 | OH/H | OH | 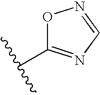 | 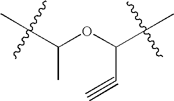 |
| 692 | OH/H | OH | 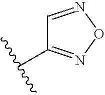 | 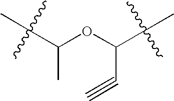 |
| 693 | OH/H | OH | 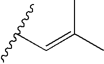 | 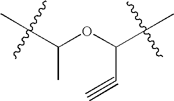 |
| 694 | OH/H | OH | 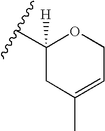 | 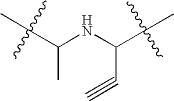 |
| 695 | OH/H | OH | 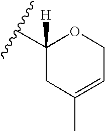 | 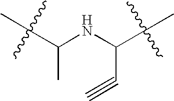 |
| 696 | OH/H | OH | 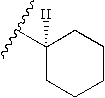 | 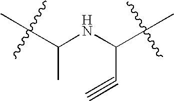 |
| 697 | OH/H | OH | 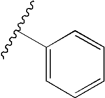 | 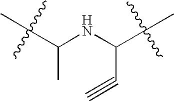 |
| 698 | OH/H | OH | 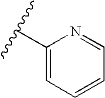 | 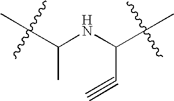 |
| 699 | OH/H | OH | 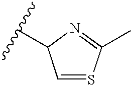 | 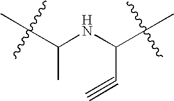 |
| 700 | OH/H | OH | 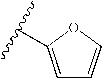 | 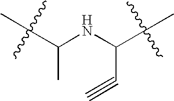 |
| 701 | OH/H | OH | 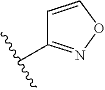 | 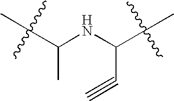 |
| 702 | OH/H | OH | 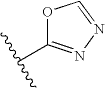 | 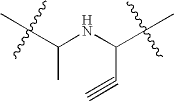 |
| 703 | OH/H | OH | 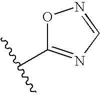 | 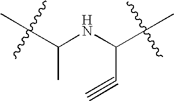 |
| 704 | OH/H | OH | 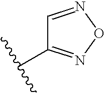 | 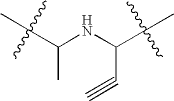 |
| 705 | OH/H | OH | 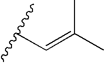 | 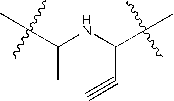 |
| 706 | OH/H | OH | 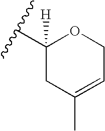 | 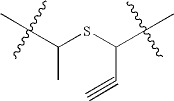 |
| 707 | OH/H | OH | 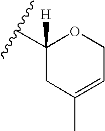 | 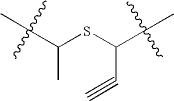 |
| 708 | OH/H | OH | 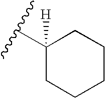 | 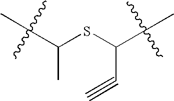 |
| 709 | OH/H | OH | 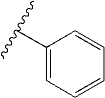 | 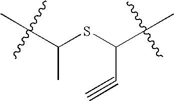 |
| 710 | OH/H | OH | 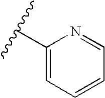 | 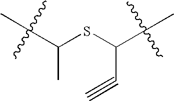 |
| 711 | OH/H | OH | 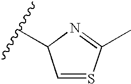 | 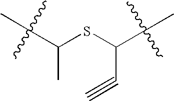 |
| 712 | OH/H | OH | 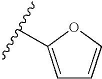 | 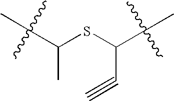 |
| 713 | OH/H | OH | 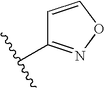 | 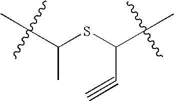 |
| 714 | OH/H | OH | 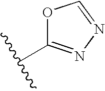 | 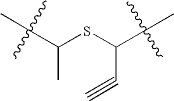 |
| 715 | OH/H | OH | 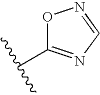 | 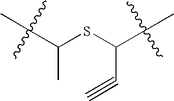 |
| 716 | OH/H | OH | 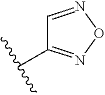 | 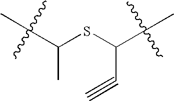 |
| 717 | OH/H | OH | 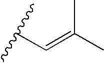 | 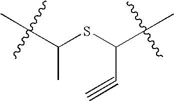 |
| 718 | OH/H | OH | 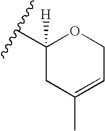 | 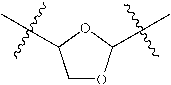 |
| 719 | OH/H | OH | 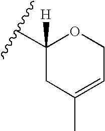 | 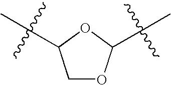 |
| 720 | OH/H | OH | 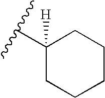 | 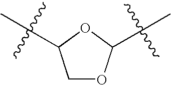 |
| 721 | OH/H | OH | 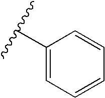 | 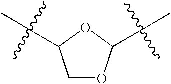 |
| 722 | OH/H | OH | 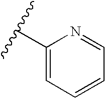 | 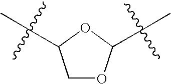 |
| 723 | OH/H | OH | 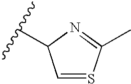 | 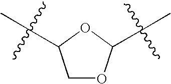 |
| 724 | OH/H | OH | 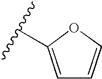 | 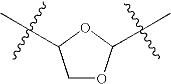 |
| 725 | OH/H | OH | 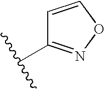 | 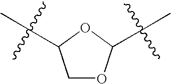 |
| 726 | OH/H | OH | 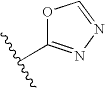 | 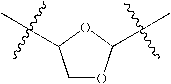 |
| 727 | OH/H | OH | 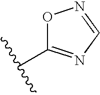 | 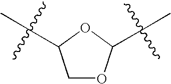 |
| 728 | OH/H | OH | 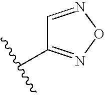 | 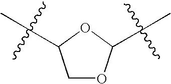 |
| 729 | OH/H | OH | 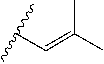 | 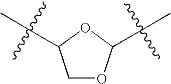 |
| 730 | OH/H | OH | 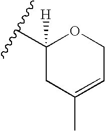 | 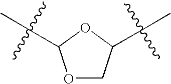 |
| 731 | OH/H | OH | 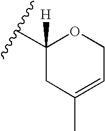 | 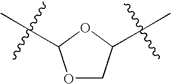 |
| 732 | OH/H | OH | 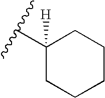 | 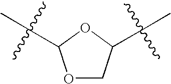 |
| 733 | OH/H | OH | 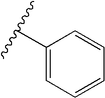 | 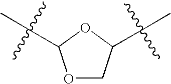 |
| 734 | OH/H | OH | 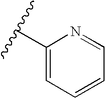 | 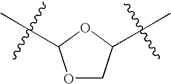 |
| 735 | OH/H | OH | 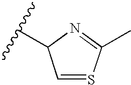 | 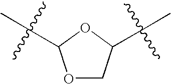 |
| 736 | OH/H | OH | 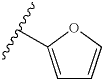 | 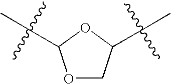 |
| 737 | OH/H | OH | 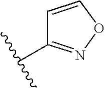 | 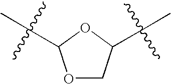 |
| 738 | OH/H | OH | 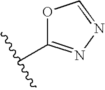 | 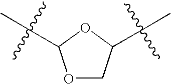 |
| 739 | OH/H | OH | 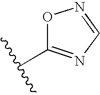 | 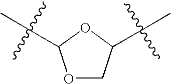 |
| 740 | OH/H | OH | 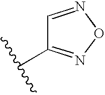 | 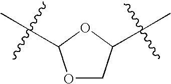 |
| 741 | OH/H | OH | 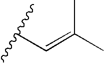 | 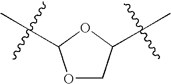 |
| 742 | OH/H | OH | 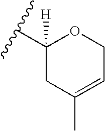 | 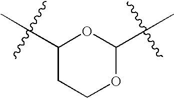 |
| 743 | OH/H | OH | 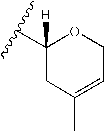 | 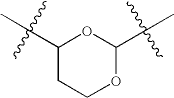 |
| 744 | OH/H | OH | 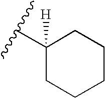 | 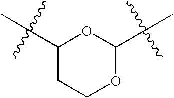 |
| 745 | OH/H | OH | 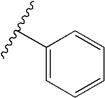 | 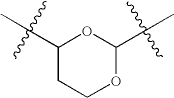 |
| 746 | OH/H | OH | 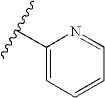 | 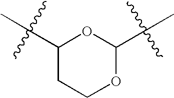 |
| 747 | OH/H | OH | 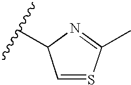 | 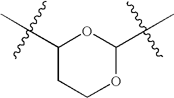 |
| 748 | OH/H | OH | 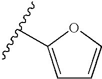 | 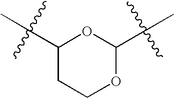 |
| 749 | OH/H | OH | 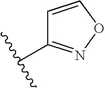 | 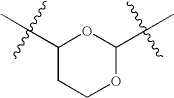 |
| 750 | OH/H | OH | 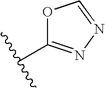 | 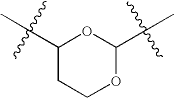 |
| 751 | OH/H | OH | 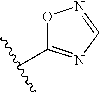 | 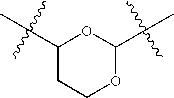 |
| 752 | OH/H | OH | 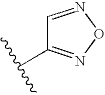 | 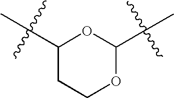 |
| 753 | OH/H | OH | 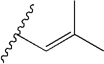 | 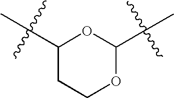 |
| 754 | OH/H | OH | 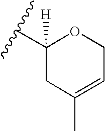 | 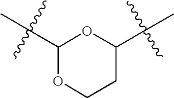 |
| 755 | OH/H | OH | 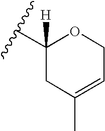 | 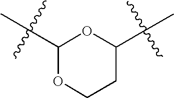 |
| 756 | OH/H | OH | 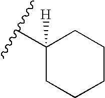 | 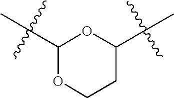 |
| 757 | OH/H | OH | 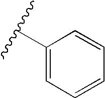 | 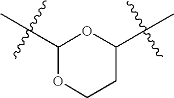 |
| 758 | OH/H | OH | 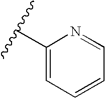 | 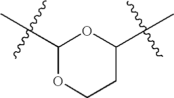 |
| 759 | OH/H | OH | 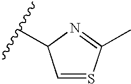 | 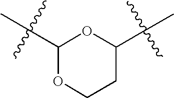 |
| 760 | OH/H | OH | 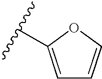 | 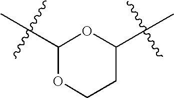 |
| 761 | OH/H | OH | 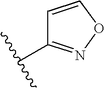 | 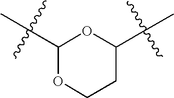 |
| 762 | OH/H | OH | 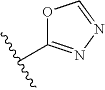 | 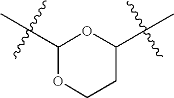 |
| 763 | OH/H | OH | 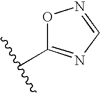 | 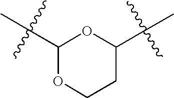 |
| 764 | OH/H | OH | 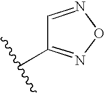 | 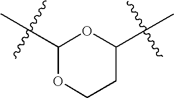 |
| 765 | OH/H | OH | 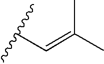 | 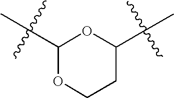 |
| 766 | OH/H | OH | 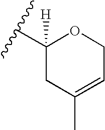 | 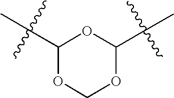 |
| 767 | OH/H | OH | 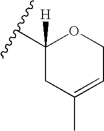 | 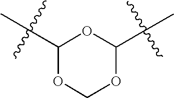 |
| 768 | OH/H | OH | 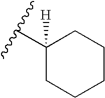 | 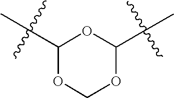 |
| 769 | OH/H | OH | 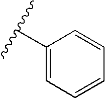 | 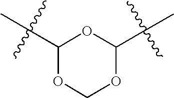 |
| 770 | OH/H | OH | 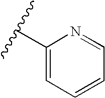 | 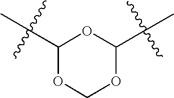 |
| 771 | OH/H | OH | 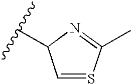 | 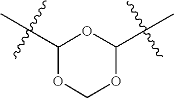 |
| 772 | OH/H | OH | 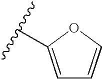 | 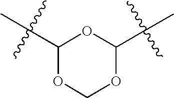 |
| 773 | OH/H | OH | 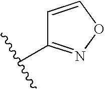 | 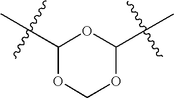 |
| 774 | OH/H | OH | 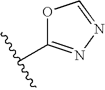 | 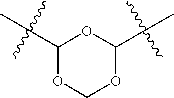 |
| 775 | OH/H | OH | 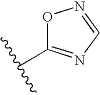 | 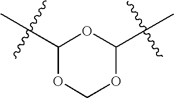 |
| 776 | OH/H | OH | 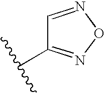 | 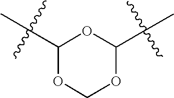 |
| 777 | OH/H | OH | 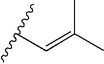 | 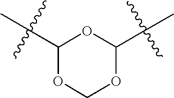 |
| 778 | OH/H | OH | 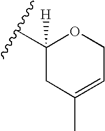 | 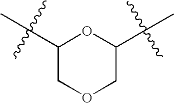 |
| 779 | OH/H | OH | 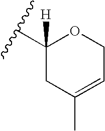 | 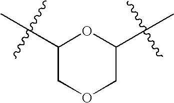 |
| 780 | OH/H | OH | 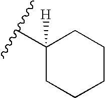 | 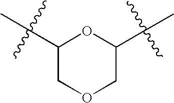 |
| 781 | OH/H | OH | 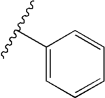 | 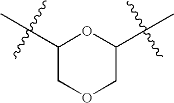 |
| 782 | OH/H | OH | 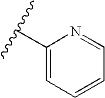 | 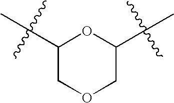 |
| 783 | OH/H | OH | 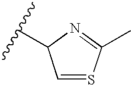 | 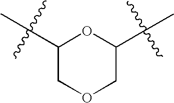 |
| 784 | OH/H | OH | 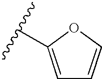 | 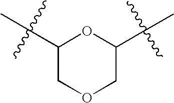 |
| 785 | OH/H | OH | 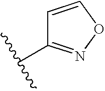 | 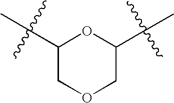 |
| 786 | OH/H | OH | 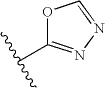 | 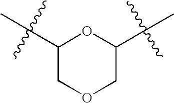 |
| 787 | OH/H | OH | 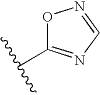 | 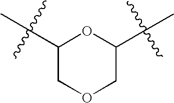 |
| 788 | OH/H | OH | 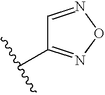 | 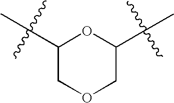 |
| 789 | OH/H | OH | 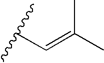 | 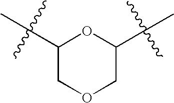 |
| 790 | OH/H | OH | 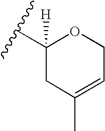 | 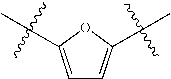 |
| 791 | OH/H | OH | 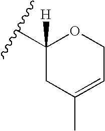 | 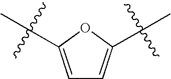 |
| 792 | OH/H | OH | 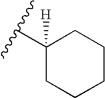 | 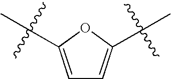 |
| 793 | OH/H | OH | 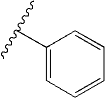 | 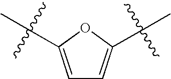 |
| 794 | OH/H | OH | 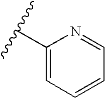 | 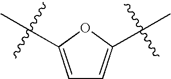 |
| 795 | OH/H | OH | 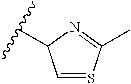 | 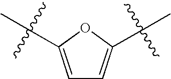 |
| 796 | OH/H | OH | 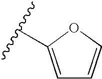 | 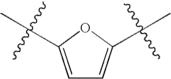 |
| 797 | OH/H | OH | 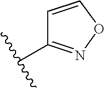 | 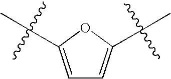 |
| 798 | OH/H | OH | 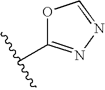 | 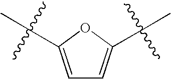 |
| 799 | OH/H | OH | 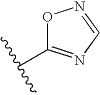 | 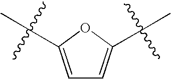 |
| 800 | OH/H | OH | 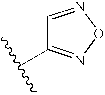 | 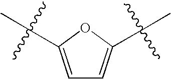 |
| 801 | OH/H | OH | 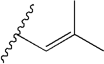 | 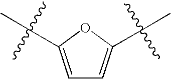 |
| 802 | OH/H | OH | 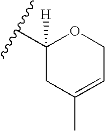 | 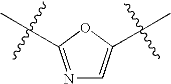 |
| 803 | OH/H | OH | 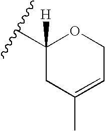 | 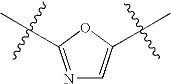 |
| 804 | OH/H | OH | 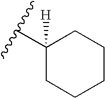 | 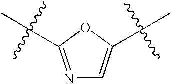 |
| 805 | OH/H | OH | 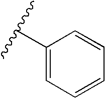 | 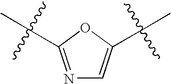 |
| 806 | OH/H | OH | 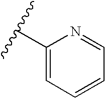 | 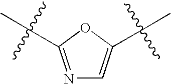 |
| 807 | OH/H | OH | 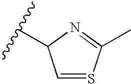 | 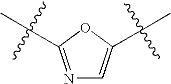 |
| 808 | OH/H | OH | 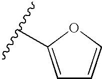 | 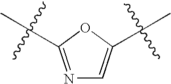 |
| 809 | OH/H | OH | 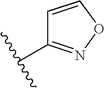 | 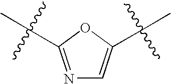 |
| 810 | OH/H | OH | 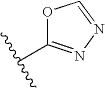 | 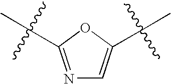 |
| 811 | OH/H | OH | 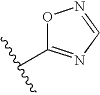 | 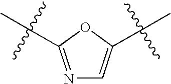 |
| 812 | OH/H | OH | 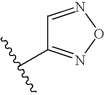 | 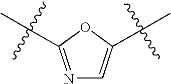 |
| 813 | OH/H | OH | 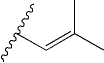 | 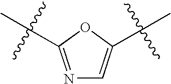 |
| 814 | OH/H | OH | 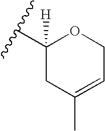 | 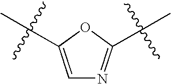 |
| 815 | OH/H | OH | 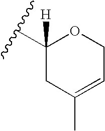 | 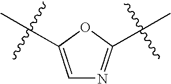 |
| 816 | OH/H | OH | 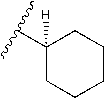 | 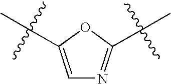 |
| 817 | OH/H | OH | 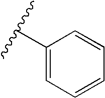 | 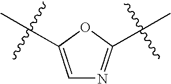 |
| 818 | OH/H | OH | 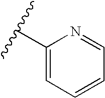 | 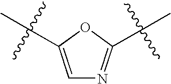 |
| 819 | OH/H | OH | 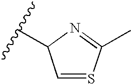 | 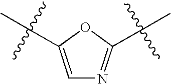 |
| 820 | OH/H | OH | 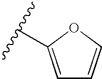 | 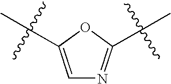 |
| 821 | OH/H | OH | 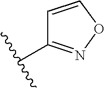 | 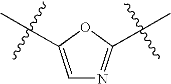 |
| 822 | OH/H | OH | 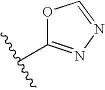 | 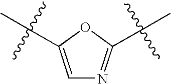 |
| 823 | OH/H | OH | 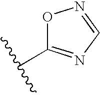 | 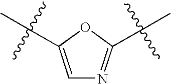 |
| 824 | OH/H | OH | 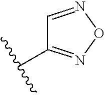 | 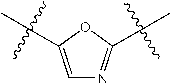 |
| 825 | OH/H | OH | 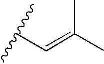 | 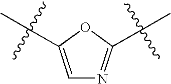 |
| 826 | OH/H | OH | 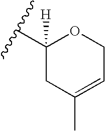 | 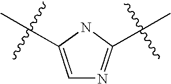 |
| 827 | OH/H | OH | 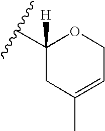 | 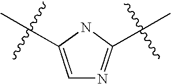 |
| 828 | OH/H | OH | 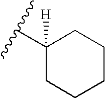 | 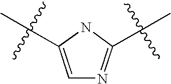 |
| 829 | OH/H | OH | 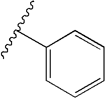 | 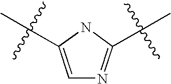 |
| 830 | OH/H | OH | 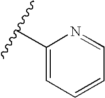 | 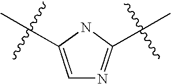 |
| 831 | OH/H | OH | 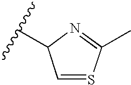 | 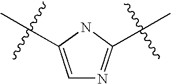 |
| 832 | OH/H | OH | 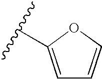 | 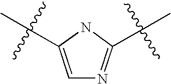 |
| 833 | OH/H | OH | 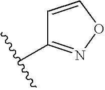 | 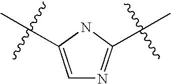 |
| 834 | OH/H | OH | 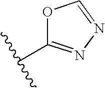 | 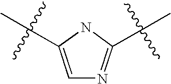 |
| 835 | OH/H | OH | 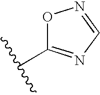 | 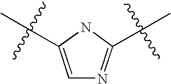 |
| 836 | OH/H | OH | 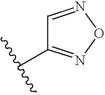 | 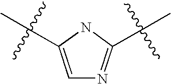 |
| 837 | OH/H | OH | 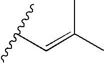 | 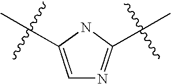 |
| 838 | OH/H | OH | 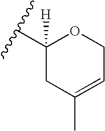 | 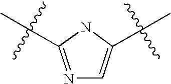 |
| 839 | OH/H | OH | 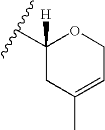 | 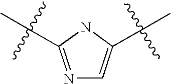 |
| 840 | OH/H | OH | 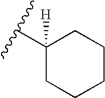 | 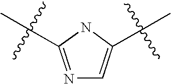 |
| 841 | OH/H | OH | 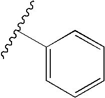 | 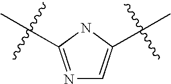 |
| 842 | OH/H | OH | 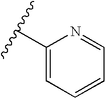 | 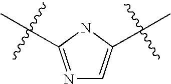 |
| 843 | OH/H | OH | 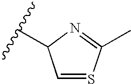 | 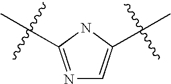 |
| 844 | OH/H | OH | 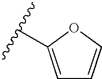 | 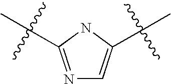 |
| 845 | OH/H | OH | 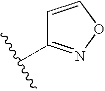 | 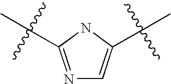 |
| 846 | OH/H | OH | 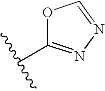 | 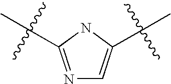 |
| 847 | OH/H | OH | 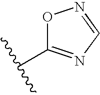 | 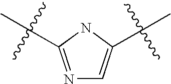 |
| 848 | OH/H | OH | 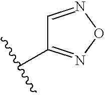 | 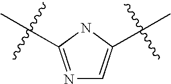 |
| 849 | OH/H | OH | 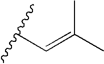 | 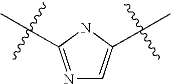 |
| 850 | OH/H | OH | 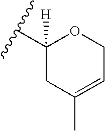 | 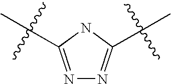 |
| 851 | OH/H | OH | 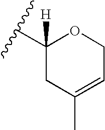 | 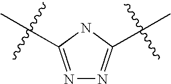 |
| 852 | OH/H | OH | 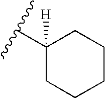 | 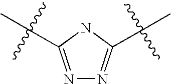 |
| 853 | OH/H | OH | 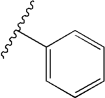 | 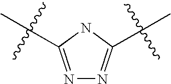 |
| 854 | OH/H | OH | 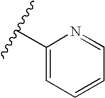 | 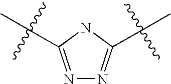 |
| 855 | OH/H | OH | 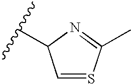 | 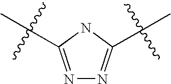 |
| 856 | OH/H | OH | 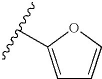 | 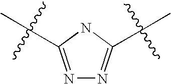 |
| 857 | OH/H | OH | 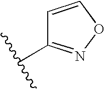 | 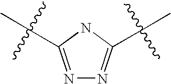 |
| 858 | OH/H | OH | 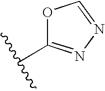 | 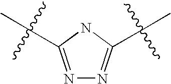 |
| 859 | OH/H | OH | 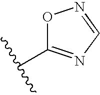 | 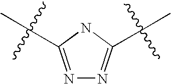 |
| 860 | OH/H | OH | 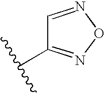 | 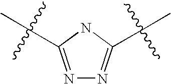 |
| 861 | OH/H | OH | 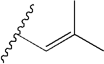 | 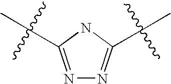 |
| 862 | OH/H | OH | 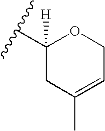 | 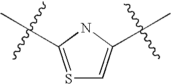 |
| 863 | OH/H | OH | 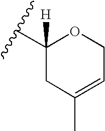 | 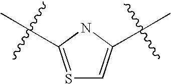 |
| 864 | OH/H | OH | 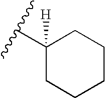 | 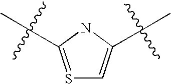 |
| 865 | OH/H | OH | 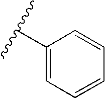 | 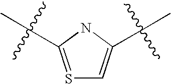 |
| 866 | OH/H | OH | 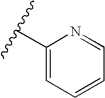 | 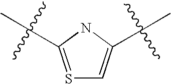 |
| 867 | OH/H | OH | 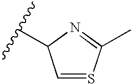 | 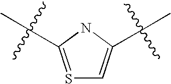 |
| 868 | OH/H | OH | 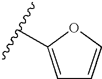 | 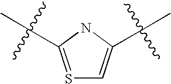 |
| 869 | OH/H | OH | 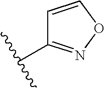 | 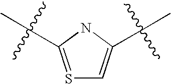 |
| 870 | OH/H | OH | 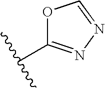 | 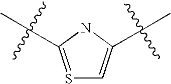 |
| 871 | OH/H | OH | 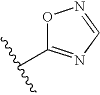 | 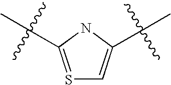 |
| 872 | OH/H | OH | 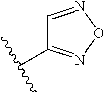 | 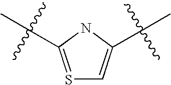 |
| 873 | OH/H | OH | 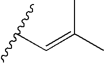 | 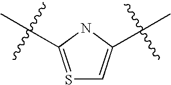 |
| 874 | OH/H | OH | 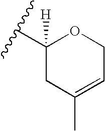 | 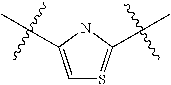 |
| 875 | OH/H | OH | 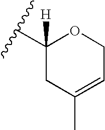 | 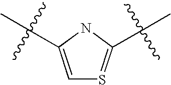 |
| 876 | OH/H | OH | 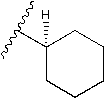 | 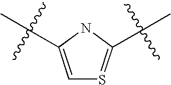 |
| 877 | OH/H | OH | 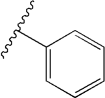 | 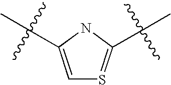 |
| 878 | OH/H | OH | 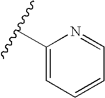 | 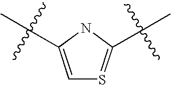 |
| 879 | OH/H | OH | 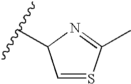 | 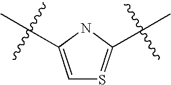 |
| 880 | OH/H | OH | 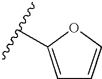 | 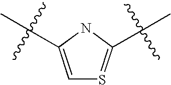 |
| 881 | OH/H | OH | 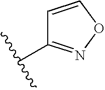 | 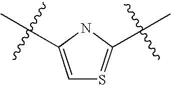 |
| 882 | OH/H | OH | 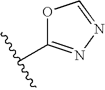 | 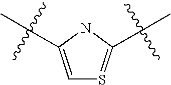 |
| 883 | OH/H | OH | 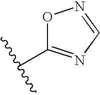 | 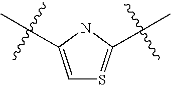 |
| 884 | OH/H | OH | 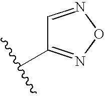 | 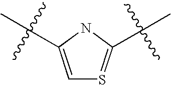 |
| 885 | OH/H | OH | 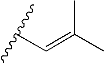 | 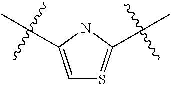 |
| 886 | OH/H | OH | 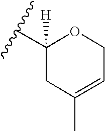 | 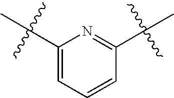 |
| 887 | OH/H | OH | 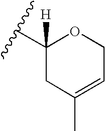 | 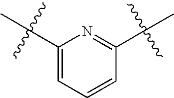 |
| 888 | OH/H | OH | 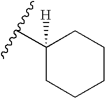 | 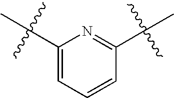 |
| 889 | OH/H | OH | 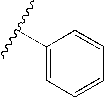 | 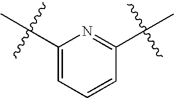 |
| 890 | OH/H | OH | 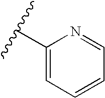 | 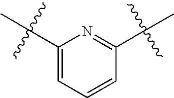 |
| 891 | OH/H | OH | 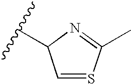 | 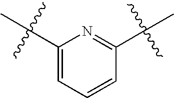 |
| 892 | OH/H | OH | 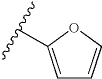 | 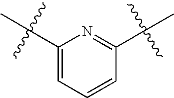 |
| 893 | OH/H | OH | 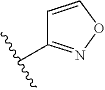 | 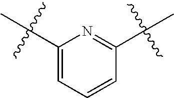 |
| 894 | OH/H | OH | 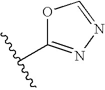 | 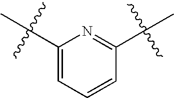 |
| 895 | OH/H | OH | 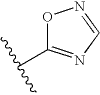 | 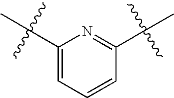 |
| 896 | OH/H | OH | 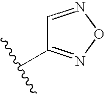 | 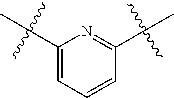 |
| 897 | OH/H | OH | 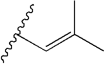 | 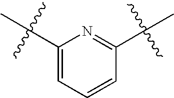 |
| 898 | OH/H | OH | 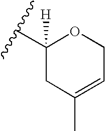 | 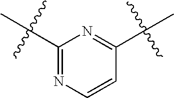 |
| 899 | OH/H | OH | 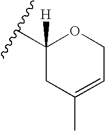 | 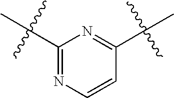 |
| 900 | OH/H | OH | 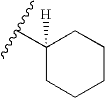 | 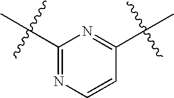 |
| 901 | OH/H | OH | 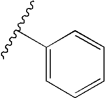 | 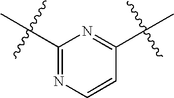 |
| 902 | OH/H | OH | 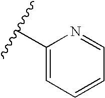 | 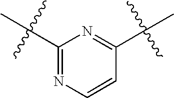 |
| 903 | OH/H | OH | 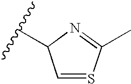 | 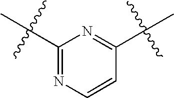 |
| 904 | OH/H | OH | 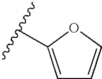 | 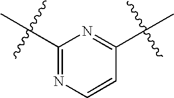 |
| 905 | OH/H | OH | 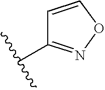 | 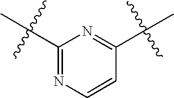 |
| 906 | OH/H | OH | 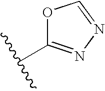 | 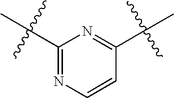 |
| 907 | OH/H | OH | 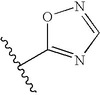 | 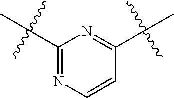 |
| 908 | OH/H | OH | 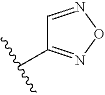 | 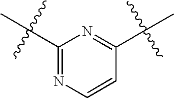 |
| 909 | OH/H | OH | 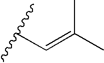 | 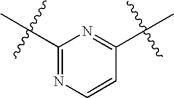 |
| 910 | OH/H | OH | 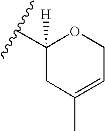 | 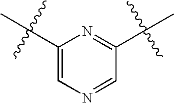 |
| 911 | OH/H | OH | 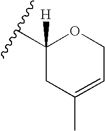 | 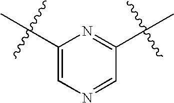 |
| 912 | OH/H | OH | 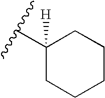 | 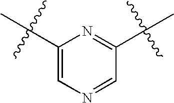 |
| 913 | OH/H | OH | 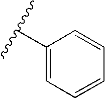 | 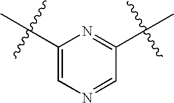 |
| 914 | OH/H | OH | 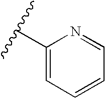 | 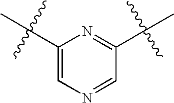 |
| 915 | OH/H | OH | 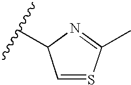 | 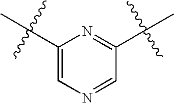 |
| 916 | OH/H | OH | 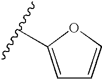 | 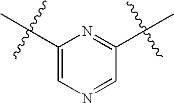 |
| 917 | OH/H | OH | 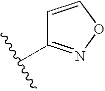 | 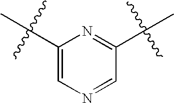 |
| 918 | OH/H | OH | 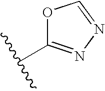 | 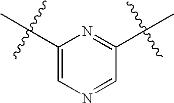 |
| 919 | OH/H | OH | 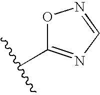 | 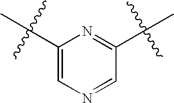 |
| 920 | OH/H | OH | 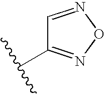 | 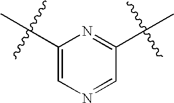 |
| 921 | OH/H | OH | 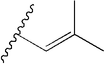 | 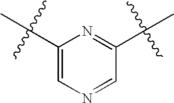 |
| 922 | OH/H | OH | 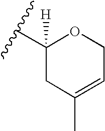 | 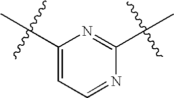 |
| 923 | OH/H | OH | 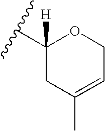 | 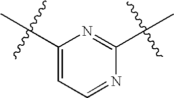 |
| 924 | OH/H | OH | 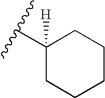 | 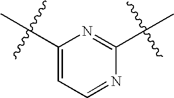 |
| 925 | OH/H | OH | 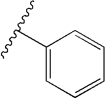 | 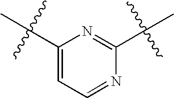 |
| 926 | OH/H | OH | 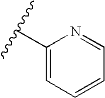 | 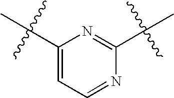 |
| 927 | OH/H | OH | 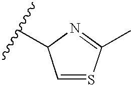 | 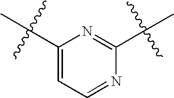 |
| 928 | OH/H | OH | 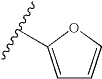 | 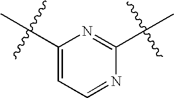 |
| 929 | OH/H | OH | 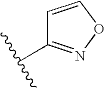 | 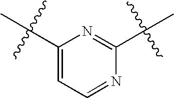 |
| 930 | OH/H | OH | 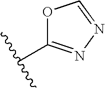 | 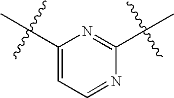 |
| 931 | OH/H | OH | 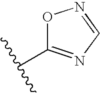 | 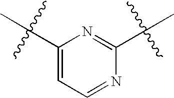 |
| 932 | OH/H | OH | 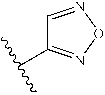 | 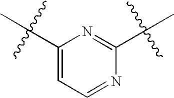 |
| 933 | OH/H | OH | 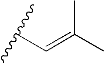 | 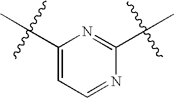 |
| 934 | OH/H | OH | 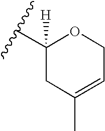 | 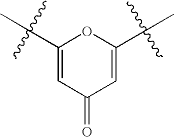 |
| 935 | OH/H | OH | 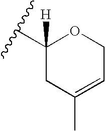 | 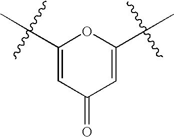 |
| 936 | OH/H | OH | 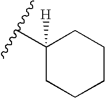 | 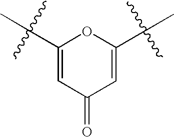 |
| 937 | OH/H | OH | 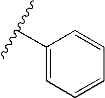 | 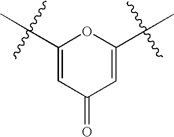 |
| 938 | OH/H | OH | 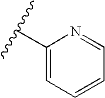 | 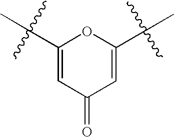 |
| 939 | OH/H | OH | 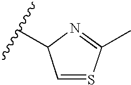 | 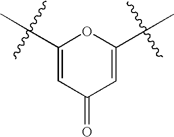 |
| 940 | OH/H | OH | 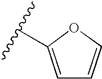 | 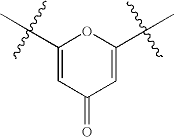 |
| 941 | OH/H | OH | 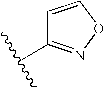 | 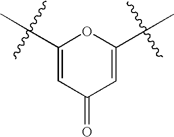 |
| 942 | OH/H | OH | 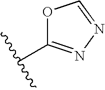 | 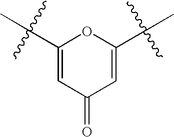 |
| 943 | OH/H | OH | 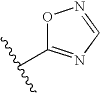 | 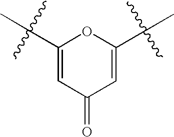 |
| 944 | OH/H | OH | 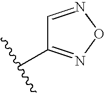 | 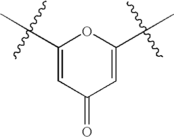 |
| 945 | OH/H | OH | 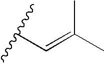 | 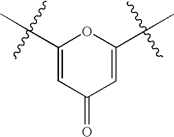 |

### EXAMPLE 3

The following macrocycles of Formula XIV are prepared, using appropriate reagents and according generally to the methods described herein.

| | |
|---|---|
| | (XIV) |
| 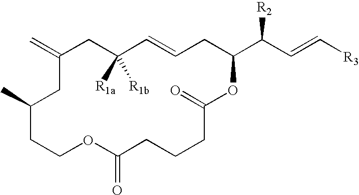 | |

| Com- | | | |
|---|---|---|---|
| pound | R1a/R1b | R2 | R3 |
| 950 | OH/H | OH | 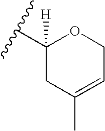 |
| 951 | OH/H | OCH3 | 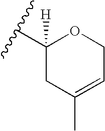 |
| 952 | OAc/H | OH | 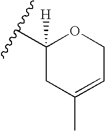 |
| 953 | p-NO2(C6H4)CO2/H | OH | 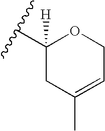 |
| 954 | OH/H | OH | 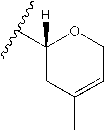 |
| 955 | OH/H | OCH3 | 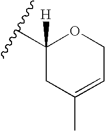 |
| 956 | OAc/H | OH | 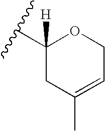 |
| 957 | p-NO2(C6H4)CO2/H | OH | 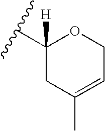 |
| 958 | OH/H | OH | 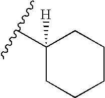 |
| 959 | OH/H | OCH3 | 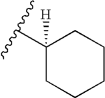 |
| 960 | OAc/H | OH | 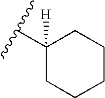 |
| 961 | p-NO2(C6H4)CO2/H | OH | 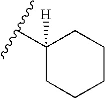 |
| 962 | OH/H | OH | 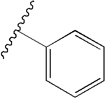 |
| 963 | OH/H | OCH3 | 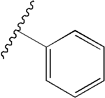 |
| 964 | OAc/H | OH | 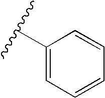 |
| 965 | p-NO2(C6H4)CO2/H | OH | 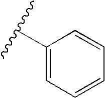 |
| 966 | OH/H | OH | 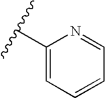 |
| 967 | OH/H | OCH3 | 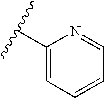 |
| 968 | OAc/H | OH | 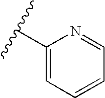 |
| 969 | p-NO2(C6H4)CO2/H | OH | 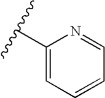 |
| 970 | OH/H | OH | 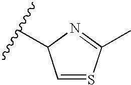 |
| 971 | OH/H | OCH3 | 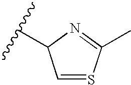 |
| 972 | OAc/H | OH | 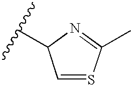 |
| 973 | p-NO2(C6H4)CO2/H | OH | 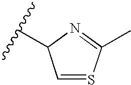 |
| 974 | OH/H | OH | 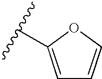 |
| 975 | OH/H | OCH3 | 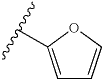 |
| 976 | OAc/H | OH | 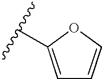 |
| 977 | p-NO2(C6H4)CO2/H | OH | 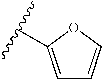 |
| 978 | OH/H | OH | 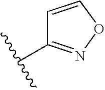 |
| 979 | OH/H | OCH3 | 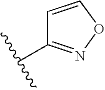 |
| 980 | OAc/H | OH | 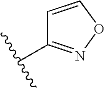 |
| 981 | p-NO2(C6H4)CO2/H | OH | 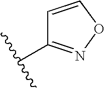 |
| 982 | OH/H | OH | 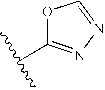 |
| 983 | OH/H | OCH3 | 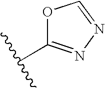 |
| 984 | OAc/H | OH | 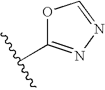 |
| 985 | p-NO2(C6H4)CO2/H | OH | 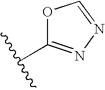 |
| 986 | OH/H | OH | 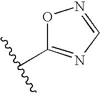 |
| 987 | OH/H | OCH3 | 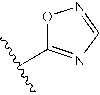 |
| 988 | OAc/H | OH | 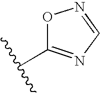 |
| 989 | p-NO2(C6H4)CO2/H | OH | 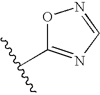 |
| 990 | OH/H | OH | 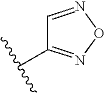 |
| 991 | OH/H | OCH3 | 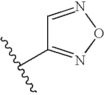 |
| 992 | OAc/H | OH | 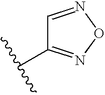 |
| 993 | p-NO2(C6H4)CO2/H | OH | 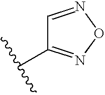 |
| 994 | OH/H | OH | 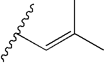 |
| 995 | OH/H | OCH3 | 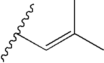 |
| 996 | OAc/H | OH | 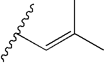 |
| 997 | p-NO2(C6H4)CO2/H | OH | 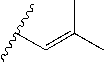 |

### EXAMPLE 4

The following macrocycles of Formula XV are prepared, using appropriate reagents and according generally to the methods described herein.

| | |
|---|---|
| | (XV) |
| 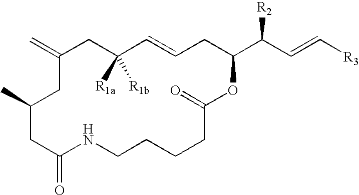 | |

| Com- | | | |
|---|---|---|---|
| pound | R1a/R1b | R2 | R3 |
| 1000 | OH/H | OH | 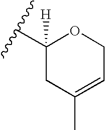 |
| 1001 | OH/H | OCH3 | 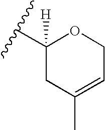 |
| 1002 | OAc/H | OH | 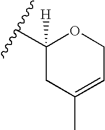 |
| 1003 | p-NO2(C6H4)CO2/H | OH | 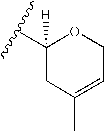 |
| 1004 | OH/H | OH | 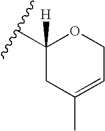 |
| 1005 | OH/H | OCH3 | 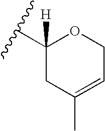 |
| 1006 | OAc/H | OH | 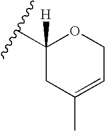 |
| 1007 | p-NO2(C6H4)CO2/H | OH | 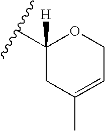 |
| 1008 | OH/H | OH | 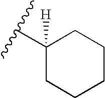 |
| 1009 | OH/H | OCH3 | 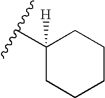 |
| 1010 | OAc/H | OH | 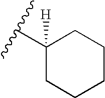 |
| 1011 | p-NO2(C6H4)CO2/H | OH | 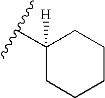 |
| 1012 | OH/H | OH | 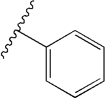 |
| 1013 | OH/H | OCH3 | 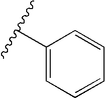 |
| 1014 | OAc/H | OH | 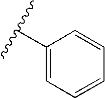 |
| 1015 | p-NO2(C6H4)CO2/H | OH | 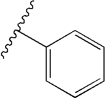 |
| 1016 | OH/H | OH | 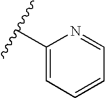 |
| 1017 | OH/H | OCH3 | 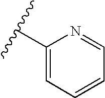 |
| 1018 | OAc/H | OH | 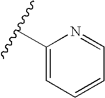 |
| 1019 | p-NO2(C6H4)CO2/H | OH | 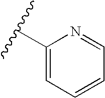 |
| 1020 | OH/H | OH | 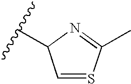 |
| 1021 | OH/H | OCH3 | 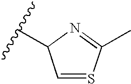 |
| 1022 | OAc/H | OH | 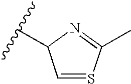 |
| 1023 | p-NO2(C6H4)CO2/H | OH | 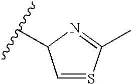 |
| 1024 | OH/H | OH | 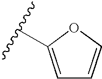 |
| 1025 | OH/H | OCH3 | 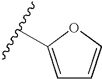 |
| 1026 | OAc/H | OH | 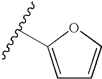 |
| 1027 | p-NO2(C6H4)CO2/H | OH | 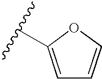 |
| 1028 | OH/H | OH | 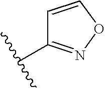 |
| 1029 | OH/H | OCH3 | 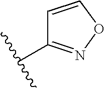 |
| 1030 | OAc/H | OH | 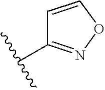 |
| 1031 | p-NO2(C6H4)CO2/H | OH | 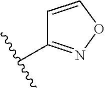 |
| 1032 | OH/H | OH | 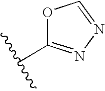 |
| 1033 | OH/H | OCH3 | 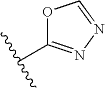 |
| 1034 | OAc/H | OH | 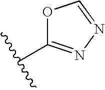 |
| 1035 | p-NO2(C6H4)CO2/H | OH | 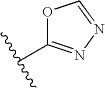 |
| 1036 | OH/H | OH | 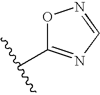 |
| 1037 | OH/H | OCH3 | 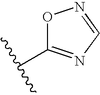 |
| 1038 | OAc/H | OH | 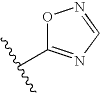 |
| 1039 | p-NO2(C6H4)CO2/H | OH | 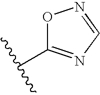 |
| 1040 | OH/H | OH | 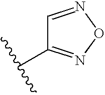 |
| 1041 | OH/H | OCH3 | 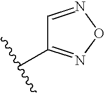 |
| 1042 | OAc/H | OH | 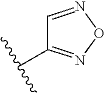 |
| 1043 | p-NO2(C6H4)CO2/H | OH | 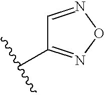 |
| 1044 | OH/H | OH | 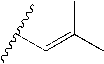 |
| 1045 | OH/H | OCH3 | 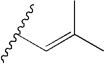 |
| 1046 | OAc/H | OH | 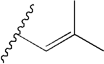 |
| 1047 | p-NO2(C6H4)CO2/H | OH | 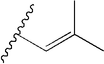 |

### EXAMPLE 5

The following macrocycles of Formula XVI are prepared, using appropriate reagents and according generally to the methods described herein.

| | |
|---|---|
| | (XVI) |
| 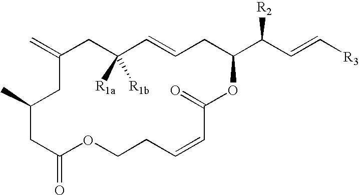 | |

| Compound | R1a/R1b | R2 | R3 |
|---|---|---|---|
| 1050 | OH/H | OH | 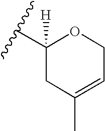 |
| 1051 | OH/H | OCH3 | 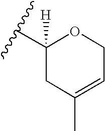 |
| 1052 | OAc/H | OH | 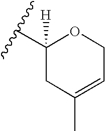 |
| 1053 | p-NO2(C6H4)CO2/H | OH | 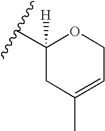 |
| 1054 | OH/H | OH | 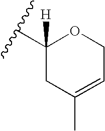 |
| 1055 | OH/H | OCH3 | 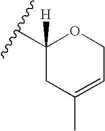 |
| 1056 | OAc/H | OH | 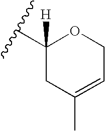 |
| 1057 | p-NO2(C6H4)CO2/H | OH | 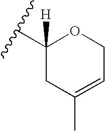 |
| 1058 | OH/H | OH | 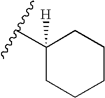 |
| 1059 | OH/H | OCH3 | 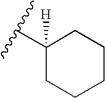 |
| 1060 | OAc/H | OH | 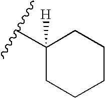 |
| 1061 | p-NO2(C6H4)CO2/H | OH | 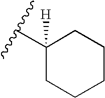 |
| 1062 | OH/H | OH | 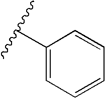 |
| 1063 | OH/H | OCH3 | 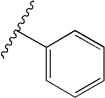 |
| 1064 | OAc/H | OH | 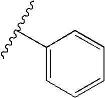 |
| 1065 | p-NO2(C6H4)CO2/H | OH | 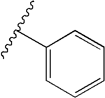 |
| 1066 | OH/H | OH | 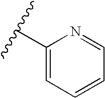 |
| 1067 | OH/H | OCH3 | 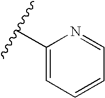 |
| 1068 | OAc/H | OH | 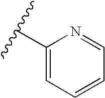 |
| 1069 | p-NO2(C6H4)CO2/H | OH | 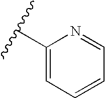 |
| 1070 | OH/H | OH | 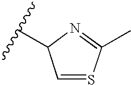 |
| 1071 | OH/H | OCH3 | 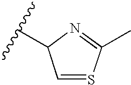 |
| 1072 | OAc/H | OH | 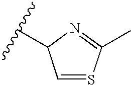 |
| 1073 | p-NO2(C6H4)CO2/H | OH | 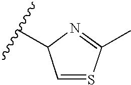 |
| 1074 | OH/H | OH | 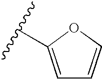 |
| 1075 | OH/H | OCH3 | 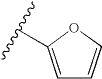 |
| 1076 | OAc/H | OH | 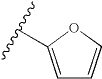 |
| 1077 | p-NO2(C6H4)CO2/H | OH | 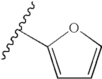 |
| 1078 | OH/H | OH | 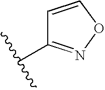 |
| 1079 | OH/H | OCH3 | 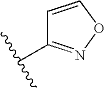 |
| 1080 | OAc/H | OH | 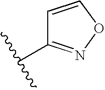 |
| 1081 | p-NO2(C6H4)CO2/H | OH | 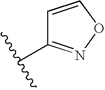 |
| 1082 | OH/H | OH | 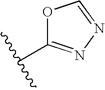 |
| 1083 | OH/H | OCH3 | 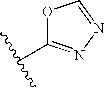 |
| 1084 | OAc/H | OH | 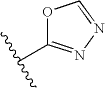 |
| 1085 | p-NO2(C6H4)CO2/H | OH | 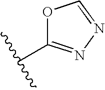 |
| 1086 | OH/H | OH | 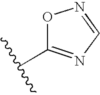 |
| 1087 | OH/H | OCH3 | 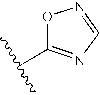 |
| 1088 | OAc/H | OH | 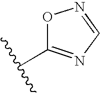 |
| 1089 | p-NO2(C6H4)CO2/H | OH | 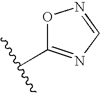 |
| 1090 | OH/H | OH | 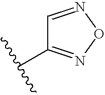 |
| 1091 | OH/H | OCH3 | 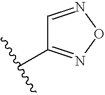 |
| 1092 | OAc/H | OH | 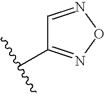 |
| 1093 | p-NO2(C6H4)CO2/H | OH | 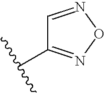 |
| 1094 | OH/H | OH | 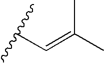 |
| 1095 | OH/H | OCH3 | 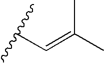 |
| 1096 | OAc/H | OH | 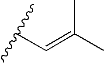 |
| 1097 | p-NO2(C6H4)CO2/H | OH | 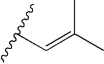 |

### EXAMPLE 6

The following macrocycles of Formula XVII are prepared, using appropriate reagents and according generally to the methods described herein.

| | |
|---|---|
| | (XVII) |
| 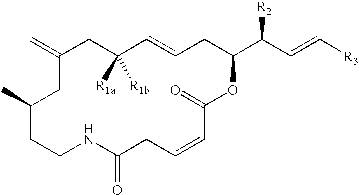 | |

| Compound | R1a/R1b | R2 | R3 |
|---|---|---|---|
| 1100 | OH/H | OH | 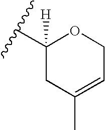 |
| 1101 | OH/H | OCH3 | 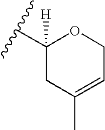 |
| 1102 | OAc/H | OH | 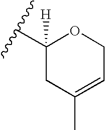 |
| 1103 | p-NO2(C6H4)CO2/H | OH | 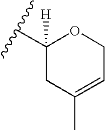 |
| 1104 | OH/H | OH | 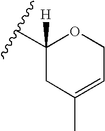 |
| 1105 | OH/H | OCH3 | 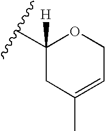 |
| 1106 | OAc/H | OH | 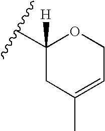 |
| 1107 | p-NO2(C6H4)CO2/H | OH | 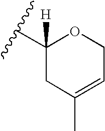 |
| 1108 | OH/H | OH | 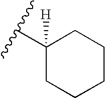 |
| 1109 | OH/H | OCH3 | 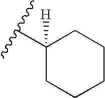 |
| 1110 | OAc/H | OH | 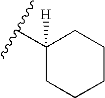 |
| 1111 | p-NO2(C6H4)CO2/H | OH | 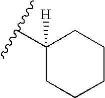 |
| 1112 | OH/H | OH | 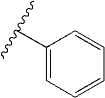 |
| 1113 | OH/H | OCH3 | 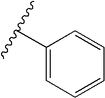 |
| 1114 | OAc/H | OH | 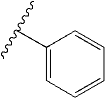 |
| 1115 | p-NO2(C6H4)CO2/H | OH | 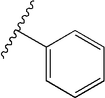 |
| 1116 | OH/H | OH | 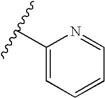 |
| 1117 | OH/H | OCH3 | 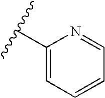 |
| 1118 | OAc/H | OH | 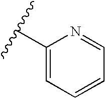 |
| 1119 | p-NO2(C6H4)CO2/H | OH | 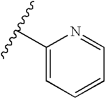 |
| 1120 | OH/H | OH | 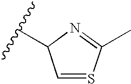 |
| 1121 | OH/H | OCH3 | 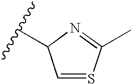 |
| 1122 | OAc/H | OH | 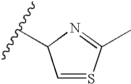 |
| 1123 | p-NO2(C6H4)CO2/H | OH | 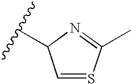 |
| 1124 | OH/H | OH | 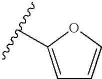 |
| 1125 | OH/H | OCH3 | 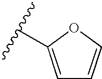 |
| 1126 | OAc/H | OH | 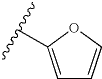 |
| 1127 | p-NO2(C6H4)CO2/H | OH | 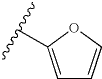 |
| 1128 | OH/H | OH | 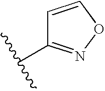 |
| 1129 | OH/H | OCH3 | 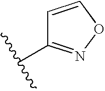 |
| 1130 | OAc/H | OH | 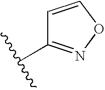 |
| 1131 | p-NO2(C6H4)CO2/H | OH | 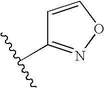 |
| 1132 | OH/H | OH | 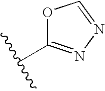 |
| 1133 | OH/H | OCH3 | 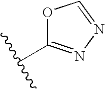 |
| 1134 | OAc/H | OH | 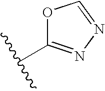 |
| 1135 | p-NO2(C6H4)CO2/H | OH | 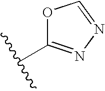 |
| 1136 | OH/H | OH | 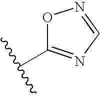 |
| 1137 | OH/H | OCH3 | 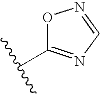 |
| 1138 | OAc/H | OH | 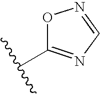 |
| 1139 | p-NO2(C6H4)CO2/H | OH | 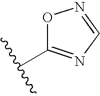 |
| 1140 | OH/H | OH | 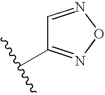 |
| 1141 | OH/H | OCH3 | 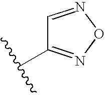 |
| 1142 | OAc/H | OH | 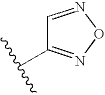 |
| 1143 | p-NO2(C6H4)CO2/H | OH | 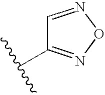 |
| 1144 | OH/H | OH | 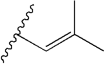 |
| 1145 | OH/H | OCH3 | 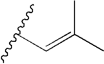 |
| 1146 | OAc/H | OH | 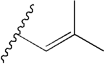 |
| 1147 | p-NO2(C6H4)CO2/H | OH | 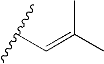 |

### EXAMPLE 7

The following macrocycles of Formula XVIII are prepared, using appropriate reagents and according generally to the methods described herein.

| | |
|---|---|
| | (XVIII) |
| 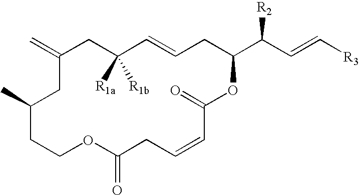 | |

| Compound | R1a/R1b | R2 | R3 |
|---|---|---|---|
| 1150 | OH/H | OH | 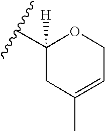 |
| 1151 | OH/H | OCH3 | 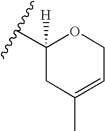 |
| 1152 | OAc/H | OH | 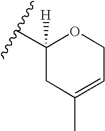 |
| 1153 | p-NO2(C6H4)CO2/H | OH | 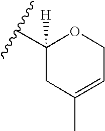 |
| 1154 | OH/H | OH | 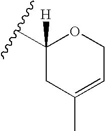 |
| 1155 | OH/H | OCH3 | 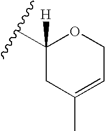 |
| 1156 | OAc/H | OH | 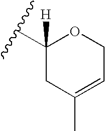 |
| 1157 | p-NO2(C6H4)CO2/H | OH | 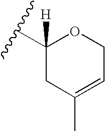 |
| 1158 | OH/H | OH | 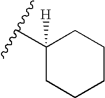 |
| 1159 | OH/H | OCH3 | 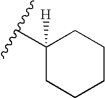 |
| 1160 | OAc/H | OH | 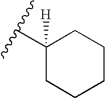 |
| 1161 | p-NO2(C6H4)CO2/H | OH | 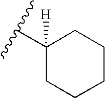 |
| 1162 | OH/H | OH | 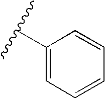 |
| 1163 | OH/H | OCH3 | 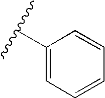 |
| 1164 | OAc/H | OH | 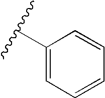 |
| 1165 | p-NO2(C6H4)CO2/H | OH | 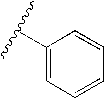 |
| 1166 | OH/H | OH | 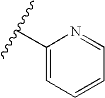 |
| 1167 | OH/H | OCH3 | 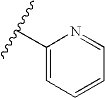 |
| 1168 | OAc/H | OH | 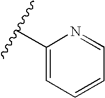 |
| 1169 | p-NO2(C6H4)CO2/H | OH | 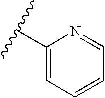 |
| 1170 | OH/H | OH | 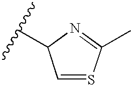 |
| 1171 | OH/H | OCH3 | 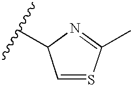 |
| 1172 | OAc/H | OH | 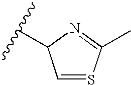 |
| 1173 | p-NO2(C6H4)CO2/H | OH | 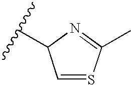 |
| 1174 | OH/H | OH | 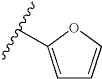 |
| 1175 | OH/H | OCH3 | 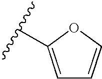 |
| 1176 | OAc/H | OH | 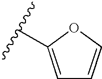 |
| 1177 | p-NO2(C6H4)CO2/H | OH | 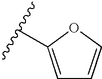 |
| 1178 | OH/H | OH | 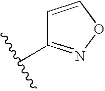 |
| 1179 | OH/H | OCH3 | 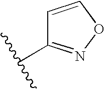 |
| 1180 | OAc/H | OH | 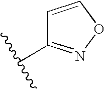 |
| 1181 | p-NO2(C6H4)CO2/H | OH | 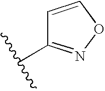 |
| 1182 | OH/H | OH | 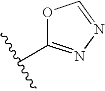 |
| 1183 | OH/H | OCH3 | 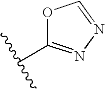 |
| 1184 | OAc/H | OH | 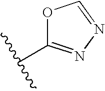 |
| 1185 | p-NO2(C6H4)CO2/H | OH | 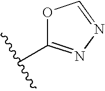 |
| 1186 | OH/H | OH | 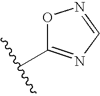 |
| 1187 | OH/H | OCH3 | 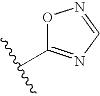 |
| 1188 | OAc/H | OH | 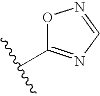 |
| 1189 | p-NO2(C6H4)CO2/H | OH | 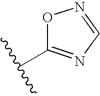 |
| 1190 | OH/H | OH | 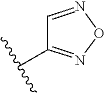 |
| 1191 | OH/H | OCH3 | 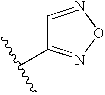 |
| 1192 | OAc/H | OH | 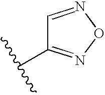 |
| 1193 | p-NO2(C6H4)CO2/H | OH | 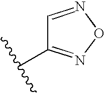 |
| 1194 | OH/H | OH | 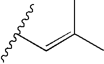 |
| 1195 | OH/H | OCH3 | 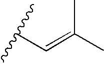 |
| 1196 | OAc/H | OH | 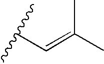 |
| 1197 | p-NO2(C6H4)CO2/H | OH | 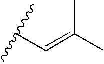 |

### EXAMPLE 8

The following macrocycles of Formula XIX are prepared, using appropriate reagents and according generally to the methods described herein.

| | |
|---|---|
| | (XIX) |
| 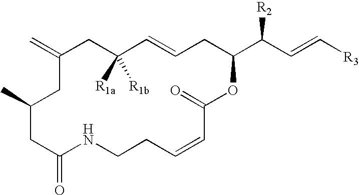 | |

| Compound | R1a/R1b | R2 | R3 |
|---|---|---|---|
| 1200 | OH/H | OH | 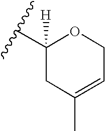 |
| 1201 | OH/H | OCH3 | 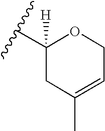 |
| 1202 | OAc/H | OH | 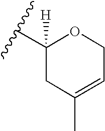 |
| 1203 | p-NO2(C6H4)CO2/H | OH | 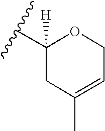 |
| 1204 | OH/H | OH | 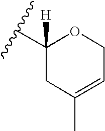 |
| 1205 | OH/H | OCH3 | 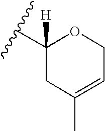 |
| 1206 | OAc/H | OH | 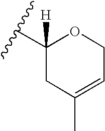 |
| 1207 | p-NO2(C6H4)CO2/H | OH | 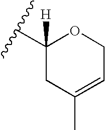 |
| 1208 | OH/H | OH | 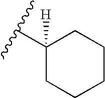 |
| 1209 | OH/H | OCH3 | 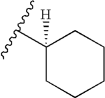 |
| 1210 | OAc/H | OH | 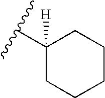 |
| 1211 | p-NO2(C6H4)CO2/H | OH | 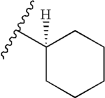 |
| 1212 | OH/H | OH | 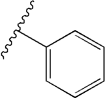 |
| 1213 | OH/H | OCH3 | 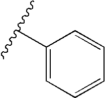 |
| 1214 | OAc/H | OH | 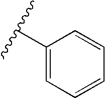 |
| 1215 | p-NO2(C6H4)CO2/H | OH | 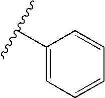 |
| 1216 | OH/H | OH | 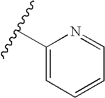 |
| 1217 | OH/H | OCH3 | 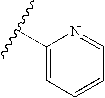 |
| 1218 | OAc/H | OH | 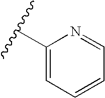 |
| 1219 | p-NO2(C6H4)CO2/H | OH | 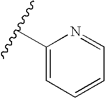 |
| 1220 | OH/H | OH | 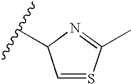 |
| 1221 | OH/H | OCH3 | 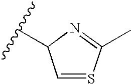 |
| 1222 | OAc/H | OH | 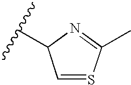 |
| 1223 | p-NO2(C6H4)CO2/H | OH | 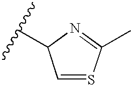 |
| 1224 | OH/H | OH | 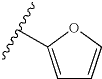 |
| 1225 | OH/H | OCH3 | 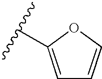 |
| 1226 | OAc/H | OH | 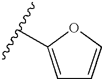 |
| 1227 | p-NO2(C6H4)CO2/H | OH | 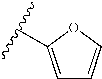 |
| 1228 | OH/H | OH | 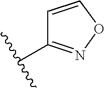 |
| 1229 | OH/H | OCH3 | 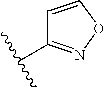 |
| 1230 | OAc/H | OH | 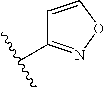 |
| 1231 | p-NO2(C6H4)CO2/H | OH | 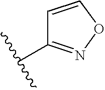 |
| 1232 | OH/H | OH | 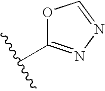 |
| 1233 | OH/H | OCH3 | 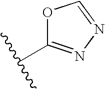 |
| 1234 | OAc/H | OH | 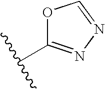 |
| 1235 | p-NO2(C6H4)CO2/H | OH | 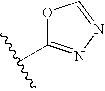 |
| 1236 | OH/H | OH | 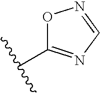 |
| 1237 | OH/H | OCH3 | 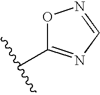 |
| 1238 | OAc/H | OH | 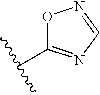 |
| 1239 | p-NO2(C6H4)CO2/H | OH | 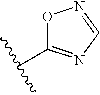 |
| 1240 | OH/H | OH | 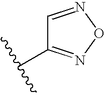 |
| 1241 | OH/H | OCH3 | 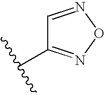 |
| 1242 | OAc/H | OH | 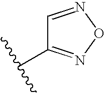 |
| 1243 | p-NO2(C6H4)CO2/H | OH | 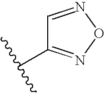 |
| 1244 | OH/H | OH | 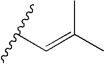 |
| 1245 | OH/H | OCH3 | 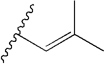 |
| 1246 | OAc/H | OH | 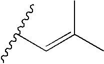 |
| 1247 | p-NO2(C6H4)CO2/H | OH | 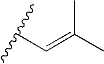 |

### EXAMPLE 9

The following macrocycles of Formula XX are prepared, using appropriate reagents and according generally to the methods described herein.

| | |
|---|---|
| | (XX) |
| 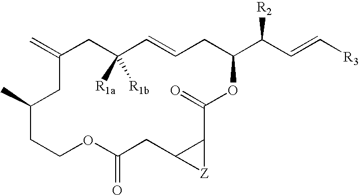 | |

| Compound | Z | R₁ₐ/R_{1b} | R₂ | R₃ |
|---|---|---|---|---|
| 1250a1250b1250c1250d | CH₂ONS | OH/H | OH | 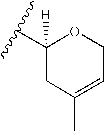 |
| 1251 | CH₂ONS | OH/H | OCH₃ | 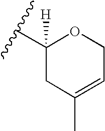 |
| 1252 | CH₂ONS | OAc/H | OH | 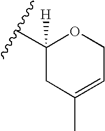 |
| 1253 | CH₂ONS | p-NO₂(C₆H₄)CO₂/H | OH | 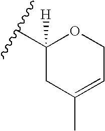 |
| 1254 | CH₂ONS | OH/H | OH | 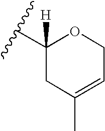 |
| 1255 | CH₂ONS | OH/H | OCH₃ | 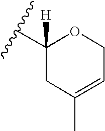 |
| 1256 | CH₂ONS | OAc/H | OH | 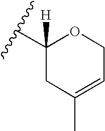 |
| 1257 | CH₂ONS | p-NO₂(C₆H₄)CO₂/H | OH | 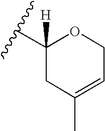 |
| 1258 | CH₂ONS | OH/H | OH | 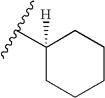 |
| 1259 | CH₂ONS | OH/H | OCH₃ | 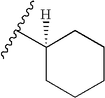 |
| 1260 | CH₂ONS | OAc/H | OH | 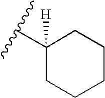 |
| 1261 | CH₂ONS | p-NO₂(C₆H₄)CO₂/H | OH | 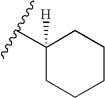 |
| 1262 | CH₂ONS | OH/H | OH | 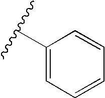 |
| 1263 | CH₂ONS | OH/H | OCH₃ | 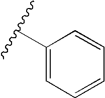 |
| 1264 | CH₂ONS | OAc/H | OH | 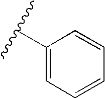 |
| 1265 | CH₂ONS | p-NO₂(C₆H₄)CO₂/H | OH | 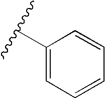 |
| 1266 | CH₂ONS | OH/H | OH | 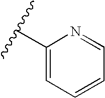 |
| 1267 | CH₂ONS | OH/H | OCH₃ | 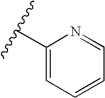 |
| 1268 | CH₂ONS | OAc/H | OH | 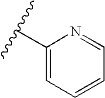 |
| 1269 | CH₂ONS | p-NO₂(C₆H₄)CO₂/H | OH | 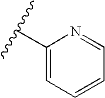 |
| 1270 | CH₂ONS | OH/H | OH | 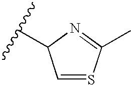 |
| 1271 | CH₂ONS | OH/H | OCH₃ | 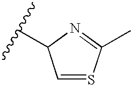 |
| 1272 | CH₂ONS | OAc/H | OH | 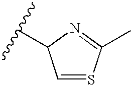 |
| 1273 | CH₂ONS | p-NO₂(C₆H₄)CO₂/H | OH | 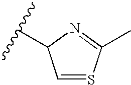 |
| 1274 | CH₂ONS | OH/H | OH | 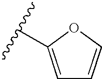 |
| 1275 | CH₂ONS | OH/H | OCH₃ | 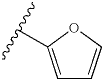 |
| 1276 | CH₂ONS | OAc/H | OH | 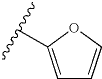 |
| 1277 | CH₂ONS | p-NO₂(C₆H₄)CO₂/H | OH | 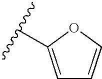 |
| 1278 | CH₂ONS | OH/H | OH | 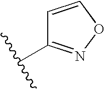 |
| 1279 | CH₂ONS | OH/H | OCH₃ | 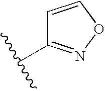 |
| 1280 | CH₂ONS | OAc/H | OH | 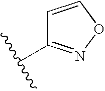 |
| 1281 | CH₂ONS | p-NO₂(C₆H₄)CO₂/H | OH | 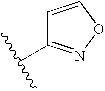 |
| 1282 | CH₂ONS | OH/H | OH | 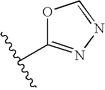 |
| 1283 | CH₂ONS | OH/H | OCH₃ | 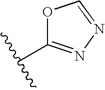 |
| 1284 | CH₂ONS | OAc/H | OH | 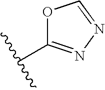 |
| 1285 | CH₂ONS | p-NO₂(C₆H₄)CO₂/H | OH | 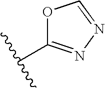 |
| 1286 | CH₂ONS | OH/H | OH | 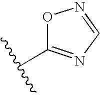 |
| 1287 | CH₂ONS | OH/H | OCH₃ | 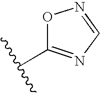 |
| 1288 | CH₂ONS | OAc/H | OH | 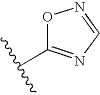 |
| 1289 | CH₂ONS | p-NO₂(C₆H₄)CO₂/H | OH | 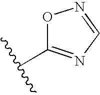 |
| 1290 | CH₂ONS | OH/H | OH | 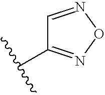 |
| 1291 | CH₂ONS | OH/H | OCH₃ | 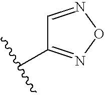 |
| 1292 | CH₂ONS | OAc/H | OH | 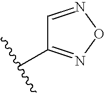 |
| 1293 | CH₂ONS | p-NO₂(C₆H₄)CO₂/H | OH | 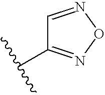 |
| 1294 | CH₂ONS | OH/H | OH | 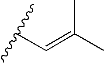 |
| 1295 | CH₂ONS | OH/H | OCH₃ | 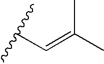 |
| 1296 | CH₂ONS | OAc/H | OH | 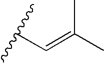 |
| 1297 | CH₂ONS | p-NO₂(C₆H₄)CO₂/H | OH | 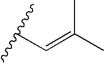 |

### EXAMPLE 10

The following macrocycles of Formula XXI are prepared, using appropriate reagents and according generally to the methods described herein.

| | |
|---|---|
| | (XXI) |
| 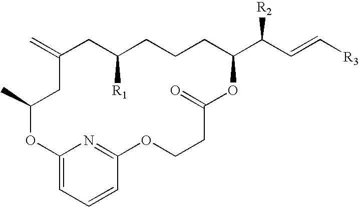 | |

| Com- | | | |
|---|---|---|---|
| pound | R1 | R2 | R3 |
| 1300 | OH | OH | 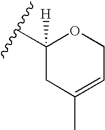 |
| 1301 | OH | OCH3 | 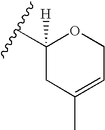 |
| 1302 | OAc | OH | 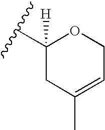 |
| 1303 | p-NO2(C6H4)CO2 | OH | 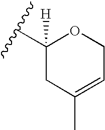 |
| 1304 | OH | OH | 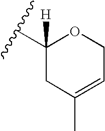 |
| 1305 | OH | OCH3 | 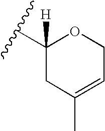 |
| 1306 | OAc | OH | 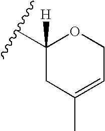 |
| 1307 | pNO2(C6H4)CO2 | OH | 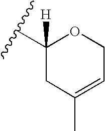 |
| 1308 | OH | OH | 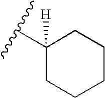 |
| 1309 | OH | OCH3 | 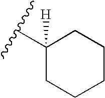 |
| 1310 | OAc | OH | 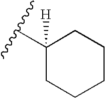 |
| 1311 | p-NO2(C6H4)CO2 | OH | 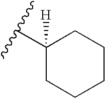 |
| 1312 | OH | OH | 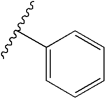 |
| 1313 | OH | OCH3 | 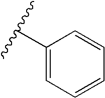 |
| 1314 | OAc | OH | 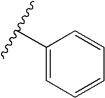 |
| 1315 | p-NO2(C6H4)CO2 | OH | 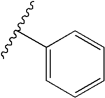 |
| 1316 | OH | OH | 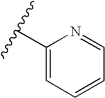 |
| 1317 | OH | OCH3 | 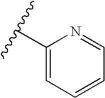 |
| 1318 | OAc | OH | 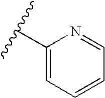 |
| 1319 | p-NO2(C6H4)CO2 | OH | 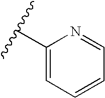 |
| 1320 | OH | OH | 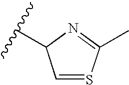 |
| 1321 | OH | OCH3 | 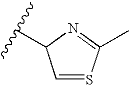 |
| 1322 | OAc | OH | 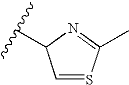 |
| 1323 | p-NO2(C6H4)CO2 | OH | 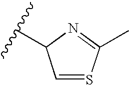 |
| 1324 | OH | OH | 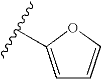 |
| 1325 | OH | OCH3 | 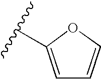 |
| 1326 | OAc | OH | 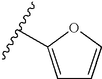 |
| 1327 | p-NO2(C6H4)CO2 | OH | 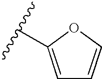 |
| 1328 | OH | OH | 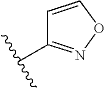 |
| 1329 | OH | OCH3 | 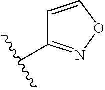 |
| 1330 | OAc | OH | 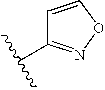 |
| 1331 | p-NO2(C6H4)CO2 | OH | 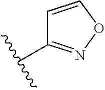 |
| 1332 | OH | OH | 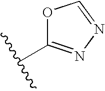 |
| 1333 | OH | OCH3 | 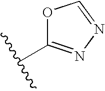 |
| 1334 | OAc | OH | 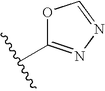 |
| 1335 | p-NO2(C6H4)CO2 | OH | 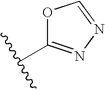 |
| 1336 | OH | OH | 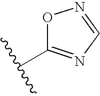 |
| 1337 | OH | OCH3 | 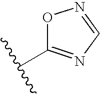 |
| 1338 | OAc | OH | 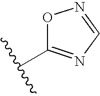 |
| 1339 | p-NO2(C6H4)CO2 | OH | 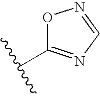 |
| 1340 | OH | OH | 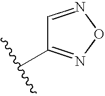 |
| 1341 | OH | OCH3 | 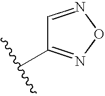 |
| 1342 | OAc | OH | 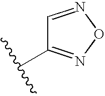 |
| 1343 | p-NO2(C6H4)CO2 | OH | 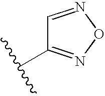 |
| 1344 | OH | OH | 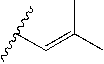 |
| 1345 | OH | OCH3 | 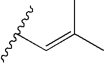 |
| 1346 | OAc | OH | 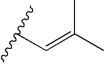 |
| 1347 | p-NO2(C6H4)CO2 | OH | 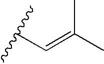 |

### EXAMPLE 11

The following macrocycles of Formula XXII are prepared, using appropriate reagents and according generally to the methods described herein.

| | |
|---|---|
| | (XXII) |
| 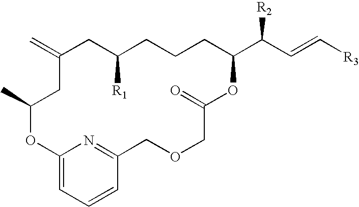 | |

| Com- | | | |
|---|---|---|---|
| pound | R1 | R2 | R3 |
| 1350 | OH | OH | 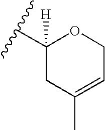 |
| 1351 | OH | OCH3 | 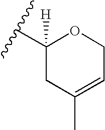 |
| 1352 | OAc | OH | 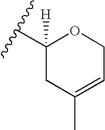 |
| 1353 | p-NO2(C6H4)CO2 | OH | 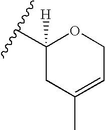 |
| 1354 | OH | OH | 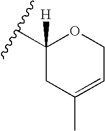 |
| 1355 | OH | OCH3 | 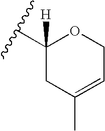 |
| 1356 | OAc | OH | 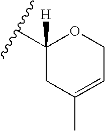 |
| 1357 | p-NO2(C6H4)CO2 | OH | 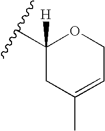 |
| 1358 | OH | OH | 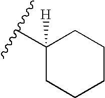 |
| 1359 | OH | OCH3 | 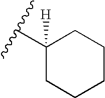 |
| 1360 | OAc | OH | 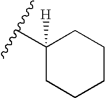 |
| 1361 | p-NO2(C6H4)CO2 | OH | 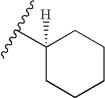 |
| 1362 | OH | OH | 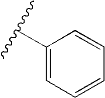 |
| 1363 | OH | OCH3 | 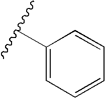 |
| 1364 | OAc | OH | 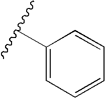 |
| 1365 | p-NO2(C6H4)CO2 | OH | 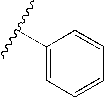 |
| 1366 | OH | OH | 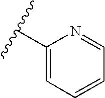 |
| 1367 | OH | OCH3 | 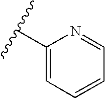 |
| 1368 | OAc | OH | 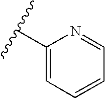 |
| 1369 | p-NO2(C6H4)CO2 | OH | 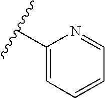 |
| 1370 | OH | OH | 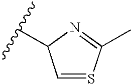 |
| 1371 | OH | OCH3 | 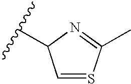 |
| 1372 | OAc | OH | 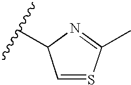 |
| 1373 | p-NO2(C6H4)CO2 | OH | 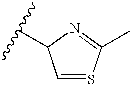 |
| 1374 | OH | OH | 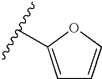 |
| 1375 | OH | OCH3 | 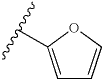 |
| 1376 | OAc | OH | 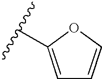 |
| 1377 | p-NO2(C6H4)CO2 | OH | 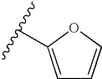 |
| 1378 | OH | OH | 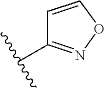 |
| 1379 | OH | OCH3 | 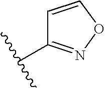 |
| 1380 | OAc | OH | 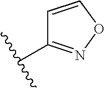 |
| 1381 | p-NO2(C6H4)CO2 | OH | 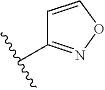 |
| 1382 | OH | OH | 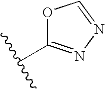 |
| 1383 | OH | OCH3 | 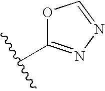 |
| 1384 | OAc | OH | 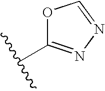 |
| 1385 | p-NO2(C6H4)CO2 | OH | 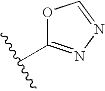 |
| 1386 | OH | OH | 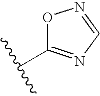 |
| 1387 | OH | OCH3 | 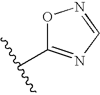 |
| 1388 | OAc | OH | 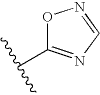 |
| 1389 | p-NO2(C6H4)CO2 | OH | 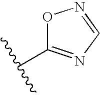 |
| 1390 | OH | OH | 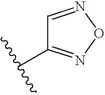 |
| 1391 | OH | OCH3 | 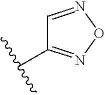 |
| 1392 | OAc | OH | 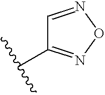 |
| 1393 | p-NO2(C6H4)CO2 | OH | 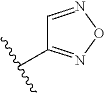 |
| 1394 | OH | OH | 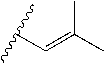 |
| 1395 | OH | OCH3 | 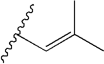 |
| 1396 | OAc | OH | 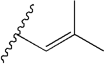 |
| 1397 | p-NO2(C6H4)CO2 | OH | 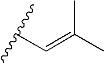 |

### EXAMPLE 12

The following macrocycles of Formula XIII are prepared, using appropriate reagents and according generally to the methods described herein.

| | |
|---|---|
| | (XXIII) |
| 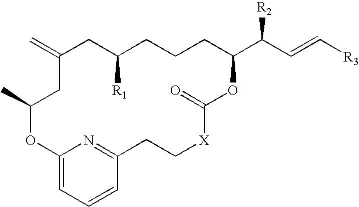 | |

| Compound | X | R₁ | R₂ | R₃ |
|---|---|---|---|---|
| 1400a1400b1400c | ONCH₂ | OH | OH | 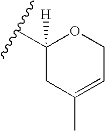 |
| 1401 | ONCH₂ | OH | OCH₃ | 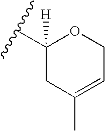 |
| 1402 | ONCH₂ | OAc | OH | 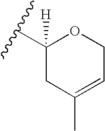 |
| 1403 | ONCH₂ | p-NO₂(C₆H₄)CO₂ | OH | 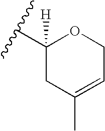 |
| 1404 | ONCH₂ | OH | OH | 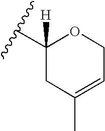 |
| 1405 | ONCH₂ | OH | OCH₃ | 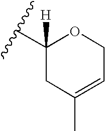 |
| 1406 | ONCH₂ | OAc | OH | 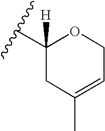 |
| 1407 | ONCH₂ | p-NO₂(C₆H₄)CO₂ | OH | 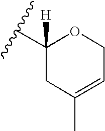 |
| 1408 | ONCH₂ | OH | OH | 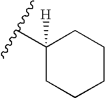 |
| 1409 | ONCH₂ | OH | OCH₃ | 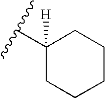 |
| 1410 | ONCH₂ | OAc | OH | 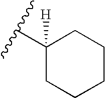 |
| 1411 | ONCH₂ | p-NO₂(C₆H₄)CO₂ | OH | 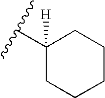 |
| 1412 | ONCH₂ | OH | OH | 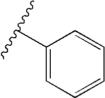 |
| 1413 | ONCH₂ | OH | OCH₃ | 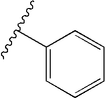 |
| 1414 | ONCH₂ | OAc | OH | 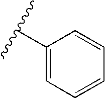 |
| 1415 | ONCH₂ | p-NO₂(C₆H₄)CO₂ | OH | 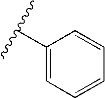 |
| 1416 | ONCH₂ | OH | OH | 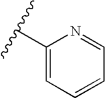 |
| 1417 | ONCH₂ | OH | OCH₃ | 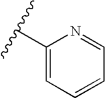 |
| 1418 | ONCH₂ | OAc | OH | 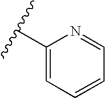 |
| 1419 | ONCH₂ | p-NO₂(C₆H₄)CO₂ | OH | 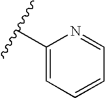 |
| 1420 | ONCH₂ | OH | OH | 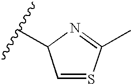 |
| 1421 | ONCH₂ | OH | OCH₃ | 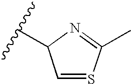 |
| 1422 | ONCH₂ | OAc | OH | 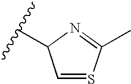 |
| 1423 | ONCH₂ | p-NO₂(C₆H₄)CO₂ | OH | 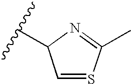 |
| 1424 | ONCH₂ | OH | OH | 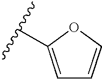 |
| 1425 | ONCH₂ | OH | OCH₃ | 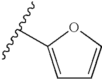 |
| 1426 | ONCH₂ | OAc | OH | 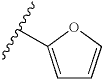 |
| 1427 | ONCH₂ | p-NO₂(C₆H₄)CO₂ | OH | 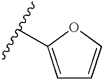 |
| 1428 | ONCH₂ | OH | OH | 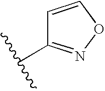 |
| 1429 | ONCH₂ | OH | OCH₃ | 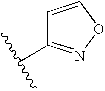 |
| 1430 | ONCH₂ | OAc | OH | 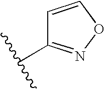 |
| 1431 | ONCH₂ | p-NO₂(C₆H₄)CO₂ | OH | 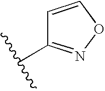 |
| 1432 | ONCH₂ | OH | OH | 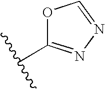 |
| 1433 | ONCH₂ | OH | OCH₃ | 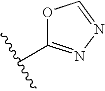 |
| 1434 | ONCH₂ | OAc | OH | 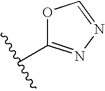 |
| 1435 | ONCH₂ | p-NO₂(C₆H₄)CO₂ | OH | 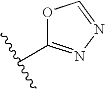 |
| 1436 | ONCH₂ | OH | OH | 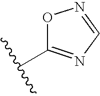 |
| 1437 | ONCH₂ | OH | OCH₃ | 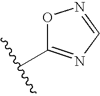 |
| 1438 | ONCH₂ | OAc | OH | 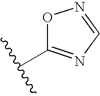 |
| 1439 | ONCH₂ | p-NO₂(C₆H₄)CO₂ | OH | 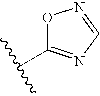 |
| 1440 | ONCH₂ | OH | OH | 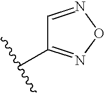 |
| 1441 | ONCH₂ | OH | OCH₃ | 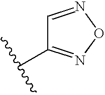 |
| 1442 | ONCH₂ | OAc | OH | 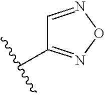 |
| 1443 | ONCH₂ | p-NO₂(C₆H₄)CO₂ | OH | 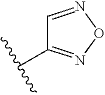 |
| 1444 | ONCH₂ | OH | OH | 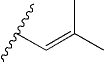 |
| 1445 | ONCH₂ | OH | OCH₃ | 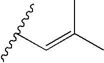 |
| 1446 | ONCH₂ | OAc | OH | 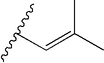 |
| 1447 | ONCH₂ | p-NO₂(C₆H₄)CO₂ | OH | 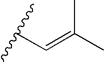 |

### EXAMPLE 13

### 80 to 81

To a well stirred LiAlH₄ suspension (1.83 g, 45.7 mmol) in THF (85 mL) at 0° C. under N₂ was added the solution of dimethyl 2,3-o-isopropylidene-L-tartrate 80 (5.00 g, 22.8 mmol, Aldrich, 97%, 98% ee) in THF (28 mL) dropwise over 30 min. The reaction mixture was heated to reflux for 26 hours and was then cooled to room temperature. The reaction mixture was quenched with H₂O (1.4 mL, 10 min), 15% NaOH aqueous solution (1.4 mL, 10 min) and H₂O (4.2 mL, 5 min) sequentially. The mixture was stirred at room temperature for 5 hours and was then filtered using Et₂O (250 mL) as eluant. The filtrate was dried with Na₂SO₄. The mixture was filtered and the solvent was removed in vacuo. The residue was directly used in the next step without further purification. ¹H NMR (300 MHz, CDCl₃): δ=3.97 (m, 2H), 3.71-3.80 (m, 4H), 2.88 (dd, J=6.6, 5.3 Hz), 1.42 (s, 6H) ppm. IR (FTIR, film) ν=3420, 2986, 2881, 1254 (s), 1220, 1057 cm⁻¹.

### EXAMPLE 14

### 81 to 82

To a well stirred NaH suspension (1.10 g, 27.4 mmol) in THF (43 mL) at room temperature under N₂ was added a solution of diol 81 (22.8 mmol) in THF (14 mL) dropwise over 20 min. After the addition was complete, the reaction mixture was stirred at room temperature for 45 min, during which time a large amount of opaque white precipitate was formed. A solution of TBSCl (3.44 g, 22.8 mmol) in THF (7 mL) was then added over 15 min and the mixture was then stirred for an additional 45 min. During stirring, the reaction mixture gradually turned to a clear solution. The reaction mixture was diluted with EtOAc (150 mL) and washed with 10% aqueous K₂CO₃ (60 mL), H₂O (60 mL), brine (60 mL) and dried with MgSO₄. The mixture was filtered and the solvent was removed in vacuo. The residue was purified via silica-gel flash chromatography (EtOAc:hexane 1:3) to give 5.97 g (97% over two steps) of 2 as a colourless oil. ¹H NMR (300 MHz, CDCl₃): δ=3.63-4.03 (m, 6H), 2.46-2.53 (m, 1H), 1.40-1.42 (m, 6H), 0.89-0.94 (m, 9H), 0.08-0.11 (m, 6H) ppm. IR (FTIR, film) ν=3475, 2986, 2955, 2931, 1254, 1217, 1089 cm⁻¹.

### EXAMPLE 15

### 82 to Aldehyde

To a cold (−78° C.), stirred solution of oxalyl chloride (2.37 mL, 27.2 mmol) in CH₂Cl₂ (125 mL) was added DMSO (3.87 mL, 54.5 mmol) dropwise over 10 min. The reaction mixture was stirred for 5 min and a solution of the TBS-monoprotected diol 82 (5.00 g, 18.1 mmol) in CH₂Cl₂ (25 mL) was then added dropwise over 15 min. The reaction mixture was stirred for an additional 30 min and Et₃N (12.77 mL, 91.0 mmol) was then added slowly over 10 min. The resultant yellowish solution was then warmed to room temperature over 1 hour and diluted with Et₂O (250 mL). The mixture washed with 1N HCl (75 mL), saturated aqueous NaHCO₃ (75 mL), H₂O (125 mL), brine (75 mL) and dried with MgSO₄. The mixture was filtered and the solvent was removed in vacuo. The residue was co-evaporated with dry benzene (3×5 mL) before use in the next step.

### EXAMPLE 16

### Aldehyde to 83 & 84

To a well-stirred suspension of the phosphonium salt 100a (11.25 g, 21.8 mmol) in 2:1 THF:HMPA (225 mL) at −70° C. under N₂ was added n-BuLi (1.6 M in hexane, 13.6 mL, 21.8 mmol) dropwise over 10 min. During the addition, the suspension turned to orange, red and eventually dark brown. The mixture was stirred at −70° C. for 1 min and a solution of the crude aldehyde in THF (13 mL) was added (dropwise over 5 min). The reaction mixture was slowly warmed up to −10° C. over 4 hours and quenched with saturated aqueous NH₄Cl (10 mL). The reaction mixture was diluted with Et₂O (250 mL) and washed with H₂O (125 mL), brine (100 mL) and dried with MgSO₄. The mixture was filtered and the solvent was removed by vacuo. The residue was then diluted with Et₂O (100 mL) and was passed through a pad of silica gel using 1:1 EtOAc:hexane (300 mL) as eluant. Removal of the solvent gave a dark red oil, which was dissolved in THF (125 mL). The solution was stirred under N₂ at room temperature and TBAF (1.0 M solution in THF, 18.7 mL, 18.7 mmol) dropwise over 10 min. After the addition was complete, the reaction mixture was stirred for an additional 30 min and was then diluted with Et₂O (375 mL). The mixture was washed with H₂O (250 mL), brine (100 mL) and dried with MgSO₄. The mixture was filtered and the solvent was removed in vacuo. The residue was purified via silica-gel flash chromatography (EtOAc:hexane 1:2) to give 2.28 g (50%) of Z-alcohol 83 and 0.56 g (12%) of E-alcohol 84 as colourless oils. ¹H NMR (Z-isomer, 300 MHz, CDCl₃): δ=5.74 (dd, J=11.5, 7.6 Hz, 1H), 5.48 (dd, J=11.5, 9.0 Hz, 1H), 5.42 (s, br, 1H), 4.74 (t, J=9.0 Hz, 1H), 4.25 (m, 1H), 4.16 (s, br, 2H), 3.72-3.81 (m, 2H), 3.55-3.62 (m, 1H), 2.53-2.58 (m, 1H), 2.00-2.13 (m, 1H), 1.78-1.84 (d, br, J=17.0 Hz, 1H), 1.67 (s, br, 3H), 1.41 (s, 6H) ppm. IR (FTIR, film) ν=3462 (br), 2981, 2931, 1241, 1135, 1061 cm⁻¹.

### EXAMPLE 17

### 83 to 84

To a solution of Z-alcohol 83 (1.00 g, 3.9 mmol) in benzene (HPLC grade, Fluka, 80 mL) in a quartz tube equipped with a rubber septa was added Bu₃SnSnBu₃ (250 mg, 0.43 mmol) in one batch. The reaction mixture was degassed by bubbling N₂ through the solution for 20 min. The tube then was sealed using Teflon® tape, fitted with a N₂ balloon on top, and then was subjected to irradiation at 300 nm with a Rayonet photoreactor for 2 hours. After the irradiation was complete, the mixture was cooled to the room temperature. The solvent was removed in vacuo and the residue was purified via silica-gel flash chromatography (EtOAc:hexane 1:2) to give 0.89 g (90%) of 84 as a colourless oil. ¹H-NMR (300 MHz, CDCl₃): δ=5.88 (dd, J=15.5, 5.1 Hz, 1H), 5.71 (dd, J=15.5, 7.3 Hz, 1H), 5.37 (s, br, 1H), 4.29 (t, J=7.5 Hz, 1H), 4.13 (s, br, 2H), 4.03 (m, 1H), 3.77-3.81 (m, 2H), 3.55-3.60 (m, 1H), 2.06 (m, 2H), 1.92 (m, 1H), 1.66 (s, br, 3H), 1.40 (s, 6H) ppm. IR (FTIR, film) ν=3439, 2984, 2935, 1382, 1238, 1133, 1049 cm⁻¹.

### EXAMPLE 18

### 84 to 85

To a cold (−78° C.), stirred solution of oxalyl chloride (0.75 mL, 8.6 mmol) in CH₂Cl₂ (56 mL) was added DMSO (1.22 mL, 17.1 mmol) slowly over 15 min. After the addition was complete, the reaction mixture was stirred for an additional 5 min and a solution of 84 (1.45 g, 5.7 mmol) in CH₂Cl₂ (5 mL) was then added dropwise over 10 min. The reaction mixture was stirred for 1 hour at −78° C. Et₃N (4.0 mL, 28.5 mmol) was then added dropwise over 10 min. The resultant yellowish solution was slowly warmed to room temperature over 1 hour and was diluted with Et₂O (100 mL). The organic layer was washed with 1N HCl (50 mL), saturated aqueous NaHCO₃ (50 mL), H₂O (50 mL), brine (40 mL) and was dried with MgSO₄. The mixture was filtered and the solvent was removed in vacuo. The residue was co-evaporated with dry benzene (3×5 mL) under vacuum before being used in the next step.

### EXAMPLE 19

### 85 to 86

To a cold (−30° C.), stirred suspension of phosphonium salt 45 (5.70 g, 11.4 mmol) in THF (65 mL) under N₂ was added NaHMDS (1.0M in THF, 11.4 mL, 11.4 mmol) dropwise over 30 min. During the course of the addition, the reaction mixture turned into a clear orange solution. The mixture was stirred at −20° C. for an additional 1 h and a solution of aldehyde 85 in THF (5 mL) was then added over 10 min. The reaction mixture was then slowly warmed to room temperature over 2 hours and stirred for an additional 3 h. Saturated aqueous NH₄Cl (5 mL) was added to quench the reaction. The reaction mixture was diluted with Et₂O (200 mL) and was washed with H₂O (100 mL), brine (80 mL) and dried with MgSO₄. The mixture was filtered and the solvent was removed in vacuo. The residue was purified via silica-gel flash chromatography (EtOAc:hexane 1:20) to give 1.95 g (84% over two steps) of 85 as a colourless oil. ¹H NMR (500 MHz, CDCl₃): δ=5.86 (dd, J=16.1, 5.0 Hz, 1H), 5.63-5.76 (m, 2H), 5.42-5.47 (m, 1H), 5. 39 (s, br, 1H), 4.46 (t, J=8.0 Hz, 1H), 4.09-4.18 (m, 2H), 3.88-4.07 (m, 2H), 3.57 (t, J=7.0 Hz, 2H), 2.23-2.39 (m, 2H), 1.86-2.07 (m, 2H), 1.68 (s, 3H), 1.42 (s, 6H), 0.85-0.89 (m, 9H), 0.03 (m, 6H) ppm. IR (FTIR, film) ν=2931, 2852, 1381, 1253, 1104, 1052 cm⁻¹.

### EXAMPLE 20

### 86 to 41

To a stirred solution of 86 (1.90 g, 4.64 mmol) in THF (82 mL) at room temperature under N₂ was added 3N HCl solution (10 mL). The reaction mixture was stirred at room temperature for 24 h. Solid NaHCO₃ was added in small portions to quench the reaction until no gas formation. The reaction mixture then was diluted with EtOAc (100 mL) and dried with Na₂SO₄. The mixture was filtered and the solvent was removed in vacuo. The residue was re-dissolved in CH₂Cl₂ (100 mL). This solution was then stirred under N₂ at −78° C. and Et₃N (2.6 mL, 18.5 mmol) was added over 5 min. The reaction mixture was stirred for 5 min and TBSOTf (4.3 mL, 18.5 mmol) was then added slowly over 15 min. After the addition was complete, the reaction mixture was warmed to room temperature over 1 hour and was quenched by saturated NH₄Cl aqueous solution (10 mL). The reaction mixture was diluted with Et₂O (200 mL) and washed with H₂O (100 mL), brine (80 mL) and dried with MgSO₄. The mixture was filtered and the solvent was removed in vacuo. The residue was purified via silica-gel flash chromatography (EtOAc:hexane 1:20) to give 2.56 g (93% over 2 steps) of 41 as a colourless oil. ¹H NMR (500 MHz, CDCl₃): δ=5.80 (dd, J=16.0, 4.6 Hz, 1H), 5.72 (dd, J=16.5, 4.9 Hz, 1H), 5.28-5.50 (m, 2H), 5.40 (s, br, 1H), 4.33 (dd, J=9.0, 5.0 Hz, 1H), 4.14-4.34 (m, 3H), 4.03 (m, 1H), 3.61 (t, J=7.1 Hz, 2H), 2.23-2.41 (m, 2H), 1.74-2.06 (m, 2H), 1.69 (s, br, 3H), 0.86-0.89 (m, 27H), 0.01-0.07 (m, 18H) ppm. IR (FTIR, film) ν=2932, 2859, 1251, 1103 cm⁻¹.

### EXAMPLE 21

### 41 to 42

To a stirred solution of 41 (2.50 g, 4.2 mmol) in 2-propanol (55 mL) at room temperature under N₂ was added Ceric Ammonium Nitrate (2.28 g, 4.2 mmol) in one portion. The resultant dark-red solution was stirred at room temperature for 24 hours, during which time the solution gradually turned light yellow. The reaction mixture was then diluted with Et₂O (200 mL) and was washed with H₂O (2×80 mL), brine (80 mL) and dried with MgSO₄. The mixture was filtered and the solvent was removed in vacuo. The residue was purified via silica-gel flash chromatography (EtOAc:hexane 1:10) to give 1.66 g (82%) of 42 as a colourless oil. ¹H NMR (500 MHz, CDCl₃): δ=5.78 (dd, J=16.0, 5.0 Hz, 1H), 5.68 (dd, J=16.0, 5.0 Hz, 1H), 5.36-5.50 (m, 2H), 5.40 (s, br, 1H), 4.34 (dd, J=8.0, 5.5 Hz, 1H), 4.10-4.18 (m, 3H), 4.03 (m, 1H), 3.57-3.64 (m, 2H), 2.24-2.40 (m, 2H), 1.69-2.05 (m, 2H), 1.61 (s, br, 1H), 1.69 (s, br, 3H), 0.89 (s, 9H), 0.86 (m, 9H), 0.02-0.07 (m, 12H) ppm. IR (FTIR, film) ν=3409, 2992, 2893, 2857, 1251, 1123, 1081 cm⁻¹.

### EXAMPLE 22

### 42 to 43

To a stirred solution of 42 (700 mg, 1.44 mmol) in CH₂Cl₂ (215 mL) at room temperature under N₂ was added NaHCO₃ (605 mg, 7.2 mmol) and Dess-Martin periodinane (1.84 g, 4.3 mmol) sequentially in one portion. The resultant milky suspension was stirred at room temperature for 30 min. TLC indicated the complete consumption of the starting material. The reaction was then cooled to 0° C. and quenched by the addition of 1:1 saturated aqueous solutions of Na₂S₂O₃ and NaHCO₃ (100 mL). The mixture was vigorously stirred at 0° C. to rt until the organic layer became clear (approximately 2 h). The mixture was then diluted with Et₂O (200 mL) and washed with H₂O (100 mL), brine (100 mL) and dried with MgSO₄. The mixture was filtered and the solvent was removed in vacuo. The residue was purified via silica-gel flash chromatography (EtOAc:hexane 1:10) to give 625 mg (90%) of the intermediate aldehyde 43 as a colourless oil. ¹H NMR (300 MHz, CDCl₃): δ=9.62 (s, br, 1H), 5.61-5.78 (m, 3H), 5.36-5.55 (m, 1H), 5.38 (s, br, 1H), 4.25 (dd, J=8.0, 5.1 Hz, 1H), 4.11-4.14 (m, 3H), 3.96-4.03 (m, 1H), 3.23 (d, J=7.0 Hz, 2H), 1.71-2.11 (m, 2H), 1.68 (s, br, 3H), 0.81-0.94 (m, 18H), −0.01-0.04 (m, 12H) ppm. IR (FTIR, film) ν=2960, 2932, 2887, 2858, 1731, 1255 (s), 1112, 1086 cm⁻¹.

### EXAMPLE 23

### 43 to 22b

To a stirred solution of β,γ-unsaturated aldehyde 43 (620 mg, 1.24 mmol) in CHCl₃ (passed through a pad of basic alumina, 56 mL) at room temperature under N₂ was added a solution of DBU (17 μL, 0.12 mmol) in CHCl₃ (2.8 mL) dropwise over 10 min. The reaction mixture was then stirred at room temperature for 2.5 hours. Saturated aqueous NH₄Cl (5 mL) was added to quench the reaction. The reaction mixture was diluted with Et₂O (100 mL) and washed with H₂O (40 mL), brine (40 mL) and dried with Na₂SO₄. The mixture was filtered and the solvent was removed in vacuo. The residue was purified via silica-gel flash chromatography (EtOAc:hexane 1:10) to give 540 mg (90%) of 22b as a yellowish oil. ¹H NMR (500 MHz, CDCl₃): δ=9.49 (d, J=7.8 Hz, 1H), 6.87 (dt, J=15.5, 8.0 Hz, 1H), 6.10 (dd, J=15.6, 8.0 Hz, 1H), 5.75-5.90 (m, 2H), 5.42 (s, br, 1H), 4.23 (m, 1H), 4.19 (m, 2H), 4.07 (m, 1H), 3.75 (m, 1H, C19-H), 2.54-2.62 (m, 1H), 2.28 (dt, J=14.4, 7.8 Hz, 1H), 1.88-2.14 (m, 2H), 1.71 (s, br, 3H), 0.88-0.91 (m, 18H), 0.03-0.07 (m, 12H) ppm. IR (FTIR, film) ν=2958, 2928, 2859, 1695, 1257, 1105 cm⁻¹.

### EXAMPLE 24

### 31 to 32

To a stirred solution of commercially available 31 in THF (9 mL) under N₂ at −20° C. was added a solution of BH₃ in THF (1.0M, 2.3 mL, 2.31 mmol) dropwise over 1.5 hr. Upon complete addition, the resulting mixture was warmed to room temperature and allowed to stir overnight. After this time, the reaction was cooled to 0° C. and H₂O added (2 mL). The solvent was removed in vacuo and the residue dissolved in Et₂O (100 mL). The resulting organics were washed with 1N HCl aq., saturated aqueous NaHCO₃, dried with MgSO₄ and concentrated in vacuo to afford 32 colourless oil (257 mg) that required no further purification. ¹H-NMR (300 MHz, CDCl₃): δ=3.75 (t, J=7.0 Hz, 2H), 3.66 (s, 3H), 1.21-2.60 (m, 6H), 0.95 (brd, J=7.1 Hz) ppm.

### EXAMPLE 25

### 32 to 33

To a stirred solution of 32 (257 mg) in CH₂Cl₂ (10 mL) under N2 at room temperature was added imidazole (180 mg, 2.65 mmol) in one portion followed by TBSCl (318 mg, 2.11 mmol) in CH₂Cl₂ (8 mL) dropwise over 20 min. Upon complete addition, the resulting mixture was allowed to stir at room temperature for a further 2 hr. The reaction was diluted with Et₂O (200 mL) and the organics washed successively with 1N HCl, saturated aqueous NaHCO₃ and brine and dried with MgSO₄. The mixture was filtered, concentrated in vacuo and purified by flash chromatography (silica gel, EtOAc:hexane 1:9) to afford 33 as a colourless oil (395 mg, 86% over 2 steps). ¹H-NMR (300 MHz, CDCl₃): 3.60 (t, J=6.8 Hz, 2H), 3.55 (s, 3H), 1.11-2.42 (m, 5H), 0.90 (d, J=6.9 Hz, 3H), 0.85 (s, 9H), 0.01 (s, 6H).

### EXAMPLE 26

### 33 to 28

An oven-dried flask containing magnetic stirrer was charged with CeCl₃ (5.0 g, 20.28 mmol). This flask was heated to 160° C. in a vacuum oven (2 torr) for 16 hr. After cooling (under Ar), THF was added (15 mL) and the resulting slurry stirred under Ar for 12 hr. This was cooled to −78° C. and TMSCH₂MgCl in Et₂O (1.0M, 20.28 mL, 20.28 mmol) added dropwise over 10 min. After a further 2 hr at this temperature, 33 (755 mg, 2.89 mmol) in THF (10 mL+4 mL wash) was added dropwise over 2 min. Upon complete addition the reaction allowed to warm to room temperature overnight. The reaction was quenched with NH₄Cl aq. (10 mL) at 0° C. and the resulting slurry partitioned between Et₂O and brine. The organic layer was separated, dried with MgSO₄ and concentrated in vacuo. The crude product was dissolved in CH₂Cl₂ (20 mL) and silica gel (1 g) added in one portion. This was stirred at room temperature for 2 hr after which time the suspension was filtered and the solvent removed in vacuo. Purification via flash chromatography (silica gel, EtOAc:hexane 1:13) furnished 28 (901 mg) in 99% yield. Optical Rotation: [α]^{D} 25.4=20.00o (c=0.11, CDCl₃). ¹H-NMR (300 MHz, CDCl₃): 4.58 (s, 1H), 4.57 (s, 1H), 3.60-3.65 (m, 2H), 1.93-0.199 (m, 1H), 1.74-1.79 (m, 2H), 1.23-1.31 (4H, m), 0.89 (s, 9H), 0.86 (d, J=6.8, 3H), 0.05 (s, 6H), 0.01 (s, 9H) ppm. ¹³CNMR (125 MHz, CDCl₃): δ=−5.3, −1.3, 19.6, 25.9, 26.2, 27.3, 39.7, 46.3, 61.3, 108.6, 146.2 ppm. FTIR δ=2955 (C—H), 2928 (C—H), 2858 (C—H), 1250 (C-Si), 1096 (C—O) cm⁻¹.

### EXAMPLE 27

### 28 to 29

The Lewis acid ligand (CAB) derived from D-tartaric acid (57 mg. 0.16 mmol) (Hansson, T., et al., J. Org. Chem., 57, 5370 (1992)) was dried in a vacuum oven (60° C., 2 torr) for 6 hr before use and was dissolved in freshly distilled propionitrile (0.2 mL). To this stirred solution under N₂ was added 3,5-bis(trifluoromethyl)phenyl boronic acid (34 mg, 0.13 mmol) in one portion. The reaction was stirred at room temperature for 2 hr and was then cooled to −70° C. To this cooled reaction mixture was added a solution of aldehyde 22b (53 mg, 0.11 mmol) in propionitrile (0.2 mL) followed by a solution of allylsilane 28 (36 mg, 0.11 mmol) in propionitrile (0.2 mL). The resulting mixture was stirred at −70° C. for 30 min and further (neat) allylsilane 28 added (36 mg, 0.1 μmol). After an additional 30 min at −70° C., a final portion of allylsilane 28 (neat) (36 mg, 0.11 mmol) was added. The mixture was stirred at −70° C. for 11 hr and was quenched by the addition of saturated aqueous NaHCO₃ (2 mL). The reaction was diluted with Et₂O (100 mL) and was washed with H₂O, brine and dried over MgSO₄. The mixture was filtered through a plug of silica (Et₂O as eluent) and the solvent removed in vacuo. The crude coupling product (74 mg) was dissolved in CH₂Cl₂ (1.7 mL) under N₂ and cooled to 0° C. To this was added diisopropylethyl amine (81 μL, 0.46 mmol) over 5 min. The mixture was stirred for an additional 5 min and then freshly prepared MOMCl (31 μL, 0.41 mmol) added over 3 min. The reaction was warmed to room temperature over 10 min and brought to reflux for 16 hr. The reaction was diluted with Et₂O (100 mL) and washed with 1N HCl, saturated aqueous NaHCO₃, H₂O, brine and dried over MgSO₄. The mixture was filtered and the solvent removed in vacuo. The residue was purified with flash chromatography (silica gel, EtOAc:hexane 1:10) to give 51 mg (60% over 2 steps) of 29 as a colourless oil. Optical Rotation: [α]^{D} 25.3=−104.71o (c=1.02, CDCl₃). ¹H NMR (500 MHz, CDCl₃): δ=5.85 (dd, J=15.5, J=4.0 Hz, 1H), 5.75 (ddd, J=15.5, J=6.0, J=1.5 Hz, 1H), 5.66 (dt, J=15.5, J=8.0 Hz, 1H), 5.41 (s(br), 1H), 5.29 (dd, J=15.5, J=6.5 Hz, 1H), 4.84 (s(br), 1H), 4.78 (s(br), 1H), 4.69 (d, JAB=7.0 Hz, 1H), 4.47 (d, JAB=7.0 Hz, 1H), 4.18 (s(br), 3H), 4.14-4.04 (m, 2H), 3.69-3.54 (m, 3H), 3.33 (s, 3H), 2.31-2.27 (m, 1H), 2.29 (dd, J=14.5, J=8.5, 1H), 2.14 (dd, J=14.0, J=5.5, 1H), 2.09-1.73 (m, 8H), 1.70 (s(br), 3H), 1.63-1.56 (m, 1H), 1.26-1.21 (m, 1H), 0.90 (s, 9H), 0.89 (s, 9H), 0.88 (s, 9H), 0.85 (d, J=6.5, 3H), 0.05-0.03 (m, 18H) ppm. ¹³C-NMR (125 MHz, CDCl₃): δ=144.3, 131.8, 131.6, 131.5, 130.9, 130.0, 119.7, 113.5, 93.4, 76.0, 75.1, 74.1, 73.8, 65.5, 61.4, 55.4, 44.3, 42.0, 39.8, 35.8, 34.4, 31.9, 29.7, 27.4, 26.0, 25.8, 23.0, 19.5, 18.3, 18.1, 18.0, −4.3, −4.5, −4.6, −4.8, −5.2, −5.3 ppm. FTIR (thin film) õ=3071w, 2954s, 2927s, 2821m, 2709w, 1698m, 1682w, 1644m, 1471s, 1463s, 1435m, 1381m, 1361s, 1255s, 1098s, 1045s, 918s, 835s, 774s, 666m cm⁻¹. HRMS: (m/z) Calculated for C₃₄H₆₂O₄Si₂ (i.e. M+-MOM): 590.4147, found 590.4186.

### EXAMPLE 28

### 29 to 30

To a stirred solution of 29 (28 mg, 36.5 μmol) in 2-propanol (0.6 mL) at room temperature under N₂ was added ceric ammonium nitrate (CAN) (20 mg, 36.5 lμmol) in one portion. The resultant dark red solution was stirred at room temperature for 24 hr, during which time the solution gradually turned light yellow. The reaction mixture was then diluted with Et₂O (100 mL) and washed with H₂O, brine and dried over MgSO₄. The mixture was then filtered and the solvent removed in vacuo. The residue was purified by flash chromatography (silica gel, EtOAc:hexane 1:10) to give 22 mg the primary alcohol as a colourless oil which was used directly in the next step. Optical Rotation: [α]^{D}=−96.74o (c=0.14, CDCl₃). ¹H NMR (500 MHz, CDCl₃): δ=5.84 (dd, J=16.5, J=4.0 Hz, 1H), 5.75 (m, 1H), 5.65 (dt, J=15.5, J=8.0 Hz, 1H), 5.41 (s(br), 1H), 5.30 (dd, J=15.0, J=8.0 Hz, 1H), 4.85 (s(br), 1H), 4.80 (s(br), 1H), 4.69 (d, JAB=6.8 Hz, 1H), 4.47 (d, JAB=6.8 Hz, 1H), 4.18 (s(br), 3H), 4.14-4.09 (m, 1H), 4.07-4.02 (m, 1H), 3.74-3.63 (m, 2H), 3.57-3.53 (m, 1H), 3.34 (s, 3H), 2.32-2.27 (m, 2H), 2.20-2.04 (m, 3H), 1.97-1.77 (m, 4H), 1.70 (s(br), 3H), 1.64-1.57 (m, 1H), 1.42-1.33 (m, 1H), 0.90 (s, 9H), 0.88 (s, 9H), 0.89 (d, J=6.5 Hz), 0.05-0.03 (m, 12H) ppm. ¹³C-NMR (125 MHz, CDCl₃): δ=144.2, 131.9, 131.5, 131.5, 129.9, 119.6, 113.8, 93.4, 76.0, 75.2, 74.1, 73.8, 65.5, 61.0, 55.4, 44.1, 42.0, 39.7, 35.7, 34.4, 29.7, 27.3, 25.8, 23.0, 19.6, 18.1, 18.0, −3.0, −4.3, −4.6, −4.8 ppm. FTIR (thin film) δ=3474, 2929, 2857, 1644, 1472, 1362, 1256, 1099, 1047, 974, 919, 836, 776 cm⁻¹.

To a stirred solution of the crude alcohol in DMF (3.5 mL) at rt under N₂ was added PDC (13.7 mg, 36.5 μmol) in one batch. The resulting mixture was stirred at rt for a further 24 hr. The reaction was filtered through a pad of celite (with EtOAc as eluant) and the filtrate concentrated in vacuo. The crude material was purified via silica-gel chromatography (MeOH:CH₂Cl₂ 4:96) to afford the required carboxylic acid 30 in a 66% yield (over 2 steps). Optical Rotation: [α]^{D}=−91.05o (c=0.32, CDCl₃). ¹H-NMR (300 MHz, CDCl₃): δ=5.82-5.91 (m, 2H), 5.66 (brm, 1H), 5.39 (brs, 1H), 5.34 (dd, J=15.4, 7.9 Hz, 1H), 4.84 (brs, 1H), 4.80 (brs, 1H), 4.65 (d, J=6.6 Hz, 1H), 4.48 (d, J=6.7 Hz, 1H), 4.07-4.18 (m, 4H), 4.02-4.05 (m, 1H), 3.50-3.53 (m, 1H), 3.33 (s, 3H), 2.34-2.45 (m, 3H), 2.04-2.09 (m, 2H), 1.79-1.92 (m, 5H), 1.70 (brs, 3H), 0.90-0.93 (m, 18H), 0.89 (d, J=6.6 Hz, 3H), 0.01-0.04 (m, 12H) ppm. ¹³C-NMR (125 MHz, CDCl₃): δ=−6.5, 6.1, 15.0, 15.1, 19.2, 20.1, 20.3, 23.8, 24.8, 37.1, 41.9, 43.3, 43.9, 44.1, 50.1, 66.7, 72.2, 77.3, 77.9, 80.7, 96.9, 105.3, 119.0, 128.7, 128.8, 129.6, 129.7, 137.4, 152.2, 177.0 ppm. FTIR (thin film): ν=3409, 2942, 2923, 1698, 1229, 1198 cm⁻¹. HRMS: (m/z) Calculated for C₃₆H₆6O₇ Si₂ M+: 667.0760, found 667.0753.

### EXAMPLE 29

### 30 to 82

To a stirred solution of 30 (11 mg, 0.0165 mmol) in CH₂Cl₂ (0.5 mL) at room temperature under N₂ was added HOBt (2.7 mg, 0.0198 mmol) followed by DCC (5.0 mg, 0.023 mmol). The resulting solution was stirred at 0° C. for 20 min before a solution of amine 50 (3.3 mg, 0.0198 mmol) and diisopropylethyl amine (6.3 μL, 0.0363 mmol) in CH₂Cl₂ (0.5 mL) was added dropwise over 2 min. Upon complete addition the reaction mixture was allowed to warm to room temperature overnight. The mixture was filtered, concentrated in vacuo and the crude material purified by flash chromatography (silica gel, EtOAc:hexane 1:4) to afford 52 as a colourless oil (12.5 mg, 97%) which was used directly in the next step. To a stirred solution of 52 (12.5 mg, 0.016 mmol) in THF (11.0 mL) under N₂ at 0° C. was added TBAF (1.0M in THF, 50 μL, 0.05 mmol) dropwise over 5 min. The resulting solution was allowed to warm to room temperature overnight. The reaction was diluted with Et₂O (50 mL) and washed successively with H₂O, brine, then dried with MgSO₄ and concentrated in vacuo. The crude oil obtained was purified with flash chromatography (silica gel, MeOH:EtOAc: 95:5) to afford 82 as a colourless oil (8.0 mg, 90%). Optical Rotation: [α]D=−85.89o (c=0.11, CDCl₃). ¹H-NMR (300 MHz, CDCl₃): δ=5.55-5.85 (m, 4H), 5.41 (brs, 1H), 5.29 (dd, J=15.5, 7.8 Hz, 1H), 4.88 (brs, 1H), 4.81 (brs, 1H), 4.68 (d, J=6.7 Hz, 1H), 4.46 (d, J=6.7 Hz, 1H), 4.18 (brs, 3H), 4.05-4.11 (m, 2H), 3.66 (s, 3H), 3.48-3.53 (m, 2H), 3.33 (s, 3H), 3.22-3.26 (m, 2H), 2.35 (t, J=6.9 Hz, 2H), 1.79-2.30 (m, 10H), 1.70 (brs, 3H), 1.21-1.65 (m, 4H), 0.89 (d, J=6.6 Hz, 3H) ppm. “CNMR (125 MHz, CDCl₃): δ=18.5, 22.8, 23.9, 24.9, 31.0, 33.0, 36.1, 41.2, 41.6, 43.8, 43.9, 44.9, 50.2, 50.3, 66.5, 72.9, 77.9, 78.1, 80.5, 96.4, 106.9, 120.0, 127.7, 127.9, 129.9, 138.4, 152.5, 172.5, 174.3 ppm. IR (FT-IR, film): ν=3395, 2929, 2857, 1733, 1689, 1173 cm⁻¹. HRMS: (m/z) Calculated for C₃₀H₄₉NO₈ M+: 551.3458, found 551.3462.

### EXAMPLE 30

### 82 to 54

To a stirred solution of diol 82 (8.0 mg, 0.0145 mmol) in 1:2 MeOH:THF (5 mL) under N₂ at room temperature was added 0.5N LiOH aqueous solution (1.31 mL, 0.384 mmol) dropwise over 5 min. The reaction was then stirred at room temperature for an additional 20 hr. 1N HCl (5 mL) was added slowly over 3 min, followed by the addition of aqueous saturated NaH₂PO₄ (10 mL) in one portion. The mixture was diluted with EtOAc (50 mL), washed with brine and dried with MgSO₄. This was filtered and the solvent removed in vacuo. The residue was co-evaporated with benzene (3×25 mL) and used directly in the next step. The resultant crude acid 53 (5.1 mg, 9.5/μmol) was dissolved in benzene (14 mL) and stirred under N₂ at room temperature. Et₃N (8.0 μL, 56.9 μmol) was added in one batch, followed by freshly distilled Yamaguchi reagent (8.8mL, 56.9 μmol) dropwise. The mixture was stirred at room temperature for 5 min before DMAP (7.0 mg, 56.9 μmol) was added. The reaction mixture turned cloudy over a 5 min period and was allowed to stir at room temperature for an additional 20 hr. The solvent was removed in vacuo and the residue diluted with Et₂O (50 mL). The organics were washed with 1N HCl, NaHCO₃ aq., H₂O and dried with MgSO₄. The crude mixture was filtered and the solvent removed in vacuo. The residue was purified by flash chromatography (silica gel, EtOAc, hexane 1:2) to give 2.1 mg of the Cl₉ macrolide along with 0.6 mg C₂₀ regioisomer. To a stirred solution of C₁₉ macrolide (2.1 mg, 4.04 μmol) in tBuOH (1 mL) under N₂ at room temperature was added PPTS (2.0 mg, 8.09 μmol) in one portion. The resulting mixture was warmed to 60° C. for 12 hr. After cooling, the reaction mixture was diluted with EtOAc (50 mL) and the organics washed successively with saturated aqueous NaHCO₃ and brine and the solvent removed in vacuo. The crude product was purified by flash chromatography (silica gel, MeOH:EtOAc, 95:5) to afford 54 as a colourless oil (1.8 mg, 27% over 3 steps). Optical Rotation: [α]^{D}=−103.75o (c=0.21, CDCl₃). ¹H-NMR (300 MHz, CDCl₃): δ=5.65-5.87 (m, 4H), 5.4 (br s, 1H), 5.31-5.34 (m, 1H), 4.73-4.77 (m, 2H), 4.48 (m, 1H), 3.20-4.10 (m, 7H), 2.13-2.24 (m, 10H), 2.01 (br m, 2H), 1.58-1.92 (m, 10H), 1.06 (d, J=6.8, 3H). ¹³C-NMR (125 MHz, CDCl₃): δ=19.1, 22.3, 24.0, 25.1, 31.3, 31.4, 33.9, 41.5, 43.0, 43.5, 46.7, 66.5, 71.9, 77.3, 77.9, 80.1, 106.1, 121.0, 128.7, 129.1, 136.9, 152.4, 172.4, 174.1 ppm. IR (FT-IR, film): ν=3389, 2935, 2914, 1722, 1688, 1170 cm⁻¹. HRMS: (m/z) Calculated for C₂₇H₄₁NO₆ M+: 475.2934, found 475.2925.

All of the compositions and/or methods and processes disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the methods described herein without departing from the concept and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the scope and concept of the invention.

1. A compound of Formula I, or a pharmaceutically acceptable salt or ester thereof, 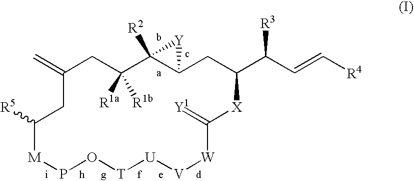
wherein:
R^{1a}, R^{1b}, and R⁵ are each independently H, C₁-C₁₀ alkyl, C₂-C₁₀ alkenyl, C₁-C₁₀ alkoxy, C₂-C₁₀ alkenoxy, C₂-C₁₀ alkynyl, C₂-C₁₀ alkynoxy, aryl, substituted aryl, heteroaryl, substituted heteroaryl, COR⁸, nitro, cyano, OH, CF₃, OCF₃, or halogen;
R² is absent (when “a” is a triple bond or when “a” is a single bond and “b” is a double bond) or is selected from the group consisting of H, C₁-C₁₀ alkyl, C₂-C₁₀ alkenyl, C₁-C₁₀ alkoxy, C₂-C₁₀ alkenoxy, C₂-C₁₀ alkynyl, C₂-C₁₀ alkynoxy, aryl, nitro, cyano, halogen, acyl, alkacyl, CHO, CO₂H, CO₂—C₁₋₁₀ alkyl, CF₃, OH, OR^{8′}, OCF₃, SH, SR^{8′}, NH₂, NHR^{8′}, NHR^{8′}R^{8′}, CON(R^{8′})₂, and CONHR^{8′};
“a” can be a single or double bond of either (E)- or (Z)-orientation, or “a” can be a triple bond when R², Y, “b” and “c” are absent;
“b” can be absent or a single bond (when R¹ is absent);
“c” can be absent, a single, or double bond of either (E)- or (Z)-orientation; such that only one of “a”, “b”, and “c” can be a double bond, when “b” and “c” are absent, then Y is absent; and
when “a” is a single or double bond, one of “b” and “c” is a single bond and one is absent, then Y is H, a straight or branched substituted or unsubstituted alkyl, alkenyl, or alkynyl, CH₃, CH₂R⁸, CHR⁸R⁸, CR⁸R⁸R⁸, CH₂F, CH₂Cl, CH₂Br, CHF₂, CHCl₂, CHBr₂, CF₃, CCl₃, CBr₃, OH, OR^{8′}, SH, SR^{8′}, NH₂, NHR^{8′}, or NR^{8′}R^{8′};
when “a”, “b”, and “c” are single bonds or when “a” is a single bond, one of “b” and “c” is a double bond and one is absent, then Y is CH₂, CHR⁸, CR⁸R⁸, CHF, CHCl, CHBr, CF₂, CCl₂, CBr₂, O, S, NH, or NR^{8′};
R³ is independently selected from H, C₁-C₁₀ alkyl, C₂-C₁₀ alkenyl, C₁-C₁₀ alkoxy, C₂-C₁₀ alkenoxy, C₂-C₁₀ alkynyl, C₂-C₁₀ alkynoxy, aryl, substituted aryl, heteroaryl, substituted heteroaryl, nitro, cyano, CF₃, OH, O-alkyl, hydroxylalkyl, O-acyl, OCF₃, SH, S-alkyl, thioalkyl, S-acyl, amine, alkylamine, NH₂, NHR⁸, NR⁸R⁸, and halogen;
R⁴ is selected from the group consisting of C₃-C₁₀ cycloalkyl, C₃-C₁₀ cycloalkenyl, heteroaryl, substituted heteroaryl, aryl, substituted aryl, C₃-C₁₀ heterocycloalkyl, adamantyl, and C₃-C₁₀ heterocycloalkenyl;
X is CH₂, CHR⁸, CR⁸R⁸, NH, NR^{8′}, O, or S; and
when “d” is a single bond, V is independently selected from the group consisting of CH₂, CHR⁸, CR⁸R⁸, NH, NR^{8′}, O, S, C═O, or C═Y², and W is independently selected from the group consisting of CH₂, CHR⁸, CR⁸R⁸, NH, NR^{8′}, O, or S;
such that V and W are not both NH, NR^{8′}, O, S, C═O, or C═Y²; W is not NH, NR^{8′}, O, or S, when X is N, NR⁵, O, or S; and V is not C═O or C═Y², when W is N, NR⁵, O, or S;
when “d” is a double bond of either (E)- or (Z)-orientation, V and W are independently selected from the group consisting of CH, CR⁸, or N such that V and W are not both N, and X and W are not both N; or when “d” is a triple bond, V and W are both carbon;
or alternatively, V and W are taken together to form an optionally substituted or unsubstituted carbocyclic ring, such as a 3-6 membered cycloalkyl ring, or an optionally substituted or unsubstituted heterocyclic ring, such as a 3-6 membered heterocyclic ring, such that only 2 adjacent ring members joined via a single or double bond (i.e., “d” is a single bond or a double bond) are part of the macrocyclic ring system; and the ring member directly adjacent to the —(C═Y¹)— moiety of the macrocycle is not a heteroatom when X is N, NR⁵, O, or S;
when “e”, “f”, “g”, “h”, or “i” is a single bond (i.e., the bond between M and P, P and Q, T and U, or U and V, is a single bond), then the respective M, P, T, U, or Q is independently CH₂, CHR⁸, CR⁸R⁸, NH, NR^{8′}, O, S, C═O, or C═Y²;
such that if one of M, P, T, U, V, or W is NH, NR^{8′}, O, or S, then its directly adjacent moieties cannot be NH, NR^{8′}, O, or S; and if one of M, P, T, U, V, or W is NH, NR^{8′}, O, or S, then its directly adjacent moieties both cannot be C═O or C═Y²; and, if one of M, P, T, U, or V is C═O or C═Y², then its directly adjacent moieties cannot be C═O or C═Y 2; and if one of M, P, T, U, or V is C═O or C═Y², then its directly adjacent moieties both cannot be NH, NR^{8′}, O, or S; or alternatively,
when “e”, “f”, “g”, “h”, or “i” is a double bond of either (E)- or (Z)-orientation, then the respective M, P, T, U, or Q is independently CH, CR⁸, or N, such that, if one of M, P, T, U, V, or W is N, then its directly adjacent moieties cannot be N, NH, NR^{8′}, O, or S; and
when “e”, “f”, “g”, “h”, or “i” is a triple bond, then the respective M, P, T, U, or Q is a carbon; wherein
when “h” and “i” are single bonds, P is CHR*, CR⁸R*, or NR*; when one of “h” and “i” is a double bond”, P is CR*; and when “g” and “f” are single bonds, T is CHR*′, CR⁸R*′, or NR*′; when one of “g” and “f” is a double bond”, T is CR*′; wherein R* and R*′ are taken together with Q to form an optionally substituted or unsubstituted carbocyclic ring, such as a 3-6 membered cycloalkyl ring, or an optionally substituted or unsubstituted heterocyclic ring, such as a 3-6 membered heterocyclic ring, such that the ring member directly adjacent to M is not a heteroatom when M is N, NR⁵, O, or S;
with the proviso that when —V—W— is —CH═CH— or —C≡C—, then —P-Q-T- is not 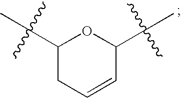
each Y¹ and Y² is independently O, S, NH, or NR^{8′};
each R⁸ is independently —H; an optionally substituted or unsubstituted straight or branched alkyl, such as a —C₁₋₈ straight or branched chain alkyl; an optionally substituted or unsubstituted straight or branched —C₂₋₈ alkenyl; an optionally substituted or unsubstituted straight or branched —C₂₋₈ alkynyl; —C₃₋₆ cycloalkyl; 3-7 membered heterocycle; -aryl; -aralkyl; -heteroaryl, -heteroarylalkyl, -halo (F, Cl, Br, I); -haloalkyl; —CF₃; —CN; —NO₂; -acyl (including but not limited to aldehydes, ketones, esters, carboxylic acids, amides, imides, thioesters), —(C═Y¹)-alkyl, —O(C═Y¹)-alkyl, —(C═Y¹)—OH, —(C═Y¹)—O-alkyl, —S—(C═Y¹)-alkyl, —(C═Y¹)—SH, —(C═Y¹)—S-alkyl, —NH(C═Y¹)-alkyl, —NR⁸ (C═Y¹)-alkyl, —(C═Y¹)—NH₂, —(C═Y¹)—NH(alkyl), —(C═Y¹)—N(alkyl)₂, —COOH; —COOC₁₋₈ alkyl; —CONH₂; —CONH—C₁₋₈ alkyl; —CON(C₁₋₈ alkyl)₂; alkacyl, -alkyl-(C═Y¹)-alkyl, -alkyl-O(C═Y¹)-alkyl, -alkyl-(C═Y¹)—OH, -alkyl-(C═Y¹)—O-alkyl, -alkyl-S—(C═Y¹)-alkyl, -alkyl-(C═Y¹)—SH, -alkyl-(C═Y¹)—S-alkyl, -alkyl-NH(C═Y¹)-alkyl, -alkyl-NR^{8′}(C═Y¹)-alkyl, -alkyl-(C═Y¹)—NH₂, -alkyl-(C═Y¹)—NH(alkyl), -alkyl-(C═Y¹)—N(alkyl)₂, -alkyl-COOH; -alkyl-COOC₁₋₈ alkyl; -alkyl-CONH₂; -alkyl-CONH—C₁₋₈ alkyl; -alkyl-CON(C₁₋₈ alkyl)₂; amino, —NH₂; —NH—C₁₋₈ alkyl; —N(C₁₋₈ alkyl)₂; —NHC(O)—C₁₋₈ alkyl; alkylamino; hydroxyl, alkylhydroxyl, alkoxy, thio; alkylthio; thioalkyl; and
each R^{8′} is independently an optionally substituted or unsubstituted straight or branched alkyl, such as a —C₁₋₈ straight or branched chain alkyl; an optionally substituted or unsubstituted straight or branched alkenyl, such as a —C₂₋₈ alkenyl; an optionally substituted or unsubstituted straight or branched alkynyl, such as a —C₂₋₈ alkynyl; a saturated or unsaturated carbocycle, such as a saturated or unsaturated —C₃₋₆ cycloalkyl; a heterocycle, such as a 3-7 membered heterocycle; aryl; or heteroaryl;
such that there is not a double or triple bond directly adjacent to a double or triple bond.

2. The compound of claim 1, wherein R^{1a}, R^{1b}, and R⁵ are either hydrogen, CH₃, or C₁-C₅ alkyl.

3. The compound of claim 1 wherein “-M-P-Q-T-U—” is selected from the group consisting of —(C═O)-Z-CH₂—CH₂—CH₂—, —(C═Y²)-Z-CH₂—CH₂—CH₂—, —(C═Y²)-Z-CHR⁸—CHR⁸—CHR⁸—, —CH₂—(C═O)-Z-CH₂—CH₂—, —CH₂—(C═Y²)-Z-CH₂—CH₂—, —CHR⁸—(C═Y²)-Z-CHR⁸—CHR⁸—, —CH₂—CH₂—(C═O)-Z-CH₂—, —CH₂—CH₂—(C═Y²)-Z-CH₂—, —CHR⁸—CHR⁸—(C═Y²)-Z-CHR⁸—, -Z-(C═O)—CH₂—CH₂—CH₂—, -Z-(C═Y²)—CH₂—CH₂—CH₂—, -Z-(C═Y²)—CHR⁸—CHR⁸—CHR⁸—, —CH₂-Z-(C═O)—CH₂—CH₂—, —CH₂-Z-(C═Y²)—CH₂—CH₂—, —CHR⁸-Z-(C═Y²)—CHR⁸—CHR⁸—, —CH₂—CH₂-Z-(C═O)—CH₂—, —CH₂—CH₂-Z-(C═Y²)—CH₂—, —CHR⁸—CHR⁸-Z-(C═Y²)—CHR⁸—, —(C═O)-Z-CH═CH—CH₂—, —(C═Y²)-Z-CH═CH—CH₂—, —(C═Y²)-Z-CR⁸═CR⁸—CHR⁸—, —(C═O)-Z-CH₂—CH═CH—, —(C═Y²)-Z-CH₂—CH═CH—, (C═Y²)-Z-CHR⁸—CR⁸═CR⁸—, —CH═CH—(C═O)-Z-CH₂—, —CH═CH—(C═Y²)-Z-CH₂—, —CR⁸═CR⁸—(C═Y²)-Z-CHR⁸—, -Z-(C═O)—CH═CH—CH₂—, -Z-(C═Y²)—CH═CH—CH₂—, -Z-(C═Y²)—CR⁸═CR⁸—CHR⁸—, -Z-(C═O)—CH₂—CH═CH—, -Z-(C═Y²)—CH₂—CH═CH—, -Z-(C═Y²)—CHR⁸—CR⁸═CR⁸—, —CH═CH-Z-(C═O)—CH₂—, —CH═CH-Z-(C═Y²)—CH₂—, —CR⁸═CR⁸-Z-(C═Y²)—CHR⁸—, —(C═O)-Z-C≡C—CH₂—, —(C═Y²)-Z-C≡C—CH₂—, —(C═Y²)-Z-C≡C—CHR⁸—, —(C═O)-Z-CH₂—C≡C—, —(C═Y²)-Z-CH₂—C≡C—, —(C═Y²)-Z-CHR⁸—C≡C—, —C≡C—(C═O)-Z-CH₂—, —C≡C—(C═Y²)-Z-CH₂—, —C≡C—(C═Y²)-Z-CHR⁸—, -Z-(C═O)—C≡C—CH₂—, -Z-(C═Y²)—C≡C—CH₂—, -Z-(C═Y²)—C≡C—CHR⁸—, -Z-(C═O)—CH₂—C≡C—, -Z-(C═Y²)—CH₂—C≡C—, -Z-(C═Y²)—CHR⁸—C≡C—, —C≡C-Z-(C═O)—CH₂—, —C≡C-Z-(C═Y²)—CH₂—, and —C≡C-Z-(C═Y²)—CHR⁸—, or at least one of “-M-P—”, “—P-Q-”, “-Q-T-” or “-T-U—” is selected from the group consisting of -Z-CHR^{8″}—, —CHR^{8″}-Z-, -Z′=CR^{8″}—, and —CR^{8″}-Z′-, or at least one of “-M-P-Q-”, “—P-Q-T-”, or “-Q-T-U—” is selected from the group consisting of —CHR^{8″}-Z-CHR^{8″}—, —CR^{8″}-Z′-CHR^{8″}—, or —CHR^{8″}-Z′=CR^{8″}—;
Z is CH₂, CHR⁸, CR⁸R⁸, O, S, NH, or NR^{8′}; and
Z′ is CH, CR⁸, or N,
provided that no heteroatom is directly adjacent to another heteroatom.

4. The compound of claim 1 wherein:
“—V—W—” is —CH═CH—, —CR⁸═CR⁸, —C≡C—, 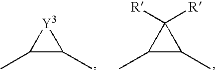
wherein Y³ is O, S, NH, or NR^{8′}, and each R′ is hydrogen, CH₃, CF₃, or halogen (F, Cl, Br, or I).

5. The compound of claim 1 wherein M, P, U, V and W are CH₂.

6. The compound of claim 1 wherein X is O or NH.

7. The compound of claim 1 wherein one of R^{1a} and R^{1b} is OH and one is H.

8. The compound of claim 1 wherein R³ is OH.

9. The compound of claim 1 wherein R⁵ is CH₃.

10. The compound of claim 1 wherein Q is O or NH and T is C(O).

11. The compound of claim 1 wherein P is C(O) and Q is NH and T is CH₂.

12. The compound of claim 1 wherein “d” is a double bond of either (E)- or (Z)-orientation and V and W are independently CH or CR⁸.

13. The compound of claim 1 wherein “h” and “g” are single bonds and P and T are CHR*, CR⁸R*, or NR*, wherein P-Q-T form an optionally substituted or unsubstituted 3-6 membered cycloalkyl, or an optionally substituted or unsubstituted 3-6 membered heterocyclic ring.

14. A compound of Formula IV, or a pharmaceutically acceptable salt or ester thereof, 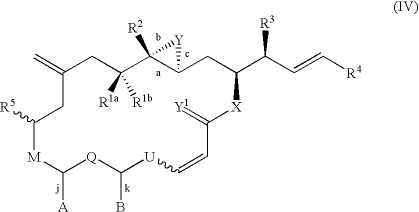
wherein:
R^{1a}, R^{1b}, and R⁵ are each independently H, C₁-C₁₀ alkyl, C₂-C₁₀ alkenyl, C₁-C₁₀ alkoxy, C₂-C₁₀ alkenoxy, C₂-C₁₀ alkynyl, C₂-C₁₀ alkynoxy, aryl, substituted aryl, heteroaryl, substituted heteroaryl, COR⁸, nitro, cyano, OH, CF₃, OCF₃, or halogen;
R² is absent (when “a” is a triple bond or when “a” is a single bond and “b” is a double bond) or is selected from the group consisting of H, C₁-C₁₀ alkyl, C₂-C₁₀ alkenyl, C₁-C₁₀ alkoxy, C₂-C₁₀ alkenoxy, C₂-C₁₀ alkynyl, C₂-C₁₀ alkynoxy, aryl, nitro, cyano, halogen, acyl, alkacyl, CHO, CO₂H, CO₂—C₁₋₁₀ alkyl, CF₃, OH, OR^{8′}, OCF₃, SH, SR^{8′}, NH₂, NHR^{8′}, NHR⁸′R^{8′}, CON(R^{8′})₂, and CONHR^{8′};
“a” can be a single or double bond of either (E)- or (Z)-orientation, or “a” can be a triple bond when R², Y, “b” and “c” are absent;
“b” can be absent or a single bond (when R² is absent);
“c” can be absent, a single, or double bond of either (E)- or (Z)-orientation; such that
only one of “a”, “b”, and “c” can be a double bond, when “b” and “c” are absent, then Y is absent; and
when “a” is a single or double bond, one of “b” and “c” is a single bond and one is absent, then Y is H, a straight or branched substituted or unsubstituted alkyl, alkenyl, or alkynyl, CH₃, CH₂R⁸, CHR⁸R⁸, CR⁸R⁸R⁸, CH₂F, CH₂Cl, CH₂Br, CHF₂, CHCl₂, CHBr₂, CF₃, CCl₃, CBr₃, OH, OR⁸, SH, SR⁸, NH₂, NHR⁸, or NR^{8′}R^{8′};
when “a”, “b”, and “c” are single bonds or when “a” is a single bond, one of “b” and “c” is a double bond and one is absent, then Y is CH₂, CHR⁸, CR⁸R⁸, CHF, CHCl, CHBr, CF₂, CCl₂, CBr₂, O, S, NH, or NR^{8′};
R³ is independently selected from H, C₁-C₁₀ alkyl, C₂-C₁₀ alkenyl, C₁-C₁₀ alkoxy, C₂-C₁₀ alkenoxy, C₂-C₁₀ alkynyl, C₂-C₁₀ alkynoxy, aryl, substituted aryl, heteroaryl, substituted heteroaryl, nitro, cyano, CF₃, OH, O-alkyl, hydroxylalkyl, O-acyl, OCF₃, SH, S-alkyl, thioalkyl, S-acyl, amine, alkylamine, NH₂, NHR⁸, NR⁸R⁸, and halogen;
R⁴ is selected from the group consisting of C₃-C₁₀ cycloalkyl, C₃-C₁₀ cycloalkenyl, heteroaryl, substituted heteroaryl, aryl, substituted aryl, C₃-C₁₀ heterocycloalkyl, adamantly, and C₃-C₁₀ heterocycloalkenyl;
X is CH₂, CHR⁸, CR⁸R⁸, N, NR^{8′}, O, or S;
each Y¹ and Y² is independently O, S, NH, or NR^{8′};
M, Q and U is each independently CH₂, CHR⁸, CR⁸R⁸, NH, NR^{8′}, O, S, C═O, or C═Y²;
such that if j and A are ═O or ═Y² or k and B are ═O or ═Y², the adjacent M or U is not C═O or C═Y² and M and Q or Q and U are not both NH, NR^{8′}, O, or S;
“j” can be a single, or double bond of either (E)- or (Z)-orientation; such that
when “j” is a single bond, then A is H; a straight or branched substituted or unsubstituted alkyl, alkenyl, or alkynyl; CH₃, CH₂R⁸, CHR⁸R⁸, CR⁸R⁸R⁸, CH₂F, CH₂Cl, CH₂Br, CHF₂, CHCl₂, CHBr₂, CF₃, CCl₃, CBr₃, OH, OR^{8′}, SH, SR^{8′}, NH₂, NHR^{8′}, or NR^{8′}R^{8′};
when “j” is a double bond, then A is CH₂, CHR⁸, CR⁸R⁸, CHF, CHCl, CHBr, CF₂, CCl₂, CBr₂, O, S, NH, or NR^{8′};
“k” can be absent, a single, or double bond of either (E)- or (Z)-orientation; such that
when “k” is absent, then B is absent;
when “k” is a single bond, then B is H; a straight or branched substituted or unsubstituted alkyl, alkenyl, or alkynyl; CH₃, CH₂R⁸, CHR⁸R⁸, CR⁸R⁸R⁸, CH₂F, CH₂Cl, CH₂Br, CHF₂, CHCl₂, CHBr₂, CF₃, CCl₃, CBr₃, OH, OR^{8′}, SH, SR^{8′}, NH₂, NHR^{8′}, or NR^{8′};
when “k” is a double bond, then B is CH₂, CHR⁸, CR⁸R⁸, CHF, CHl, CHBr, CF₂, CCl₂, CBr₂, O, S, NH, or NR^{8′};
each R⁸ is independently —H; an optionally substituted or unsubstituted straight or branched alkyl, such as a —C₁₋₈ straight or branched chain alkyl; an optionally substituted or unsubstituted straight or branched —C₂₋₈ alkenyl; an optionally substituted or unsubstituted straight or branched —C₂₋₈ alkynyl; —C₃₋₆ cycloalkyl; 3-7 membered heterocycle; -aryl; -aralkyl; -heteroaryl, -heteroarylalkyl, -halo (F, Cl, Br, I); -haloalkyl; —CF₃; —CN; —NO₂; -acyl (including but not limited to aldehydes, ketones, esters, carboxylic acids, amides, imides, thioesters), —(C═Y¹)-alkyl, —O(C═Y¹)-alkyl, —(C═Y¹)—OH, —(C═Y¹)—O-alkyl, —S—(C═Y¹)-alkyl, —(C═Y¹)—SH, —(C═Y¹)—S-alkyl, —NH(C═Y¹)-alkyl, —NR^{8′}(C═Y¹)-alkyl, —(C═Y¹)—NH₂, —(C═Y¹)—NH(alkyl), —(C═Y¹)—N(alkyl)₂, —COOH; —COOC₁₋₈ alkyl; —CONH₂; —CONH—C₁₋₈ alkyl; —CON(C₁₋₈ alkyl)₂; alkacyl, -alkyl-(C═Y¹)-alkyl, -alkyl-O(C═Y¹)-alkyl, -alkyl-(C═Y¹)—OH, -alkyl-(C═Y¹)—O-alkyl, -alkyl-S—(C═Y¹)-alkyl, -alkyl-(C═Y¹)—SH, -alkyl-(C═Y¹)—S-alkyl, -alkyl-NH(C═Y¹)-alkyl, -alkyl-NR^{8′}(C═Y¹)-alkyl, -alkyl-(C═Y¹)—NH₂, -alkyl-(C═Y¹)—NH(alkyl), -alkyl-(C═Y¹)—N(alkyl)₂, -alkyl-COOH; -alkyl-COOC₁₋₈ alkyl; -alkyl-CONH₂; -alkyl-CONH—C₁₋₈ alkyl; -alkyl-CON(C₁₋₈ alkyl)₂; amino, —NH₂; —NH—C₁₋₈ alkyl; —N(C₁₋₈ alkyl)₂; —NHC(O)—C₁₋₈ alkyl; alkylamino; hydroxyl, alkylhydroxyl, alkoxy, thio; alkylthio; thioalkyl; and
each R^{8′} is independently an optionally substituted or unsubstituted straight or branched alkyl, such as a —C₁₋₈ straight or branched chain alkyl; an optionally substituted or unsubstituted straight or branched alkenyl, such as a —C₂₋₈ alkenyl; an optionally substituted or unsubstituted straight or branched alkynyl, such as a —C₂₋₈ alkynyl; a saturated or unsaturated carbocycle, such as a saturated or unsaturated —C₃₋₆ cycloalkyl; a heterocycle, such as a 3-7 membered heterocycle; aryl; or heteroaryl;
such that there is not a double or triple bond directly adjacent to a double or triple bond.

15. The compound of claim 14 wherein R^{1a}, R^{1b}, and R⁵ are either hydrogen, CH₃, or C₁-C₅ alkyl.

16. The compound of claim 14 wherein “a”, “b”, and “c” are all single bonds and Y is O, S, NH, NR^{8′}, CH₂, CHR′, or CR′R′, wherein each R′ is hydrogen, CH₃, CF₃, or halogen (F, Cl, Br, or I).

17. The compound of claim 14 wherein “a” is a double bond of either (E)- or (Z)-orientation, and one of “b” or “c” is a single bond and the other is absent.

18. The compound of claim 14 wherein one of “j” and “k” is a double bond of either (E)- or (Z)-orientation.

19. The compound of claim 14 wherein one, and only one, of “j” and “k” is a double bond of either (E)- or (Z)-orientation.

20. The compound of claim 14 wherein one of “j” and “k” is a double bond of either (E)- or (Z)-orientation; and if “j” is the double bond; then A is CH₂, CHR⁸, CR⁸R⁸, O, S, NH or NR^{8′}; or if “k” is the double bond; then B is CH₂, CHR⁸, CR⁸R⁸, O, S, NH or NR^{8′}.

21. The compound of claim 14 wherein only one of “j” and “k” is a double bond of either (E)- or (Z)-orientation and if “j” is the double bond; then A is O, S, NH or NR^{8′}; or if “k” is the double bond; then B is O, S, NH or NR^{8′}.

22. The compound of claim 14 wherein both of “j” and “k” are single bonds; and at least one of A and B is a straight or branched substituted or unsubstituted alkenyl or alkynyl.

23. The compound of claim 14 wherein both of “j” and “k” are single bonds; and at least one of A and B is a C₂ to C₄ alk-1-ene, alk-2-ene, alk-1-yne, or alk-2-yne.

24. A compound of Formula VIII or IX, or a pharmaceutically acceptable salt or ester thereof, 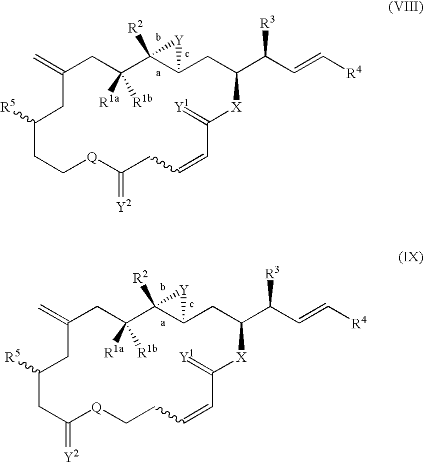
wherein:
R^{1a}, R^{1b}, and R⁵ are each independently H, C₁-C₁₀ alkyl, C₂-C₁₀ alkenyl, C₁-C₁₀ alkoxy, C₂-C₁₀ alkenoxy, C₂-C₁₀ alkynyl, C₂-C₁₀ alkynoxy, aryl, substituted aryl, heteroaryl, substituted heteroaryl, COR⁸, nitro, cyano, OH, CF₃, OCF₃, or halogen;
R² is absent (when “a” is a triple bond or when “a” is a single bond and “b” is a double bond) or is selected from the group consisting of H, C₁-C₁₀ alkyl, C₂-C₁₀ alkenyl, C₁-C₁₀ alkoxy, C₂-C₁₀ alkenoxy, C₂-C₁₀ alkynyl, C₂-C₁₀ alkynoxy, aryl, nitro, cyano, halogen, acyl, alkacyl, CHO, CO₂H, CO₂—C₁₋₁₀ alkyl, CF₃, OH, OR^{8′}, OCF₃, SH, SR^{8′}, NH₂, NHR^{8′}, NHR^{8′}R^{8′}, CON(R^{8′})₂, and CONHR^{8′};
“a” can be a single or double bond of either (E)- or (Z)-orientation, or “a” can be a triple bond when R², Y, “b” and “c” are absent;
“b” can be absent or a single bond (when R² is absent);
“c” can be absent, a single, or double bond of either (E)- or (Z)-orientation; such that
only one of “a”, “b”, and “c” can be a double bond, when “b” and “c” are absent, then Y is absent; and
when “a” is a single or double bond, one of “b” and “c” is a single bond and one is absent, then Y is H, a straight or branched substituted or unsubstituted alkyl, alkenyl, or alkynyl, CH₃, CH₂R⁸, CHR⁸R⁸, CR⁸R⁸R⁸, CH₂F, CH₂Cl, CH₂Br, CHF₂, CHCl₂, CHBr₂, CF₃, CCl₃, CBr₃, OH, OR^{8′}, SH, SR^{8′}, NH₂, NHR⁸, or NR^{8′}R^{8′};
when “a”, “b”, and “c” are single bonds or when “a” is a single bond, one of “b” and “c” is a double bond and one is absent, then Y is CH₂, CHR⁸, CR⁸R⁸, CHF, CHCl, CHBr, CF₂, CCl₂, CBr₂, O, S, NH, or NR^{8′};
R³ is independently selected from H, C₁-C₁₀ alkyl, C₂-C₁₀ alkenyl, C₁-C₁₀ alkoxy, C₂-C₁₀ alkenoxy, C₂-C₁₀ alkynyl, C₂-C₁₀ alkynoxy, aryl, substituted aryl, heteroaryl, substituted heteroaryl, nitro, cyano, CF₃, OH, O-alkyl, hydroxylalkyl, O-acyl, OCF₃, SH, S-alkyl, thioalkyl, S-acyl, amine, alkylamine, NH₂, NHR⁸, NR⁸R⁸, and halogen;
R⁴ is selected from the group consisting of C₃-C₁₀ cycloalkyl, C₃-C₁₀ cycloalkenyl, heteroaryl, substituted heteroaryl, aryl, substituted aryl, C₃-C₁₀ heterocycloalkyl, adamantly, and C₃-C₁₀ heterocycloalkenyl;
X is CH₂, CHR⁸, CR⁸R⁸, N, NR^{8′}, O, or S;
each Y¹ and Y² is independently O, S, NH, or NR⁸′;
Q is independently CH₂, CHR⁸, CR⁸R⁸, NH, NR^{8′}, O, S, C═O, or C═Y²;
each R⁸ is independently —H; an optionally substituted or unsubstituted straight or branched alkyl, such as a —C₁₋₈ straight or branched chain alkyl; an optionally substituted or unsubstituted straight or branched —C₂₋₈ alkenyl; an optionally substituted or unsubstituted straight or branched —C₂₋₈ alkynyl; —C₃₋₆ cycloalkyl; 3-7 membered heterocycle; -aryl; -aralkyl; -heteroaryl, -heteroarylalkyl, -halo (F, Cl, Br, I); -haloalkyl; —CF₃; —CN; —NO₂; -acyl (including but not limited to aldehydes, ketones, esters, carboxylic acids, amides, imides, thioesters), —(C═Y¹)-alkyl, —O(C═Y¹)-alkyl, —(C═Y¹)—OH, —(C═Y¹)—O-alkyl, —S—(C═Y¹)-alkyl, —(C═Y¹)—SH, —(C═Y¹)—S-alkyl, —NH(C═Y¹)-alkyl, —NR⁸ (C═Y¹)-alkyl, —(C═Y¹)—NH₂, —(C═Y¹)—NH(alkyl), —(C═Y¹)—N(alkyl)₂, —COOH; —COOC₁₋₈ alkyl; —CONH₂; —CONH—C₁₋₁₈ alkyl; —CON(C₁₋₅ alkyl)₂; alkacyl, -alkyl-(C═Y¹)-alkyl, -alkyl-O(C═Y¹)-alkyl, -alkyl-(C═Y¹)—OH, -alkyl-(C═Y¹)—O-alkyl, -alkyl-S—(C═Y¹)-alkyl, -alkyl-(C═Y¹)—SH, -alkyl-(C═Y¹)—S-alkyl, -alkyl-NH(C═Y¹)-alkyl, -alkyl-NR^{8′}(C═Y¹)-alkyl, -alkyl-(C═Y¹)—NH₂, -alkyl-(C═Y¹)—NH(alkyl), -alkyl-(C═Y¹)—N(alkyl)₂, -alkyl-COOH; -alkyl-COOC₁₋₈ alkyl; -alkyl-CONH₂; -alkyl-CONH—C₁₋₈ alkyl; -alkyl-CON(C₁₋₈ alkyl)₂; amino, —NH₂; —NH—C₁₋₁₈ alkyl; —N(C₁₋₈ alkyl)₂; —NHC(O)—C₁₋₈ alkyl; alkylamino; hydroxyl, alkylhydroxyl, alkoxy, thio; alkylthio; thioalkyl; and
each R^{8′} is independently an optionally substituted or unsubstituted straight or branched alkyl, such as a —C₁₋₈ straight or branched chain alkyl; an optionally substituted or unsubstituted straight or branched alkenyl, such as a —C₂₋₈ alkenyl; an optionally substituted or unsubstituted straight or branched alkynyl, such as a —C₂₋₈ alkynyl; a saturated or unsaturated carbocycle, such as a saturated or unsaturated —C₃₋₆ cycloalkyl; a heterocycle, such as a 3-7 membered heterocycle; aryl; or heteroaryl.

25. The compound of claim 24, wherein R^{1a}, R^{1b}, and R⁵ are either hydrogen, CH₃, or C₁-C₅ alkyl.

26. The compound of claim 24, wherein “a”, “b”, and “c” are all single bonds and Y is O, S, NH, NR^{8′}, CH₂, CHR′, or CR′R′, wherein each R′ is hydrogen, CH₃, CF₃, or halogen (F, Cl, Br, or I).

27. The compound of claim 24, wherein “a” is a double bond of either (E)- or (Z)-orientation, and one of “b” or “c” is a single bond and the other is absent.

28. The compound of claim 24, wherein Q is O, S, NH, or NR^{8′}.

29. The compound of claim 24, wherein X is O.

30. The compound of claim 24, wherein at least one of Y¹ and Y² is O.

31. A compound of Formula III, or a pharmaceutically acceptable salt or ester thereof, 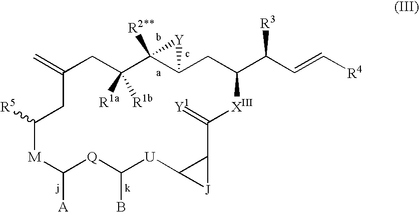
wherein:
R^{1a}, R^{1b}, and R⁵ are each independently H, C₁-C₁₀ alkyl, C₂-C₁₀ alkenyl, C₁-C₁₀ alkoxy, C₂-C₁₀ alkenoxy, C₂-C₁₀ alkynyl, C₂-C₁₀ alkynoxy, aryl, substituted aryl, heteroaryl, substituted heteroaryl, COR⁸, nitro, cyano, OH, CF₃, OCF₃, or halogen;
“a” can be a single or double bond of either (E)- or (Z)-orientation, or “a” can be a triple bond when R², Y, “b” and “c” are absent;
“b” can be absent or a single bond (when R² is absent);
“c” can be absent, a single, or double bond of either (E)- or (Z)-orientation; such that only one of “a”, “b”, and “c” can be a double bond, when “b” and “c” are absent, then Y is absent; and
when “a” is a single or double bond, one of “b” and “c” is a single bond and one is absent, then Y is H, a straight or branched substituted or unsubstituted alkyl, alkenyl, or alkynyl, CH₃, CH₂R⁸, CHR⁸R⁸, CR⁸R⁸R⁸, CH₂F, CH₂Cl, CH₂Br, CHF₂, CHCl₂, CHBr₂, CF₃, CCl₃, CBr₃, OH, OR^{8′}, SH, SR⁸, NH₂, NHR^{8′}, or NR^{8′}R^{8′};
when “a”, “b”, and “c” are single bonds or when “a” is a single bond, one of “b” and “c” is a double bond and one is absent, then Y is CH₂, CHR⁸, CR⁸R⁸, CHF, CHCl, CHBr, CF₂, CCl₂, CBr₂, O, S, NH, or NR^{8′};
R³ is independently selected from H, C₁-C₁₀ alkyl, C₂-C₁₀ alkenyl, C₁-C₁₀ alkoxy, C₂-C₁₀ alkenoxy, C₂-C₁₀ alkynyl, C₂-C₁₀ alkynoxy, aryl, substituted aryl, heteroaryl, substituted heteroaryl, nitro, cyano, CF₃, OH, O-alkyl, hydroxylalkyl, O-acyl, OCF₃, SH, S-alkyl, thioalkyl, S-acyl, amine, alkylamine, NH₂, NHR⁸, NR⁸R⁸, and halogen;
R⁴ is selected from the group consisting of C₃-C₁₀ cycloalkyl, C₃-C₁₀ cycloalkenyl, heteroaryl, substituted heteroaryl, aryl, substituted aryl, C₃-C₁₀ heterocycloalkyl, adamantly, and C₃-C₁₀ heterocycloalkenyl;
R²** is a radical selected from the group consisting of H, C₁-C₁₀ alkyl, C₂-C₁₀ alkenyl, C₁-C₁₀ alkoxy, C₂-C₁₀ alkenoxy, C₂-C₁₀ alkynyl, C₂-C₁₀ alkynoxy, aryl, nitro, cyano, halogen, CHO, CO₂H, CO₂—C₁₋₁₀ alkyl, CF₃, OCF₃, CON(R⁶)₂, or CONHR⁶;
X^{III} is CH₂, N, NR⁵, O, or S;
each Y¹ and Y² is independently O, S, NH, or NR^{8′};
J is CH₂, CHR⁸, CR⁸R⁸, CHF, CHCl, CHBr, CF₂, CCl₂, CBr₂, O, S, NH, or NR^{8′};
M and U are independently selected from the group consisting of CH₂ or CHR⁸;
Q is CH₂, CHR⁸, NR^{8′}, O or S;
“j” can be a single, or double bond of either (E)- or (Z)-orientation; such that
when “j” is a single bond, then A is H; a straight or branched substituted or unsubstituted alkyl, alkenyl, or alkynyl; CH₃, CH₂R⁸, CHR⁸R⁸, CR⁸R⁸R⁸, CH₂F, CH₂Cl, CH₂Br, CHF₂, CHCl₂, CHBr₂, CF₃, CCl₃, CBr₃, OH, OR⁸, SH, SR^{8′}, NH₂, NHR^{8′}, or NR^{8′}R^{8′};
when “j” is a double bond, then A is CH₂, CHR⁸, CR⁸R⁸, CHF, CHCl, CHBr, CF₂, CCl₂, CBr₂, O, S, NH, or NR^{8′};
“k” can be absent, a single, or double bond of either (E)- or (Z)-orientation; such that
when “k” is absent, then B is absent;
when “k” is a single bond, then B is H; a straight or branched substituted or unsubstituted alkyl, alkenyl, or alkynyl; CH₃, CH₂R⁸, CHR⁸R⁸, CR⁸R⁸R⁸, CH₂F, CH₂Cl, CH₂Br, CHF₂, CHCl₂, CHBr₂, CF₃, CCl₃, CBr₃, OH, OR^{8′}, SH, SR^{8′}, NH₂, NHR^{8′}, or NR^{8′}R^{8′};
when “k” is a double bond, then B is CH₂, CHR⁸, CR⁸R⁸, CHF, CHCl, CHBr, CF₂, CCl₂, CBr₂, O, S, NH, or NR^{8′}
each R⁸ is independently —H; an optionally substituted or unsubstituted straight or branched alkyl, such as a —C₁₋₈ straight or branched chain alkyl; an optionally substituted or unsubstituted straight or branched —C₂₋₈ alkenyl; an optionally substituted or unsubstituted straight or branched —C₂₋₈ alkynyl; —C₃₋₆ cycloalkyl; 3-7 membered heterocycle; -aryl; -aralkyl; -heteroaryl, -heteroarylalkyl, -halo (F, Cl, Br, I); -haloalkyl; —CF₃; —CN; —NO₂; -acyl (including but not limited to aldehydes, ketones, esters, carboxylic acids, amides, imides, thioesters), —(C═Y¹)-alkyl, —O(C═Y₁)-alkyl, —(C═Y¹)—OH, —(C═Y¹)—O-alkyl, —S—(C═Y¹)-alkyl, —(C═Y¹)—SH, —(C═Y¹)—S-alkyl, —NH(C═Y¹)-alkyl, —NR^{8′}(C═Y¹)-alkyl, —(C═Y¹)—NH₂, —(C═Y¹)—NH(alkyl), —(C═Y¹)—N(alkyl)₂, —COOH; —COOC₁₋₈ alkyl; —CONH₂; —CONH—C₁₋₅ alkyl; —CON(C₁₋₁₈ alkyl)₂; alkacyl, -alkyl-(C═Y¹)-alkyl, -alkyl-O(C═Y¹)-alkyl, -alkyl-(C═Y¹)—OH, -alkyl-(C═Y¹)—O-alkyl, -alkyl-S—(C═Y¹)-alkyl, -alkyl-(C═Y¹)—SH, -alkyl-(C═Y¹)—S-alkyl, -alkyl-NH(C═Y¹)-alkyl, -alkyl-NR^{8′}(C═Y¹)-alkyl, -alkyl-(C═Y¹)—NH₂, -alkyl-(C═Y¹)—NH(alkyl), -alkyl-(C═Y¹)—N(alkyl)₂, -alkyl-COOH; -alkyl-COOC₁₋₈ alkyl; -alkyl-CONH₂; -alkyl-CONH—C₁₋₈ alkyl; -alkyl-CON(C₁₋₈ alkyl)₂; amino, —NH₂; —NH—C₁₋₁₈ alkyl; —N(C₁₋₈ alkyl)₂; —NHC(O)—C₁₋₈ alkyl; alkylamino; hydroxyl, alkylhydroxyl, alkoxy, thio; alkylthio; thioalkyl; and
each R^{8′} is independently an optionally substituted or unsubstituted straight or branched alkyl, such as a —C₁₋₈ straight or branched chain alkyl; an optionally substituted or unsubstituted straight or branched alkenyl, such as a —C₂₋₈ alkenyl; an optionally substituted or unsubstituted straight or branched alkynyl, such as a —C₂₋₈ alkynyl; a saturated or unsaturated carbocycle, such as a saturated or unsaturated —C₃₋₆ cycloalkyl; a heterocycle, such as a 3-7 membered heterocycle; aryl; or heteroaryl.

32. The compound of claim 31, wherein R^{1a}, R^{1b}, and R⁵ are either hydrogen, CH₃, or C₁-C₅ alkyl.

33. The compound of claim 31, wherein “a”, “b”, and “c” are all single bonds and Y is O, S, NH, NR^{8′}, CH₂, CHR′, or CR′R′, wherein each R′ is hydrogen, CH₃, CF₃, or halogen (F, Cl, Br, or I).

34. The compound of claim 31, wherein “a” is a double bond of either (E)- or (Z)-orientation, and one of “b” or “c” is a single bond and the other is absent.

35. The compound of claim 31, wherein one of “j” and “k” is a double bond of either (E)- or (Z)-orientation.

36. The compound of claim 35, wherein if “j” is the double bond; then A is CH₂, CHR⁸, CR⁸R⁸, O, S, NH or NR^{8′}; or if “k” is the double bond; then B is CH₂, CHR⁸, CR⁸R⁸, O, S, NH or NR^{8′}.

37. The compound of claim 35, wherein if “j” is the double bond; then A is O; or if “k” is the double bond; then B is O.

38. A compound of Formula VI or VII, or a pharmaceutically acceptable salt or ester thereof, 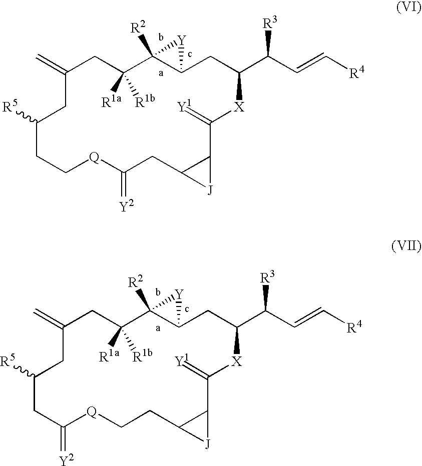
wherein:
R^{1a}, R^{1b}, and R⁵ are each independently H, C₁-C₁₀ alkyl, C₂-C₁₀ alkenyl, C₁-C₁₀ alkoxy, C₂-C₁₀ alkenoxy, C₂-C₁₀ alkynyl, C₂-C₁₀ alkynoxy, aryl, substituted aryl, heteroaryl, substituted heteroaryl, COR⁸, nitro, cyano, OH, CF₃, OCF₃, or halogen;
R² is absent (when “a” is a triple bond or when “a” is a single bond and “b” is a double bond) or is selected from the group consisting of H, C₁-C₁₀ alkyl, C₂-C₁₀ alkenyl, C₁-C₁₀ alkoxy, C₂-C₁₀ alkenoxy, C₂-C₁₀ alkynyl, C₂-C₁₀ alkynoxy, aryl, nitro, cyano, halogen, acyl, alkacyl, CHO, CO₂H, CO₂—C₁₋₁₀ alkyl, CF₃, OH, OR^{8′}, OCF₃, SH, SR^{8′}, NH₂, NHR⁸, NHR^{8′}R^{8′}, CON(R^{8′})₂, and CONHR^{8′};
“a” can be a single or double bond of either (E)- or (Z)-orientation, or “a” can be a triple bond when R², Y, “b” and “c” are absent;
“b” can be absent or a single bond (when R² is absent);
“c” can be absent, a single, or double bond of either (E)- or (Z)-orientation; such that only one of “a”, “b”, and “c” can be a double bond, when “b” and “c” are absent, then Y is absent; and
when “a” is a single or double bond, one of “b” and “c” is a single bond and one is absent, then Y is H, a straight or branched substituted or unsubstituted alkyl, alkenyl, or alkynyl, CH₃, CH₂R⁸, CHR⁸R⁸, CR⁸R⁸R⁸, CH₂F, CH₂Cl, CH₂Br, CHF₂, CHCl₂, CHBr₂, CF₃, CCl₃, CBr₃, OH, OR^{8′}, SH, SR^{8′}, NH₂, NHR^{8′}, or NR^{8′}R^{8′};
when “a”, “b”, and “c” are single bonds or when “a” is a single bond, one of “b” and “c” is a double bond and one is absent, then Y is CH₂, CHR⁸, CR⁸R⁸, CHF, CHCl, CHBr, CF₂, CCl₂, CBr₂, O, S, NH, or NR^{8′};
R³ is independently selected from H, C₁-C₁₀ alkyl, C₂-C₁₀ alkenyl, C₁-C₁₀ alkoxy, C₂-C₁₀ alkenoxy, C₂-C₁₀ alkynyl, C₂-C₁₀ alkynoxy, aryl, substituted aryl, heteroaryl, substituted heteroaryl, nitro, cyano, CF₃, OH, O-alkyl, hydroxylalkyl, O-acyl, OCF₃, SH, S-alkyl, thioalkyl, S-acyl, amine, alkylamine, NH₂, NHR⁸, NR⁸R⁸, and halogen;
R⁴ is selected from the group consisting of C₃-C₁₀ cycloalkyl, C₃-C₁₀ cycloalkenyl, heteroaryl, substituted heteroaryl, aryl, substituted aryl, C₃-C₁₀ heterocycloalkyl, adamantly, and C₃-C₁₀ heterocycloalkenyl;
X is CH₂, CHR⁸, CR⁸R⁸, N, NR^{8′}, O, or S; and
each Y¹ and Y² is independently O, S, NH, or NR^{8′};
J is CH₂, CHR⁸, CR⁸R⁸, CHF, CHCl, CHBr, CF₂, CCl₂, CBr₂, O, S, NH, or NR^{8′};
Q is independently CH₂, CHR⁸, CR⁸R⁸, NH, NR^{8′}, O, S, C═O, or C═Y²
each R⁸ is independently —H; an optionally substituted or unsubstituted straight or branched alkyl, such as a —C₁₋₈ straight or branched chain alkyl; an optionally substituted or unsubstituted straight or branched —C₂₋₈ alkenyl; an optionally substituted or unsubstituted straight or branched —C₂₋₈ alkynyl; —C₃₋₆ cycloalkyl; 3-7 membered heterocycle; -aryl; -aralkyl; -heteroaryl, -heteroarylalkyl, -halo (F, Cl, Br, I); -haloalkyl; —CF₃; —CN; —NO₂; -acyl (including but not limited to aldehydes, ketones, esters, carboxylic acids, amides, imides, thioesters), —(C═Y¹)-alkyl, —O(C═Y¹)-alkyl, —(C═Y¹)—OH, —(C═Y¹)—O-alkyl, —S—(C═Y¹)-alkyl, —(C═Y¹)—SH, —(C═Y¹)—S-alkyl, —NH(C═Y¹)-alkyl, —NR¹ (C═Y¹)-alkyl, —(C═Y¹)—NH₂, —(C═Y¹)—NH(alkyl), —(C═Y¹)—N(alkyl)₂, —COOH; —COOC₁₋₈ alkyl; —CONH₂; —CONH—C₁₋₈ alkyl; —CON(C₁₋₈ alkyl)₂; alkacyl, -alkyl-(C═Y¹)-alkyl, -alkyl-O(C═Y¹)-alkyl, -alkyl-(C═Y¹)—OH, -alkyl-(C═Y¹)—O-alkyl, -alkyl-S—(C═Y¹)-alkyl, -alkyl-(C═Y¹)—SH, -alkyl-(C═Y¹)—S-alkyl, -alkyl-NH(C═Y¹)-alkyl, -alkyl-NR^{8′}(C═Y¹)-alkyl, -alkyl-(C═Y¹)—NH₂, -alkyl-(C═Y¹)—NH(alkyl), -alkyl-(C═Y¹)—N(alkyl)₂, -alkyl-COOH; -alkyl-COOC₁₋₈ alkyl; -alkyl-CONH₂; -alkyl-CONH—C₁₋₈ alkyl; -alkyl-CON(C₁₋₈ alkyl)₂; amino, —NH₂; —NH—C₁₋₈ alkyl; —N(C₁₋₁₈ alkyl)₂; —NHC(O)—C₁₋₈ alkyl; alkylamino; hydroxyl, alkylhydroxyl, alkoxy, thio; alkylthio; thioalkyl; and
each R^{8′} is independently an optionally substituted or unsubstituted straight or branched alkyl, such as a —C₁₋₈ straight or branched chain alkyl; an optionally substituted or unsubstituted straight or branched alkenyl, such as a —C₂₋₈ alkenyl; an optionally substituted or unsubstituted straight or branched alkynyl, such as a —C₂₋₈ alkynyl; a saturated or unsaturated carbocycle, such as a saturated or unsaturated —C₃₋₆ cycloalkyl; a heterocycle, such as a 3-7 membered heterocycle; aryl; or heteroaryl.

39. The compound of claim 38, wherein R^{1a}, R^{1b}, and R⁵ are either hydrogen, CH₃, or C₁-C₅ alkyl.

40. The compound of claim 38, wherein “a”, “b”, and “c” are all single bonds and Y is O, S, NH, NR^{8′}, CH₂, CHR′, or CR′R′, wherein each R′ is hydrogen, CH₃, CF₃, or halogen (F, Cl, Br, or I).

41. The compound of claim 38, wherein “a” is a double bond of either (E)- or (Z)-orientation, and one of “b” or “c” is a single bond and the other is absent.

42. The compound of claim 38, wherein Q is O, S, NH, or NR^{8′}.

43. The compound of claim 38, wherein X is O.

44. The compound of claim 38, wherein at least one of Y¹ or Y² is O.

45. The compound of claim 1 of the structure 10: 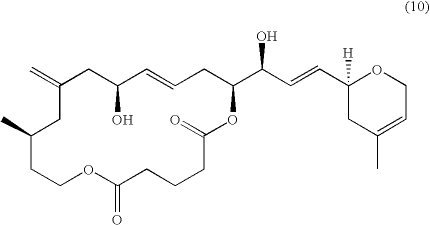
or a pharmaceutically acceptable salt or ester thereof.

46. The compound of claim 1 of the structure 12: 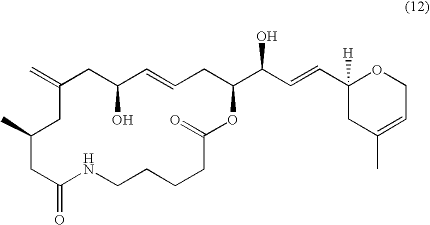
or a pharmaceutically acceptable salt or ester thereof.

47. The compound of claim 1 of the structure 14: 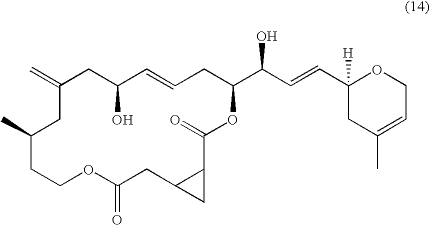
or a pharmaceutically acceptable salt or ester thereof.

48. The compound of claim 1 of the structure 16: 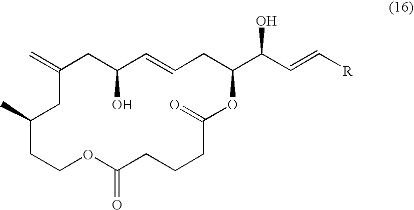
or a pharmaceutically acceptable salt or ester thereof.

49. The compound of claim 1 of the structure 18: 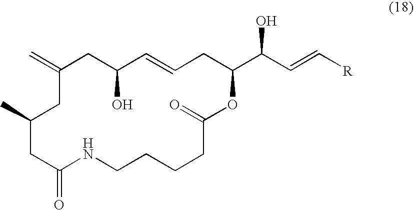
or a pharmaceutically acceptable salt or ester thereof.

50. A pharmaceutical composition comprising a compound of one of claim 1 to 13 in a pharmaceutically acceptable carrier.

51. The pharmaceutical composition of claim 50, additionally comprising at least one additional active agent.

52. The composition of claim 51 wherein the active agent is paclitaxel or an estrogen.

53. The composition of claim 52 wherein the estrogen is 2-methoxyestradiol.

54. Use of a compound of claim 1, optionally in a pharmaceutical carrier, for the preparation of a medicament for treating or preventing abnormal cell proliferation in a host.

55. The use of claim 54, further comprising administering at least one additional active agent in combination or alternation with the compound.

56. The use of claim 55, wherein the active agent is paclitaxel or an estrogen.

57. The use of claim 56 wherein the estrogen is 2-methoxyestradiol.
